(19) United States
(12) Patent Application Publication
(10) Pub. No.: US 2009/0010873 A1
(43) Pub. Date: Jan. 8, 2009

(54) Methods of Cell Therapy, Neurogenesis and Oligodendrogenesis

(75) Inventors: Michal Eisenbach-Schwartz, Rehovot (IL); Oleg Butovsky, Beer Sheva (IL); Yaniv Ziv, Ramar Gan (IL); Jonathan Kifnis, Modiin (IL); Noga Ron, Katzir (IL)

Correspondence Address: BROWDY AND NEIMARK, P.L.L.C.;624 NINTH STREET, NW, SUITE 300, WASHINGTON, DC, 20001-5303, US

(73) Assignee: YEDA RESEARCH AND DEVELOPMENT CO. LTD., Rehovot (IL)

(21) Appl. No.: 11/720,417

(22) PCT Filed: Nov. 29, 2005
(86) PCT No.: PCT/IL05/01270

§ 371 Date: Jul. 3, 2008

Related U.S. Application Data
(60) Provisional application No. 60/631,163, filed on Nov. 29, 2004.

(51) Int. Cl. A61K 38/20; A61K 38/21; A61K 35/12; A61P 25/00; A61K 31/138; A61K 38/00

(52) U.S. Cl. 424/85.2; 424/85.5; 424/93.7; 424/93.71; 424/93.1; 424/93.3; 514/653; 514/2

(57) ABSTRACT

A method is provided for inducing and enhancing neurogenesis and/or oligodendrogenesis from endogenous as well as from exogenously administered stem cells, which comprises administering to an individual in need a neuroprotective agent such as a nervous system (NS)-specific antigen, a peptide derived therefrom, T cells activated therewith, poly-YE, microglia activated by IFN-γ and/or IL-4 and combinations thereof. The method includes stem cell therapy in combination with the neuroprotective agent.

### FIELD OF THE INVENTION

The present invention relates to methods and compositions for induction and/or enhancement of endogenous neurogenesis and oligodendrogenesis and for enhancement of engraftment, survival and differentiation of stem cells injected or transplanted into an individual suffering from a disease or disorder associated with the central nervous system (CNS) or peripheral nervous system (PNS). The invention in particular relates to immune-based manipulations in combination with exogenously applied stem cells of different origins for the treatment of injuries, diseases, disorders and conditions of the CNS and PNS.

Abbreviations: Aβ, β-amyloid; ACSF, artificial cerebrospinal fluid; AD, Alzheimer's disease; BDNF, brain-derived neurotrophic factor; BMS, Basso motor score; BrdU, 5-bromo-2′-deoxyuridine; CFA, complete Freund's adjuvant; CNS, central nervous system; DCX, doublecortin; EAE, experimental autoimmune encephalomyelitis; EGF, epidermal growth factor; EPSP, excitatory postsynaptic potential; FCS, fetal calf serum; FGF, fibroblast growth factor; i.c.v., intracerebroventricular; GA, glatiramer acetate; GFAP, glial fibrillary acidic protein; GFP, green fluorescent protein; IB4, isolectin B4; IFA, incomplete Freund's adjuvant; IGF-I, insulin-like growth factor 1; IFN, interferon; IL, interleukin; LPS, lipopolysaccharide; LTP, long-term potentiation; MBP, myelin basic protein; MG, microglia; MHC-II, class II major histocompatibility complex molecules; MOG, myelin oligodendrocyte glycoprotein; MWM, Morris water maze; NeuN, neuronal nuclear antigen; NPC, neural stem/progenitor cell; PBS, phosphate-buffered saline; PDL, poly-D-lysine; SCI, spinal cord injury; SGZ, subgranular zone; TBS, theta-burst stimulation; TNF, tumor necrosis factor

### BACKGROUND OF THE INVENTION

The central nervous system (CNS) is particularly vulnerable to insults that result in cell death or damage in part because cells of the CNS have a limited capacity for repair. Since damaged brain tissue does not regenerate, recovery must come from the remaining intact brain.

Poor recovery from acute insults or chronic degenerative disorders in the CNS has been attributed to lack of neurogenesis, limited regeneration of injured nerves, and extreme vulnerability to degenerative conditions. The absence of neurogenesis was explained by the assumption that soon after birth the CNS reaches a permanently stable state, needed to maintain the equilibrium of the brain's complex tissue network. Research during the last decade showed, however, that the brain is capable of neurogenesis throughout life, albeit to a limited extent (Morshead et al., 1994). In the inflamed brain, neurogenesis is blocked (Ekdahl et al., 2003; Monje et al., 2003). This latter finding strengthened the traditional view that local immune cells in the CNS have an adverse effect on neurogenesis. Likewise, the limited regeneration and excessive vulnerability of CNS neurons under inflammatory conditions or after an acute insult were put down to the poor ability of the CNS to tolerate the immune-derived defensive activity that is often associated with local inflammation and cytotoxicity mediated, for example, by tumor necrosis factor (TNF)-α or nitric oxide (Merrill et al., 1993). More recent studies have shown, however, that although an uncontrolled local immune response indeed impairs neuronal survival and blocks repair processes, a local immune response that is properly controlled can support survival and promote recovery (Hauben and Schwartz, 2003; Schwartz et al., 2003). It was further shown that after an injury to the CNS, a local immune response that is well controlled in time, space, and intensity by peripheral adaptive immune processes (in which CD4⁺ helper T cells are directed against self-antigens residing at the site of the lesion) is a critical requirement for post-traumatic neuronal survival and repair (Moalem et al., 1999; Butovsky et al., 2001; Schwartz et al., 2003; Shaked et al., 2004). These and other results led our group to formulate the concept of ‘protective autoimmunity’ (Moalem et al., 1999).

Neurogenesis occurs throughout life in adult individuals, albeit to a limited extent. Most of the newly formed cells die within the first 2-3 weeks after proliferation and only a few survive as mature neurons. Little is known about the mechanism(s) underlying the existence of neural stem/progenitor cells (NPCs) in an adult brain and why these cells are restricted in amount and limited to certain areas. Moreover, very little is known about how neurogenesis from an endogenous NPC pool can be physiologically increased. Knowledge of the factors allowing such stem cells to exist, proliferate, and differentiate in the adult individual is a prerequisite for understanding and promoting the conditions conducive to CNS repair. This in turn can be expected to lead to the development of interventions aimed at boosting neural cell renewal from the endogenous stem-cell pool or from exogenously applied stem cells.

Experiments with rat and mouse models in our laboratory have shown that well-controlled implantation of specifically activated blood-borne macrophages (Rapalino et al., 1998) or dendritic cells (Hauben et al., 2003) promotes recovery from spinal cord injury (SCI). Other studies showed that the well-controlled activity of autoimmune T cells reactive to CNS antigens residing in the lesioned site can promote recovery from axonal insults (Hauben et al., 2000; Moalem et al., 1999). It was also shown that neuroprotection, mediated by T cells directed specifically to CNS-related autoantigens, is the body's physiological response to CNS injury (Yoles et al., 2001a, 2001b).

Under normal conditions in the adult brain, new neurons are formed in the neurogenic niches of the subventricular zone of the lateral ventricles and the subgranular zone of the hippocampal dentate gyrus (Kempermann et al., 2004). Under pathological conditions some neurogenesis can also be induced in non-neurogenic brain areas. Several studies have demonstrated, for example, that injury to the CNS in animals is followed by recruitment of endogenous NPCs, which can undergo differentiation to neurons and glia at the injured site (Nakatomi et al., 2002; Imitola et al., 2004a). However, this injury-triggered cell renewal from endogenous progenitors is limited in extent and is not sufficient for full replacement of the damaged tissue. To overcome the deficit, scientists are currently seeking ways to promote recovery by transplanting cultured adult NPCs (aNPCs) (McDonald et al., 2004). Exogenous aNPCs might contribute to recovery by acting as a source of new neurons and glia in the injured CNS (Cummings et al., 2005; Lepore and Fischer, 2005) or by secreting factors that directly or indirectly promote neuroprotection (Lu et al., 2005) and neurogenesis from endogenous stem-cell pools (Enzmann et al., 2005).

A potential approach for treatment of CNS damage includes the use of adult neural stem cells or any type of stem cells. The adult neural stem cells are progenitor cells present in the mature mammalian brain that have the ability of self-renewal and, given the appropriate stimulation, can differentiate into brain neurons, astrocytes and oligodendrocytes. Stem cells (from other tissues) have classically been defined as pluripotent and having the ability to self-renew, to proliferate, and to differentiate into multiple different phenotype lineages. Hematopoietic stem cells are defined as stem cells that can give rise to cells of at least one of the major hematopoietic lineages in addition to producing daughter cells of equivalent potential. Three major lineages of blood cells include the lymphoid lineage, e.g. B-cells and T-cells, the myeloid lineage, e.g. monocytes, granulocytes and megakaryocytes, and the erythroid lineage, e.g. red blood cells. Certain hematopoietic stem cells are capable of differentiating to other cell types, including brain cells.

Separation and cloning of neural stem cell lines from both the murine and human brain have been reported (Gage et al., 1995; Kuhn et al., 1997). Human CNS neural stem cells, like their rodent homologues, when maintained in a mitogen-containing (typically epidermal growth factor or epidermal growth factor plus basic fibroblast growth factor), serum-free culture medium, grow in suspension culture to form aggregates of cells known as neurospheres. Upon removal of the mitogens and provision of a substrate, the stem cells differentiate into neurons, astrocytes and oligodendrocytes. When such stem cells are reintroduced into the developing or mature brain, they can undergo through division, migration and growth processes, and assume neural phenotypes, including expression of neurotransmitters and growth factors normally elaborated by neurons. Thus, use of neural stem cells may be advantageous for CNS damage recovery in at least two ways: (1) by the stem cells partially repopulating dead areas and reestablishing neural connections lost by CNS damage, and (2) by secretion of important neurotransmitters and growth factors required by the brain to rewire after CNS damage. Efforts to promote recovery from brain injury in animals using neural stem cells have been reported (Park et al., 1999).

Recently, a renewable source of neural stem cells was discovered in the adult human brain (Eriksson et al., 1998). These cells may be a candidate for cell-replacement therapy for nervous system disorders. The ability to isolate these cells from the adult human brain raises the possibility of performing autologous neural stem cell transplantation. It has been reported that clinical trials with adult human neural stem cells have been initiated for treatment of Parkinson's disease patients (Fricker et al., 1999). If adult neural stem cells are to be used in clinical trials they must be amenable to expansion into clinically significant quantities. Unfortunately, these cells seem to have a limited life-span in the culture dish (Kukekov et al., 1999) and it remains to be determined whether they are stable at later passages and capable of generating useful numbers of neurons.

The brain has long been viewed as an immune-privileged site. However, autoimmune T cells (controlled with respect to the onset, duration, and intensity of their activity) were recently shown to exert a beneficial effect on neuronal survival after CNS injury (Schwartz et al., 2003), as well as in cases of mental dysfunction (Kipnis et al., 2004). Moreover, in-depth understanding of the mechanisms underlying the beneficial effect of T cells for degenerative neural tissue has pointed out that the T cells instruct the microglia, at the injured area, to acquire a phenotype supportive of neural tissue. In addition, it appeared that several immune-based intervention can boost this protective response, all of which converts to microglial activation (Shaked et al., 2004). The type of damage does not determine the choice of the approach, it is the site which determines it. Some antigens cross-react with numerous antigens and thus can overcome tissue specificity barrier.

According to the present invention, we show that the same manipulation that leads to neuronal survival leads to neurogenesis and oligodendrogenesis. It appears that the same microglia, activated by T cells or by their cytokines, not only support neuronal survival but also support oligodendrogenesis and neurogenesis. These results indicate that T-cell-based manipulation will create conditions in damaged neural tissue that favor cell renewal not only from endogenous stem cell resources but also from exogenously applied stem cells.

### SUMMARY OF THE INVENTION

Based on the hypothesis that the same immune response that causes cell loss under neurodegenerative conditions also blocks neurogenesis in the adult CNS and that an immune response that protects against cell loss also supports neurogenesis and oligodendrogenesis, we postulated that both neurogenesis and oligo-dendrogenesis can be induced by microglia that encounter well-controlled levels of cytokines associated with adaptive immunity, but are blocked by microglia that encounter endotoxin, which is associated with an uncontrolled local immune response that impairs neuronal survival and blocks repair processes.

In one aspect, the present invention relates to a method for inducing and enhancing neurogenesis and/or oligodendrogenesis from endogenous as well as from exogenously administered stem cells, which comprises administering to an individual in need a neuroprotective agent selected from the group consisting of:
(i) a nervous system (NS)-specific antigen or an analog thereof;
(ii) a peptide derived from an NS-specific antigen or from an analog thereof, or an analog or derivative of said peptide;
(iii) poly-YE and poly-YE related peptides;
(iv) T cells activated with an agent (i) to (iii);
(v) antigen-presenting cells that have been pulsed with an agent (i) to (iii);
(vi) activated mononuclear phagocytic leukocytes;
(vii) dopamine, a dopamine precursor, an agonist of the dopamine receptor type 1 family (D1-R agonist), a combination of dopamine and a dopamine precursor, or a combination of dopamine, a dopamine precursor, or a D1-R agonist with an antagonist of the dopamine receptor type 2 family (D2-R antagonist);
(viii) microglia activated by IFN-γ or IL-4, or both;
(ix) IFN-γ or IL-4, or both; and
(x) any combination of (i) to (ix).

In another aspect, the present invention provides a method of stem cell therapy comprising transplantation of stem cells in combination with a neuroprotective agent to an individual that suffers from an injury, disease, disorder or condition of the central nervous system (CNS) or peripheral nervous system (PNS). The neuroprotective agent is selected from the group consisting of the agents (i) to (x) defined above.

In a further aspect, the invention relates to the use of a neuroprotective agent selected from the group consisting of the agents (i) to (x) defined above for the preparation of a pharmaceutical composition for inducing and enhancing neurogenesis and/or oligodendrogenesis from endogenous as well as from exogenous stem cells administered to a patient.

### BRIEF DESCRIPTION OF THE FIGURES

FIGS. 1A-1F show that differentiation of NPCs into neurons can be either induced or blocked by microglia, depending on how they are activated. Green fluorescent protein (GFP)-expressing NPCs (green) were co-cultured with differently activated microglia from mice for 5 days. Quantification of β-III-tubulin⁺ cells (expressed as a percentage of GFP⁺ cells) obtained from confocal images, without (−Ins) or with insulin (+Ins), is summarized in FIGS. 1A and 1B, respectively. FIG. 1C shows results of the effect of rTNF-α on the number of β-III-tubulin⁺ cells, expressed as a percentage of GFP⁺ cells, in co-cultures of NPCs and MG_{(IFN-γ)} in the presence of insulin. Error bars represent means±SD. Data are from one of at least three independent experiments in replicate cultures. Asterisks above bars express differences relative to untreated (Control) NPCs (*P<0.05; ***P<0.001; ANOVA). FIG. 1D shows representative confocal images of GFP-expressing NPCs (green), in the absence of insulin without microglia (Control); with untreated microglia (MG₍₋₎); with LPS-activated microglia (MG_{(LPS)}); with IL-4-activated microglia (MG_{(IL-4)}); and in the presence of insulin with IFN-γ-activated microglia (MG_{(IFN-γ)}+Ins) or IFN-γ-activated microglia (MG_{(IFN-γ)}+Ins) and ãTNF. FIG. 1E shows GFP-expressing NPCs co-expressing β-III-tubulin and Nestin. FIG. 1F shows that newly formed neurons from NPCs are positively stained for glutamic acid decarboxylase (GAD) 67 (β-III-tubulin⁺/GFP⁺/GAD⁺). Note, confocal channels are presented separately.

FIGS. 2A-2D show that microglia activated with IFN-γ or IL-4 induce differentiation of NPCs into doublecortin (DCX)-expressing neurons with different morphology. GFP-expressing NPCs (green) were co-cultured with differently activated microglia as described in FIG. 1, and stained for the neuronal marker DCX. FIG. 2A depicts two representative confocal images of GFP-expressing NPCs (green) co-cultured for 5 days with MG_{(IL-4)} in the absence of insulin (left panel) or with MG_{(IFN-γ)} in the presence of insulin (MG_{(IFN-γ)+}Ins, right panel). FIG. 2B shows representative confocal images of GFP-expressing NPCs co-expressing DCX. FIG. 2C shows representative confocal images of β-III-tubulin⁺ cells co-expressing DCX. Note, confocal channels are presented separately. FIG. 2D shows quantification of DCX⁺ cells (expressed as a percentage of GFP⁺ cells) obtained from confocal images, without or with insulin. Error bars represent means±SD. Data are from one of at least three independent experiments in replicate cultures. Asterisks above bars express differences relative to untreated (control) NPCs (*P<0.05; **P<0.01; ***P<0.001; ANOVA).

FIGS. 3A-3E show that differentiation of NPCs into oligodendrocytes can be either induced or blocked by microglia, depending on how they are activated. GFP-expressing NPCs (green) were cultured alone (Control) or co-cultured with differently activated microglia as described in FIG. 1. Histograms showing quantification of NG2⁺ or RIP⁺ cells (expressed as a percentage of GFP⁺ cells) obtained from confocal images, co-cultures after 5 days (3A) in insulin-free medium (−Ins) or (3B) in insulin-containing medium (+Ins). The data shown are from one of three independent experiments in replicate cultures, with bars representing means±SD. Asterisks above bars express differences relative to untreated (Control) NPCs (**P<0.01; ***P<0.001; ANOVA). FIG. 3C shows 4 representative confocal images of GFP-expressing NPCs (green) and NG2⁺ (red) cells: without microglia (Control); co-cultured with untreated microglia (MG₍₋₎); co-cultured with IFN-γ-activated microglia in the presence of insulin (MG_{(IFNγ)+}Ins); co-cultured with IL-4-activated microglia (MG_{(IL-4)}), for 5 days. FIG. 3D shows confocal images showing co-localization of GFP, NG2 and Nestin cells. Note, confocal channels are presented separately. FIG. 3E shows that NG2⁺ cells are seen adjacent to MAC1⁺ cells.

FIGS. 4A-4G show differentiation and maturation of NPCs in the presence of MG_{(IFN-γ)} or MG_{(IL-4)} after 10 days in culture. Cultures of untreated NPCs (Control) or of NPCs co-cultured with MG_{(IFN-γ)} or MG_{(IL-4)} were analyzed after 10 days. FIG. 4A depict the numbers of NG2⁺, RIP⁺, GalC⁺, GFAP⁺ or β-III-tubulin⁺ cells expressed as percentages of GFP⁺ cells. Values are means±SD (*P<0.05; **P<0.01; P<0.001; ANOVA). FIGS. 4B-4F are representative confocal images of NPCs in the presence of MG_{(IL-4)} after 10 days in culture. FIG. 4B shows that increased branching of processes stained with NG2 was seen after 10 days (compare FIG. 4B with FIG. 3C, MG_{(IL-4)}). Contact is seen to be formed between an NG2⁺ process and an adjacent cell (high magnification of boxed area). FIG. 4C shows that staining of the same cultures for mature oligodendrocytes (GalC⁺) and neurons (β-III-tubulin⁺) shows contacts between neurons and highly branched oligodendrocytes (high magnification of boxed area). FIG. 4D shows that no overlapping is seen between labeling for neurons (DCX⁺) and for oligodendrocytes (RIP⁺). FIGS. 4E and 4F show that no overlapping is seen between GFAP and NG2 labeling or between GFAP and DCX labeling, respectively. FIG. 4G shows neurites length of MG(IFN) and MG_{(IL-4)} cells. Values are means±SD (***P<0.001; ANOVA).

FIGS. 5A-5D show the role of IGF-I and TNF-α in induction of oligodendrogenesis by IL-4- and IFN-γ-activated microglia. (5A) GFP-expressing NPCs (green) were cultured alone (control), in the presence of ãIGF-I, in co-cultures with MG_{(IL-4)} in the absence or presence of ãIGF-I (5 μg/ml), or in the presence of ãTNF-α (1 ng/ml). (5B) In an independent experiment, NPCs were cultured in the presence of rIGF-I (500 ng/ml). In FIGS. 5A and 5B no insulin was added to the media. (5C) NPCs were cultured alone (control), with ãTNF-α (1 ng/ml), or with MG_{(IFN-γ)} in the absence or presence of ãTNF-α. (5D) In an independent experiment, NPCs were cultured with MG_{(IFN-γ)} in the presence of insulin and rTNF-α (10 ng/ml). Error bars represent means±SD. Asterisks above bars express differences relative to untreated (control) NPCs (*P<0.05; **P<0.01; ***P<0.001; ANOVA).

FIGS. 6A-6C show that IFN-γ, unlike IL-4, transiently induced TNF-α and reduced IGF-I expression in microglia. (6A) Microglia treated with IL-4 (10 ng/ml), IFN-γ (20 ng/ml), or LPS (100 ng/ml) for 24 h were analyzed for TNF-α and IGF-I mRNA by semi-quantitative RT-PCR. Representative results of one of three independent experiments are shown. (6B) Time courses of TNF-α and IGF-I mRNA expression by MG_{(IL-4)} and MG_{(IFN-γ)}. PCR at each time point was performed with the same reverse-transcription mixtures for all cDNA species. Values represent relative amounts of amplified mRNA normalized against m-actin in the same sample, and are represented as fold of induction relative to control (means±SD). The linear working range of amplifications was ascertained before the experiments were carried out. Each sample was tested in three replicates, and similar results were obtained in three different microglial cultures. (6C) Statistical analysis of IGF-I expression demonstrates fluorescence intensity per cell, calculated as a percentage of increased intensity relative to MG₍₋₎ (control) (means±SD; obtained in two independent experiments, each repeated four times). Note, relative to the untreated control, MG_{(IL-4)} showed a significant increase in IGF-I. Asterisks above bar express differences relative to MG₍₋₎ (*P<0.05; **P<0.001; two-tailed Student's t-test).

FIGS. 7A-7F show that intraventricularly injected MG_{(IL-4)} induces oligodendrogenesis in adult rats. (7A) Schematic representation of the analyzed area scanned from three coronal sections (Bregma −3.8 mm in the middle) at 500-μm intervals. Confocal images of coronal slices from the amygdalohippocampal area, with the anterolateral part adjacent to the lateral ventricle of injected side stained for oligodendrocyte progenitors (NG2; red) and microglia (ED1; blue) in an MG_{(IL-4)}-injected rat (7B), an MG_{(LPS)}-injected rat (7C), and a PBS-injected rat (7D), 28 days after stereotaxic injection. Most of the BrdU⁺ (green) cells are co-labeled with NG2 (7B; boxed area). The numbers of NG2⁺ cells, co-labeled NG2⁺ and BrdU⁺ cells (7E), and ED1⁺ cells (7F) in the described area (means±SD) are calculated from the injected side of each brain (two-tailed Student's t-test; n=4 rats per group) in three sections (Bregma −3.8 mm in the middle) at 500-μm intervals. Data are expressed as identified cells per mm² in 25-μm sections. Asterisks above bars express the differences relative to control (PBS-injected) rats (**, P<0.01; ***, P<0.001).

FIGS. 8A-8C show that high-dose IFN-γ, by inducing production of TNF-α, decreases the ability of microglia to support neurogenesis, and this inhibitory effect is counteracted by IL-4. (8A) In-vitro treatment paradigm; (8B) GFP-expressing NPCs (green) were cultured for 10 days without microglia (control), or were co-cultured for 10 days with untreated microglia MG₍₋₎, or with microglia activated by IFN-γ (10 ng/ml) (MG_{(IFNγ, 10 ng/ml)}) or IFN-γ (100 ng/ml) (MG_{(IFNγ,100 ng/ml)}), or with MG_{(IFNγ,100 ng/ml)} in the presence of IL-4 (10 ng/ml) (MG_{(IFNγ,100 ng/ml)}+IL-4), or with MG_{(IFNγ,100 ng/ml)} in the presence of ãTNT-α (1 ng/ml) (MG_{(IFNγ,100 ng/ml)}+ãTNF-α), or without microglia in the presence of ãTNF-α (1 ng/ml) or IL-4 (10 ng/ml). (8C) Quantification of cells labeled with GFP and β-II-tubulin (expressed as a percentage of GFP⁺ cells) obtained from confocal images. Data are from one of three independent experiments in replicate cultures, with bars representing means±SD. Asterisks above bars express differences relative to untreated (control) NPCs (*P<0.05; **P<0.01; ***P<0.001; ANOVA). Horizontal lines with P values above them show differences between the indicated groups.

FIGS. 9A-9B show that IL-4-activated microglia can counteract the adverse effect of high-dose IFN-γ. GFP-expressing NPCs (green) were cultured alone or were co-cultured with or without microglia activated by MG_{(IFNγ,100 ng/ml)} in the presence of IL-4 (10 ng/ml) or in the presence of MG_{(IFNγ,100 ng/ml)}+MG_{(IL-4)}), as indicated. (9A) Histograms showing quantification of GFP⁺ cells and GFP⁺/β-III-tubulin⁺ cells (expressed as a percentage of total GFP⁺ cells) obtained from confocal images after 10 days in co-culture. Data are from one of three independent experiments in replicate cultures, with bars representing means±SD. Asterisks above bars express differences relative to untreated NPCs (*P<0.05; **P<0.01; ***P<0.001; ANOVA). Horizontal lines with P values above them show differences between the indicated groups. (9B) Representative confocal images of GFP-expressing NPCs (green) and β-III-tubulin (red) cells, co-cultured for 10 days with MG_{(IFNγ,100 ng/ml)} or (MG_{(IFNγ,100 ng/ml)}+MG_{(IL-4)}).

FIGS. 10A-10H show that neurogenesis in the dentate gyrus of naïve adult rats is promoted by intraventricular injection of MG_{(IL-4)}. (10A-10G) MG₍₋₎ or MG_{(IL-4)} were injected stereotaxically (1×10⁵ cells in 5 μl PBS for 5 min) into the CSF of the right lateral ventricle (Bregma −0.8, L 1.2, V 4.5) (10A). Starting 1 day later, BrdU was injected intraperiteneally (i.p.) twice daily for 2.5 days to label proliferating cells. Control rats were injected with PBS only. (10B) Schematic representation of the analyzed area scanned from nine coronal sections (Bregma −3.8 mm in the middle) at 300-μm intervals. (10C-10F) Representative confocal microscopy of the dentate gyrus, 28 days after the stereotaxic injection, shows stained mature neurons (NeuN⁺, red) and proliferating NPCs (BrdU⁺, green) in rats treated with MG_{(IL-4)} (10C, 10D) or PBS (10E, 10F). (10G) The specificity of BrdU⁺/NeuN⁺ co-expression in three dimensions is demonstrated by the orthogonal projection (25 μm) in optical sections at 1-μm intervals for cells indicated by a number in (10D). (10H) The numbers of BrdU⁺/NeuN⁺ cells per dentate gyrus are presented as means±SEM, and are calculated from both sides of each brain (two-tailed Student's t-test; i=4 rats per group) from nine coronal sections. Asterisks above bars express the significance of differences in the numbers of BrdU⁺ and BrdU⁺/NeuN⁺ cells relative to PBS-injected rats (*P<0.05; two-tailed Student's t-test).

FIGS. 11A-11F show the effect of MG_{(IFN-γ)} and MG_{(IL-4)} on proliferation and survival of newly formed neurons in the dentate gyrus of naïve Lewis rats. (11A-11D) Quantitative analysis of the results analyzed 1 day or 28 days after the last BrdU injection. The rats in this experiment were divided into 4 groups: naïve, or injected intraventricularly with PBS, or with MG_{(IFN-γ)}, or with MG_{(IL-4)}. (11A) Number of surviving BrdU⁺ cells 1 day or (11B) 28 days after the last BrdU injection, showing that MG_{(IL-4)} promote proliferation strongly while having a moderate effect on survival, whereas MG_{(IFN-γ)} strongly promote survival and do not affect proliferation. (11C) Number (mean±SEM) of BrdU⁺/DCX⁺ cells expressed as a fraction of the total number of proliferating BrdU⁺ cells, 1 day after the last BrdU injection. (11D) Number (mean±SEM) of BrdU⁺/NeuN⁺ cells expressed as a fraction of the total number of BrdU⁺ cells, 28 days after the last BrdU injection (*P<0.05; **P<0.01; two-tailed Student's t-test). (11E) Representative confocal images of dentate gyrus of a rat injected with MG_{(IL-4)} and of a rat injected with PBS, analyzed 1 and 28 day after the last BrdU injections and stained for BrdU, which marks proliferating cells (green), and DCX, a marker of early differentiation of the neuronal lineage (red) and NeuN, a marker of mature neurons (blue). (11F) MHC-II⁺ microglia expressing IGF-I, present in the hippocampus of MG_{(IL-4)}-injected rat (box).

FIGS. 12A-12F show distribution of ED1⁺ cells in brains of rats injected intraventricularly with differently activated microglia. Localization of ED1⁺ cells (green) in the 3^{rd} ventricle (3V) and lateral ventricle (LV) of rats injected with PBS (12A and 12B, respectively) or MG_{(IL-4)} (12C and 12D, respectively) rats. (12B) Boxed area represents ED1⁺ cells (red) in the lateral ventricles of a PBS-injected rat. Note, the hippocampal area in the MG_{(IL-4)}-injected rat is heavily populated by MG_{(IL-4)} (12D; arrows). (12E) Schematic view of a coronal section divided into three analyzed areas of distribution (defined by different colors) of ED1⁺ cells. (12F) Density of ED1⁺ cells in the three defined areas, calculated from three coronal sections per brain (Bregma −3.8 mm in the middle) at 500-μm intervals (n=4 per group). Data are expressed as means±SD per mm² in 25-μm sections.

FIGS. 13A-13G show that immunization with an MBP-derived encephalitogenic peptide results in increased neurogenesis in the rat hippocampus. Acute (monophasic) EAE was induced in two groups of adult Lewis rats (n=8 in each group) by immunization with MBP peptide emulsified in CFA. Seven days later, each rat received a bilateral stereotaxic intraventricularly injection (Bregma −0.8, L 1.2, V 4.5) with syngeneic MG_{(IL-4)} (1×10⁵ cells in 5 μl PBS for 5 min) or with PBS. From day 14 after MBP immunization, BrdU was injected i.p. twice daily for 2.5 days to label proliferating cells. Naïve rats (n=4) received the same regimen of BrdU injections. One week after the last BrdU injection, brains were excised and their hippocampi were analyzed for BrdU, DCX, and NeuN. (13A) Proliferating cells (BrdU⁺) and their differentiation to pre-mature neurons (BrdU⁺/DCX⁺) are more abundant in immunized rats injected with either MG_{(IL-4)} or with PBS than in naïve rats, and newly formed mature neurons (BrdU⁺/NeuN⁺) are significantly more abundant in immunized rats injected with MG_{(IL-4)} than in PBS-injected immunized rats or in naïve rats. Numbers of BrdU⁺, BrdU⁺/DCX⁺, or BrdU⁺/NeuN⁺ cells per dentate gyrus, calculated from both sides of each brain, are presented as means±SEM from nine coronal sections (two-tailed Student's t-test). Asterisks above bars express the significance of differences in the numbers of BrdU⁺, BrdU⁺/DCX⁺, and BrdU⁺/NeuN⁺ cells relative to naïve rats (*P<0.05; **P<0.01; **P<0.001; two-tailed Student's t-test). (13B) Rates of differentiation of pre-mature neurons (BrdU⁺/DCX⁺) do not differ significantly between the groups. (13C) Significantly more BrdU⁺/NeuN⁺ cells are seen in the MG_{(IL-4)}-injected rats than in the PBS-injected controls or naïve rats. (13D) In another set of experiments immunized or naïve rats were injected stereotaxically with PBS (n=5 in each group) and after 7 days were injected with BrdU, as described above. Seven days after the rats had received the first BrdU injection their dentate gyri were analyzed for BrdU⁺, BrdU⁺/DCX⁺, and BrdU⁺/NeuN⁺ cells (*P<0.05; **P<0.01; two-tailed Student's t-test). (13E) Significantly more BrdU⁺/IB4⁺ or BrdU⁺/MHC-II⁺/IB4⁺ were found in sections from the MBP-vaccinated rats injected with MG_{(IL-4)} than from MBP-vaccinated rats injected with PBS. (13F) Number of BrdU⁺/MHC-II⁺/IB4⁺ cells expressed as a fraction of the total number of BrdU⁺ cells. Numbers of BrdU⁺/MHC-II⁺/IB4 cells in the rat dentate gyrus are calculated from both sides of each brain (n=5 rats per group) and presented as means±SEM. Asterisks above bars denote the significance of differences relative to controls (*p<0.01; ***p<0.001; two-tailed Student's t-test). Results were quantified using a stereological counting procedure. (13G) Representative confocal microscopy of the dentate gyrus, 14 days after stereotaxic injection, shows stained early differentiated neurons (DCX⁺; blue), mature neurons (NeuN⁺, red), and proliferating NPCs (BrdU⁺, green) in MG_{(IL-4)}- or PBS-injected and naïve rats (upper panels). The lower panels are representative confocal images showing proliferating microglia (BrdU⁺/IB4⁺) co-stained with MHC-II of the dentate gyrus of MPB-immunized rats. MHC-II⁺ cells co-labeled with the microglial marker IB4 and BrdU adjacent to the subgranular zone (SGZ) in MG_{(IL-4)}-injected rat are shown in boxed areas.

FIGS. 14A-14C show that high-dose IFN-γ, by inducing TNF-α, inhibits the ability of microglia to support oligodendrogenesis, whereas microglia activated by IL-4 can overcome the inhibition. (14A) GFP-expressing NPCs (green) were cultured for 10 days without microglia (Control), or co-cultured for 10 days with MG_{(IL-4)} (10 ng/ml), or with MG_{(IFN-γ,10 ng/ml)} or MG_{(IFN-γ,100 ng/ml)}, or with microglia activated by both IFN-γ (100 ng/ml) and IL-4 (10 ng/ml) (MG_{(IFNγ,100 ng/ml+IL-4)}), or with MG_{(IFN-γ,100 ng/ml)} in the presence of ãTNF-α (1 ng/ml) (MG_{(IFN-γ,100 ng/ml+ãTNF-α}). (14B) Co-localization of GFP, NG2, and RIP. Arrows show the same cells expressing the indicated markers. Arrowhead shows a highly branched GFP⁺/RIP⁺ oligodendrocyte. Separate confocal channels are presented to the right. (14C) Quantification of NG2⁺ or RIP⁺ cells (expressed as a percentage of GFP⁺ cells) obtained from confocal images. The data shown are from one of two independent experiments in replicate cultures, with bars representing means±SD. Asterisks above bars express differences relative to untreated (control) NPCs (*P<0.05; **P<0.01; ***P<0.001; ANOVA).

FIGS. 15A-15B show that IL-4 partially reverses down-regulation of IGF-I expression and up-regulation of TNF-α expression in microglia activated by IFN-γ (100 ng/ml). (15A) Quantitative real-time PCR (Q-PCR) of microglia identical to those described in FIG. 14, 24 h after treatment, revealed a significant increase in IGF-I in microglia activated by IL-4 (10 ng/ml). (15B) TNF-α transcripts are decreased in microglia activated by IFN-γ (100 ng/ml) concomitantly with IL-4 (10 ng/ml), compared to IFN-γ-activated microglia. Values represent the relative amounts of amplified mRNA normalized against β-actin and are expressed as fold of induction relative to untreated (control) microglia. Groups were compared by ANOVA and significant differences between groups are expressed as P values. Results of one of two independent experiments are shown in triplicate, with bars representing means±SD. Asterisks above bars express differences relative to control (*P<0.05; **P<0.01; ***P<0.001; ANOVA).

FIGS. 16A-16D show that intraventricularly injected MG_{(IL-4)} significantly improves the clinical symptoms of acute EAE and induces oligodendrogenesis in rats. Acute (monophasic) EAE was induced by active vaccination with MBP peptide emulsified in CFA. On day 7, rats were stereotaxically injected bilaterally into brain lateral ventricles with PBS or with syngeneic MG_{(IL-4)} (n=8 in each group). From day 14, BrdU was injected i.p. twice daily for 2.5 days to label proliferating cells. Naïve rats received the same course of BrdU injection (n=4). Spinal cords were excised 5 days after the last BrdU injection (21 days after immunization), by which time disease in the rats of both groups was resolved. (16A) EAE scores in rats injected stereotaxically, 1 day before onset of EAE symptoms (on day 7), either with MG_{(IL-4)} or with PBS (n=8 in each group). Asterisks designate differences (Student's t-test) between the groups at the indicated time points (16B) EAE scores of individual rats 16 days after vaccination. (16C) NG2⁺ or RIP⁺ cells co-labeled with BrdU⁺ cells were quantitatively analyzed in both gray matter (GM) and white matter (WM) at 300-μm intervals along longitudinal 30-μm sagittal sections of spinal cord (T8-T9) (n=8 per group). Data are expressed as means±SEM per mm³. Asterisks above bars express differences relative to nonvaccinated (naïve) rats (*P<0.05; **P<0.01; ***P<0.001; ANOVA). (16D) Representative confocal images of an area taken from the white matter of an MG_{(IL-4)}-treated rat. Newly formed oligodendrocytes are identifiable by co-localization of BrdU and NG2 or RIP (arrows). Separate confocal channels are presented to the right.

FIGS. 17A-17D show that rats injected intraventricularly with MG_{(IL-4)} exhibit increased microglial proliferation and MHC-II expression. The spinal cords analyzed in FIG. 16 were also examined for microgliogenesis. (17A) Quantitative analysis of IB4⁺ and IB4⁺/MHC-II⁺ cells co-labeled with BrdU⁺ (means±SEM) from the gray matter (GM) and white matter (WM) per mm³ (two-tailed Student's t-test; n=8 per group). Asterisks above bars express the significance of differences relative to PBS-injected rats (*P<0.05; ***P<0.001; two-tailed Student's t-test). (17B) Representative confocal microscopy of longitudinal sagittal sections of spinal cords (T8-T9), stained with BrdU and co-stained with IB4 for microglia and MHC-II, 21 days after injection with PBS or with MG_{(IL-4)}. The most abundant populations of BrdU⁺ cells were immunoreactive to IB4 in both control and MG_{(IL-4)}-injected rats. Significantly more MHC-II⁺ cells are seen, especially in the gray matter, in slices from MG_{(IL-4)}-injected rats than from PBS-injected rats (control). Note that the images are from areas that include both gray matter and white matter; a dashed line shows the border between the two areas. (17C) Most of the newly formed microglia (IB4⁺/BrdU⁺) in MG_{(IL-4)}-treated rats co-express MHC-II and (17D) IGF-I (arrows).

FIGS. 18A-18E show that in mice with chronic EAE, intraventricularly injected MG_{(IL-4)} significantly improves clinical features and induces oligodendrogenesis. Chronic EAE was induced in C57BL/6J mice. On day 10, the mice received bilateral stereotaxic injections of PBS or syngeneic MG_{(IL-4)} into the cerebrospinal fluid of the brain lateral ventricles. From day 20, BrdU was injected i.p. twice daily for 2.5 days to label proliferating cells. Spinal cords were excised 12 days after the last BrdU injection. (18A) Chronic EAE scores in mice injected stereotaxically, 2 days after onset of EAE symptoms (on day 10), with either MG_{(IL-4)} or PBS (n=15 in each group). (18B) Scores of individual mice 17 days after vaccination. (18C) NG2⁺ or RIP⁺ cells co-labeled with BrdU⁺ cells were quantitatively analyzed at 300-μm intervals in both gray matter (GM) and white matter (WM) of the spinal cord (i=4 per group). Data are expressed as means±SEM per mm³. Asterisks above bars express differences relative to PBS-injected mice (*P<0.05; two-tailed Student's t-test). (18D) Representative confocal images of longitudinal sections of spinal cords stained with BrdU and co-stained for NG2 and RIP, 34 days after immunization of MG_{(IL-4)}-treated and PBS-treated mice. Separate confocal channels are presented to the right. (18E) Newly formed oligodendrocytes are identifiable by co-localization of BrdU, NG2, and RIP in the white matter of MG_{(IL-4)}-treated mice.

FIGS. 19A-19H show that a myelin-specific autoimmune response operates synergistically with transplanted aNPC transplantation in promoting functional recovery from spinal cord injury (SCI). Recovery of motor function after SCI (200 kdynes for 1 s) in male C57B1/6J mice. (n=6-9 in each group). (19A) Mice were immunized with MOG peptide or PBS emulsified in CFA containing 1% Mycobacterium tuberculosis (MOG-CFA and PBS-CFA, respectively). One week after SCI, aNPCs were transplanted into their lateral ventricles (MOG-CFA/aNPC or PBS-CFA/aNPC). The lateral ventricles of mice in similarly injured and immunized control groups were treated with PBS (MOG-CFA/PBS or PBS-CFA/PBS). Values of the Basso motor score (BMS) rating scale are presented. (19B) BMS scores of individual mice described in (19A) on day 28 of the experiment. (19C) Recovery of motor function after SCI (200 kdynes for 1 s) in male C57B1/6J mice (n=6-9 in each group) immunized with MOG peptide 45D emulsified in CFA containing 2.5% Mycobacterium tuberculosis. One week after SCI, aNPCs were transplanted into the lateral ventricles. Similarly injured and immunized control groups, instead of being transplanted with aNPCs, were injected with PBS. BMS values are presented. (19D) Recorded BMS scores of individual mice described in (19C) on day 28 of the experiment. (19E) Injury and aNPC transplantation were as in (19A), but immunization was carried out 7 days prior to SCI and the mice were immunized with MOG-IFA or injected with PBS (control). (19F) Injury and aNPC transplantation were as in (19A), but immunization was carried out 7 days prior to SCI and the mice were immunized with OVA/CFA. (19G, 19H) Injury and aNPC transplantation were as in (19A), but immunization was carried out 7 days prior to SCI and the mice were immunized with MOG peptide and CFA containing 2.5% Mycobacterium tuberculosis. Results in all groups are means±SEM. Asterisks show differences at the indicated time points, analyzed by two-tailed Student's t-test. (*,p<0.05; **,p<0.01; ***,p<0.001).

FIGS. 20A-20F show that GFP-labeled aNPCs are found in the parenchyma of the spinal cord after dual treatment with MOG immunization and aNPC transplantation. Immunohistochemical staining of longitudinal paraffin sections of spinal cords excised 7 or 60 days after transplantation of aNPCs to the lateral ventricles. Sections were stained with anti-GFP antibody and counterstained with Hoechst to detect nuclei. They were then scanned by fluorescence microscopy for the presence of GFP⁺ cells. Representative micrographs of GFP-immunolabeled cells in areas adjacent to the lesion site 7 days after transplantation (20A-20F) and 60 days after transplantation of aNPCs (20A-20F) are shown.

FIGS. 21A-21F show histological analysis of spinal cords from injured C57B1/6J mice after dual treatment with MOG/CFA immunization and aNPC transplantation. Spinal cords were excised 1 week after cell transplantation. SCI C57B1/6J mice (n=3-4 in each group) were subjected to SCI (200 kdynes for 1 s) and immunized on the day of SCI with MOG peptide emulsified in CFA containing 1% Mycobacterium tuberculosis. One week after SCI, the lateral ventricles of MOG-CFA-immunized mice transplanted aNPCs or injected PBS. (21A, 21B), GFAP staining of longitudinal sections of injured spinal cords shows significantly smaller areas of scar tissue after treatment with MOG-CFA/aNPC than in any of the other groups. (21A) Representative micrographs of spinal cords from mice treated with MOG-CFA/aNPC, MOG-CFA/PBS, PBS-CFA/aNPC, or PBS-CFA/PBS are shown. (21B) Quantification of the area delineated by GFAP staining (*p<0.05, **p<0.01, ***p<0.001, two-tailed Student's t-test; n=4 analyzed slices from each mouse). (21C, 21D) Longitudinal paraffin sections of spinal cords excised and stained for IB4 7 days after cell transplantation and 14 days after contusive SCI show significantly less staining in MOG/CFA/aNPC-treated mice than in any of the other groups. (21D) Quantification of area occupied by IB4 staining (*p<0.05, **p<0.01 ***p<0.001, two-tailed Student's t-test). (21E, 21F) Staining with anti-CD3 antibody to identify infiltrating T cells at the site of injury. (21E) Representative micrographs of spinal cords from mice treated with MOG-CFA/aNPC, MOG-CFA/PBS, PBS-CFA/aNPC, or PBS-CFA/PBS. Manual counting of cells in four slices from each mouse, obtained from four areas surrounding the site of injury, disclosed significantly more CD3⁺ cells in the group treated with MOG-CFA/aNPC than in any of the other groups (*p<0.05, **p<0.01 ***p<0.001, two-tailed Student's t-test).

FIGS. 22A-22D show histological analysis of BDNF and noggin expression in spinal cords from injured C57B1/6J mice after dual treatment with MOG/CFA immunization and aNPC transplantation. C57B1/6J mice were subjected to SCI (n=3-4 in each group) and immunized with pMOG 35-55 emulsified in CFA (1% Mycobacterium tuberculosis) on the day of SCI. One week after SCI, the lateral ventricles of MOG-CFA-immunized mice transplanted with aNPCs or injected PBS. Longitudinal sections of spinal cords excised 7 days after cell transplantation and 14 days after SCI (n=3-4 in each group) were stained for BDNF. (22A) Quantification of area stained for BDNF (*p<0.05, **p<0.01, ***p<0.001, two-tailed Student's t-test). Staining for BDNF is significantly more intense in mice treated with MOG-CFA/aNPC than in any of the other groups. (22B) Double staining for BDNF and IB4 shows that IB4+ microglia/macrophages are a major source of BDNF. (22C) Significantly more intense staining for noggin was found in mice treated with MOG/CFA/aNPCs than in any of the other groups. (22D) Quantification of area stained with noggin (*p<0.05, **p<0.01***, p<0.001, two-tailed Student's t-test). Double staining for noggin and IB4 shows that IB4+ microglia are a major source of noggin.

FIGS. 23A-23E show, increase in BrdU/DCX double staining in the vicinity of the site of injury after dual treatment with immunization and aNPC transplantation. SCI and aNPCs transplantation as in FIG. 19. One week after aNPCs transplantation mice were injected twice daily for 3 days with BrdU. Longitudinal sections of spinal cords excised 14 days after cell transplantation and 28 days after contusive SCI (n=3-4 in each group) were stained for BrdU and DCX. Significantly more BrdU⁺/DCX⁺ cells were found in mice treated with MOG-CFA/aNPC than in any of the other groups.

FIGS. 24A-24F show that T cells induce neuronal differentiation from aNPCs in vitro. (24A) Quantification of β-III-tubulin⁺ cells (expressed as a percentage of DAPI cells) after 5 days in culture alone (control), or in co-culture with pre-activated CD4+ T cells, or with resting CD4+ T cells (**p<0.01; ***p<0.001; ANOVA). (24B) Representative images showing β-III-tubulin expression in aNPCs after 5 days in culture alone (control), or in co-culture with pre-activated CD4+ T cells. (24C) Quantification of β-III-tubulin⁺ cells (expressed as a percentage of DAPI cells) after 5 days in culture of aNPCs in the presence of medium conditioned by activated T cells. (24D) Representative images showing branched, elongating β-III-tubulin labeled fibers. (24E) Quantification of β-III-tubulin⁺ cells in aNPCs after 5 days in culture with different concentrations of IFN-γ or IL-4 (***p<0.001; ANOVA). (24F) Quantitative RT-PCR showing a fivefold reduction in Hes-5 expression in aNPCs cultured with medium conditioned for 24 h by activated CD4+ T-cells.

FIGS. 25A-25C show that aNPCs inhibit T-cell proliferation and modulate cytokine production. (25A) Proliferation was assayed 96 h after activation by incorporation of [³H]-thymidine into CD4+ T cells co-cultured with aNPCs. Recorded values are from one of three representative experiments and are expressed as means±SD of four replicates. (25B) Proliferation of CD4+ T cells cultured alone, or in the presence of aNPCs (co-culture), or with aNPCs in the upper chamber of a transwell. (25C) Cytokine concentrations (pg/ml) in the growth medium 72 h after activation of CD4+ T cells alone or in co-culture with aNPCs.

FIGS. 26A-26N show the effects of an enriched environment on newly generated neurons and microglia in the dentate gyrus of adult rats. After 6 weeks of housing, rats received daily i.p injection of BrdU for 5 days and were euthanized 7 days after the last injection. (26A-26F) Significantly more newly generated BrdU⁺/NeuN⁺ (26A) and BrdU⁺/IB4⁺ cells (26B) were observed in the dentate gyrus of rats housed in an enriched environment than of control rats housed under standard conditions (**P<0.01; ***P<0.001; t-test, n=6 per group). (26C-26F) Confocal micrographs of newly generated microglia (IB4⁺ cells, blue) and neurons (NeuN⁺ cells, red) among the newly formed cells (BrdU⁺ cells, green) enriched or standard environmental conditions. Significantly more MHC-II⁺ microglia were observed in rats housed in the enriched environment (26H, boxed area in 26G) than in control rats (26J, boxed area in 26I); scale bar indicates 500 μm. (26K) All MHC-II⁺ cells were co-labeled with the microglial marker IB4; scale bar indicates 100 μm. (26L) Co-localization of IGF-I⁺ cells with MHC-II⁺ cells in the dentate gyrus; scale bar indicates 10 μm. (26M) Quantification of MHC-II⁺/BrdU⁺ cells in the dentate gyrus (*P<0.01, **P<0.01, t-test; n=6 per group). (26N) T cells were detected in the dentate gyrus of rats housed in the enriched environment; scale bar indicates 5 μm. Data are means±s.e.m. SGZ, subgranular zone; GCL, granular cell layer.

FIGS. 27A-27H show that neurogenesis is impaired in mice with severe combined immune deficiency (SCID). C57B1/6J mice (SCID and wild type) were injected i.p. with BrdU and euthanized 2, 7, or 28 days after the first injection. (27A) Quantification of BrdU-labeled cells in the subgranular zone (SGZ) of the dentate gyrus 2 days after the first injection (*P<0.05, t-test; n=3 per group; one of three independent experiments). (27B) Quantification of BrdU/DCX double-labeled cells in the dentate gyrus 7 d after the first BrdU injection (**P<0.01, t-test; n=5 and n=6 for wild type and SCID mice, respectively). (27C) Quantification of BrdU/NeuN double-labeled cells in the dentate gyrus 28 days after the first BrdU injection (**P<0.01, t-test; n=5 per group). (27D) Representative confocal micrographs of the dentate gyri of wild-type and SCID mice that were double-stained for BrdU (red) and DCX (green) 7 days after the first BrdU injection. (27E) Representative confocal micrographs of the dentate gyrus of wild-type and SCID mice that were double-stained for BrdU (green) and NeuN (red) 28 days after the first BrdU injection. (27F) Quantification of BrdU/DCX double-labeled cells in the dentate gyrus 7 days after the first BrdU injection in SCID mice pre-treated by i.v. injection of syngeneic wild-type splenocytes or with splenocytes depleted of T cells (*P<0.05, t-test; n=5 per group). (27G) Quantification of BrdU-labeled cells in the unilateral SVZ 2 days after the first injection (***P<0.001, t-test; n=5 per group). (27H) Representative confocal micrographs of the SVZ of wild-type and SCID mice stained for BrdU. Data are means±s.e.m; scale bar indicates 100 μm.

FIGS. 28A-28B show that an enriched environment stimulates neurogenesis in wild-type but not in SCID mice. Two groups each (from an enriched environment and from standard housing) of wild-type and SCID mice were analyzed. After 6 weeks of housing, mice were given one i.p injection of BrdU daily for 5 days and were euthanized 7 days after the last injection. Quantification of BrdU-labeled cells (gray bars) and BrdU/DCX double-labeled cells (black bars) in the dentate gyrus of (28A) wild type (***P<0.001, t-test; n=6 per group) and (28B) SCID mice (P=0.55, t-test; n=5 per group).

FIGS. 29A-29I show impaired neurogenesis in T cell-deficient mice. (29A) Quantification of BrdU-labeled cells (gray bars) and BrdU/DCX double-labeled cells (black bars) in the dentate gyrus of nude and wild-type (WT) mice 7 days after the first BrdU injection (***P<0.001, t-test; n=4 per group). (29B) Numbers of newly formed neurons (BrdU⁺/DCX⁺), expressed as percentages of the total number of newly formed cells (BrdU⁺) in the dentate gyrus (***P<0.001, t-test; n=4 per group). (29C) Staining for DCX in the dentate gyrus reveals significantly shorter dendrites and significantly less dendritic arborization in nude mice (right panel) than in the wild-type (left panel). Scale bar indicates 20 μm. (29D) Nude mice received splenocytes 10 days before BrdU injections, and were killed 7 days later. The numbers of newly formed neurons (BrdU⁺/DCX⁺) were counted and expressed as percentages of the total numbers of newly formed (BrdU⁺) cells (**P<0.05, ANOVA; n=5 per group). (29E) Total numbers of proliferating (PCNA⁺) cells in the dentate gyrus of wild-type mice, splenocyte-replenished nude mice, and nonreplenished nude (control) mice (*P<0.05, ANOVA; n=5 per group). Staining for T cells (CD3+) in brain sections of wild-type and replenished nude mice revealed their presence mainly on the walls of the lateral and third ventricles and in the adjacent parenchyma. (29F) CD3⁺ cells in the brain of a replenished nude mouse. (29G) Higher magnification of the marked area in (29F); scale bar indicates 50 μm. (29H) CD3⁺ cells lining the wall of the 3^{rd} ventricle in the brain of a wild-type mouse and (29I) in the parenchyma adjacent to a blood vessel in the brain of a replenished nude mouse; scale bar indicates 25 μm. Data are means±s.e.m.; LV, lateral ventricle; CA3, CA3 of the hippocampus.

FIGS. 30A-30C show that neurogenesis is maintained by T cells specific to myelin basic protein (MBP) but not to ovalbumin (OVA). Mice received four injections of BrdU (50 mg/kg, i.p.) at 12-h intervals and were euthanized 7 days after the first injection. (30A) BrdU/DCX double-labeled cells in the dentate gyrus of B10.PL wild-type and T_{MBP} transgenic mice 7 days after the first BrdU injection (*P<0.05, t-test; n=5 and n=8 respectively; one of two independent experiments). (30B) BrdU/DCX double-labeled cells in the dentate gyri of Balb/c wild-type mice and T_{OVA} transgenic mice 7 days after the first BrdU injection (**P<0.01, t-test; n=5 per group). (30C) BrdU/DCX double-labeled cells in the dentate gyri of T_{MBP} transgenic mice that were treated daily with minocycline or with PBS. Seven days after the first injection mice received four injections of BrdU (50 mg/kg, i.p.) at 12-h intervals and were euthanized 7 days after the first injection (*P<0.05, t-test; n 3 per group). Data are means±s.e.m.

FIGS. 31A-31H show that spatial learning and memory are maintained by T cells specific to MBP but not to ovalbumin. (31A-31C) T_{MBP} transgenic mice and their wild-type controls were monitored while_attempting a spatial learning/memory task in the Morris water maze (MWM). T_{MBP} transgenic mice performed significantly better than the controls in (31A) the acquisition phase of the task (3-way ANOVA, repeated measures—groups: F(1,9)=6.9, P<0.03; trials: F(3,27)=21.3, P<0.0001; days: F(3,27)=6.8, P<0.002; interactions: not significant); (31B) in the extinction phase (1-way ANOVA, F(1,9)=35.1, P<0.0003); and (31C) in the reversal phase (3-way ANOVA, repeated measures—groups: F(1,9)=5.3, P<0.05; trials: F(3,27)=4.5, P<0.01; days: F(1,9)=18.1, P<0.003; interactions: not significant). (31D-31F) T_{OVA} transgenic mice were monitored while attempting a spatial learning/memory task in the MWM. T_{OVA} transgenic mice performed significantly worse than their wild-type counterparts in (31D) the acquisition phase of the task (3-way ANOVA, repeated measures—groups: F(1,18)=24.4, P<0.0003; trials: F(3,54)=5.8, P<0.002; days: not significant; groups×trials: F(3,54)=6.3, P<0.0002; groups×days: F(3,54)=3.2, P<0.035; trials×days: not significant; groups×trials×days: not significant), (31E) in the extinction phase (1-way ANOVA—F(1,19)=5.3, P<0.035); and (31F) in the reversal phase of the task (3-way ANOVA, repeated measures—groups: F(1,18)=63.1, P<0.0001; trials: F(3,54)=3.3, P<0.03; days: not significant; interactions: not significant). (31G) Average swimming path lengths during training sessions of T_{MBP} transgenic mice and their wild-type controls (in the acquisition phase, 2-way ANOVA, repeated measures—groups: F(1,9)=73.5, P<0.0001; trials: F(3,27)=14.9, P<0.0001; interactions: not significant; and in the reversal phase, 2-way ANOVA, repeated measures—groups: F(1,9)=45.07, P<0.0001; trials: not significant; interactions, not significant). (31H) Average swimming path lengths during training sessions of T_{OVA} transgenic mice and their wild-type controls (in the acquisition phase, 2-way ANOVA, repeated measures—groups: F(1,18)=105.6, P<0.0001; trials: F(3,54)=19.2, P<0.0001; groups×trials: F(3,54)=11.4, P<0.0001; in the reversal phase, 2-way ANOVA, repeated measures—groups: F(1,18)=60.49, P<0.0001; trials: F(3,54)=29.59, P<0.0001; interactions, not significant). In swimming speed, no significant intergroup differences were found. *P<0.05, analysis by Scheffe's test, indicating significant post-hoc differences between individual groups. Data are means±s.e.m.

FIGS. 32A-32E show that expression of brain-derived neurotrophic factor (BDNF) in the hippocampus correlates with hippocampal neurogenesis and spatial learning/memory. Coronal sections of the hippocampus were stained for BDNF. The figure shows quantification of BDNF immunoreactivity (in arbitrary units) in the dentate gyrus of (32A) C57B1/6J wild-type and SCID mice (n=3 per group), (32B) Balb/c wild-type and T_{OVA} transgenic mice (n=4 and n=3 respectively), and (32C) B10.PL wild-type and T_{MBP} transgenic mice (n=3 per group) (*P<0.05, t-test). (32D) BDNF staining in the dentate gyrus of B10.PL wild-type and T_{MBP} transgenic mice. (32E) Confocal images showing co-localization of BDNF and NeuN in the dentate gyrus. Data are means±s.e.m; scale bar indicates 100 μm.

FIGS. 33A-33C show lack of long-term potentiation (LTP) in immune-deficient mice. LTP was induced in C57B1/6J (33A) mice by theta-burst stimulation (TBS) of the Schaffer collaterals, and was recorded at the CA1 region of the hippocampus. Wild-type mice showed a normal LTP pattern, whereas immune-deficient (SCID) mice showed potentiation of the excitatory postsynaptic potential (EPSP) slope that lasted no more than 20 min. These differences between groups were significant across the different time points relative to TBS [F(1,4)=129.378, P<0.0001]. (33B) Representative traces of C57B1/6J and their SCID counterparts are shown (i) prior to TBS, (ii) immediately after TBS, (iii) 10 min after TBS, and (iv) 20 min after TBS. (33C) As in the wild type, T_{MBP}/RAG^{-/-} mice showed a robust and significant LTP [F(1,4)=280.311, P<0.0001] that lasted for at least 30 min, whereas RAC^{-/-} mice showed only transient potentiation.

FIGS. 34A-34C show that autoimmune T cells directed to MBP beneficially affect brain plasticity. (34A) RAG^{-/-} (immune-deficient) mice and transgenic mice expressing T cells reactive to MBP on a background of RAG^{-/-} (T_{MBP}/RAG^{-/-} mice) were monitored while attempting a spatial learning/memory task in the MWM. During the acquisition (days 1-4, four trials per day) and reversal (days 6-7, four trials per day) phases of the task, T_{MBP}/RAG^{-/-} mice took significantly less time than RAG^{-/-} mice to acquire the spatial learning needed to reach the platform. On day 5 mice were exposed to a probe trial (not shown). Differences were analyzed by three-way ANOVA, repeated measures: groups: df(1,23), F=22.4, P<0.0001; trials: df(3,69), F=24.1, P<0.0001; days: df(3,69), F=20.3, P<0.0001, for the acquisition phase; and groups: df(1,23), F=74.1, P<0.0001; trials: df(3,69), F=11.9, P<0.0001; days: df(3,69), F=24.3, P<0.0001, for the reversal phase. Data are from one of two experiments performed. (34B) Balb/c/OLA wild-type and T_{OVA} transgenic mice on the same background were monitored while attempting a spatial learning/memory MWM task. T_{OVA} transgenic mice performed significantly worse than their wild-type counterparts (3-way ANOVA, repeated measures: groups: df(1,18), F=24.4, P<0.0003; trials: df(3,54), F=5.8, P<0.002; days: non-statistical, for the acquisition phase; and groups: df(1,18), F=63.1, P<0.0001; trials: df(3,54), F=3.3, P<0.03; days: non-statistical, for the reversal phase). (34C) In the MWM task, T_{MBP} transgenic mice performed slightly better than wild-type mice on the same background (3-way ANOVA, repeated measures: groups: df(1,25), F=3.85, P<0.06; trials: df(3,75), F=56.8, P<0.0001; days: df(1,75), F=10.96, P<0.0001, for the acquisition phase; and groups: df(1,25), F=5.2, P<0.03; trials: df(3,75), F=8.995, P<0.0001; days: df(1,25), F=28.2, P<0.0001, for the reversal phase).

FIGS. 35A-35E show that neurogenesis is impaired in immune-deficient mice and is restored by autoimmune T cells specific to MBP. Mice were injected with BrdU (50 mg/kg i.p.) at 12-hourly intervals and were killed 2 days (four BrdU injections), 7 days, or 28 days (five BrdU injections) after the first injection. Two coronal sections of the hippocampus (Bregma −1.9 mm) from each mouse were analyzed by immunofluorescence staining and confocal microscopy. (35A) Mean number (±SEM) of BrdU-labeled cells per section from the subgranular zone of the dentate gyrus. Differences were analyzed by two-tailed Student's t-test (*P<0.05, **P<0.01; n=3). (35B) Mean number (±SEM) of BrdU-labeled cells (gray bars) and BrdU/DCX double-labeled cells (black bars) in the dentate gyrus on day 7 after BrdU injection (**P<0.01; n=5). (35C) Mean number (±SEM) of BrdU-labeled cells (gray bars) and BrdU/NeuN double-labeled cells (black bars) in the dentate gyrus on day 28 after BrdU injection (**P<0.01; n=5). (35D, 35E) Representative confocal micrographs of the dentate gyrus of SCID (i), wild-type (ii), T_{MBP} transgenic (iii), and T_{MBP} transgenic/Rag1^{-/-} mice (iv), stained for BrdU (red) and DCX (green) 7 days (d) and for BrdU (green) and NeuN (red) 28 days after BrdU injection (e). Bar indicates 100 μm.

FIGS. 36A-36C show that glatiramer acetate (GA) vaccination counteracts cognitive loss in the APP/PS1 Tg mouse model of Alzheimer's disease (AD). Hippocampal-dependent cognitive activity was tested in the MWM. (36A to 36C) GA-vaccinated Tg mice (diamond; n=6) showed significantly better learning/memory ability than untreated Tg mice (square, n=7) during the acquisition and reversal phases but not the extinction phase of the test. Untreated Tg mice showed consistent and long-lasting impairments in spatial memory tasks. In contrast, performance of the MWM test by the GA-vaccinated Tg mice was rather similar, on average, to that of their age-matched naïve non-Tg littermates (triangle; n=6) (3-way ANOVA, repeated measures: groups, df (2,16), F=22.3, P<0.0002; trials, df (3,48), F=67.9, P<0.0001; days, df (3,48), F=3.1, P<0.035, for the acquisition phase; and groups, df (2,16), F=14.9, P<0.0003; trials, df (3,48), F=21.7, P<0.0001; days, df (1,16), F=16.9, P<0.0008, for the reversal phase).

FIGS. 37A-37J show that T cell-based vaccination with GA leads to a reduction in β-amyloid (Aβ) and counteracts hippocampal neuronal loss in the brains of Tg mice: key role of microglia. (37A) Representative confocal microscopic images of brain hippocampal slices from non-Tg, untreated-Tg, and GA-vaccinated Tg littermates stained for NeuN (mature neurons) and human Aβ. The non-Tg mouse shows no staining for human Aβ. The untreated Tg mouse shows an abundance of extracellular Aβ plaques, whereas in the GA-vaccinated Tg mouse Aβ-immunoreactivity is low. Weak NeuN⁺ staining is seen in the hippocampal CA1 and DG regions of the untreated Tg mouse relative to its non-Tg littermate, whereas NeuN⁺ staining in the GA-vaccinated Tg mouse is almost normal. (37B) Staining for activated microglia using anti-CD11b antibodies. Images at low and high magnification show a high incidence of cells double-immunostained for Aβ and CD11b in the CA1 and DG regions of the hippocampus of an untreated Tg mouse, but only a minor presence of CD11b⁺ microglia in the GA-vaccinated Tg mouse. Arrows indicate areas of high magnification, shown below. (37C) CD11b⁺ microglia, associated with an Aβ-plaque, expressing high levels of TNF-α⁺ in an untreated Tg mouse. (37D) Staining for MHC-II (a marker of antigen presentation) in a cryosection taken from a GA-vaccinated Tg mouse in an area that stained positively for Aβ shows a high incidence of MHC-II⁺ microglia and almost no TNF-α⁺ microglia. (37E) MHC-II⁺ microglia in the GA-vaccinated mouse co-express IGF-I. (37F) CD3⁺ T cells are seen in close proximity to MHC-II⁺ microglia associated with Aβ-immunoreactivity. Boxed area shows high magnification of an immunological synapse between a T cell (CD3⁺) and a microglial cell expressing MHC-II. (37G) Histogram showing the total number of Aβ-plaques (in a 30-μm hippocampal slice). (37H) Histogram showing the total stained Aβ-immunoreactive cells. Note, the significant differences between GA-vaccinated and untreated Tg mice, and verifies the decreased presence of Aβ-plaques in the vaccinated Tg mice. (37I) Histogram showing a remarkable reduction in cells stained for CD11b, indicative of activated microglia and inflammation, in the GA-vaccinated Tg mice relative to untreated Tg mice. Note the increase in CD11⁺ microglia with age in the non-Tg littermates. (37J) Histogram showing increased survival rate of NeuN⁺ neurons in the DGs of GA-vaccinated Tg mice relative to untreated Tg mice. Error bars indicate means±SEM. Asterisks above bars express the significance of differences in the immunostaining (*P<0.05; **P<0.01; ***P<0.001; two-tailed Student's t-test). Note, all the mice in this study were included in the analysis (6-8 sections per mouse).

FIGS. 38A-38E show enhanced cell renewal induced by T cell-based vaccination with glatiramer acetate (GA) in the hippocampus of adult Tg mice. Three weeks after the first GA vaccination, mice in each experimental group were injected i.p. with BrdU twice daily for 2.5 days. Three weeks after the last injection their brains were excised and the hippocampi analyzed for BrdU, DCX, and NeuN. (38A to 38C) Histograms showing quantification of the proliferating cells (BrdU⁺) (38A), newly formed mature neurons (BrdU⁺/NeuN⁺) (38B), and all pre-mature (DCX⁺-stained) neurons (38C). Numbers of BrdU⁺, BrdU⁺/NeuN⁺, and DCX⁺ cells per DG, calculated from six equally spaced coronal sections (30 μm) from both sides of the brains of all the mice tested in this study. Error bars represent means±SEM. Asterisks above bars denote the significance of differences relative to non-Tg littermates (**P<0.01; ***P<0.001; two-tailed Student's t-test). Horizontal lines with P values above them show differences between the indicated groups (ANOVA). (38D) Representative confocal microscopic images of the DG showing immunostaining for BrdU/DCX/NeuN in a GA-vaccinated Tg mouse and in a non-Tg littermate relative to that in an untreated Tg mouse. (38E) Branched DCX⁺ cells are found near MHC-II⁺ microglia located in the subgranular zone of the hippocampal DG of a GA-vaccinated Tg mouse.

FIGS. 39A-39D show that IL-4 can counteract the adverse effect of aggregated Aβ on microglial toxicity and promotion of neurogenesis. (39A) In-vitro treatment paradigm. (39B) Representative confocal images of NPCs expressing GFP and β-III-tubulin, co-cultured for 10 days without microglia (control), or with untreated microglia, or with microglia that were pre-activated with Aβ₍₁₋₄₀₎ (5 μM) (MG_{(Aβ1-40)}) for 48 h and subsequently activated with IFN-γ (10 ng/ml) (MG_{(Aβ1-40/IFNγ,10 ng/ml)}), or with IL-4 (10 ng/ml) (MG_{(Aβ1-40/IL-4)}), or with both IFN-γ (10 ng/ml) and IL-4 (10 ng/ml) (MG_{(Aβ1-40/IFNγ+IL-4)}). Note, aggregated Aβ induces microglia to adopt an ameboid-like morphology, but after IL-4 was added these microglia exhibited a ramified-like structure. (39C) Separate confocal images of NPCs co-expressing GFP and P-II-tubulin adjacent to CD11b⁺ microglia. (39D) Quantification of cells double-labeled with GFP and β-III-tubulin (expressed as a percentage of GFP⁺ cells) obtained from confocal images. Results are of three independent experiments in replicate cultures; bars represent means±SEM. Asterisks above bars denote the significance of differences relative to untreated (control) NPCs (*P<0.05; ***P<0.001; two-tailed Student's t-test). Horizontal lines with P values above them show differences between the indicated groups (ANOVA).

FIGS. 40A-40E show increased hippocampal neurogenesis in PN277-treated rats. Starting on day 7 after MCAO, PN277-treated and PBS-treated rats were injected twice daily for 3 days with BrdU. On day 28 the rats were killed and their brains were analyzed for hippocampal neurogenesis. (40A) Quantification of BrdU⁺/NeuN⁺ double-labeled cells in the dentate gyrus 28 days after middle cerebral artery occlusion (MCAO). (40B) Numbers of BrdU⁺/NeuN⁺ cells expressed as a percentage of the total number of BrdU⁺ cells in the rat dentate gyrus after MCAO. Data are means±SEM. Representative confocal micrographs of (40C) the dentate gyrus of PN277-treated and (40D) PBS-treated rats double-stained for BrdU (green) and NeuN (red) (40E) Higher magnification images of two 3-week-old neurons in the granular cell layer of the dentate gyrus of a PN277-treated rat showing BrdU (green) and NeuN (red) staining separately or as a merged image.

FIGS. 41A-41J show stroke-induced cortical neurogenesis. (41A) Sections containing cortical structures affected by the stroke were stained for BrdU (red), Nestin (blue) and MAP-2 (green) (bar=200 μm). Nestin⁺ cells delineate the cortical lesion and separate it from less affected areas in which most cortical neurons were MAP-2⁺. (41B) Schematic depicting of the anatomic location of the cortical lesion (pink) and the area presented in (41A). (41C) Orthogonal confocal image of a new immature neuron found in proximity to the lesion site, labeled for BrdU (red), β-III-tubulin (green) and express low levels of MAP2 (blue) (bar=20 μm). Cells with a typical neuron morphology that were labeled with BrdU and expressed high levels of MAP-2 were defined as mature newly formed neurons. (41D) Some of these BrdU⁺/MAP-2+ neurons had short processes and appeared mainly adjacent to the lesion area. (41E) Higher magnification of the area marked in (41D). (41F) BrdU⁺/MAP-2⁺ cells with long processes and a pyramidal morphology appeared in areas which are more remote from the lesion (˜150-400 μm away) (bar=20 μm). Such newly formed neurons were often seen as an integral part of the local neuronal network. (41G). Representative sequential confocal images of a BrdU⁺/MAP-2⁺ cell along the ‘z’ plane demonstrate these morphological features (bar=20 μm). (41H) A minority (<1%) of the BrdU⁺/MAP-2⁺ cells appeared to contain two BrdU labeled nucleus. Representative sequential confocal images of such cell along the ‘z’ plane are presented.

FIGS. 42A-42B show that treatment with PN277 enhances cortical neurogenesis. For quantitative analysis sections were stained for BrdU, nestin and MAP-2. 3-D confocal scanning was used to verify co-localization of each BrdU⁺/MAP-2⁺ and BrdU⁺/Nestin⁺ cell. (42A) Quantification of BrdU⁺/MAP-2⁺ cells in the vicinity of the cortical lesion (*P<0.05, t-test). (42B) Quantification of BrdU⁺/Nestin⁺ cells in the vicinity of the cortical lesion. Data are means±SEM.

FIGS. 43A-43C show microglia phenotype in the striatum. (43A) Ratio of surface occupied by MHC-II⁺ cells to the surface occupied by IB4+ cells. Data are means±SEM (43B) Staining for MHC-II and IB4⁺ cells in the ipsilateral striatum of PN277 treated and PBS treated rats 28 days after MCAO. (43C) staining for BrdU and Nestin in the ipsilateral striatum of PN277 treated and PBS treated rats 28 days after MCAO.

FIG. 44 shows increased survival in mouse model of ALS treated with Cop-1 vaccination combined with adult neural stem cells. ALS mice (59 days old) were treated as follows: group 1 (Cop-1+NPC) immunized with 100 mg/200 ml Copaxon 1 twice a week for 2 weeks and received thereafter one immunization per week until euthanization. With the third immunization the mice received 100,000 NPCGFP i.c.v (CSF) into the right cerebral ventricle; group 2 (Cop-1) immunized with 100 mg/200 ml Copaxon 1 twice a week for 2 weeks and received thereafter one immunization per week until euthanization; group 3 (control) immunized with 200 ml PBS twice a week for 2 weeks and received thereafter one immunization per week until euthanization. The results obtained show that in the control group, 4 out of 5 mice died by days 117, 118 and 121. Whereas, in the Cop-1+NPC group, the first animal out of 4 animals died on day 127.

### DETAILED DESCRIPTION OF THE INVENTION

Research during the last decade has disclosed that the brain is potentially capable of cell renewal throughout life, albeit to a limited extent (Morshead et al., 1994). However, the mechanisms that might restrict or favor the renewal of adult neural cells are not known. Recent studies from the laboratory of the present inventors have shown that after an injury to the CNS a local immune response that is properly controlled in time, space, and intensity by the peripheral adaptive immunity is a pivotal requirement for posttraumatic neuronal survival (Moalem et al., 1999; Butovslky et al., 2001; Schwartz et al., 2003; Shaked et al., 2004). We therefore envisaged the possibility that the lack of neurogenesis and the restricted recovery might be attributable to a common factor, which might in turn be related to the local immune response.

The present invention is based on the assumption that well-regulated adaptive immunity is needed for cell renewal in the brain. We postulated that neurogenesis and oligodendrogenesis are induced and supported by microglia that encounter cytokines associated with adaptive immunity, but are not supported by naïve microglia and are blocked by microglia that encounter endotoxin.

In fact, it is shown herein that certain specifically activated microglia can induce and support neural cell renewal. Thus, both neurogenesis and oligodendrogenesis were induced and supported in NPCs co-cultured with microglia activated by the cytokines IL-4 and IFN-γ, both associated with adaptive immunity. In contrast, microglia exposed to LPS blocked both neurogenesis and oligodendrogenesis, in line with previous reports that MG_{(LPS)} block cell renewal (Monje et al., 2003).

Defense mechanisms in the form of activated microglia are often seen in acute and chronic neurodegenerative conditions, and the CNS is poorly equipped to tolerate them (Dijkstra et al., 1992). As a result, activated microglia have generally been viewed as a uniformly hostile cell population that causes inflammation, interferes with cell survival (Popovich et al., 2002), and blocks cell renewal (Monje et al., 2002, 2003).

Recent studies have shown, however, that the type of activation determines microglial activity, and that just as their effects can be inimical to cell survival in some circumstances, they can be protective in others. Thus, for example, microglia that encountered adaptive immunity (CD4⁺ T cells) were shown to acquire a protective phenotype (Butovsky et al., 2001). Among the cytokines that are produced by such T cells and can endow microglia with a neuroprotective phenotype are IFN-γ and IL-4, characteristic of Th1 and Th2 cells, respectively. Thus, microglia exposed to activated Th1 cells or to IFN-γ show increased uptake of glutamate, a key player in neurodegenerative disorders (Shaked et al., unpublished observation), while their exposure to IL-4 results in down-regulation of TNF-α, a common player in the destructive microglial phenotype, and up-regulation of insulin-like growth factor (IGF-1) (shown herein in the examples), which promotes differentiation of oligodendrocytes from multipotent adult neural progenitor cells (Hsieh et al., 2004). In addition, IGF-1 prevents the acute destructive effect of glutamate-mediated toxicity on oligodendrocytes in vitro (Ness et al., 2002) and inhibits apoptosis of mature oligodendrocytes during primary demyelination (Mason et al., 2000). These and other findings strongly suggest that the outcome of the local immune response (in terms of its effect on the microglia) in the damaged CNS will be either beneficial or harmful, depending on how the microglia interpret the threat.

In general, tissue repair is a process that is well synchronized in time and space, and in which immune activity is needed to clear the site of the lesion and create the conditions for migration, proliferation, and differentiation of progenitor cells for renewal. In light of the well-known fact that constitutive cell renewal is limited in the CNS, as well as the reported observations that treatment with MG_{(LPS)} causes neuronal loss (Boje et al., 1992) and interferes with the homing and differentiation of NPCs (Monje et al., 2003), and that adaptively activated microglia can support neuronal survival, it is not surprising to discover that immune conditions favoring neuronal survival will also support cell renewal. MG_{(LPS)} produce excessive amounts of NO (causing oxidative stress) and TNF-α, as well as other cytotoxic elements, leading to a spiral of worsening neurotoxicity (Boje et al., 1992). NO was found to act as an important negative regulator of cell proliferation and neurogenesis in the adult mammalian brain (Packer et al., 2003), and TNF-α has an inhibitory effect on oligodendrogenesis (Cammer et al., 1999).

The results of the present invention show that MG_{(LPS)} are indeed detrimental to NPC survival and differentiation, but that when microglia are activated by cells or cytokines possessing adaptive immune function, not only are they not cytotoxic but they even exert a positive effect on NPC proliferation, inducing and supporting their differentiation into neurons or oligodendrocytes. In vivo, injection of MG_{(IL-4)} into rat brain lateral ventricles resulted in no neuronal loss, minimal migration of microglia to the CNS parenchyma, and the appearance of new neurons and oligodendrocytes (indicated by the double-staining of BrdU⁺ cells with markers of neurons or oligodendrocytes). Staining for microglia revealed significant invasion of the healthy CNS by MG_{(LPS)}, with consequent massive tissue loss, unlike in the case of MG_{(IL-4)} or MG₍₋₎. Interestingly, in the non-injected hippocampus, resident microglia were found adjacent to the subventricular zone. It is tempting to speculate that these might be the cells responsible for controlling neurogenesis, restraining it when in their resting state (as found in the present work using MG₍₋₎), but inducing and supporting it when suitably activated.

Our findings are supported by the observation that in mice with experimental autoimmune encephalomyelitis (EAE), NPCs migrate to sites of CNS damage (Pluchino et al., 2003). They are also in line with the common experience that cell renewal is favored by injury, since they imply that in the absence of injury the conditions that might favor renewal do not exist.

Renewal of cells and their replenishment by new growth is the common procedure for tissue repair in most tissues of the body. It was thought that in the brain those processes do not occur, and therefore that any loss of neurons, being irreplaceable, results in functional deficits that range from minor to devastating. Since an insult to the CNS, whether acute or chronic, is often followed by the postinjury spread of neuronal damage, much research has been devoted to finding ways to minimize this secondary degeneration by rescuing as many neurons as possible.

The results of the present invention leads us to an intriguing conclusion. First, under pathological conditions (when cell renewal is critical), not only do the microglia not favor cell renewal, but they interfere with it. Secondly, this paradoxical situation can be remedied by well-controlled adaptive immunity, which shapes the microglia in such a way that their activity is not cytotoxic but is both protective and conducive to renewal. This indicates that in those cases in which protective autoimmunity leads to improved recovery, both neurogenesis and gliogenesis are likely to occur. These data can also explain the lack of cell renewal in autoimmune diseases; in such cases, it is likely that the quantity of circulating autoimmune T cells exceeds the threshold above which TNF-α production, due to an excess of IFN-γ, does not allow the microglia to acquire a protective phenotype. They can also explain why steroids are not helpful, as their anti-inflammatory activity masks not only the destructive but also the beneficial adaptive immunity. The therapy of choice for both autoimmune diseases and neurodegenerative conditions would therefore appear to be immunomodulation in which, after the acute phase of disease, the surviving tissue can be maintained by relatively small quantities of T cells.

The findings of the present invention indicate that the limitation of spontaneous, endogenous neurogenesis and oligodendrogenesis in the adult brain is, at least in part, an outcome of the local immune activity, and that harnessing of adaptive immunity rather than immunosuppression is the path to choose in designing ways to promote cell renewal in the CNS.

Cell renewal in the adult mammalian CNS is limited. Recent studies suggest that it is arrested by inflammation. That view is challenged by the findings of the present invention that microglia, depending on environmental stimulation, can either induce and support or block such renewal. In vitro, neurogenesis and oligodendrogenesis from neural progenitor cells were shown herein to be promoted by mouse microglia that encountered T-cell-associated cytokines (IFN-γ, IL-4), but were blocked by microglia that encountered endotoxin. Anti-IGF-1 antibodies neutralized the IL-4 effect, while anti-TNF-α antibodies augmented the effect of IFN-γ. Injection of IL-4-activated microglia into cerebral ventricles of adult rats induced significant hippocampal neurogenesis and cortical oligodendrogenesis, whereas endotoxin-activated microglia caused neuronal loss and blocked neurogenesis and oligodendrogenesis. These results strengthen our assumption that controlled adaptive immunity, unlike uncontrolled (e.g. endotoxin-induced) inflammation, activates microglia to induce and support neuronal and oligodendrocyte survival and renewal. Thus, to promote cell renewal in the CNS, well-controlled immunity is needed and should not be suppressed.

Studies from the inventors' laboratory over the last few years have shown that the controlled activity of T cells directed to auto-antigens in the CNS is needed for postinjury survival and repair (Moalem et al., 1999; Yoles et al., 2001; Kipnis et al., 2002; Schwartz and Kipnis, 2001). These results led us to suspect that a fundamental role of autoimmune T cells, known to be present in healthy individuals, is to help maintain the integrity of the CNS, and that their remedial effect in a neurodegenerative environment is a manifestation of the same restorative role under extreme conditions. Moreover, accumulating evidence attesting to the participation of such autoimmune T cells in postinjury neuronal survival led us to postulate that if they have a similar role in the healthy CNS, it might well have to do with neurogenesis in adult life, possibly by maintaining the conditions needed for such cell renewal.

Severe immune deficiency in mice was shown here to impair three aspects of hippocampal plasticity at adulthood: long-term potentiation (LTP), spatial learning/memory, and adult neurogenesis. Using immune-defficient mice and transgenic mice that express only T cells specific to the abundant brain self-antigen MBP, we show herein that hippocampus-dependent activities (LTP and spatial learning and memory), and adult neurogenesis could be controlled by a single population of T cells recognizing MBP, namely, MBP-specific autoimmune T cells.

The results of the present invention show that T cells directed to a specific CNS self-antigen play a key role in maintaining the functional activity of the hippocampus. Accordingly, we postulated that under non-pathological conditions a primary role of such autoimmune T cells is to maintain the plasticity of the adult brain, and thus the observed beneficial effect of antigen-specific anti-self T cells under degenerative conditions is an extension of their role in the healthy brain.

The present findings that autoimmune T cells (T_{MBP}) in the adult hippocampus affect not only neurogenesis but also other aspects of plasticity (LTP and spatial learning/memory) might suggest that a common mechanism underlies them all. Alternatively, autoimmune T cells might be of direct benefit to neurogenesis only, while their observed effect on LTP and spatial learning and memory might be an indirect result of this neurogenesis. This possibility appears to be in line with accumulating information that some aspects of LTP and hippocampal activities, such as performance of the MWM task, are related to neurogenesis in the dentate gyrus (Zhao et al., 2003).

The T-cell dependence of brain plasticity observed here in SCID mice as well as transgenic mice expressing monospecific T cells, was attributed to autoimmune T cells. The function of such T cells in the wild type is implied by the superior performance of these mice relative to SCID mice and by the established presence of autoimmune T cells in healthy animals. The findings of the present invention thus provide new clues to the nature of the process or processes underlying neuronal cell renewal in adulthood, and might explain the age-related loss of certain cognitive activities in terms of an aging immune system. They might also point to novel ways to maintain brain plasticity, promote neurogenesis, and prevent functional decay in the aging brain by systemic manipulation of the peripheral immune system via a mechanism that allows autoimmunity to be safely promoted without inducing an autoimmune disease.

According to the present invention, we examined how the nature of microglial activation affects neurogenesis in the adult rat hippocampus under physiological and pathological conditions associated with brain inflammation. Transient inflammatory conditions associated with transient accumulation of myelin-specific Th1 cells promoted neurogenesis. Injection of microglia (MG) activated by IFN-γ (MG_{(IFNγ)}) or by IL-4 (MG_{(IL-4)}) into the lateral ventricles of the brains of healthy rats promoted neurogenesis. In rats that developed monophasic (transient) EAE, the induced neurogenesis was further promoted by MG_{(IL-4)}. Our results in vitro showed that MG_{(IFN-γ)} supported neurogenesis from adult rat NPCs as long as the IFN-γ concentration was low. The impediment to neurogenesis imposed by high-dose IFN-γ could be counteracted by IL-4. Neurogenesis induced by IL-4 was weaker, however, than that induced by low-dose IFN-γ or by high-dose IFN-γ administered in combination with IL-4.

We also examined whether IL-4, via modulation of microglia both in vitro and in vivo, can overcome the destructive effects of high-dose IFN-γ, known to be associated with EAE. In vitro, a high dose of IFN-γ, but not a low dose, impaired the ability of microglia to support oligodendrogenesis from adult neural stem cells/progenitor cells (NPCs). IL-4 counteracted the interference with oligodendrogenesis caused by high IFN-γ concentrations. When IL-4-activated microglia were stereotaxically injected through the cerebral ventricles into the cerebrospinal fluid (CSF) of rats with acute EAE or of mice with a remitting-relapsing autoimmune disease, the animals demonstrated significantly more oligodendrogenesis and significantly less neurological deficit than did their vehicle-injected diseased controls.

The injection of IL-4-activated microglia into the CSF of rats suffering from acute or chronic EAE caused an increase in the numbers of newly formed microglia. Most of the new microglia expressed MHC-II and IGF-I. Since IGF-I is known to play an important role in the differentiation and survival of oligodendrocytes, and to be beneficial in the treatment of EAE, it seems reasonable to assume that the IGF-I produced by IL-4-activated microglia is responsible, at least in part, for the shift to a Th2 phenotype and thus for the increased number of MHC-II⁺ microglia as well as of the newly formed BrdU⁺/MHC-II⁺ microglia expressing IGF-I. The increased oligodendrogenesis was found to correlate with a higher incidence of newly formed MHC-II⁺ microglia, suggesting that these microglia exert their effects on oligodendrogenesis by acting as antigen-presenting cells for CD4⁺ T-helper cells.

The results of the present invention indicate a novel role for microglia in ameliorating EAE and promoting differentiation of oligodendrocytes from adult NPCs (Hsieh et al., 2004), and suggest a link between the known beneficial effect of IL-4 in ameliorating EAE, the role of IGF-I derived from IL-4-activated microglia, and the requirement of viable microglia for remyelination. Our findings thus support a key role for microglia in promoting cell renewal from endogenous progenitors under pathological conditions. Based on the present findings, as well as our previously reported results, we suggest that the cross-talk between T cells and microglia lays the foundation for protection and repair in the adult CNS. We further believe that immunosuppressive treatment, if administered alone, while ameliorating clinical signs at an early stage, could eventually have devastating results. We therefore suggest that rather than suppression, immunomodulation aimed at appropriate and well-controlled activation of microglia might be the approach to adopt in designing ways to promote cell renewal under neurodegenerative conditions.

We have identified herein cellular elements in the CNS that can respond to local environmental changes and needs, and consequently can support the formation of new cells from adult aNPCs. We demonstrated that once the microglia become suitably activated by circulating T cell-derived cytokines, they can induce neuronal and oligodendroglial differentiation from aNPCs. In view of that observation, and our previous demonstration in rodents that a T cell-based vaccination promotes recovery from contusive spinal cord injury (SCI), we postulated that translation of those findings into a therapeutic approach might benefit the repair process by creating a niche-like neurogenic/glidgenic environment at the injured site. Thus, we expected to find that supplementing the vaccination by transplantation of homologous aNPCs would further promote functional recovery after SCI. In the present invention we in fact demonstrated, using a mouse model, synergistic interaction between T cell-based immune activation and transplanted aNPCs in promoting functional motor recovery after contusive injury of the spinal cord.

Previous studies by the present inventors have shown that local and systemic immune activities have multiple tasks; they are spontaneously evoked after a traumatic injury to the CNS, and are crucial for post-traumatic survival and repair (Yoles et al., 2001a). Immunization with an antigen (such as MOG) that resides in the lesion site can enhance this immune-dependent beneficial effect. The relevant autoimmune T cells evoked by the immunization exert their protective effect in part by interacting with resident APCs such as microglia/macrophages. In line with these studies, our present results showed that the T cells needed for synergistic action with aNPCs were specific to a peptide derived from a CNS-myelin protein; no synergistic effect was obtained following a T cell-mediated response to an irrelevant antigen (ovalbumin). It should be noted that immunization with encephalitogenic peptides was utilized here for proof of concept; we do not advocate such immunization for therapeutic purposes as it carries the risk in some individuals or strains (e.g., those susceptible to autoimmune diseases) of inducing an overwhelming inflammatory response that is detrimental for recovery. For purposes of immunization, weak agonists of encephalitogenic antigens or synthetic antigens that weakly cross-react with self-antigens can replace the encephalitogenic peptides. We found that immunization with MOG peptide emulsified in IFA rather than in CFA failed to demonstrate synergism with aNPCs, suggesting that the T-cell response should be biased towards Th1, as we have previously proposed (Kipnis et al., 2002a).

We also demonstrate herein, using a rat model of environmental enrichment (in which neurogenesis in adults is physiologically augmented, see Kempermann et al., 1997), a spatial association between T cells, microglial activity, and hippocampal neurogenesis. We further show that neurogenesis is impaired in immune-deficient mice under both regular and environmental enriched conditions, and can be restored by T cells directed to brain autoantigens but not by T cells directed to irrelevant (nonself) antigens. Similar dependence on T cells was found with respect to spatial learning/memory, an additional aspect of hippocampal plasticity.

The findings of the present invention indicate that T cells affect adult neurogenesis, both in the dentate gyrus and in the SVZ, primarily via their effect on progenitor cell proliferation. The finding that relative to the wild type the T_{MBP} transgenic mice exhibited increased neurogenesis, taken together with the improved spatial-learning abilities in these mice, whereas both neurogenesis and spatial learning were impaired in T_{OVA} mice, suggests that the T cells, in order to function properly in brain plasticity, should become activated by their cognate brain antigens. Thus, the mere presence of T cells is not enough to maintain neurogenesis and learning abilities; these T cells need, in addition, to be directed to CNS antigens such as, but not limited to, MBP, MOG and others.

According to our view, the T cells interact locally with microglia and possibly also with other cellular components (endothelial cells, astrocytes) of the special microenvironment known as the ‘stem-cell niche’. The finding that microglia expressing MHC-II proteins can be seen adjacent to a site of increased neurogenesis (induced by an enriched environment) further supports the notion that. T cells can locally interact with microglia through these proteins. Some of the MIC-II+ microglia were found to express IGF-I, a growth factor known to be associated with cell renewal in the CNS. Co-expression of MHC-II and IGF-I can be observed in the examples herein in microglia that encounter T cell-derived cytokines.

The reduction in neurogenesis observed in T_{MBP}-transgenic mice after treatment with minocycline (which suppresses microglial activation) supports our suggestion that T cells can benefit neurogenesis by activating microglia (or other CNS-resident antigen-presenting cells). This does not argue against reported observations that severe inflammation, whether induced experimentally (by LPS) or as a result of neurodegenerative conditions (e.g., Alzheimer's disease or severe chronic multiple sclerosis), impairs neurogenesis (Ekdahl et al., 2003). On the other hand, our results might explain why pathological conditions such as acute CNS insult and experimental autoimmune diseases might be accompanied by an increase in neurogenesis as well as by induction of neurogenesis in non-neurogenic areas (Pluchino et al., 2003).

It is feasible that ‘resting’ microglia, recently described as highly dynamic surveillants of the brain, function in the dentate gyrus as stand-by cells for purposes not only of housekeeping and repair, but also of support for neurogenesis.

Our observation that environmental enrichment fails to enhance neurogenesis in mice suffering from immune deficiency strongly suggests that the availability of T cells that recognize CNS antigens is a critical requirement for an activity-induced increase in neurogenesis. Presumably these T cells are recruited when neurogenesis is needed. The active participation of CNS-specific T cells in brain-cell renewal does not exclude the possibility that T cells are needed for neurogenesis during development as well.

The results herein might partially explain the age-related loss of certain cognitive activities, by viewing the decline in terms of an aging immune system. They might also explain why conditions of immune compromise (e.g. AIDS) result in cognitive impairment (HIV-associated dementia).

Previous studies by the present inventors have shown that systemic manipulations of the immune system, based on increasing the numbers of T cells directed to weak agonists of autoantigens, beneficially affect neurodegenerative conditions by promoting neuronal survival (Moalem et al., 1999; Hauben et al., 2001; Schwartz and Kipnis, 2002). The same manipulations, for example, T cell-based vaccination, is proposed here for increasing neurogenesis, yielding novel ways to maintain the integrity of the aging brain and the diseased mind.

It thus seems that maintenance and repair of brain cells necessitate a dialog between CNS-autoreactive T cells and brain-resident microglia. This dialog cannot take place, however, unless the microglia are able to act as APCs, presenting the relevant antigens to the homing T cells. We therefore postulated that in order to halt the progression of Alzheimer disease (AD), T cells that recognize CNS-specific antigens other than aggregated amyloid-β (Aβ) must target sites of aggregated Aβ plaques in the brain. On reaching these sites they become activated by the encounter with their specific antigens, presented to them by microglia acting as APCs. Such activation enables these T cells to offset the negative effect of aggregated Aβ on locally resident microglia, thus preventing the latter from becoming cytotoxic to neurons and blocking neurogenesis. We tested this hypothesis by vaccinating AD mice with glatiramer acetate (GA, also known as copolymer 1 or Cop-1), a synthetic copolymer approved by the FDA for treatment of multiple sclerosis, and capable of weakly cross-reacting with a wide range of CNS-resident autoantigens (Kipnis et al., 2000). GA-activated T cells, after infiltrating the CNS, have the potential to become locally activated without risk of the overwhelming proliferation that is likely to cause an autoimmune disease. Studies by the present inventors and others have shown that GA can simulate the protective and reparative effects of autoreactive T cells (Kipnis et al., 2000; Benner et al., 2004).

In the present invention, APP/PS1 double-transgenic AD mice (which coexpress mutated human presenilin 1 and amyloid-β precursor protein) suffering from decline in cognition and accumulation of Aβ plaques, a T cell-based vaccination, by altering the microglial phenotype, ameliorated cognitive performance, reduced plaque formation, rescued cortical and hippocampal neurons, and induced hippocampal neurogenesis.

We show here that vaccination of Tg mice with GA reduced plaque formation, and prevented and even partially reversed cognitive decline, even if the vaccination was given after some loss of cognition and some plaque formation had already occurred. It should be noted that vaccination with GA was effective not only in preventing disease progression but also—when administered after onset of the clinical symptoms of learning/memory loss and pathological appearance of plaques—in promoting tissue repair. The above findings are in line with our observation that in mice deficient in CNS-autoreactive T cells the expression of brain-derived neurotrophic factor (BDNF), known to be associated with both cognitive activity and cell renewal, is impaired. They are also in accord with our observation that T cells are needed for the maintenance of cognitive functioning in the healthy as well as in the diseased brain (Kipnis et al., 2004). Since aggregated Aβ evidently interferes with the ability of microglia to engage in dialog with T cells, its presence in the brain can be expected to cause loss of cognitive ability and impairment of neurogenesis. Homing of CNS-autoreactive T cells to the site of disease or damage in such cases is critical, but will be effective only if those T cells can counterbalance the destructive activity of the aggregated Aβ. Myelin-presenting microglia, with which myelin-specific T cells can readily hold a dialog, are likely to be present in abundance. Myelin-related antigens, or antigens (such as GA) that are weakly cross-reactive with myelin, are therefore likely to be the antigens of choice for the therapeutic vaccination. Myelin-specific T cells will then home to the CNS and, upon encountering their relevant APCs there, will become locally activated to supply the cytokines and growth factors needed for appropriate modulation of harmful microglia like those activated by aggregated Aβ. The resulting synapse between T cells and microglia will create a supportive niche for cell renewal by promoting neurogenesis from the pool of adult stem cells, thereby overcoming the age-related impairment induced in the inflammatory brain.

In another embodiment, we used the immunomodulator PN277, a random synthetic copolymer composed of glutamic acid and tyrosine residues (also known as polyYE and poly-Glu, Tyr), in a model of stroke. Boosting T-cell mediated immune response following insult to the CNS can be done by either vaccinating with a CNS-specific antigen or by abolishing regulatory T cells (Treg) activity (Moalem et al., 1999; Hauben et al., 2000; Schwartz et al., 2003). PN277 was recently shown to down-regulate the inhibitory effects of naturally occurring regulatory T cells and to be capable of reducing Treg suppressive activity and confer neuroprotection (WO 2005/055920). While vaccination induce T-cell response primarily to one antigen, reducing Treg suppressive activity facilitate a broader T-cell response against various antigens residing in the injured site. Thus, following stroke, mostly T cells specific for various CNS-antigens would be propagated in response to PN277 treatment.

The finding of enhanced hippocampal neurogenesis in the PN277-treated rats is consistent with our finding that neurogenesis was enhanced in transgenic mice over-expressing T-cell receptor for MBP. The fact that this effect was seen in both ipsilateral and contralateral sides further imply that elevation in neurogenesis is caused by a systemic event (T-cell response). Such autoimmune T cells can locally interact with microglia, which in turn can recruit NPCs and direct their differentiation. This scenario is supported by the findings herein showing that neurogenesis from aNPCs could be induced by microglia activated by cytokines (IL-4 or IFN-γ) associated with T-helper cells. The effect of PN277 treatment on neurogenesis was even more robust with regard to cortical neurogenesis. In conclusion, the results of this study suggest that proper immune modulation can increase neurogenesis, and that lack of neurogenic permissiveness in brain regions such as the cortex can be changed by the local immune response. These findings introduce a new therapeutic approach for augmenting spontaneously occurring neurogenesis following an ischemic insult.

The present invention thus relates, in one aspect, to a method for inducing and enhancing neurogenesis and/or oligodendrogenesis from endogenous as well as from exogenously administered stem cells, which comprises implanting stem cells into an individual in need, and administering to said individual a neuroprotective agent selected from the group consisting of:
(i) a nervous system (NS)-specific antigen or an analog thereof;
(ii) a peptide derived from an NS-specific antigen or from an analog thereof, or an analog or derivative of said peptide;
(iii) poly-YE and poly-YE related peptides;
(iv) T cells activated with an agent (i) to (iii);
(v) antigen-presenting cells that have been pulsed with an agent (i) to (iii);
(vi) activated mononuclear phagocytic leukocytes;
(vii) dopamine, a dopamine precursor, an agonist of the dopamine receptor type 1 family (D1-R agonist), a combination of dopamine and a dopamine precursor, or a combination of dopamine, a dopamine precursor, or a D1-R agonist with an antagonist of the dopamine receptor type 2 family (D2-R antagonist);
(viii) microglia activated by IFN-γ or IL-4, or both;
(ix) IFN-γ or IL-4, or both; and
(x) any combination of (i) to (ix).

In one embodiment, the present invention relates to a method for inducing and enhancing neurogenesis from endogenous or exogenously applied stem cells, by immune modulation, which comprises administering to an individual in need a neuroprotective agent selected from the group consisting of the agents (i) to (x) defined above.

In another embodiment, the present invention relates to a method for inducing and enhancing oligodendrogenesis from endogenous or exogenously applied stem cells, by immune modulation, which comprises administering to an individual in need a neuroprotective agent selected from the group consisting of the agents (i) to (x) defined above.

The stem cells for use in the present invention are stem cells of any origin used today or to be used in the future in stem cell therapy and include, without limitation, adult stem cells (found in various tissues of an adult organism that remain in an undifferentiated, or unspecialized, state, and can renew themselves and differentiate to yield all the specialized cell types of the tissue from which it originated and other types of cells), embryonic stem cells, umbilical cord blood stem cells, hematopoietic stem cells, peripheral blood stem cells, mesenchimal stem cells, multipotent stem cells, neural stem cells, stromal cells, progenitor cells (cells that can differentiate into a limited number of cell types, but cannot make more stem cells or renew itself), and any other type of stem cells and precursors thereof.

The nervous system (NS)-specific antigens and analogs thereof defined herein as agent (i), the peptides derived from NS-specific antigens or from analogs thereof, and analogs or derivatives of said peptides defined as agent (ii), and T cells activated with (i) or (ii), include the agents described in U.S. patent application Ser. No. 10/810,653 and PCT Publications WO 99/60021 and WO 02/055010 of the applicant, all these applications being herewith incorporated by reference as if fully disclosed herein.

The term “nervous system (NS)-specific antigen” as used herein refers to an antigen of the central or peripheral nervous system that specifically activates T cells such that following activation the activated T cells accumulate at a site of injury or disease in the NS of the patient. In one embodiment, the agent for use in the invention is a NS-antigen of mammal, preferably human, origin, such as, but not limited to, myelin basic protein (MBP), myelin oligodendrocyte glycoprotein (MOG), proteolipid protein (PLP), myelin-associated glycoprotein (IAG), S-100, β-amyloid, Thy-1, a peripheral myelin protein such as P0, P2 and PMP22, neurotransmitter receptors, the protein Nogo (Nogo-A, Nogo-B and Nogo-C) and the Nogo receptor (NgR), or an analog thereof. This definition also includes analogs of said NS-specific antigens in which one or more amino acid residues of the NS-antigen have been deleted or replaced by different amino acid residues and/or amino acid residues have been added to the antigen sequence.

In another embodiment, the agent is a peptide derived from an NS-specific antigen or from an analog thereof, as defined hereinabove. As used herein, the term “a peptide derived from an NS-specific antigen” relates to a peptide that has a sequence comprised within the NS-antigen sequence. In one embodiment, the peptide is an immunogenic epitope or a cryptic epitope derived from said antigen. Examples of such peptides include the MBP peptides p11-30, p51-70, p91-110, p131-150, and p151-170 of MBP.

In a further embodiment, the agent is an analog of an NS-specific antigen peptide obtained by modification of a self-peptide derived from a CNS-specific antigen, which modification consists in the replacement of one or more amino acid residues of the self-peptide by different amino acid residues or by deletion or addition of one or more amino acid residues, said modified CNS peptide still being capable of recognizing the T-cell receptor recognized by the self-peptide but with less affinity (hereinafter “modified CNS peptide” or “altered peptide”).

The modified CNS peptide preferably is immunogenic but not encephalitogenic. The most suitable peptides for this purpose are those in which an encephalitogenic self-peptide is modified at the T cell receptor (TCR) binding site and not at the MHC binding site(s), so that the immune response is activated but not anergized. The invention also encompasses peptides derived from any encephalitogenic epitopes in which critical amino acids in their TCR binding site but not MHC binding site are altered as long as they are non-encephalitogenic and still recognize the T-cell receptor.

In one embodiment, the modified peptide is derived from the residues 86 to 99 of human MBP by alteration of positions 91, 95 or 97 as disclosed in U.S. Pat. No. 5,948,764. In another embodiment, the modified peptide is a peptide disclosed in WO 02/055010, derived from the residues 87-99 of human MBP, in which the lysine residue 91 is replaced by glycine (G91) or alanine (A91), or the proline residue 96 is replaced by alanine (A96). In a further embodiment, the peptide is derived from the encephalitogenic MOG peptide p35-55 of the SEQ ID NO:1 such as the peptide obtained by deletion of 1 amino acid from either the N- or C-terminus of the truncated peptide pMOG₄₄₋₅₄ or the modified MOG peptide of SEQ ID NO:2. In a further embodiment, the modified peptide is an analog of the peptide 95-117 of PLP.

In another embodiment, the agent for use in the invention are T cells activated by a NS-specific antigen, an analog of said antigen, a peptide derived from said antigen or antigen analog or an analog or derivative of said peptide, all as defined above. The T cells can be endogenous and activated in vivo by administration of the antigen or peptide, thereby producing a population of T cells that accumulate at a site of injury or disease of the CNS or PNS.

In one embodiment, the T cells are prepared from T lymphocytes isolated from the blood and then sensitized to the NS-antigen or peptide, preferably to a modified peptide as defined herein. The T cells are preferably autologous, most preferably of the CD4 and/or CD8 phenotypes, but they may also be allogeneic T cells from related donors, e.g., siblings, parents, children, or HLA-matched or partially matched, semi-allogeneic or fully allogeneic donors. Methods for the preparation of said T cells are described in the above-mentioned WO 99/60021.

In another embodiment, the agent is PN277 (Poly-YE), a random copolymer of Tyr and Glu that may contain the amino acids Glu and Tyr in any available ratio such as, for example, poly-(Glu, Tyr) 1:1 and poly(Glu, Tyr) 4:1. The modulation of the immune response and modulation of autoimmunity response by poly-YE is described in U.S. Pat. No. 6,838,711, U.S. application Ser. No. 10/807,414, WO 03/002140, WO 2005/055920, and WO 2005/089787, all these applications being herewith incorporated by reference as if fully disclosed herein. As used herein, the term “poly-YE related peptides” refers to random copolymers of Tyr and Glu with different ratios of Glu and Tyr and/or lower or higher molecular weight and to random peptides containing several residues of Tyr and Glu.

In another embodiment, the antigen-presenting cells (APCs) for use in the invention are APCs that have been pulsed with a NS-specific antigen or an analog thereof, a peptide derived from a NS-specific antigen or from an analog thereof, or an analog or derivative of said peptide, or Poly-YE, as described in WO 03/105750, herewith incorporated by reference as if fully disclosed herein. The APCs for use in the invention are preferably human APCs and are selected from the group consisting of monocytes, macrophages, B cells and, preferably, dendritic cells. The human dendritic cells can be obtained from skin, spleen, thymus, bone marrow, lymph nodes or peripheral blood of an individual, and cultured as described in WO 03/105750, preferably in a medium containing IL-4, GM-CSF, or both IL-4 and GM-CSF.

In a further embodiment, the agent is activated mononuclear phagocytic leukocytes, such as the activated cells described in U.S. Pat. No. 5,800,812, No. 6,117,424 and No. 6,267,955, and WO 03/044037, all these patents and applications being herewith incorporated by reference as if fully disclosed herein. The activated mammalian mononuclear phagocytes are obtained by culturing the cells together with at least one stimulatory tissue such as skin, dermis and a nerve, e.g. peripheral, nerve, segment, or with stimulatory cells derived from skin, dermis, and a nerve segment, or with medium conditioned by skin, dermis, and/or a nerve segment, a medium conditioned by at least one stimulatory tissue or cells, or with medium to which at least one stimulatory biologically active agent has been added. The stimulatory biologically active agent may be neurotrophic factor 3 (NT-3), nerve growth factor (NGF), brain-derived neurotrophic factor (BDNF) and transforming growth factor-β (TGF-β).

In another embodiment the agent is dopamine or a pharmaceutically acceptable salt thereof, optionally in combination with the dopamine precursor levodopa, and further optionally in combination with carbidopa. The D1-R agonist may be selected from the group consisting of SKF-82958, SKF-38393, SKF-77434, SKF-81297, A-77636, fenoldopam and dihydrexidine. The dopamine, dopamine precursor or D1-R agonist may be in combination with a dopamine D2-R antagonist selected from the group consisting of amisulpride, eticlopride, raclopride, remoxipride, sulpride, tropapride, domperidone, iloperidone, risperidone, spiperone, haloperidol, spiroperidol, clozapine, olanzapine, sertindole, mazapertine succinate, zetidoline, CP-96345, LU111995, SDZ-HDC-912, and YM 09151-2. All these agents are as described in WO 2005/055920, herewith incorporated by reference as if fully disclosed herein. These agents cause down-regulation of the suppressive activity of CD4⁺ CD25⁺ regulatory T (Treg) cells on CD4⁺ CD25⁻ effector T cells (Teff) and thus, they modulate the immune response and/or the autoimmune response

In a further embodiment, the agent is microglia, a major glial component of the CNS that play a critical role as resident immunocompetent cells and phagocytic cells in the CNS, wherein the microglia are activated by IFN-γ or IL-4. Administration of the cytokine alone, IL-4 or low dose of IFN-γ, will also cause the activation of endogenous microglia.

In further embodiments, the present invention provides a combination of any two or more of the agents (i) to (ix). In some embodiments, the combination comprises T-cell vaccination by administration of T cells and the NS-antigen to which the cells were sensitized, or administration of T cells and microglia, as defined herein.

The methods of the invention are useful for inducing and enhancing neurogenesis and/or oligodendrogenesis both from endogenous and exogenously administered stem cells and may assist in solving the problems found today with the poor results of stem cell transplantation, particularly in the cases of injuries, diseases, disorders and conditions of the nervous system, both the CNS and PNS.

In one embodiment, the method of the invention is applied to induce and enhance neurogenesis and/or oligodendrogenesis from endogenous pools of neural stem/progenitor cells. Thus, the immunomodulators used in the present invention will by themselves boost endogenous neurogenesis and oligodendrogenesis in damaged tissues, supporting also the survival of the new neurons and oligodendrocytes.

In another embodiment, the method of the invention is applied to induce and enhance neurogenesis and/or oligodendrogenesis from both endogenous and exogenous stem cells administered to the patient. The administration of a neuroprotective/immunomodulator agent (i) to (ix) or a combination thereof will assist to enhance the successful engraftment of the implanted stem cells, cell renewal and differentiation of the stem cells into neurons and/or oligodendrocytes, while at the same time inducing the endogenous neurogenesis and oligodendrogenesis in the damaged tissues and supporting the survival of the new neurons and oligodendrocytes.

The method of cell therapy according to the present invention may be carried out by different routes. In one embodiment, the stem cells are injected/transplanted to the patient, followed by vaccination with the neuroprotective/immunomodulator agent. In another embodiment, a combination of the stem cells with the neuroprotective/immunomodulator agent is injected/transplanted to the patient. In a further embodiment, the stem cells can be cultured in vitro (artificially) with the neuroprotective/immunomodulator agent and differentiated prior to transplantation

In one embodiment, the method of the invention comprises stem cell therapy by transplantation of stem cells in combination with a neuroprotective agent (i) to (x), to an individual that suffers from an injury in the CNS such as spinal cord injury, closed head injury, blunt trauma, penetrating trauma, hemorrhagic stroke, ischemic stroke, cerebral ischemia, optic nerve injury, myocardial infarction and injury caused by tumor excision. The transplanted stem cells will migrate to the region of the injury where cells had died (for example, due to ischaemia) and will differentiate into neurons and/or oligodendrocytes.

In another embodiment, the method of the invention comprises stem cell therapy by administration of stem cells in combination with a neuroprotective agent (i) to (x), to a patient suffering from of a neurodegenerative disease or disorder such as Parkinson's disease and Parkinsonian disorders, Huntington's disease, Alzheimer's disease, multiple sclerosis, or amyotrophic lateral sclerosis (ALS). For ALS, the neuroprotective agent is preferably a peripheral myelin or a peptide derived from a peripheral myelin or an analog thereof.

In another embodiment, the method of the invention comprises stem cell therapy by administration of stem cells in combination with a neuroprotective agent (i) to (x), to a patient suffering from a disease, disorder or condition of the CNS or PNS such as facial nerve (Bell's) palsy, glaucoma, Alper's disease, Batten disease, Cockayne syndrome, Guillain-Barré syndrome, Lewy body disease, Creutzfeldt-Jakob disease, or a peripheral neuropathy such as a mononeuropathy or polyneuropathy selected from the group consisting of adrenomyeloneuropathy, alcoholic neuropathy, amyloid neuropathy or polyneuropathy, axonal neuropathy, chronic sensory ataxic neuropathy associated with Sjogren's syndrome, diabetic neuropathy, an entrapment neuropathy nerve compression syndrome, carpal tunnel syndrome, a nerve root compression that may follow cervical or lumbar intervertebral disc herniation, giant axonal neuropathy, hepatic neuropathy, ischemic neuropathy, nutritional polyneuropathy due to vitamin deficiency, malabsorption syndromes or alcoholism, porphyric polyneuropathy, a toxic neuropathy caused by organophosphates, uremic polyneuropathy, a neuropathy associated with a disease or disorder selected from the group consisting of acromegaly, ataxia telangiectasia, Charcot-Marie-Tooth disease, chronic obstructive pulmonary diseases, Fabry's disease, Friedreich ataxia, Guillain-Barré syndrome, hypoglycemia, IgG or IgA monoclonal gammopathy (non-malignant or associated with multiple myeloma or with osteosclerotic myeloma), lipoproteinemia, polycythemia vera, Refsum's syndrome, Reye's syndrome, and Sjogren-Larsson syndrome, a polyneuropathy associated with various drugs, with hypoglycemia, with infections such as HIV infection, or with cancer. For peripheral neuropathies, the neuroprotective agent is preferably a peripheral myelin or a peptide derived from a peripheral myelin or an analog thereof.

In a further embodiment, the method of the invention comprises stem cell therapy by administration of stem cells in combination with a neuroprotective agent (i) to (x), to a patient suffering from epilepsy, amnesia, anxiety, hyperalgesia, psychosis, seizures, oxidative stress, opiate tolerance and dependence, and for the treatment of a psychosis or psychiatric disorder selected from the group consisting of an anxiety disorder, a mood disorder, schizophrenia or a schizophrenia-related disorder, drug use and dependence and withdrawal, and a memory loss or cognitive disorder.

The stem cells that can be injected or transplanted to an individual according to the method of the invention include, but are not limited to, adult stem cells, neural precursors, neural stem cells, neural adult cells, neural progenitor cells, hematopoietic stem cells, mesenchimal stem cells, embryonic stem cells, stromal stem cells, pluripotent stem cells and any other type of stem cells and precursors thereof, that may be found suitable for the purpose of the present invention. Examples of such cells include the CNS neural stems cells disclosed in U.S. Pat. No. 6,777,233 and U.S. Pat. No. 6,680,198; the neural stem cells and hematopoietic cells disclosed in U.S. Pat. No. 6,749,850 for administration with neural stimulants; and the stromal cells disclosed in U.S. Pat. No. 6,653,134 for treatment of CNS diseases.

As used herein, the term “neural stem cell” is used to describe a single cell derived from tissue of the central nervous system, or the developing nervous system, that can give rise in vitro and/or in vivo to at least one of the following fundamental neural lineages: neurons (of multiple types), oligodendroglia and astroglia as well as new neural stem cells with similar potential. “Multipotent” or “pluripotent” neural stem cells are capable of giving rise to all of the above neural lineages as well as cells of equivalent developmental potential.

In a more preferred embodiment, the neural stem cells are human neural stem cells that can be isolated from both the developing and adult CNS, and can be successfully grown in culture, are self-renewable, and can generate mature neuronal and glial progeny. Embryonic human neural stem cells can be induced to differentiate into specific neuronal phenotypes. Human neural stem cells integrate into the host environment after transplantation into the developing or adult CNS. Human neural stem cells transplanted into animal models of Parkinson's disease and spinal cord injury have induced functional recovery. However, there are still problems with the engraftment of said cells and the present invention will enhance the successful engraftment, survival and further differentiation of the implanted cells. In a most preferred embodiment, the neural stem cells are autologous.

As used herein, the term “hematopoietic stem cells” refer to stem cells that can give rise to cells of at least one of the major hematopoietic lineages in addition to producing daughter cells of equivalent potential. Certain hematopoietic stem cells are capable of giving rise to many other cell types including brain cells.

The stem cells, once isolated, are cultured by methods known in the art, for example as described in U.S. Pat. No. 5,958,767, U.S. Pat. No. 5,270,191, U.S. Pat. No. 5,753,506, all of these patents being herewith incorporated by reference as if fully disclosed herein.

The treatment regimen according to the invention is carried out, in terms of administration mode, timing of the administration, and dosage, depending on the type and severity of the injury, disease or disorder and the age and condition of the patient. The immunomodulator may be administered concomitanly with, before or after the injection or implantation of the cells.

The administration of the cells may be carried out by various methods. In certain embodiments, the cells are preferably administered directly into the stroke cavity, the spinal fluid, e.g., intraventricularly, intrathecally, or intracistemally. The stem cells can be formulated in a pharmaceutically acceptable liquid medium, which can contain the immunomodulator molecule (i) to (iii), (vii) or (ix) or the immunomodulator cells (iv) to (vi) and (wiii) as well. Cells may also be injected into the region of the brain surrounding the areas of damage, and cells may be given systemically, given the ability of certain stem cells to migrate to the appropriate position in the brain.

The invention will now be illustrated by the following non-limiting examples.

### EXAMPLES

Animals. The animals used in the experiments, if not indicated differently, were supplied by the Animal Breeding Center of the Weizmann Institute of Science (Rehovot, Israel). All animals were handled according to the regulations formulated by the Weizmann Institute's Animal Care and Use Committee.

Reagents. Lipopolysaccharide (LPS) (containing<1% contaminating proteins) was obtained from Escherichia coli 0127:B8 (Sigma-Aldrich, St. Louis, Mo.). Recombinant mouse tumor necrosis factor (TNF)-α and insulin-like growth factor (IGF)-I (both containing endotoxin at a concentration below 1 EU per μg of cytokine), recombinant rat and mouse interferon (IFN)-γ and interleukin (IL)-4 (both containing endotoxin at a concentration below 0.1 ng per μg of cytokine), goat anti-mouse neutralizing anti-TNF-α antibodies (ãTNF-α; containing endotoxin at a concentration below 0.001 EU per μg of Ab), and goat anti-mouse/rat neutralizing anti-IGF-I (ãIGF-I; containing endotoxin at a concentration below 0.1 EU per μg of Ab) used in the Examples hereinafter were obtained from R&D Systems (Minneapolis, Minn.).

### Example 1

### Microglia Induce Neural Cell Renewal

### Microglia Activated by IL-4 or IFN-γ Differentially Induce Neurogenesis and Oligodendrogenesis from Adult Stem/Progenitor Cells

### Materials and Methods

(i) Neuralprogenitor cell (NPC) culture. Coronal sections (2 mm thick) of tissue containing the subventricular zone of the lateral ventricle were obtained from the brains of adult C57B16/J mice. The tissue was minced and then incubated for digestion at 37° C., 5% CO₂ for 45 mine in Earle's balanced salt solution containing 0.94 mg/ml papain (Worthington, Lakewood, N.J.) and 0.18 mg/ml of L-cysteine and EDTA. After centrifugation at 110×g for 15 min at room temperature, the tissue was mechanically dissociated by pipette trituration. Cells obtained from single-cell suspensions were plated (3500 cells/cm²) in 75-cm² Falcon tissue-culture flasks (BD Biosciences, Franklin Lakes, N.J.), in NPC-culturing medium [Dulbecco's modified Eagles's medium (DMEM)/F12 medium (Gibco/Invitrogen, Carlsbad, Calif.) containing 2 mM L-glutamine, 0.6% glucose, 9.6 μg/ml putrescine, 6.3 ng/ml progesterone, 5.2 ng/ml sodium selenite, 0.02 mg/ml insulin, 0.1 mg/ml transferrin, 2 μg/ml heparin (all from Sigma-Aldrich, Rehovot, Israel), fibroblast growth factor-2 (human recombinant, 20 ng/ml), and epidermal growth factor (human recombinant, 20 ng/ml; both from Peprotech, Rocky Hill, N.J.)]. Spheres were passaged every 4-6 days and replated as single cells. Green fluorescent protein (GFP)-expressing neural progenitor cells (NPCs) were obtained as previously described (Pluchino et al., 2003).

(ii) Primary microglial culture. Brains from neonatal (P0-P1) C57B1/6J mice were stripped of their meninges and minced with scissors under a dissecting microscope (Zeiss, Stemi DV4, Germany) in Leibovitz-15 medium (Biological Industries, Beit Ha-Emek, Israel). After trypsinization (0.5% trypsin, 10 min, 37° C./5% CO₂), the tissue was triturated. The cell suspension was washed in culture medium for glial cells [DMEM supplemented with 10% fetal calf serum (FCS; Sigma-Aldrich, Rehovot), L-glutamine (1 mM), sodium pyruvate (1 mM), penicillin (100 U/ml), and streptomycin (100 mg/ml)] and cultured at 37° C./5% CO₂ in 75-cm² Falcon tissue-culture flasks (BD Biosciences) coated with poly-D-lysine (PDL) (10 mg/ml; Sigma-Aldrich, Rehovot) in borate buffer (2.37 g borax and 1.55 g boric acid dissolved in 500 ml sterile water, pH 8.4) for 1 h, then rinsed thoroughly with sterile, glass-distilled water. Half of the medium was changed after 6 h in culture and every 2^{nd} day thereafter, starting on day 2, for a total culture time of 10-14 days. Microglia were shaken off the primary mixed brain glial cell cultures (150 rpm, 37° C., 6 h) with maximum yields between days 10 and 14, seeded (10⁵ cells/ml) onto PDL-pretreated 24-well plates (1 ml/well; Corning, Corning, N.Y.), and grown in culture medium for microglia [RPMI-1640 medium (Sigma-Aldrich, Rehovot) supplemented with 10% FCS, L-glutamine (1 mM), sodium pyruvate (1 mM), β-mercaptoethanol (50 mM), penicillin (100 U/ml), and streptomycin (100 mg/ml)]. The cells were allowed to adhere to the surface of a PDL-coated culture flask (1 h, 37° C./5% CO₂), and non-adherent cells were rinsed off.

(iii) Co-culturing of neural progenitor cells (NPCs) and mouse microglia. Microglia were treated for 24 h with cytokines (IFN-γ, 20 ng/ml; IL-4, 10 ng/ml) or LPS (100 ng/ml). Cultures of treated or untreated microglia were washed twice with fresh NPC-differentiation medium (same as the culture medium for NPCs but without growth factors and with 2.5% FCS) to remove all traces of the tested reagents, then incubated on ice for 15 min, and shaken at 350 rpm for 20 min at room temperature. Microglia were removed from the flasks and immediately co-cultured (5×10⁴ cells/well) with NPCs (5×10⁴ cells/well) for 5 or 10 days on cover slips coated with Matrigel (BD Biosciences) in 24-well plates, in the presence of NPC differentiation medium, with or without insulin. The cultures were then fixed with 2.5% paraformaldehyde in PBS for 30 min at room temperature and stained for neuronal and glial markers. Cell proliferation rates and cell survival in vitro were determined by staining with 5-bromo-2′-deoxyuridine (BrdU, 2.5 μM; Sigma-Aldrich, St. Louis). For quantification of live and dead cells, live cultures were stained with 1 μg/ml propidium iodide (Molecular Probes, Invitrogen, Carlsbad, Calif.) and 1 μg/ml Hoechst 33342 (Sigma-Aldrich, St. Louis), and cells were counted using Image-Pro (Media Cybernetics, Silver Spring, Md.), as described (Hsieh et al., 2004)

(iv) Immunocytochemistry. Cover slips from co-cultures of NPCs and mouse microglia were washed with PBS, fixed as described above, treated with a permeabilization/blocking solution containing 10% FCS, 2% bovine serum albumin, 1% glycine, and 0.1% Triton X-100 (Sigma-Aldrich, Rehovot) and stained with a combination of the mouse or rabbit anti-tubulin β-III-isoform C-terminus antibodies (β-II-tubulin; 1:500), rabbit anti-NG2 chondroitin sulfate proteoglycan (NG2; 1:500), mouse anti-RIP (RIP; 1:2000), mouse anti-galactocerebroside (GalC; 1:250), mouse anti-glutamic acid decarboxylase 67 (GAD; 1:1000), mouse anti-nestin (Nestin; 1:1000), rat anti-myelin basic protein (MBP; 1:300) (all from Chemicon, Temecula, Calif.), goat anti-double cortin (DCX; 1:400, Santa Cruz Biotechnology, Santa Cruz, Calif.) and mouse anti-glial fibrillary acidic protein (GFAP; 1:100, Sigma-Aldrich, St. Louis). For labeling of microglia we used either rat andi-CD11b (MAC1; 1:50, BD-Pharmingen, NJ) or FITC-conjugated Bandeiraea simplicifolia isolectin B4 (IB4; 1:50, Sigma-Aldrich, Rehovot). Expression of IGF-1 was detected by goat anti-IGF-1 (1:20, R&D Systems).

(v) RNA purification, cDNA synthesis, and reverse-transcription PCR analysis. Cells were lysed with TRI reagent (MRC, Cincinnati, Ohio), and total cellular RNA was purified from lysates using the RNeasy kit (Qiagen, Hilden, Germany) according to the manufacturer's instructions. Residual genomic DNA was removed during the purification process by incubation with RNase-free DNase (Qiagen). RNA was stored in RNase-free water (Qiagen) at −80° C. RNA (1 μg) was converted to cDNA using SuperScript II (Promega, Madison, Wis.), as recommended by the manufacturer. The cDNA mixture was diluted 1:5 with PCR-grade water.

We assayed the expression of specific mRNAs using semi-quantitative reverse transcription PCR(RT-PCR) with selected gene-specific primer pairs, using OLIGO v6.4 (Molecular Biology Insights, Cascade, Colo.).

The primers used were:

| | |
|---|---|
| TNF-α, sense | |
| 5′-AGGAGGCGCTCCCCAAAAAGATGGG-3′, | (SEQ ID NO: 3) |
| antisense | |
| 5′-GTACATGGGCTCATACCAGGGCTTG-3′ | (SEQ ID NO: 4) |
| (target size, 551 bp); | |
| IGF-I, sense | |
| 5′-CAGGCTCCTAGCATACCTGC-3′, | (SEQ ID NO: 5) |
| antisense | |
| 5′-GCTGGTAAAGGTGAGCAAGC-3′ | (SEQ ID NO: 6) |
| (target size, 244 bp); | |
| and | |
| β-actin, sense | |
| 5′-TTGTAACCAACTGGGACGATATGG-3′, | (SEQ ID NO: 7) |
| antisense | |
| 5′-GATCTTGATCTTCATGGTGCTAGG-3′ | (SEQ ID NO: 8) |
| (target size, 764 bp). | |

The RT-PCR reactions were carried out using 1 μg of cDNA, 35 nmol of each primer, and ReadyMix PCR Master Mix (ABgene, Epsom, UK) in 30-μl reactions. PCR reactions were carried out in an Eppendorf PCR system with cycles (usually 25-30) of 95° C. for 30 s, 60° C. for 1 min, 72° C. for 1 min, and 72° C. for 5 min, and then kept at 4° C. As an internal standard for the amount of cDNA synthesized, we used β-actin mRNA. PCR products were subjected to agarose gel analysis and visualized by ethidium bromide staining. Signals were quantified using a Gel-Pro analyzer 3.1 (Media Cybernetics). In all cases one product was observed with each primer set, and the observed product had an amplicon size that matched the size predicted from published cDNA sequences.

(vi) Quantification. For microscopic analysis we used a Zeiss LSM 510 confocal laser scanning microscope (40× magnification). For experiments in vitro we scanned fields of 0.053 mm² (n=8-16 from at least two different coverslips) for each experimental group. For each marker, 500-1000 cells were sampled. Cells co-expressing GFP and β-III-tubulin, NG2, RIP, GalC, and GFAP were counted.

(vii) Statistical analysis. The results were analyzed by the Tukey-Kramer multiple comparisons test (ANOVA) and are expressed as means±SD (unless differently indicated).

### Example 1(1)

### Effect of Microglia on Neurogenesis In Vitro

### Microglia Pretreated with IL-4 or IFN-γ Induce and Support Neuronal Differentiation from Neural Progenitor Cells (NPCs) in Vitro

Adaptive immunity, in the form of a well-controlled Th1 or a Th2 response to a CNS insult, induces microglia (MG) to adopt a phenotype that facilitates neuronal protection and neuronal tissue repair (Butovsky et al., 2001). Here we examined the ability of adaptive immunity, via activation of microglia, to induce or support the differentiation of NPCs. Neurogenesis is reportedly blocked by the inflammation caused by microglia activated with endotoxin (such as lipopolysaccharide, LPS) (Ekdahl et al., 2003). We therefore compared the effects on NPCs of microglia exposed to LPS (MG_{(LPS)}) with the effects of microglia exposed to the low levels of characteristic Th1 (pro-inflammatory) and Th2 (anti-inflammatory) cytokines, IFN-γ (MG_{(IFN-γ)}) and IL-4 (MG_{(IL-4γ)}), respectively, shown herein to be supportive of neural survival. We used NPCs expressing green fluorescent protein (GFP) to verify that any neural cell differentiation seen in the culture was derived from the NPCs rather than from contamination of the primary microglial culture.

Microglia were grown in their optimal growth medium (Zielasek et al., 1992) and were then treated for 24 h with IL-4, IFN-γ (low level), or LPS. Residues of the growth medium and the cytokines were washed off, and each of the treated microglial preparations, as well as a preparation of untreated microglia (MG₍₋₎), was freshly co-cultured with dissociated NPC spheres in the presence of differentiation medium. We examined the effects of both IFN-γ-activated and IL-4-activated microglia. After 5 days in culture, GFP⁺ cells that expressed the neuronal marker β-III tubulin were identified as neurons.

Since we recently showed that IL-4-activated microglia (MG_{(IL-4)}) produce a high level of IGF-1 (Butovsky et al., 2005), and because IGF-1 is reportedly a key factor in neural cell renewal (O'Kusky et al., 2000), we envisioned a situation in which IGF-1 might be one of the factors in the effect of IL-4-activated microglia. Therefore, the following experiments were carried out both in insulin-free (to allow detection, if exists, of the effect of insulin-related factors secreted by the activated microglia) and in insulin-containing differentiation media.

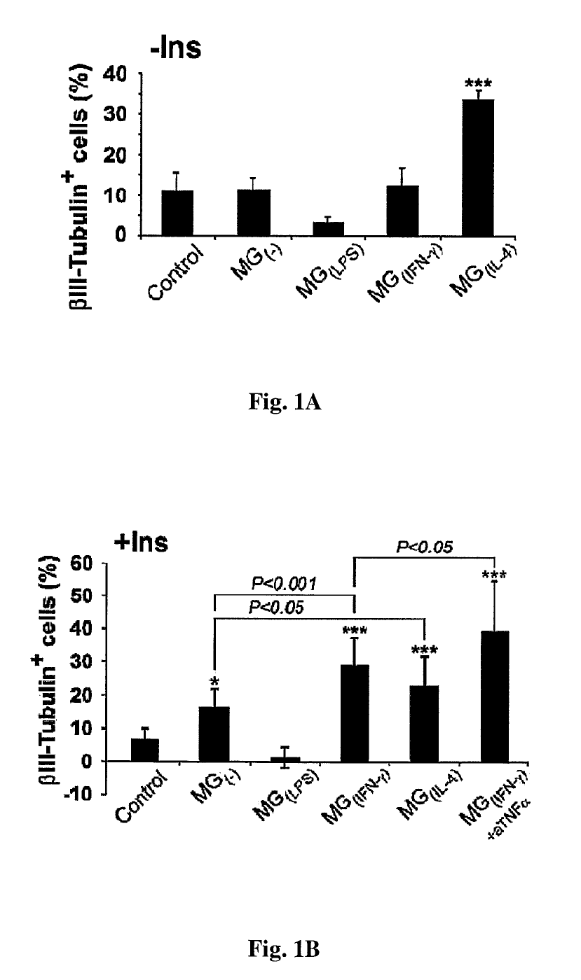

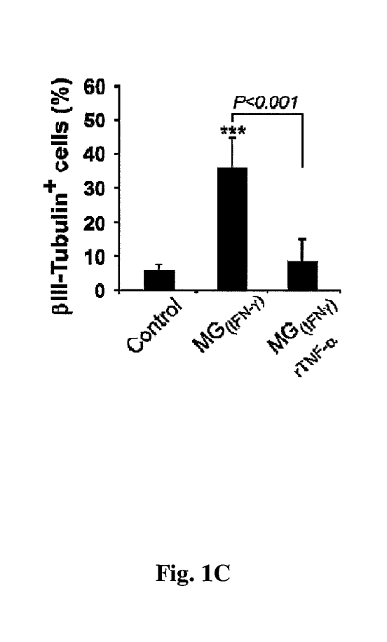

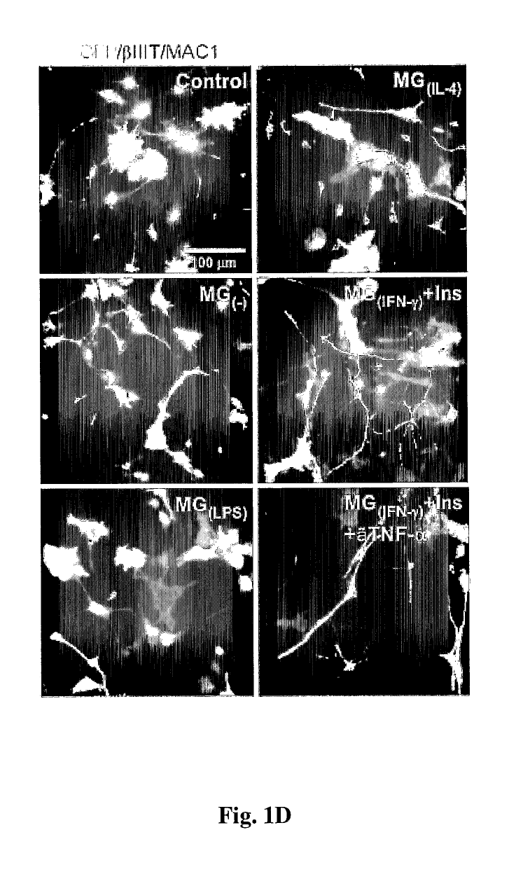

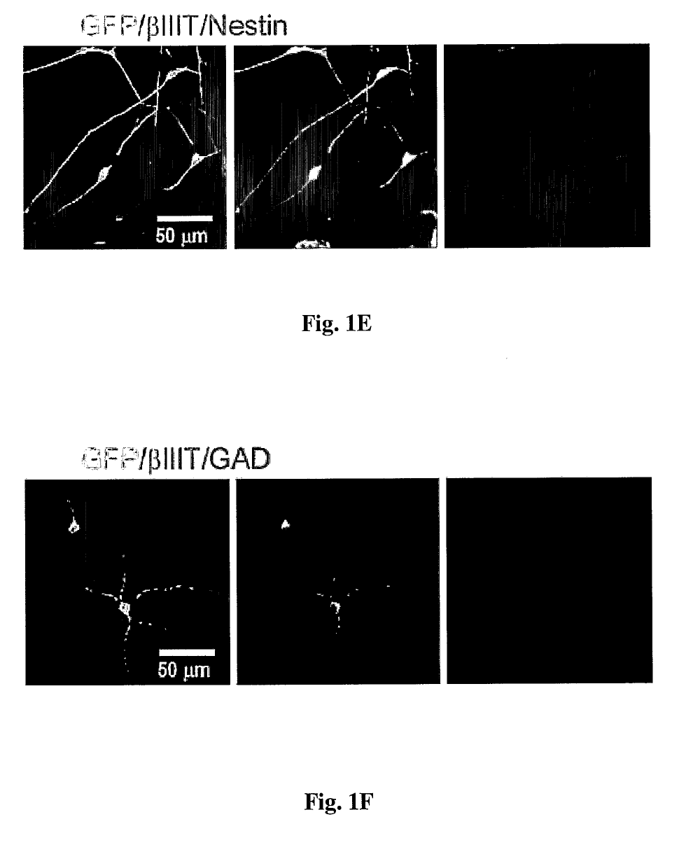

Quantitative analysis revealed that neurogenesis, in the absence of insulin, was only minimally supported by MG_{(IFN-γ)} and was impaired by MG_{(LPS)}, but was almost 3-fold higher in NPCs co-cultured with MG_{(IL-4)} than in controls (FIG. 1A). In the presence of insulin the picture was somewhat different: MG_{(IFNγ)} were significantly more effective than MG₍₋₎ in inducing neurogenesis, whereas the inductive effect of MG_{(IL-4)} and the blocking effect of MG_{(LPS)} on neurogenesis were similar to their effects in the absence of insulin (compare FIG. 1B). In the absence of microglia, addition of insulin (0.02 mg/ml) did not increase the numbers of GFP⁺/P-III-tubulin⁺ cells in NPC cultures (FIG. 1A).

In co-cultures of NPCs with MG₍₋₎, however, addition of insulin increased the percentage of GFP⁺/β-III-tubulin⁺ cells (FIG. 1B) relative to their percentage in such co-cultures without insulin (FIG. 1B) or in control (microglia-free) cultures in insulin-containing medium (FIG. 1B). In the presence of insulin, the number of neurons in NPCs co-cultured with MG_{(IFN-γ)} (FIG. 1B) was greater than in NPCs co-cultured with MG₍₋₎ (FIG. 1B), and even greater if the NPCs were co-cultured with MG_{(IFN-γ)} containing neutralizing anti-TNF-α antibodies (ãTNF-α) (FIG. 1B). To verify that the observed beneficial effect of ãTNF-α in the MG_{(IFN-γ)} co-cultures (FIG. 1B) was due to neutralization of the adverse effect of TNF-α on neurogenesis, we added recombinant mouse TNF-α (rTNF-α) to NPCs freshly co-cultured with MG_{(IFN-γ)}. FIG. 1C shows that in the presence of rTNF-α the numbers of GFP⁺/β-III-tubulin⁺ cells were similar to those in control (untreated) NPC cultures.

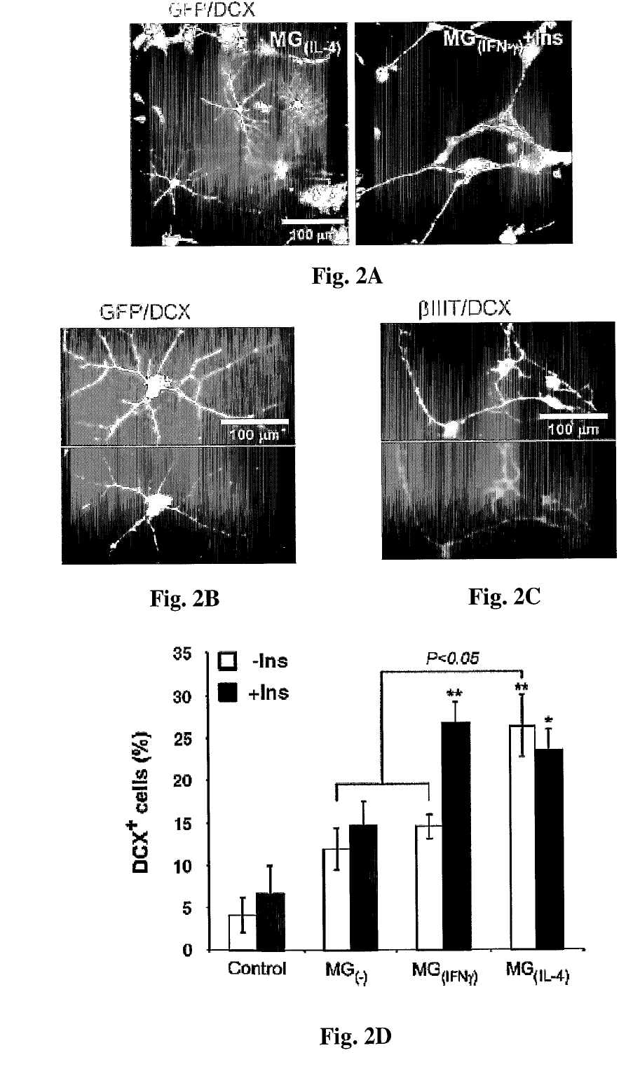

Morphological differences were observed between the newly differentiating neurons in NPCs co-cultured with MG_{(IFN-γ)} and those generated in co-cultures with MG_{(IL-4)} (FIG. 1D). Co-expression of GFP with β-III-tubulin is shown in FIG. 1E. The newly differentiating neurons were positively labeled for GAD67 (glutamic acid decarboxylase 67), an enzyme responsible for the synthesis of GABA, the major inhibitory transmitter in higher brain regions, and were also found to be co-labeled with GFP (β-III-tubulin⁺/GFP⁺/GAD⁺) (FIG. 1F). In another set of experiments we prepared cultures similar to those described above and stained them with doublecortin (DCX; FIG. 2), a marker of early differentiation of the neuronal lineage. This staining revealed a similar effect of the various microglial preparations to that seen with staining for β-III-tubulin. Striking differences in the morphology of newly differentiating neurons were seen between NPCs co-cultured with MG_{(IL-4)} and those co-cultured with MG_{(IFN-γ)} (FIG. 2A); the former showed significant branching, whereas in the latter the neurons were polarized and had long processes (FIG. 2A). These differences suggested that the mechanisms activated in microglia by the two cytokines are not identical. Co-expression of GFP with DCX is shown in FIG. 2B. In cultures stained for both DCX and β-III-tubulin, these two neuronal markers were found to be co-localized (FIG. 2C). In all of the above experiments microglial viability, assayed by propidium iodide staining of live cells (Hsieh et al., 2004), was unaffected by the co-culturing conditions. Quantitative analysis of GFP⁺/DCX⁺-stained cells, shown in FIG. 2D, yielded similar results to those obtained when β-III-tubulin was used as the neuronal marker (FIGS. 1A, 1B).

### Example 1(2)

### Effect of Microglia on Oligodendrogenesis In Vitro

### Differentiation of NPCs into Oligodendrocytes is Induced by co-culturing with IL-4 Pretreated Microglia (MG_{(IL-4)})

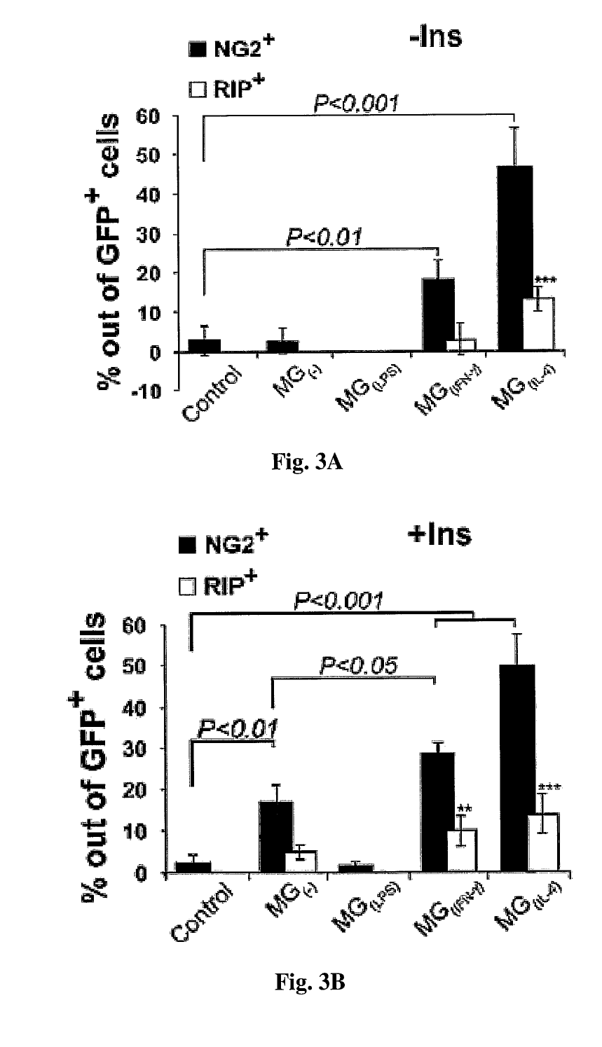

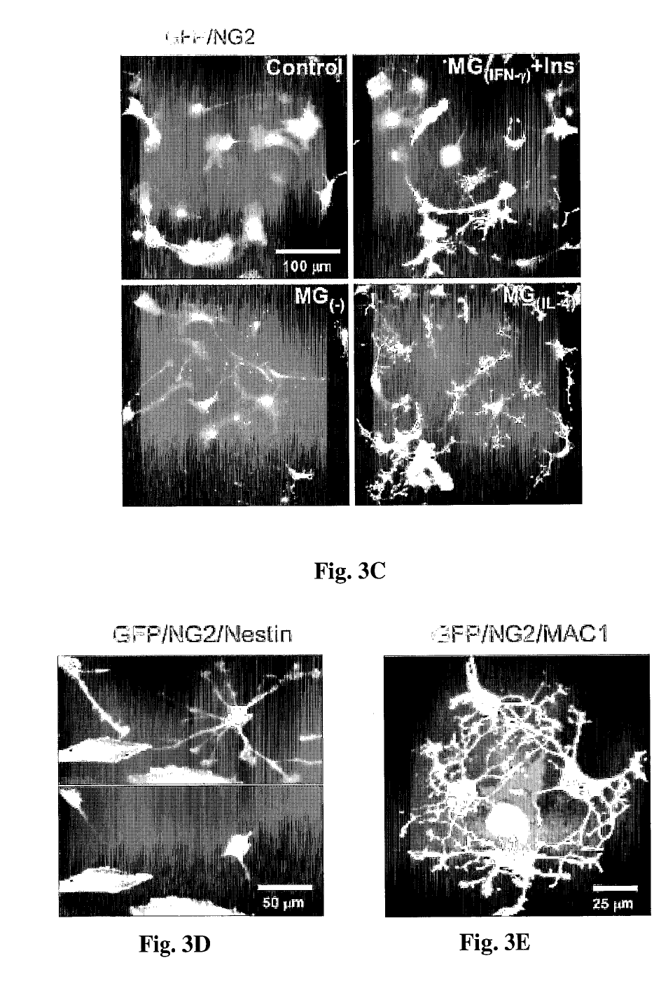

Next we examined whether, under the same experimental conditions, microglia would also induce NPCs to differentiate into oligodendrocytes. Under high magnification, we were able to detect newly formed oligodendrocytes. In attempting to detect possible differentiation of NPCs to oligodendrocytes, we first looked for GFP-labeled cells co-expressing oligodendrocyte progenitor marker NG2. Quantitative analysis confirmed that both MG_{(IL-4)} and (to a lesser extent) MG_{(IFN-γ)} induced differentiation of NG2⁺ cells from co-cultured NPCs (FIGS. 3A, 3B). In both MG₍₋₎ and MG_{(IFN-γ)} co-cultured with NPCs, significantly fewer NG2⁺-expressing cells were seen in the absence of insulin (FIG. 3A) than in its presence (FIG. 3B). Unlike in the case of neurogenesis (FIG. 1), MG_{(IFN-γ)}—even in the presence of insulin—was significantly less effective than MG_{(IL-4)} in inducing the appearance of newly differentiating oligodendrocytes (NG2⁺). A significant proportion of the NG2⁺ cells were also labeled for RIP [a monoclonal antibody that specifically labels the cytoplasm of the cell body and processes of premature and mature oligodendrocytes at the pre-ensheathing stage (Hsieh et al., 2004)] in both the MG_{(IFN-γ)} and the MG_{(IL-4)} co-cultures (FIGS. 3A, 3B). In the absence of insulin, almost no GFP⁺/NG2⁺ cells were seen in control medium containing no microglia (FIG. 3A). A few GFP⁺/NG2⁺ cells were seen in co-cultures of NPCs with MG₍₋₎ (FIG. 3A), but none in co-cultures with MG_{(LPS)} (FIG. 3A). A dramatic increase in the numbers of these cells was seen in co-cultures with MG_{(IL-4)} (FIGS. 3A, 3C).

Addition of insulin to the NPC cultures did not affect the incidence of NG2⁺ cells in the absence of microglia (control; FIG. 3B); it did, however, cause an increase in the numbers of NG2⁺ cells in NPCs co-cultured with MG₍₋₎ (FIG. 3B). Moreover, in the presence of insulin the blocking effect of MG_{(LPS)} on newly differentiating NG2⁺ cells was not altered (FIG. 3B), whereas the numbers of NG2⁺ cells in co-cultures with MG_{(IFN-γ)} were increased (FIGS. 3B, 3C). In each of the co-cultures, all NG2⁺ cells were also found to be labeled with GFP (FIG. 3D). An interesting observation was the close spatial association between the microglia and the newly differentiating oligodendrocytes (FIG. 3E).

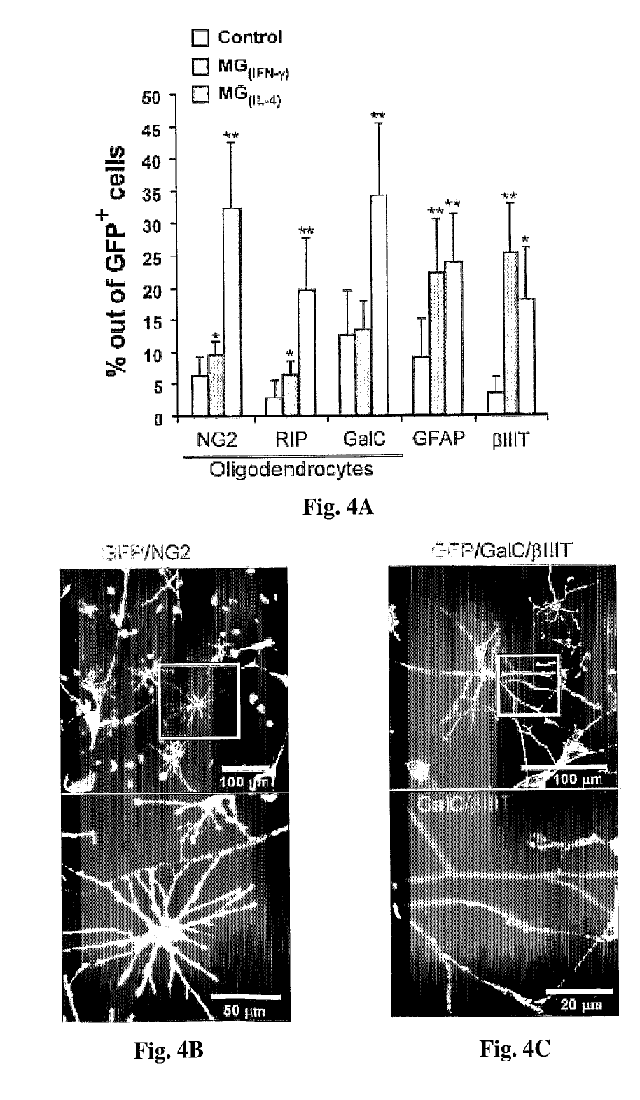

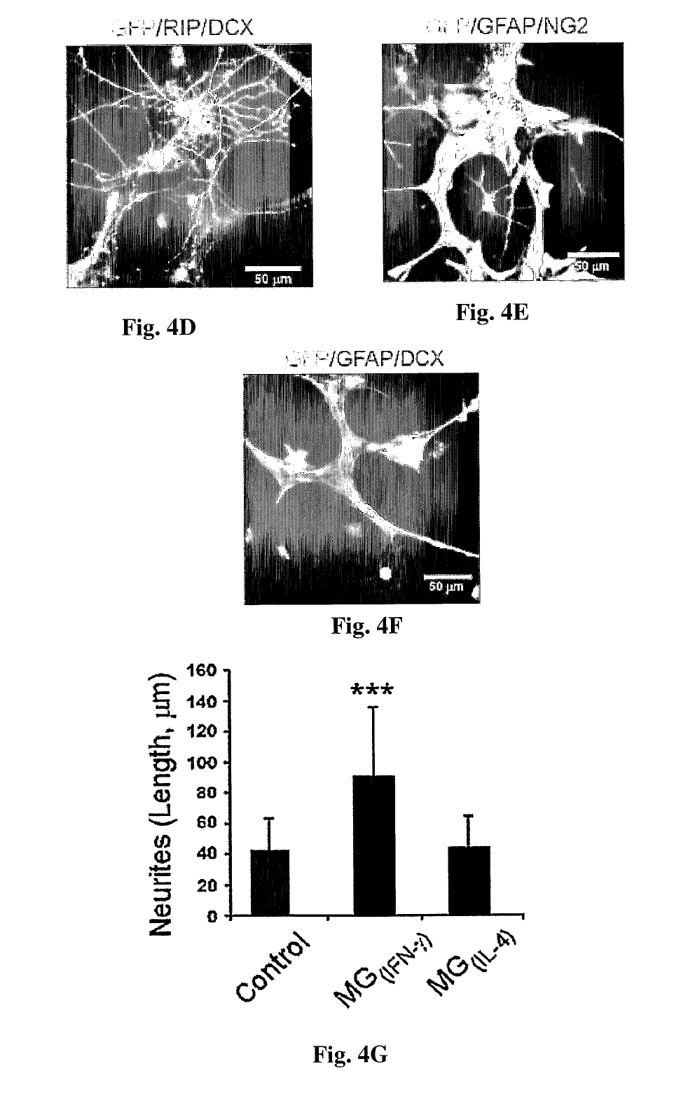

In light of the observed early differences between the effects of MG_{(IL-4)} and MG_{(IFN-γ)} on both neurogenesis and oligodendrogenesis, we examined NPCs co-cultured with the cytokine-activated microglia after 10 days in co-culture. As on day 5, few NG2⁺ cells were seen in the absence of microglia (FIG. 4A). Quantitative analysis of these cultures disclosed striking differences: while both of the cytokine-activated microglial preparations induced differentiation to both oligodendrocytes (NG2⁺, RIP⁺, GalC⁺) and neurons (P-III-tubulin⁺), MG_{(IL-4)} showed a positive bias towards mature oligodendrocytes and MG_{(IFN-γ)} towards mature neurons. Analysis of the incidence of astrocytes (GFAP⁺ cells) in these cultures (after 10 days of co-culturing) disclosed no significant differences between co-cultures of NPCs with MG_{(IL-4)} and with MG_{(IFN-γ)}; in both, however, GFAP⁺ cells were more numerous than in NPC cultures without microglia (FIG. 4A). The results suggested that these two types of cytokine-activated microglia affect the three neural cell lineages, and that they have different effects on the neuronal and oligodendrocyte lineages but not on the astrocytic lineage (FIG. 4A).

In the presence of MG_{(IL-4)}, the GFP⁺/NG2⁺ cells were more branched at 10 days (FIG. 4B) than at 5 days (FIG. 3C). The branching cells appeared to be forming contacts with cells that looked like neurons (FIG. 4B). Staining of these cultures for galactocerebroside (GalC), a marker of mature oligodendrocytes, and for the neuronal marker β-III-tubulin, verified that the contact-forming cells were newly formed oligodendrocytes and neurons (FIG. 4C). Analysis of the same cultures for DCX and RIP (FIG. 4D) revealed that none of the newly differentiating cells expressed both of these markers together. Moreover, there was no overlapping in expression of the astrocyte marker glial fibrillary acid protein (GFAP) and NG2 (FIG. 4E) or of GFAP and DCX (FIG. 4F). Analysis of neurite length induced by the cytokine-activated microglia (after 10 days of co-culturing) revealed that neurites of the newly formed neurons in NPCs co-cultured with MG_{(IFN-γ)} were significantly longer than in NPCs co-cultured with MG_{(IL-4)} or in NPCs alone, with no significant differences between neurite lengths in the latter two (FIG. 4G). Interestingly, there were no significant differences between the absolute numbers of GFP⁺ cells counted in these three groups (NPCs alone: 90.2±32.0; co-cultured with MG_{(IFNγ)}: 70.5±23.0; co-cultured with MG_{(IL-4)}: 66.1±10.4). This raises a question: do the activated microglia, besides affecting differentiation, also affect NPC proliferation and/or survival?

Table 1 records the proliferation of NPCs co-cultured with non-activated, IL-4-activated, or IFN-γ-activated microglia. Cultures of untreated NPCs (control) or NPCs co-cultured with MG₍₋₎ or MG_{(IL-4)} or MG_{(IFN-γ)} or MG_{(LPS)}, with or without insulin, were analyzed for proliferation and cell death 24, 48, or 72 h after plating. For the proliferation assay, a pulse of BrdU was applied 12 h before each time point. Numbers of BrdU⁺ cells are expressed as percentages of GFP⁺ cells (mean±SEM from three independent experiments in duplicate) and analyzed by ANOVA. Cell death with and without insulin was determined by live staining with 1 μg/ml propidium iodide and 1 μg/ml Hoechst 33342 (mean±SEM from two independent experiments in duplicate; *P<0.05; ***P<0.001; ANOVA).

As shown in Table 1, comparisons of proliferation at 24 h and 48 h of culture revealed no differences. After 72 h a slight but non-significant difference was seen between NPCs alone and NPCs co-cultured with MG₍₋₎ or MG_{(IFN-γ)}, possibly because of decreased proliferation in the culture of NPCs alone rather than any increase in the co-cultures. In the absence of insulin there were no significant differences at any time in culture between NPCs alone and NPCs co-cultured with MG₍₋₎ or with MG_{(IL-4)}. A reduction in proliferation was observed in NPCs co-cultured with MG_{LPS}, with or without insulin. After 5 days, no proliferation was detectable in any of the co-cultures (data not shown). To identify dead or dying cells we stained live cultures with 1 μg/ml propidium iodide, which stains dead cells, and 1 μg/ml Hoechst 33342, which stains both live and dead cells (Hsieh et al., 2004). Significant cell death was observed in NPCs co-cultured with MG_{(LPS)}, both in the absence and in the presence of insulin, whereas in NPCs cultured alone or with MG_{(IFN-γ)} or MG_{(IL-4)} the percentage of cell death was low and did not differ significantly from that seen in cultures of NPCs alone (Table 1). These results suggested that the primary effect of the cytokine-activated microglia on the fate of NPCs in vitro occurs via a mechanism that is instructive rather than selective.

TABLE 1
Proliferation and survival of neural progenitor cells (NPCs) in co-
cultures with microglia
% BrdU⁺ cells out of GFP⁺ cells

| | | | |
|---|---|---|---|
| Treatment | 24 h | 48 h | 72 h |

+Insulin

| | | | |
|---|---|---|---|
| Control | 5.8 ± 0.6 | 5.4 ± 1.5 | 1.4 ± 0.9 |
| MG₍₋₎ | 6.2 ± 0.5 | 6.9 ± 1.9 | 5.6 ± 1.4 |
| MG_{(IFN-γ)} | 5.8 ± 1.5 | 7.1 ± 2.5 | 5.6 ± 1.1 |
| MG_{(LPS)} | 3.1 ± 0.8 | 1.7 ± 0.4 | 1.1 ± 0.1 |

−Insulin

| | | | |
|---|---|---|---|
| Control | 4.1 ± 0.6 | 3.2 ± 0.5 | 1.2 ± 0.7 |
| MG₍₋₎ | 6.5 ± 1.5 | 3.5 ± 0.7 | 3.3 ± 2.5 |
| MG_{(IFN-γ)} | 6.2 ± 1.1 | 5.4 ± 1.9 | 3.3 ± 2.2 |
| MG_{(LPS)} | 2.1 ± 0.2 | 1.7 ± 0.5 | 1.1 ± 0.6 |

% PI⁺ cells out of GFP⁺ cells

| | | | |
|---|---|---|---|
| Treatment | 24 h | 48 h | 72 h |

+Insulin

| | | | |
|---|---|---|---|
| Control | 3.3 ± 0.9 | 1.7 ± 0.4 | 1.7 ± 0.7 |
| MG₍₋₎ | 4.0 ± 0.5 | 2.8 ± 0.7 | 2.8 ± 0.9 |
| MG_{(IFN-γ)} | 5.9 ± 0.6 | 4.2 ± 1.5 | 3.6 ± 2.1 |
| MG_{(LPS)} | 14.0 ± 0.1*** | 7.3 ± 1.9 | 4.1 ± 0.2 |

−Insulin

| | | | |
|---|---|---|---|
| Control | 3.7 ± 0.2 | 2.3 ± 0.8 | 2.2 ± 1.1. |
| MG₍₋₎ | 5.2 ± 3.0 | 3.7 ± 0.3 | 3.0 ± 1.0 |
| MG_{(IFN-γ)} | 3.7 ± 2.0 | 2.9 ± 0.4 | 2.5 ± 0.3 |
| MG_{(LPS)} | 15.8 ± 1.9* | 7.0 ± 5.0 | 4.8 ± 0.8 |

Proliferation and survival of neural progenitor cells (NPCs) in co-cultures with microglia. Cultures of untreated NPCs (control) or NPCs co-cultured with MG₍₋₎ or MG_{(IL-4)} or MG_{(IFN-γ)} or MG_{(LPS)}, with or without insulin, were analyzed for proliferation and cell death 24, 48, or 72 h after plating. For the proliferation assay a pulse of BrdU was applied 12 h before each time point. Numbers of BrdU⁺ cells are expressed as percentages of GFP⁺ cells (mean±SEM from three independent experiments in duplicate) and analyzed by ANOVA. Cell death with and without insulin was determined by live staining with 1 μg/ml propidium iodide and 1 μg/ml Hoechst 33342 (mean±SEM from two independent experiments in duplicate; *P<0.05; ***P<0.001; ANOVA).

### Example 1(3)

### Possible Mechanism of Oligodendrogenesis Induction by IL-4- and IFN-γ-Activated Microglia

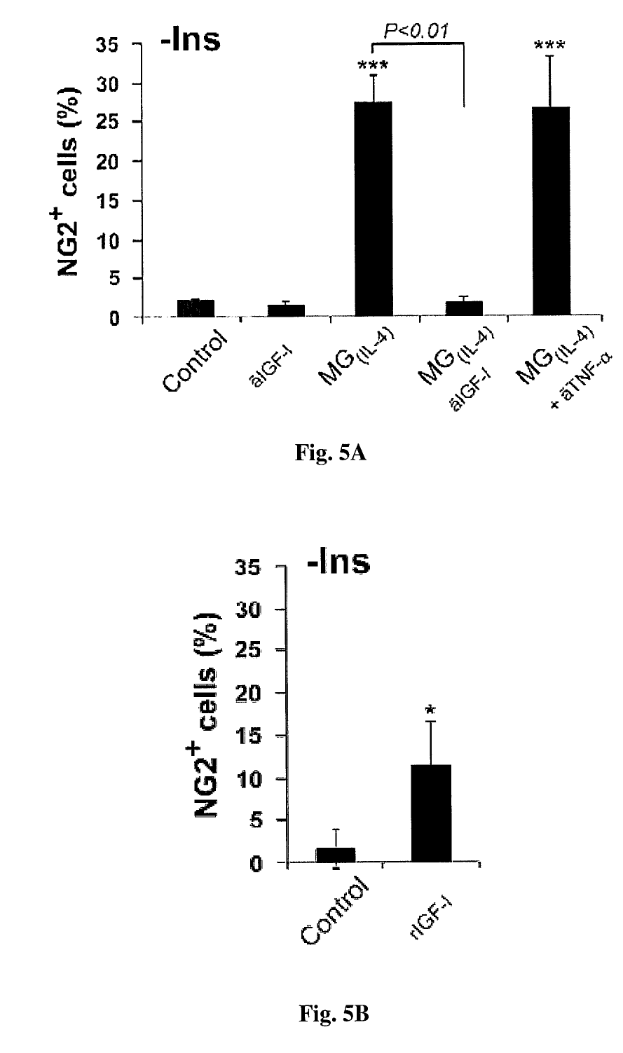

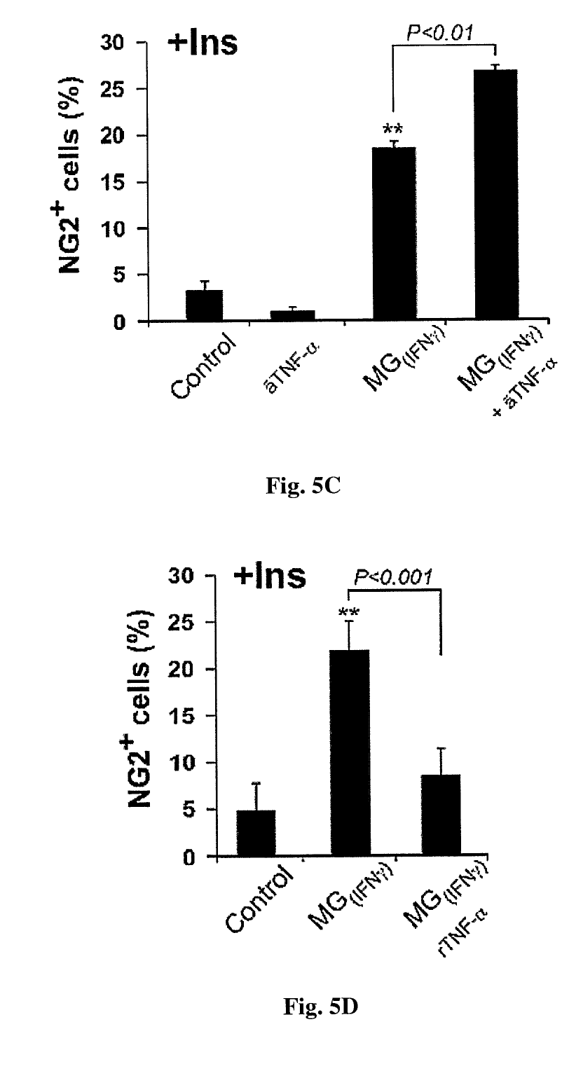

Insulin-like growth factor (IGF)-I is reportedly a key factor in neurogenesis and oligodendrogenesis (Aberg et al., 2000; O'Kusky et al., 2000; Hsieh et al., 2004). To determine whether the beneficial effect of the cytokine-activated microglia on the differentiation of NPCs is mediated, at least in part, by the ability of the microglia to produce IGF-I, we added neutralizing antibodies specific to IGF-I (aIGF-I) to the NPCs co-cultured with activated microglia. ãIGF-I blocked the MG_{(IL-4)}-induced effect on oligodendrogenesis (FIG. 5A), indicating that the effect of IL-4-activated microglia on oligodendrogenesis is dependent on IGF-I. Direct addition of recombinant IGF-I (rIGF-I; 500 ng/ml) to NPCs resulted in their significant differentiation to NG2-expressing cells (FIG. 5B). Such differentiation, however, was less extensive than that observed in NPCs co-cultured with MG_{(IL-4)} (FIG. 5A), suggesting that the MG_{(IL-4)} effect is mediated through additional (possibly soluble) factors, or by cell-cell interaction, or both. ãIGF-I had no effect on oligodendrogenesis induced by MG_{(IFN-γ)} (data not shown). We also examined the effect of ãIGF-I on MG_{(IL-4)}-induced neurogenesis by assessing β-III-tubulin expression. The percentage of GFP⁺/β-III-tubulin⁺ cells was 21.9±2.9% in NPCs co-cultured with MG_{(IL-4)} and 19.7±4.5%, (P=0.3) when ãIGF-I was added to those co-cultures. These results suggested that MG_{(IL-4)} produces additional potent neurogenic factors besides IGF-I.

In light of the observed beneficial effect of ãTNF-α on the outcome of MG_{(IFN-γ)}-induced neurogenesis (FIG. 1), we examined whether neutralization of TNF-α would promote MG_{(IFN-γ)}-induced oligodendrogenesis as well. Oligodendrogenesis was indeed enhanced by ãTNF-α in NPCs co-cultured with MG_{(IFN-γ)} (FIG. 5C). The implied negative effect of TNF-α was substantiated by direct addition of TNF-α to NPCs co-cultured with MG_{(IFN-γ)} (FIG. 5D).

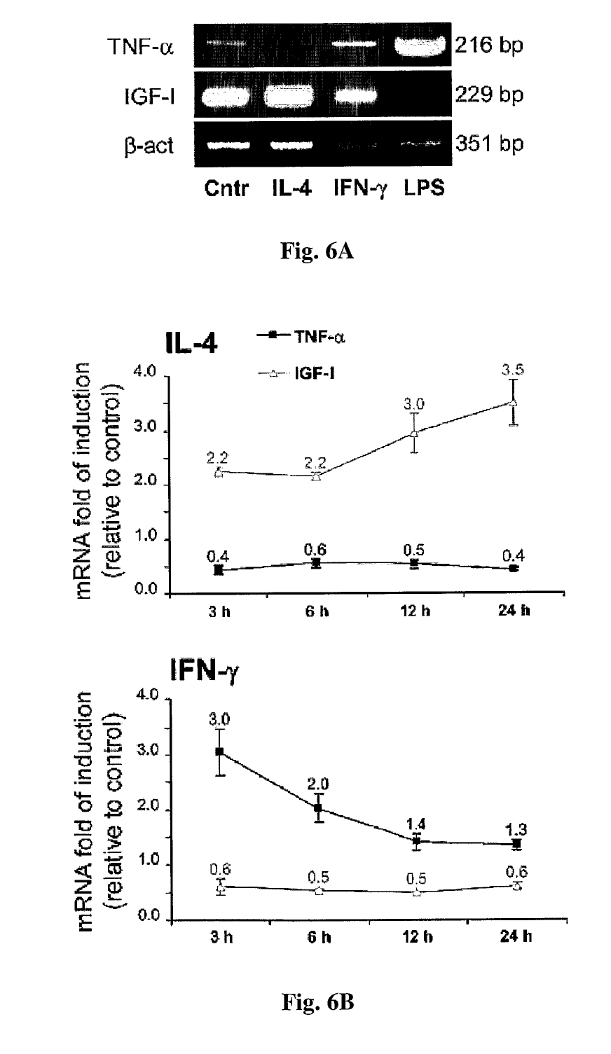

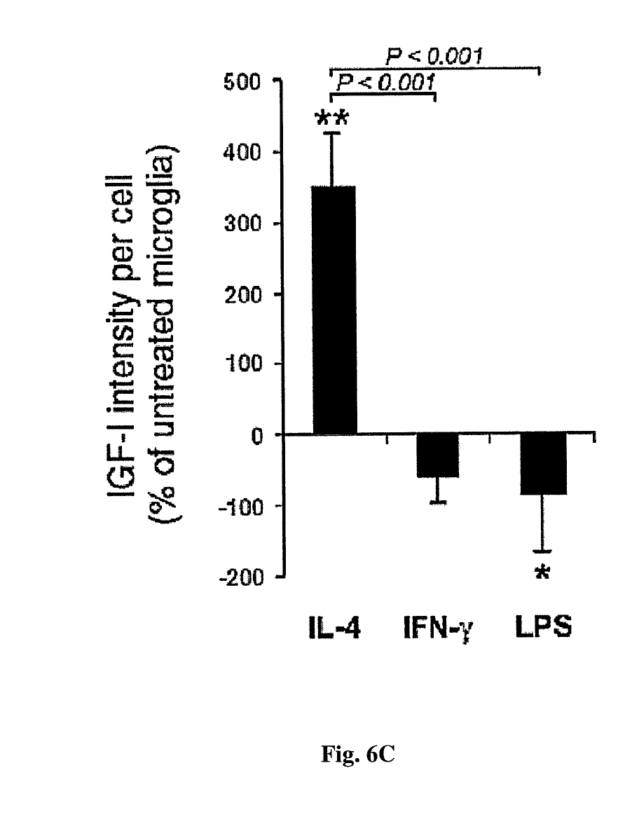

Comparative RT-PCR analyses of microglial mRNA disclosed that in the absence of activation the microglia produced both IGF-I and low levels of TNF-α. Analysis of TNF-α and IGF-I production as a function of time revealed that IFN-γ, unlike IL-4, caused a transient increase in TNF-α production and down-regulation of IGF-I (FIGS. 6A, 6B). At the protein level, quantitative immunocytochemical analysis also disclosed up-regulation of the expression of IGF-I by MG_{(IL-4)}. LPS completely blocked the production of IGF-I (FIG. 6C).

### Discussion

The results of this study strongly suggest that certain specifically activated microglia can induce neural cell renewal in the adult CNS. The findings showed that microglia can determine the fate of differentiating adult NPCs. Both neurogenesis and oligodendrogenesis were induced in NPCs co-cultured with MG_{(IL-4)}, and MG_{(IFN-γ)}, whereas both were blocked by MG_{(LPS)}, in line with reports that inflammation associated with LPS blocks adult neurogenesis (Ekdahl et al., 2003; Monje et al., 2003). NPCs co-cultured with MG_{(IL-4)} showed a bias towards oligodendrogenesis, whereas NPCs co-cultured with MG_{(IFN-γ)} were biased towards neurogenesis.

### Example 2

### Adult Hippocampal Neurogenesis in Health and Disease is Promoted by Activated Microglia Associated with Adaptive Immunity

Neurogenesis in the hippocampal dental gyrus occurs throughout life and is reportedly increased by social, mental, or physical challenges and impeded by inflammation. Here we show that hippocampal neurogenesis is promoted by conditions characterized by transient accumulation of proinflammatory T cells (Th1), such as experimental autoimmune encephalomyelitis (EAE). In vitro, we showed that whereas the Th1-associated cytokine IFN-γ at a low concentration was able to endow rat microglia with a phenotype supportive of neurogenesis from adult stems, a high concentration of this cytokine impeded neurogenesis. IL-4, antibodies specific to TNF-α, and IL-4-activated microglia could all counteract the negative effect of microglia activated by high-dose IFN-γ; and the resultant neurogenesis was significantly greater than that shown by microglia encountering IL-4 only. Injection of IL-4- or IFN-γ-activated microglia into the cerebrospinal fluid enhanced hippocampal neurogenesis in healthy rats and in rats with EAE.

### Materials and Methods

(viii) Neural progenitor cell culture was prepared as described in section (i) in Materials and Methods above in Example 1.

(ix) Primary microglial culture was prepared from brains from neonatal (P0-P1) C57B1/6J mice or Lewis rats as described in section (ii) in Materials and Methods above in Example 1.

(x) Co-culturing of mouse neural progenitor cells and mouse microglia was carried out as described in section (iii) in Materials and Methods above in Example 1. Mouse microglia were treated for 24 hours with cytokines (IFN-γ, 10 ng/ml or 100 ng/ml; IL-4, 10 ng/ml), and co-cultured with NPCs for 10 days, without insulin.

(xi) Induction of acute EAE in rats. To induce monophasic EAE, we immunized adult male Lewis rats s.c. in the hind footpad with 50 μg MBP peptide 68-86, emulsified (1:1) in 100 μl of complete Freund's adjuvant (CFA) containing 2 mg of Mycobacterium tuberculosis (strain H37Ra, Difco).

(xii) Stereotaxic injection of activated microglia. Seven days after MBP immunization, Lewis rats with monophasic EAE were injected bilaterally with syngeneic MG_{(IL-4)} (10 ng/ml) or PBS stereotaxically (1×10⁵ cells in 5 μl PBS for 5 min) into the CSF via the brain lateral ventricles (Bregma −0.8, L 1.2, V 4.5).

(xiii) Administration of 5-bromo-2′-deoxyuridine and tissue preparation. The cell-proliferation marker BrdU was dissolved by sonication in PBS and injected i.p. (50 mg/kg body weight) every 12 hours for 2.5 days, starting on day 14 after MBP immunization. One week after the first BrdU injection, the animals were deeply anesthetized and perfused transcardially, first with PBS and then with 4% paraformaldehyde. Their spinal cords were removed, postfixed overnight, and then equilibrated in phosphate-buffered 30% sucrose. Free-floating 30-μm sections were collected on a freezing microtome (Leica SM2000R) and stored at 4° C. prior to immunohistochemistry.

To study the potential effects of injected microglia on proliferation, differentiation, and survival of progenitor cells in vivo, rats in another group received BrdU injections (every 12 hours for 2.5 days). The rats were killed and their dentate gyri were analyzed 1 day or 28 days after the last BrdU injection.

(xiv) Immunocytochemistry and immunohistochemistry. Immuno-cytochemistry was carried out as described in section (iv) in Materials and Methods above in Example 1. The staining was performed with a combination of the mouse anti-tubulin β-III-isoform C-terminus antibodies (β-III-tubulin; 1:500) and goat anti-doublecortin (DCX; 1:400; Santa Cruz Biotechnology, Santa Cruz, Calif.).

For immunohistochemistry, tissue sections were treated with a permeabilization/blocking solution containing 10% FCS, 2% bovine serum albumin, 1% glycine, and 0.05% Triton X-100 (Sigma-Aldrich, St. Louis). Primary antibodies were applied for 1 hour in a humidified chamber at room temperature. For BrdU staining, sections were washed with PBS and incubated in 2N HCl at 37° C. for 30 min. Sections were blocked for 1 hour with blocking solution. The tissue sections were stained overnight with specified combinations of the following primary antibodies: rat anti-BrdU (1:200; Oxford Biotechnology, Kidlington, Oxfordshire, UK), goat anti-DCX (1:400; Santa Cruz Biotechnology), and mouse anti-NeuN (1:200; Chemicon, Temecula, Calif.). For labeling of microglia we used FITC-conjugated Bandeiraea simplicifolia isolectin B4 (IB4, 1:50; Sigma-Aldrich, Rehovot). For labeling of activated microglia stereotaxically injected in naïve rats we used mouse anti-EDI (ED1; 1:250; Oxford, Serotec, UK). To detect expression of cell-surface MHC-II proteins we used anti-MHC-II Abs (mouse, 1:50; IQ Products, Groningen, The Netherlands). Expression of IGF-I was detected by goat anti-IGF-I Abs (1:20; R&D Systems). Secondary antibodies used were FITC-conjugated donkey anti-goat, Cy-3-conjugated donkey anti-mouse and Cy-3- or Cy-5-conjugated donkey anti-rat (1:200; Jackson ImmunoResearch, West Grove, Pa.). Control sections (not treated with primary antibody) were used to distinguish specific staining from staining of non-specific antibodies or autofluorescent components. Sections were then washed with PBS and coverslipped in polyvinyl alcohol with diazabicylo-octane as anti-fading agent.

(xv) Quantification and stereological counting procedure. For microscopic analysis we used a Zeiss LSM 510 confocal laser scanning microscope (40× magnification). For experiments in vitro we scanned fields of 0.053 mm² (n=8-16 from at least two different coverslips) for each experimental group. For each marker, 500-1000 cells were sampled. Cells co-expressing GFP and β-III-tubulin were counted. For in-vivo experiments we analyzed nine coronal sections (30 μm) throughout the entire dentate gyrus at 300-μm intervals in each rat. Proliferation and neurogenesis in the dentate gyrus was evaluated by automatic counting of BrdU⁺, BrdU⁺/DCX⁺ or BrdU⁺/NeuN⁺ cells using Image-Pro Plus 4.5 software (Media Cybernetics). Specificity of BrdU⁺/NeuN⁺ co-expression was assayed using the confocal microscope (LSM 510) in optical sections at 1-μm intervals.

(xvi) Statistical analysis. The in-vitro results were analyzed by the Tukey-Kramer multiple comparisons test (ANOVA) and are expressed as means±SD. In-vivo results were analyzed by Student's t-test or one-way ANOVA and are expressed as means±SEM.

### Example 2(1)

### MG_{(IL-4)} Induces Oligodendrogenesis in Adult Rats

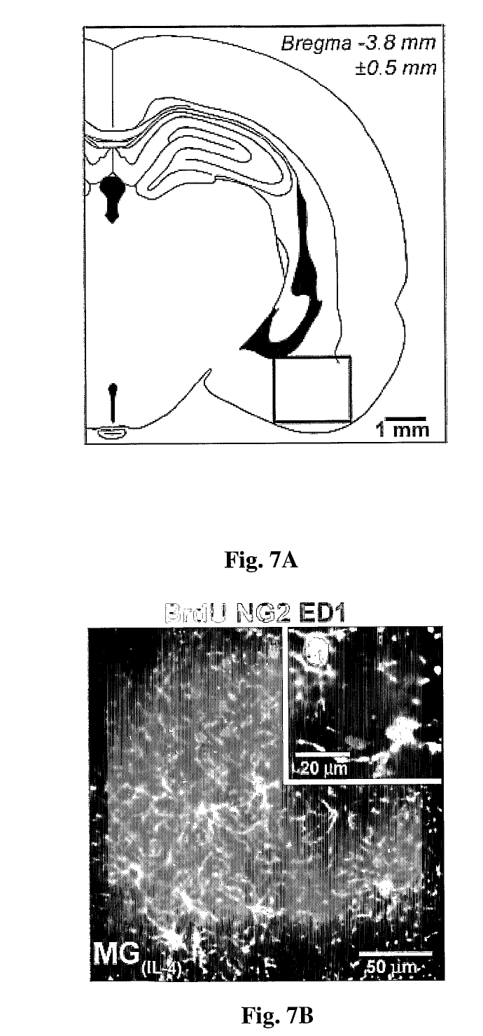

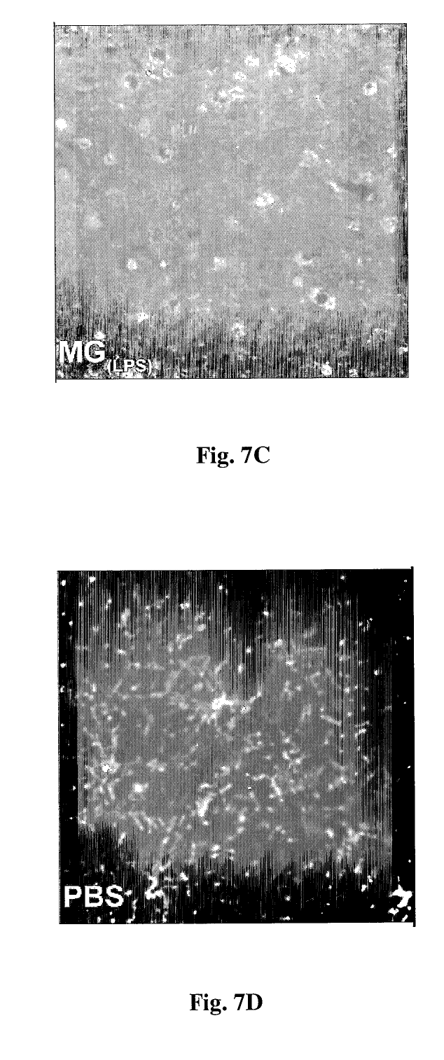

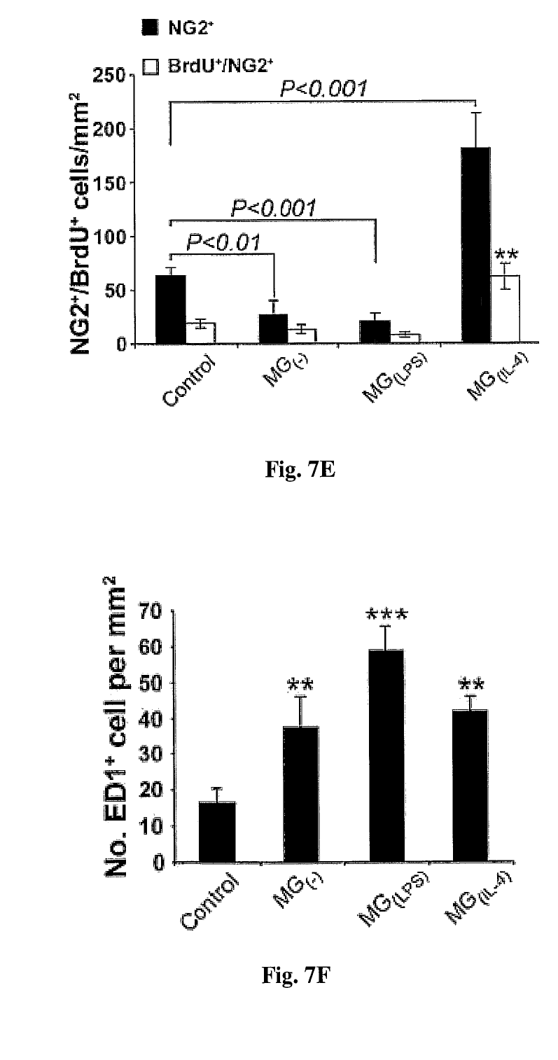

To determine whether MG_{(IL-4)} would create conditions conducive to oligodendrogenesis in vivo, we injected MG_{(LPS)}, MG_{(IL-4)} or MG₍₋₎ into the right lateral ventricle of the rat (SPD rats, 10-12 weeks old) brain and examined whether injected activated microglia have any effect on oligodendrogenesis. Control rats were injected with PBS instead of microglia. Twenty-four hours later rats were injected with BddU intraperitoneally every 12 hours for 3.5 days. After 28 days, the brains were excised and coronal sections taken from the hippocampus were analyzed. Newly formed oligodendrocytes were found mainly in the brain parenchyma adjacent to the injected ventricle (FIG. 7A). All of the differently activated microglia (ED1⁺), but mostly MG_{(LPS)}, were seen in this area. However, as in the case of neurogenesis, these microglia blocked oligodendrogenesis. In contrast, rats injected with MG_{(IL-4)} showed an increase in the number of NG2⁺ cells that were also stained with BrdU (NG2⁺/BrdU⁺) (FIG. 7B). Such cells, representing newly formed immature oligodendrocyte (Dawson et al., 2003), were found in close proximity to activated microglia (ED1⁺), but none of them co-expressed NG2 and ED1 (FIG. 7B). No NG2⁺/BrdU⁺ cells were detected in MG_{(LPS)}-treated rats (FIG. 7C). Interestingly, in the PBS-injected control rats we detected activated microglia adjacent to NG2⁺ cells (FIG. 7D). Since NG2 also stains the extracellular matrix protein chondroitin sulfate proteoglycan (Jones et al., 2002), we took special care in our quantitative analysis to count only the double-labeled cells (NG2⁺/BrdU⁺). NG2⁺ cells were found only on the side of the microglial injection. Quantitative analysis revealed that oligodendrogenesis was induced by MG_{(IL-4)} (FIG. 7E). FIG. 7F shows the density of the differently activated microglia at the examined areas.

### Example 2(2)

### Microglial Ability to Induce Neurogenesis from Adult Neural Stem/Progenitor Cells Correlates with Balanced Levels of IFN-γ

We showed in Example 1 above that both neurogenesis and oligodendrogenesis of adult NPCs in vitro are blocked by inflammation-associated (endotoxin-activated) microglia but induced by microglia activated by cytokines (IFN-γ or IL-4) associated with Th cells. The same microglia were found, respectively, to be capable of blocking neuronal cell survival and of protecting neurons against degeneration. In the case of microglial activation by IFN-γ, however, the effect was beneficial only over a narrow range of concentrations, beyond which it turned destructive. The observation that inflammation in patients suffering from autoimmune diseases of the CNS is characterized by excessive quantities of Th1, and therefore of IFN-γ, prompted us to examine whether such situations might also be associated with impaired neurogenesis, and if so, whether there are ways to reverse them.

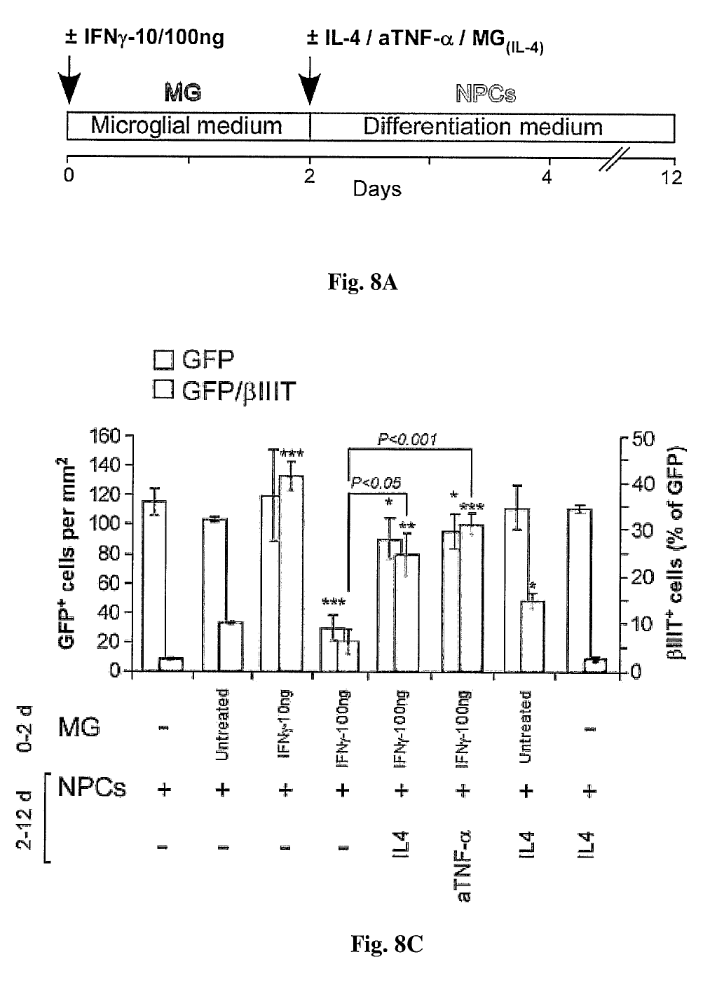

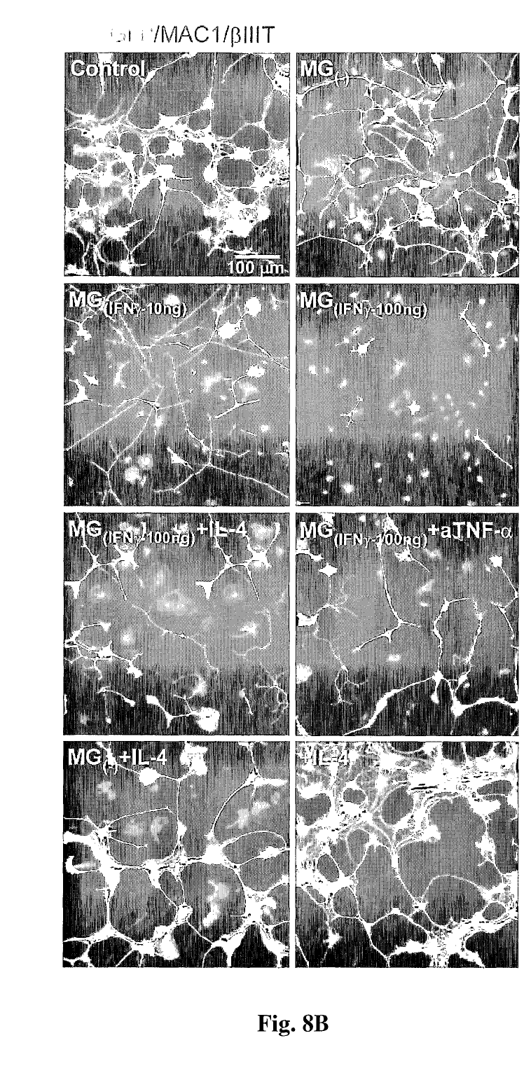

We examine here the effects on NPCs of microglia that were pre-incubated in their optimal growth media for 48 hours in the presence of a low dose (10 ng/ml) or a high dose (100 ng/ml) of IFN-γ. NPCs without microglia were tested as a control. After the growth media and cytokine residues were washed off, each of the treated microglial preparations was freshly co-cultured with dissociated NPC spheres on coverslips coated with Matrigel® in the presence of differentiation medium, with each of the treated microglial preparations as schematically depicted in FIG. 8A. We used NPCs labeled with GFP to verify that any differentiation of neurons seen in the cultures was derived from the NPCs themselves rather than from contamination of the primary microglial cultures. After 10 days we could discern GFP-expressing NPCs co-labeled with the neuronal marker P-1-tubulin (FIG. 8B). In NPCs cultured without microglia (control) or with nonactivated microglia (MG₍₋₎), only a few cells positive for both GFP and β-III-tubulin (GFP⁺/β-III-tubulin⁺ cells) were detectable (FIG. 8B). In NPCs co-cultured with microglia activated by low-dose IFN-γ (MG_{(IFN-γ,10 ng)}), the increase in numbers of GFP⁺/β-III-tubulin⁺ cells was dramatic. In contrast, in co-cultures with microglia activated by high-dose IFN-γ (MG_{(IFN-γ,100 ng)}) the numbers of NPCs were significantly decreased and no differentiation could be detected, indicating an impediment to neurogenesis. This negative effect could be partially counteracted by the addition of IL-4 (MG_{(IFN-γ,100 ng)}+IL-4), and the resulting neurogenesis, in terms of morphological maturation, was more robust than that induced by MG₍₋₎ to which IL-4 was added. Direct addition of IL-4 to NPCs co-cultured with MG₍₋₎ caused a slight but significant increase in the expression of the neuronal marker, β-III-tubulin on NPCs, but had no effect on NPCs cultured alone (FIG. 8B).

Addition of neutralizing anti-TNF-α antibodies (ãTNF-α) to MG_{(IFN-γ,100 ng)} resulted in a significant increase in the number of β-III-tubulin-positive cells in their co-cultured NPCs (FIG. 8B). In contrast, ãTNF-α were ineffective when added to co-cultures of NPCs and MG_{(IL-4)}. Quantitative analysis verified that ãTNF-α and (to a lesser extent) IL-4 had partially counteracted the inhibitory effect of MG_{(IFN-γ,100 ng)} on neurogenesis (FIG. 8C).

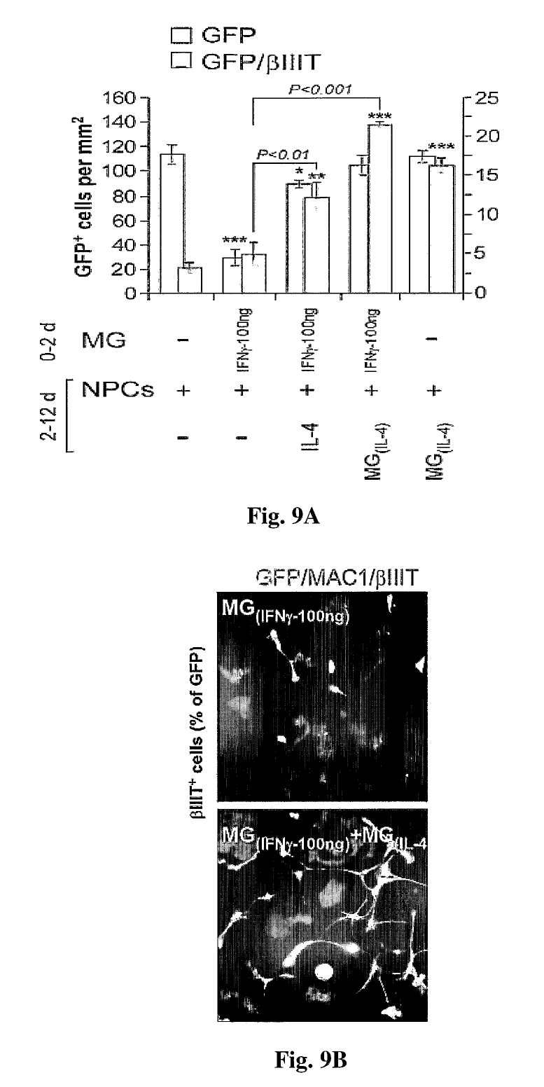

We demonstrate hereinafter that IL-4-activated microglia exhibit neuroprotective features, at least in part via production of IGF-I. Because MG_{(IFN-γ,100 ng)} decreased NPC survival (FIG. 8), we suspected that MG_{(IL-4)} might be capable of reversing that detrimental effect. We therefore examined whether not only IL-4 (as shown above) but also MG_{(IL-4)} would be sufficient to partially counteract the effect of high-dose IFN-γ. FIG. 9 shows that MG_{(IL-4)} were indeed effective in partially counteracting the negative effect of IFN-γ-activated microglia in terms of NPC survival and differentiation.

### Example 2(2)

### Microglia Activated by IL-4 or by IFN-γ can Promote Neurogenesis from Endogenous Stem/Progenitor Pools in Naïve Adult Rats

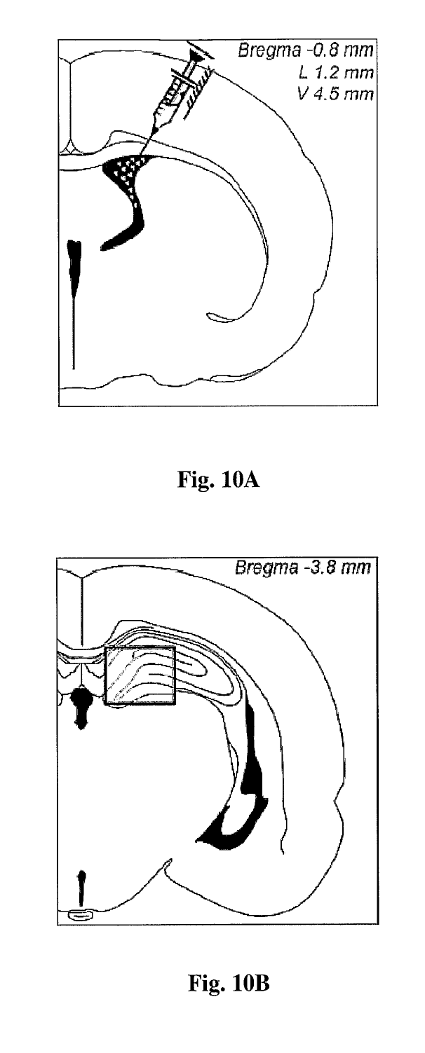

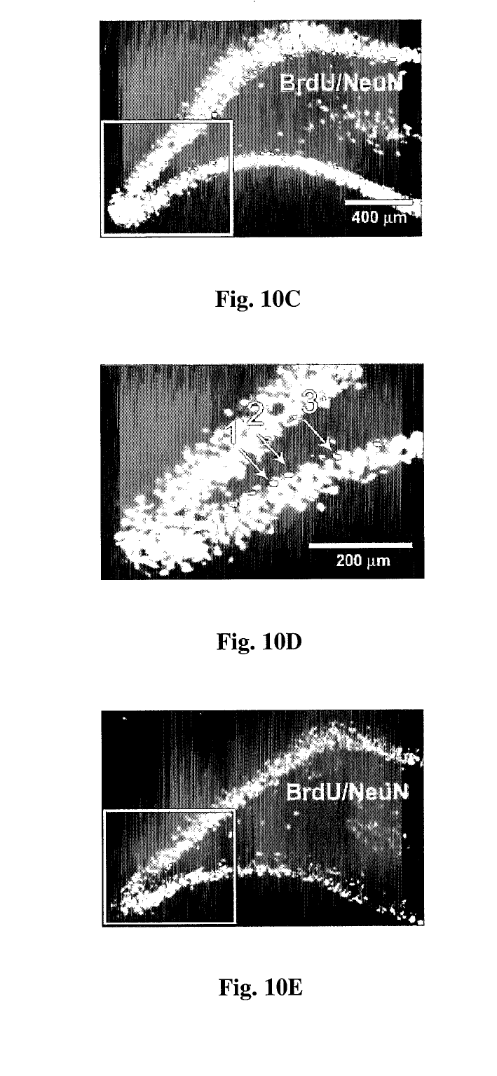

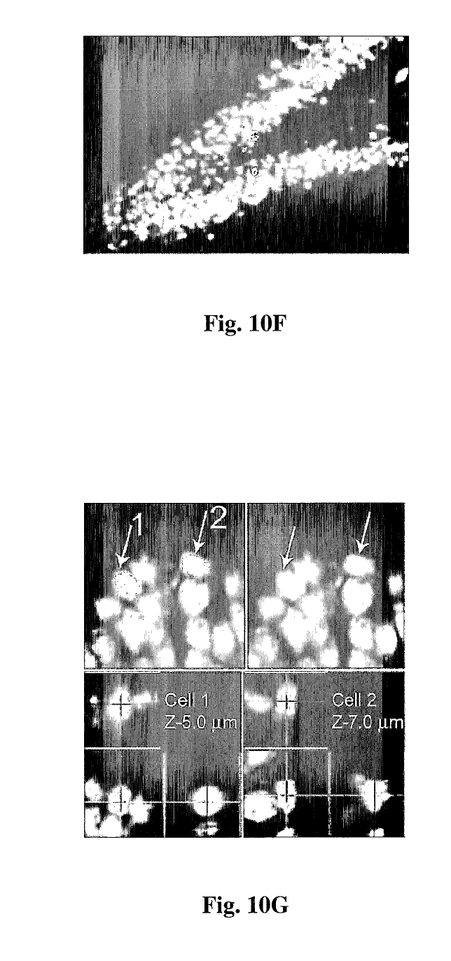

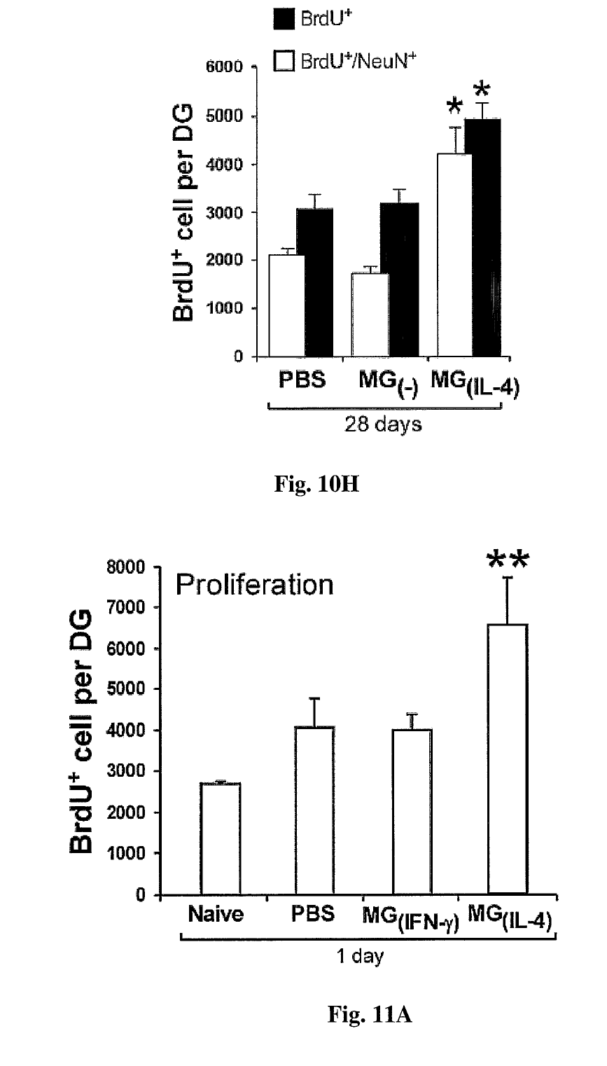

The above results prompted us to examine whether MG_{(IL-4)} are beneficial in vivo under pathological conditions of impeded neurogenesis associated with an excess of Th1 cells. The right lateral ventricles of healthy rat brains were injected with rat microglia (MG₍₋₎ or MG_{(IL-4)}) (FIG. 10A) or with PBS. Starting 24 hours later, the rats were injected intraperitoneally (i.p.) with BrdU every 12 hours for 3.5 days to identify proliferating cells. After 28 days the brains were excised and coronal sections were taken from the hippocampus and analyzed (FIG. 10B). Sections from the hippocampal dentate gyrus of rats injected with MG_{(IL-4)} showed significantly more proliferating cells (FIGS. 10C, 10D) than did corresponding sections from PBS-injected rats (FIGS. 10E, 10F). Many of the newly proliferating cells had differentiated into mature neurons, as indicated by their double labeling with neuronal nuclear antigen (NeuN), a marker of mature neurons, and BrdU (FIG. 10G). Quantitative analysis 28 days after BrdU injection disclosed no significant differences in hippocampal neurogenesis between naïve rats injected with MG₍₋₎ and those injected with PBS (FIG. 10H), but showed a significant effect of MG_{(IL-4)}.

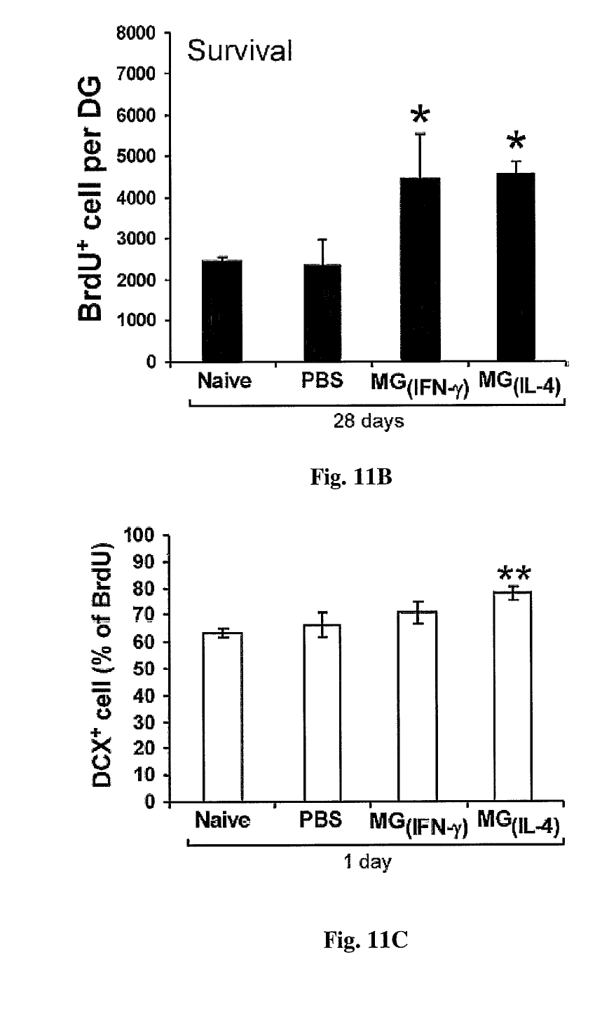

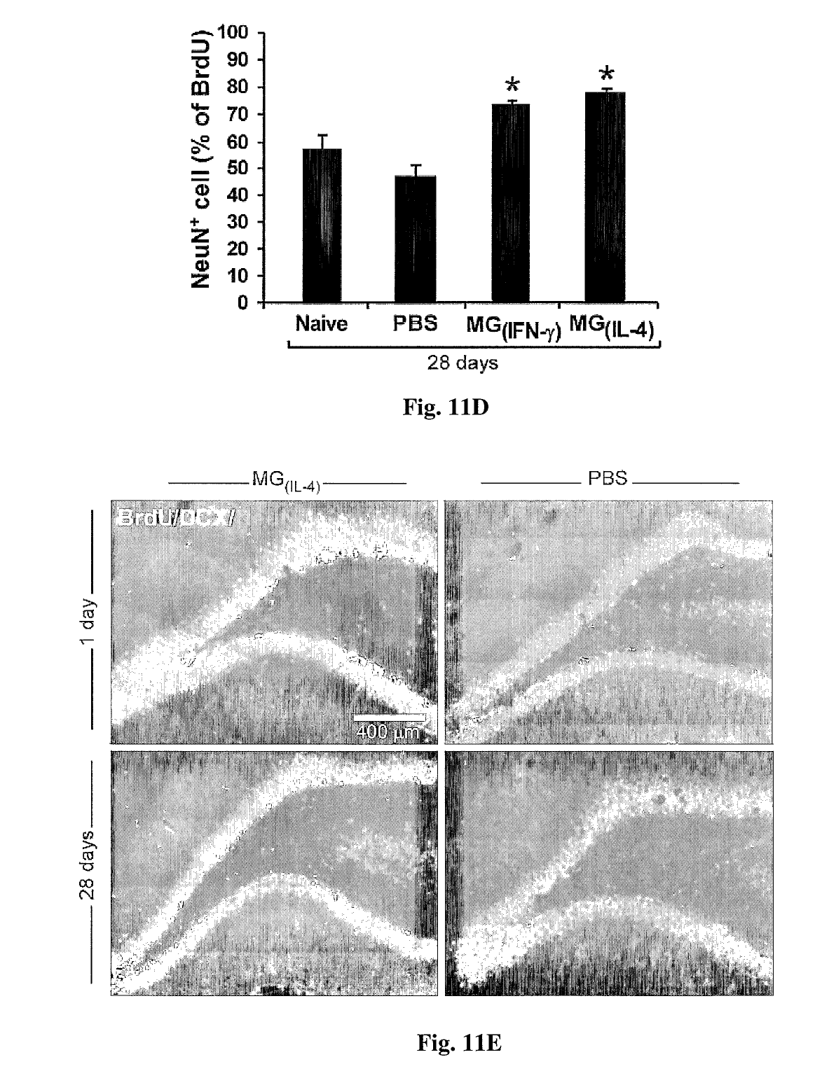

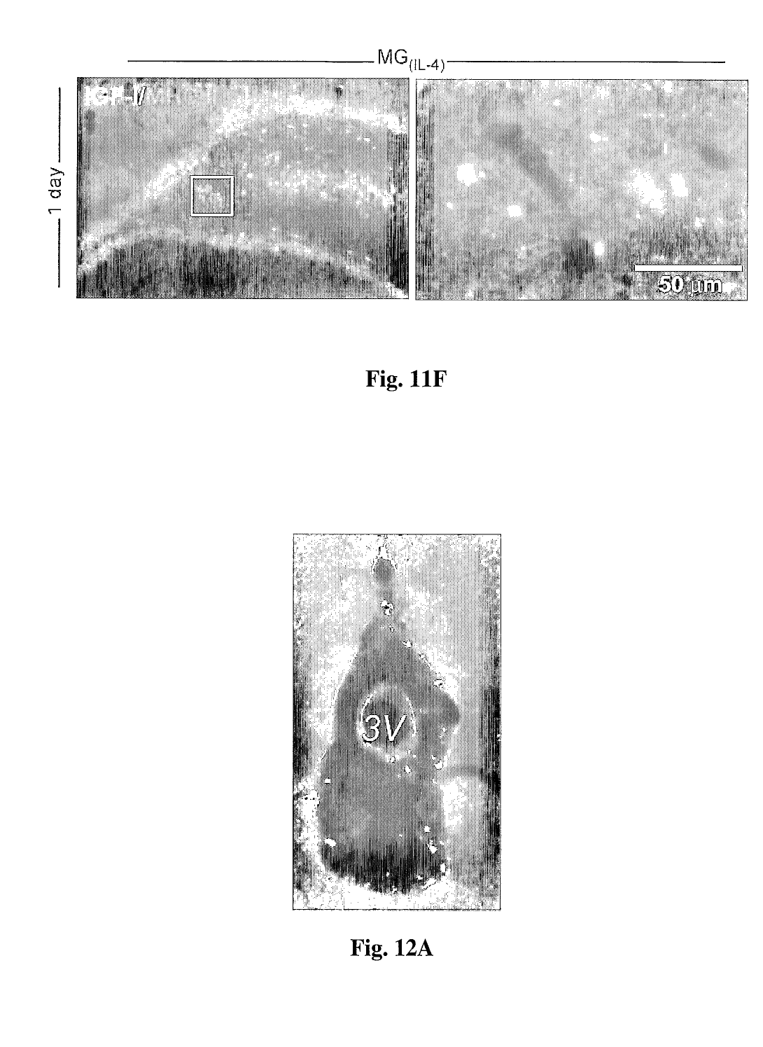

To determine whether the MG_{(IL-4)}-induced increase in neurogenesis occurs via an effect on proliferation and differentiation or on survival of the newly formed neurons, we repeated the above experiments and also examined the effect of MG_{(IFN-γ)} (FIG. 11). From each group we examined five rats for proliferation and differentiation 24 hours after the last BrdU injection, and another five rats 28 days later for differentiation and survival. At the 24-hour examination there were already significantly more proliferating (BrdU⁺) cells (approx. 30% more) in the MG_{(IL-4)}-injected rats than in the PBS-injected controls (FIG. 11A). Analysis at 28 days also disclosed significantly more surviving cells in the MG_{(IFN-γ)}- and MG_{(IL-4)}-injected rats than in the controls (FIG. 11B). At each of the tested time points we calculated the percentages of differentiation of BrdU⁺ cells into pre-mature (DCX⁺ cells) and mature (NeuN⁺ cells), 1 day and 28 days after the last BrdU injection, respectively (FIGS. 11C, 11D). Comparison of the mean number of newly differentiated BrdU⁺/DCX⁺ cells 1 day after the last BrdU injection, expressed as a percentage of the total number of BrdU⁺ cells, indicated that the only group in which the percentage was significantly higher than in the PBS-treated control group was the group treated with MG_{(IL-4)} (FIG. 11C). Similar analysis at 28 days, when we used the NeuN⁺ cell number expressed as a percentage of the total number of BrdU⁺ cells as a measure of survival of newly formed neurons, disclosed that the percentages in both the MG_{(IFN-γ)}-treated and the MG_{(IL-4)}-treated groups were higher than in either the PBS-treated control group or naïve rats (FIG. 1D). These results suggested that although the injected MG_{(IL-4)} had some beneficial effect on survival of the newly formed cells their effects on proliferation and differentiation were significantly stronger, whereas the main beneficial effect of MG_{(IFN-γ)} was on survival and possibly also on differentiation of the newly formed neurons, not on their proliferation. Representative confocal images of hippocampal slices of rat brains excised 1 and 28 days after BrdU injection illustrates the effect of MG_{(IL-4)} on proliferation and survival of newly formed neurons (FIG. 1E). In attempting to a find a link between the increased neurogenesis and microglial activation, we searched for microglia expressing both class II major histocompatibility complex proteins (MHC-II) and IGF-I in hippocampal sections that were analyzed 24 hours after BrdU injection (corresponding to 5 days after microglial injection). Microglia exhibiting this dual expression were detected in the hippocampal dentate gyri of rats injected with MG_{(IL-4)}, and only rarely in naïve or PBS-injected rats (FIG. 11F).

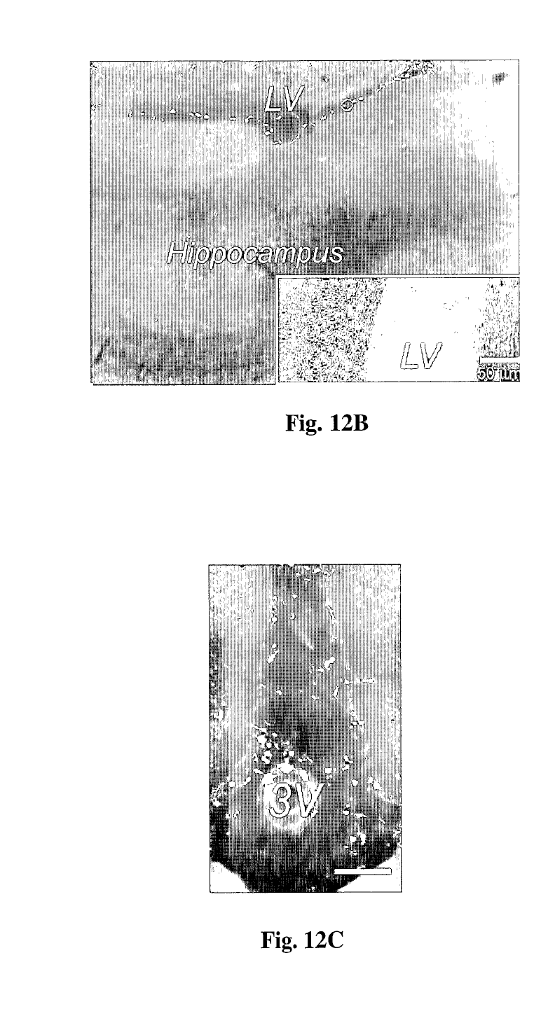

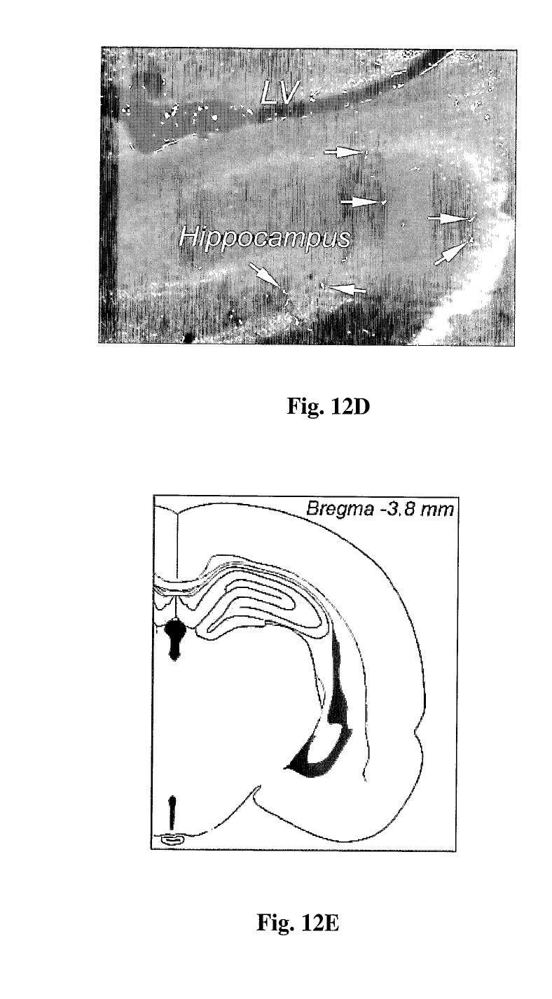

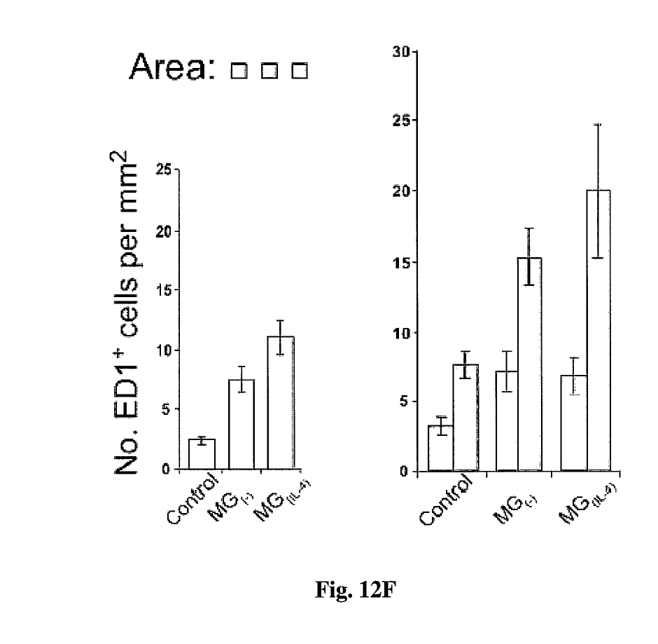

We also examined the distribution of activated microglia (stained with activated microglial marker EDI) adjacent to the site of microglial injection in these brain sections. Analysis of brains in the first set of experiments (FIGS. 10C-10F) for distribution of EDI-stained activated microglia (both resident and injected cells) disclosed a large number of ED1 cells in the hippocampi of rats in the groups injected with microglia (FIGS. 12A-F). These findings encouraged us to investigate the effects of such microglia under conditions of acute EAE.

### Example 2(3)

### Effect of MG_{(IL-4)} on Neurogenesis in Rats with Acute EAE

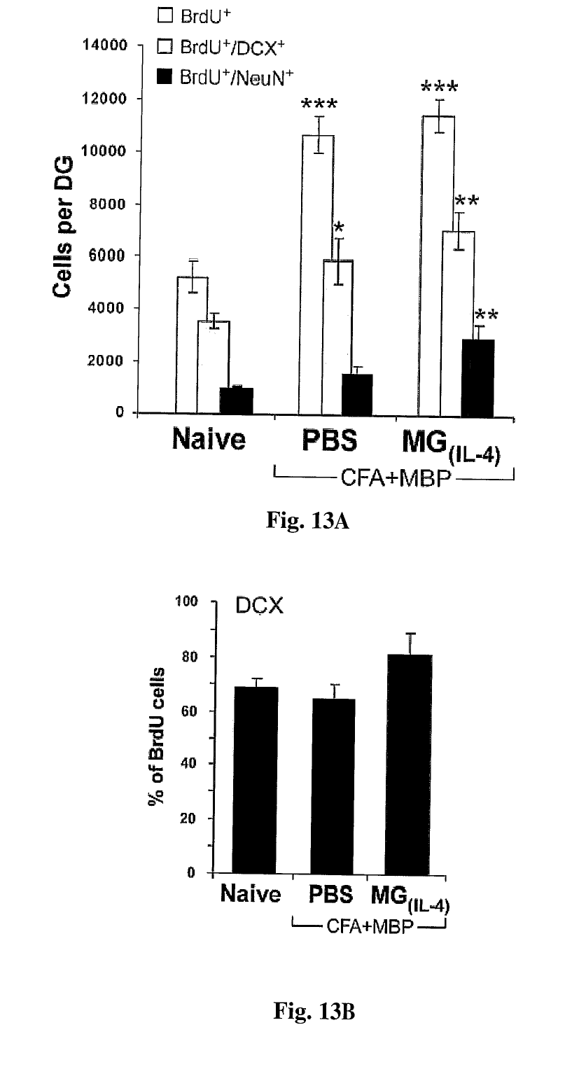

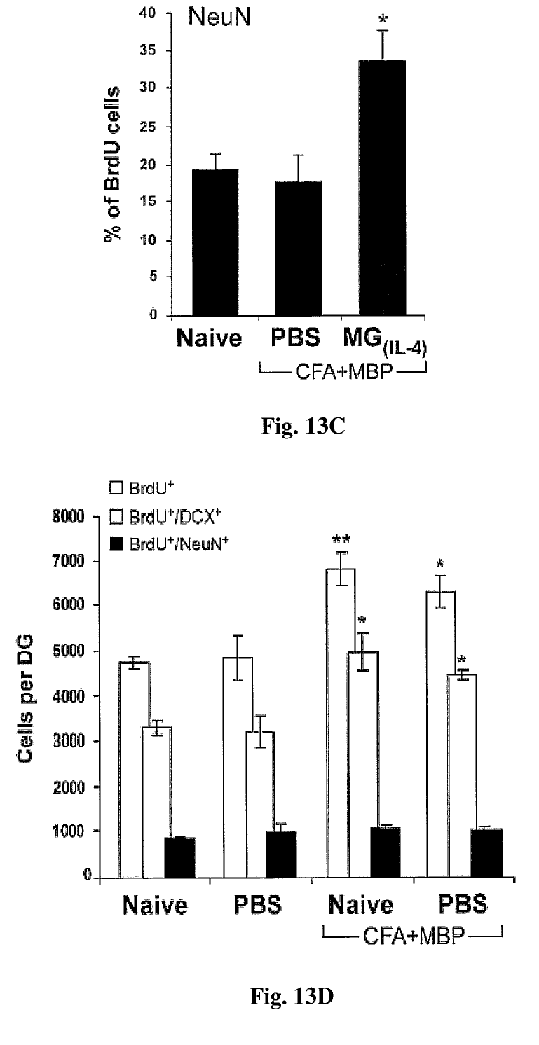

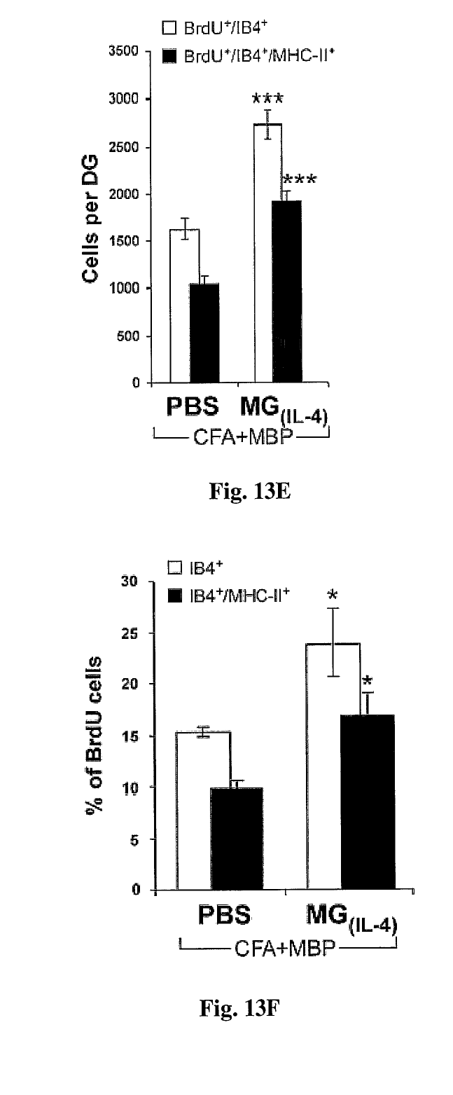

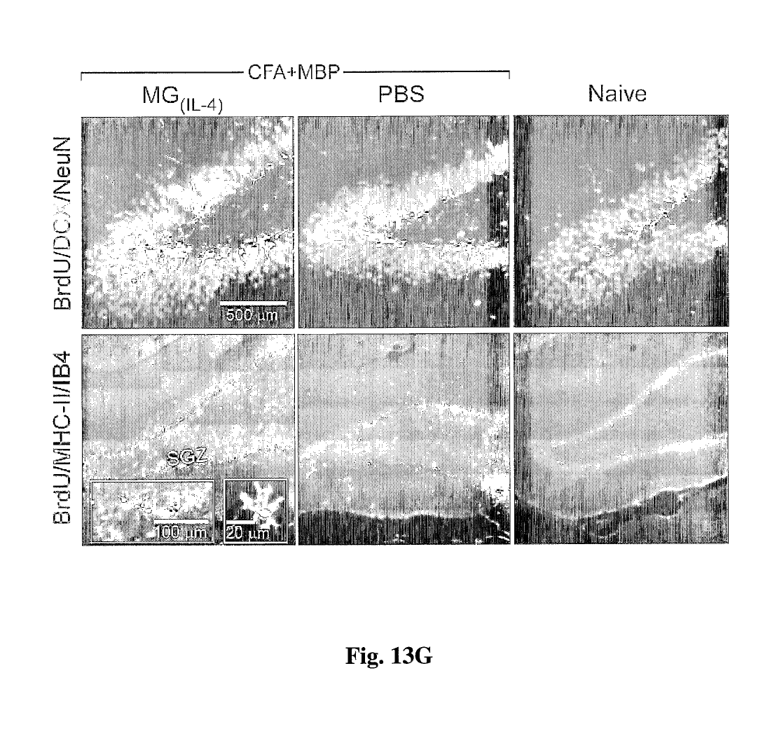

Acute EAE is known to be associated with a transient increase in accumulation of encephalitogenic Th1 cells that recognize myelin antigens. We immunized 16 adult Lewis rats with MBP emulsified in CFA. The resulting transient hindlimb paralysis peaked on day 14 and then gradually resolved. Seven days after the immunization (1 day earlier than the reported average time of clinical onset of induced EAE), we subjected eight rats to bilateral stereotaxic injection of syngeneic MG_{(IL-4)} into the cerebrospinal fluid (CSF) lateral ventricles. The other eight rats were similarly injected with PBS. On day 14 after immunization (7 days after MG_{(IL-4)} injection) the rats were injected intraperitoneally (i.p.) with BrdU every 12 hours for 2.5 days to identify proliferating cells. Seven days after the first BrdU injection the dentate gyrus of each rat was examined immunohistochemically for evidence of newly formed neurons. The dentate gyrus of each rat was subjected to quantitative immunohistochemical analysis for evidence of newly formed neurons, which revealed an increase in proliferating (BrdU⁺) cells. Differentiation of these NPCs into neurons was indicated by their double labeling with DCX. The number of newly-formed neurons (BrdU⁺/DCX⁺ cells) in sections from the dentate gyrus was significantly higher in the MBP-immunized rats than in naïve rats, and was further increased by injection of MG_{(IL-4)} (FIG. 13A). When their BrdU⁺/DCX⁺ cells were expressed as percentages of their total numbers of BrdU⁺ cells, however, the MBP-immunized rats did not differ from the naïve rats (FIG. 13B), suggesting that the immunization had affected the numbers of proliferating NPCs but not their rates of differentiation. In contrast, the percentage seen in the MG_{(IL-4)}-injected rats was significantly higher than in the PBS-injected controls (FIG. 13C), suggesting that the effect of MG_{(IL-4)} on neurogenesis was exerted via both proliferation and differentiation. To exclude the possibility that the increased neurogenesis observed in the MBP-immunized, PBS-injected rats relative to naïve rats was not attributable to the PBS injection (due to the intervention in the lateral ventricle), we repeated the above experiment with four groups: naïve rats with or without PBS injection and MBP-immunized rats with or without PBS injection. No neurogenesis was seen in either group of naïve rats, whereas both of the MBP-immunized groups showed increased neurogenesis as before (FIG. 13D).

To substantiate our suspicion that activated microglia would be found among the newly proliferating cells in the immunized rats, we stained the hippocampal sections for the microglial marker IB4 and for MHC-II (FIG. 13E). More than 60% of the newly formed microglia in PBS-injected rats and more than 70% in the MG_{(IL-4)}-injected rats showed co-labeling of these markers (FIG. 13E). No newly formed microglia were seen in naïve rats. The number of IB4+/MHC-II⁺ cells, expressed as a percentage of the total number of BrdU⁺ cells, was significantly higher in rats injected with MG_{(IL-4)} than with PBS (FIG. 13F). The observed microglia were located in the vicinity of the subgranular zone of the dentate gyrus (FIG. 13G).

We concluded that a transient local immune response induced by pro-inflammatory cytokines is capable of supporting neurogenesis.

### Discussion

The results of this study showed that whereas IFN-γ at a low concentration was able to endow rat microglia in vitro with a phenotype supportive of adult neurogenesis from stem-cell pools, a high concentration of this cytokine impeded neurogenesis. IL-4, ãTNF-α, and MG_{(IL-4)} could all counteract the negative effect of high-dose MG_{(IFNγ)} on neurogenesis. It thus became apparent that moderate amounts of IFN-γ activate microglia to create conditions that are more favorable for differentiation of adult NPCs to neurons than those created by IL-4, but that IL-4 or MG_{(IL-4)} can counteract the negative side effect of an overabundance of IFN-γ, at least in vitro. In apparent correlation in vivo, acute EAE promoted adult neurogenesis in the rat hippocampal dentate gyrus. Moreover, in naïve rats or in rats with acute EAE an injection of MG_{(IL-4)} into the CSF increased neurogenesis from stem-cell pools.

### Example 3

### Induction and Blockage of Oligodendrogenesis from Endogenous Adult Neural Stem Cells by Differently Activated Microglia: Implications for Multiple Sclerosis

The role of activated microglia in demyelinating neurodegenerative diseases is controversial. We show that high levels but not low levels of interferon-γ (IFN-γ, a cytokine associated with inflammatory autoimmune diseases) confer on rodent microglia a phenotype that impedes oligodendrogenesis from adult neural stem/progenitor cells. Interleukin (IL)-4, reversed the impediment, attenuated TNF-α production, and overcame blockage of insulin like growth factor (IGF)-I production caused by IFN-γ. In rodents with acute or chronic experimental autoimmune encephalomyelitis, injection of IL-4-activated microglia into the cerebral spinal fluid resulted in improved clinical symptoms and increased oligodendrogenesis in the spinal cord, spatially associated with microglia expressing MHC-II and IGF-I. These results point to a novel role for microglia in oligodendrogenesis from the endogenous stem cell pool.

Here we examined whether IL-4, via modulation of microglia both in vitro and in vivo, can overcome the destructive effects of high-dose IFN-γ, known to be associated with EAE. In vitro, a high dose of IFN-γ, but not a low dose, impaired the ability of microglia to support oligodendrogenesis from adult neural stem cells/progenitor cells (NPCs). IL-4 counteracted the interference with oligodendrogenesis caused by high IFN-γ concentrations. When IL-4-activated microglia were stereotaxically injected through the cerebral ventricles into the cerebrospinal fluid (CSF) of rats with acute EAE or of mice with a remitting-relapsing autoimmune disease, the animals demonstrated significantly more oligodendrogenesis and significantly less neurological deficit than did their vehicle-injected diseased controls.

### Materials and Methods

(xvii) Neural progenitor cell culture was prepared as described in section (i) in Materials and Methods above in Example 1.

(xviii) Primary microglial culture was prepared from brains from neonatal (P0-P1) C57B1/6J mice or Lewis rats as described in section (ii) in Materials and Methods above in Example 1.

(xix) Co-culturing of mouse neural progenitor cells and mouse microglia was carried out as described in section (iii) in Materials and Methods above in Example 1.

(xx) Induction and evaluation of acute and chronic EAE. To induce chronic EAE we injected adult male C57B1/6J mice s.c. with 200 μg (300 μl) of myelin oligodendrocyte glycoprotein peptide 35-55 (MOG)₃₅₋₅₅ (Multiple Peptide System) in incomplete Freund's adjuvant containing 8 mg/ml Mycobacterium tuberculosis (strain H37Ra; Difco). Pertussis toxin (Sigma Aldrich; 500 ng) was injected on the day of the immunization and again 2 days later.

Induction of monophasic EAE in adult male Lewis rats was carried out as described in section (xi) of Example 2 above. Clinical signs were evaluated in a blinded fashion by at least two investigators. Body weight and clinical score were recorded daily (0=healthy; 1=limb tail paralysis; 2=ataxia and/or paresis of hindlimbs; 3=paralysis of hindlimbs and/or paresis of forelimbs; 4=tetraparalysis; 5=moribund state or death).

(xxi) Stereotaxic injection of activated microglia. Seven days after the vaccination, Lewis rats with monophasic EAE were injected bilaterally with syngeneic MG_{(IL-4)} (10 ng/ml) or PBS stereotaxically (1×10⁵ cells in 5 μl PBS for 5 min) into the CSF via the brain lateral ventricles (Bregma −0.8, L 1.2, V 4.5). Ten days after the vaccination, C57BL/6J mice with chronic EAE received bilateral stereotaxic injections of syngeneic MG_{(IL-4)} (10 ng/ml) or PBS (1×10⁵ cells in 3 μl PBS for 3 min) into the CSF via the brain lateral ventricles (Bregma −0.4, L 0.8, V 2.5).

(xxii) Administration of 5-bromo-2′-deoxyuridine and tissue preparation were carried out as described in section (xiii) of Example 2 above. BrdU was injected i.p. starting on day 14 after MBP vaccination in adult male Lewis rats or on day 20 after MOG vaccination in adult male C57BL/6J mice. One week (rats) or 2 weeks (mice) after the first BrdU injection, the animals were deeply anesthetized and perfused transcardially as described.

(xxiii) Immunocytochemistry and immunohistochemistry. For immunocytochemistry, co-cultures of NPCs and mouse microglia treated as described in section (iv) of Example 1 and stained with a combination of the rabbit anti-NG2 chondroitin sulfate proteoglycan (NG2; 1:500) and mouse anti-RIP (RIP; 1:2000). To capture the microglia we used FITC-conjugated Bandeiraea simplicifolia isolectin B4 (IB4; 1:50; Sigma-Aldrich). Expression of IGF-I was detected by goat anti-IGF-I (1:20; R&D Systems).

For immunohistochemistry, tissue sections were treated as described in section (xiv) of Example 2. After blocking the sections for 1 h with blocking solution, the tissue was then stained with rat anti-BrdU (1:200; Oxford Biotechnology) in combination with rabbit anti-NG2 (1:300) and mouse anti-RIP (1:1000) antibodies diluted in PBS containing 0.05% Triton X100, 0.1% Tween 20, and 2% horse serum. For labeling of microglia we used IB4 (1:50). To detect expression of cell-surface MHC-II proteins we used mouse anti MHC-II Abs (1:50; IQ Products). Expression of IGF-I was detected by goat anti IGF-I Abs (1:10-1:100; R&D Systems). Sections were incubated with the primary antibody for 24 h at 4° C., washed with PBS, and incubated with the secondary antibodies in PBS for 1 h at room temperature while protected from light. Secondary antibodies used for both immunocytochemistry and immunohistochemistry were Cy-3-conjugated donkey anti-mouse, Cy-3-conjugated goat anti-rabbit, Cy-5-conjugated goat anti-rat, Cy-2-conjugated goat anti-rat, and Cy-5-conjugated donkey anti-goat. Of Example 1 abovetibodies were purchased from Jackson ImmunoResearch Laboratories and used at a dilution of 1:250-500.

(xxiv) Real-time quantitative PCR analysis. Total cellular RNA purification and cDNA synthesis was performed as described in section (v) of Example 1 above. We assayed the expression of specific mRNAs using real-time quantitative PCR (Q-PCR) with selected gene-specific primer pairs. Q-PCR reactions were performed with a high-speed thermal cycler (LightCycler; Roche Diagnostics), and the product was detected by FastStart Master SYBR Green I (Roche Molecular Biochemicals) according to the manufacturer's instructions. The amplification cycle was 95° C. for 10 s, 60° C. for 5 s, and 72° C. for 10 s. The primers used were:

| | |
|---|---|
| for IGF-I, sense, 5′-CCGGACCAGAGACCCTTTG-3′ | (SEQ ID NO:9), |
| antisense 5′-CCTGTGGGCTTGTTGAAGTAAAA-3′ | (SEQ ID NO:10); |
| for TNF-α, sense 5′-ACAAGGCTGCCCCGACTAT-3′ | (SEQ ID NO:11), |
| antisense 5′-CTCCTGGTATGAAGTGGCAAATC-3′ | (SEQ ID NO:12). |

Melting curve analysis confirmed that only one product was amplified.

(xxv) Quantification and stereological counting procedure. For microscopic analysis we used a Zeiss LSM 510 confocal laser scanning microscope (40× magnification). For experiments in vitro we scanned fields of 0.053 mm² (n 8-16 from at least two different coverslips) for each experimental group. For each marker, 500-1000 cells were sampled. Cells co-expressing GFP, NG2 and RIP were counted.

For in-vivo experiments with rats and mice with EAE, oligodendrogenesis and proliferation of microglia in the spinal cord were evaluated by counting of cells that were double- or triple-labeled with BrdU and markers of premature oligodendrocytes (NG2) and a pre-ensheathing marker of oligodendrocytes (RIP), microglia (IB4), or antigen-presenting cells (MHC-II) from sagittal longitudinal sections at segment T8-T9 of the spinal cord. The numbers of cells per mm³ were counted at 300-μm intervals in gray and white matter in each rat or mouse (n=8 per group). Specificity of BrdU+/NG2⁺ or BrdU⁺/RIP⁺ or BrdU⁺/IB4⁺ or BrdU⁺/IB4⁺/MHC-II⁺ co-expression was assayed using the confocal microscope (LSM 510) in optical sections at 1-μm intervals. Counting was evaluated automatically using Image-Pro Plus 4.5 software (Media Cybernetics).

(xxvi) Statistical analysis. The in-vitro results were analyzed by the Tukey-Kramer multiple comparisons test (ANOVA) and are expressed as means±SD. In-vivo results were analyzed by Student's t-test or one-way ANOVA and are expressed as means±SEM. Significance of the EAE score was analyzed by Mann-Whitney test, two-factor repeated measures (ANOVA).

### Example 3(1)

### Effects of Microglia on Oligodendrogenesis In Vitro: the Dual Effect of IFN-γ

In the present study we first examined whether microglia that encounter a high dose of IFN-γ adopt a phenotype that interferes with oligodendrogenesis from NPCs, and if so, whether IL-4 can counteract this negative effect.

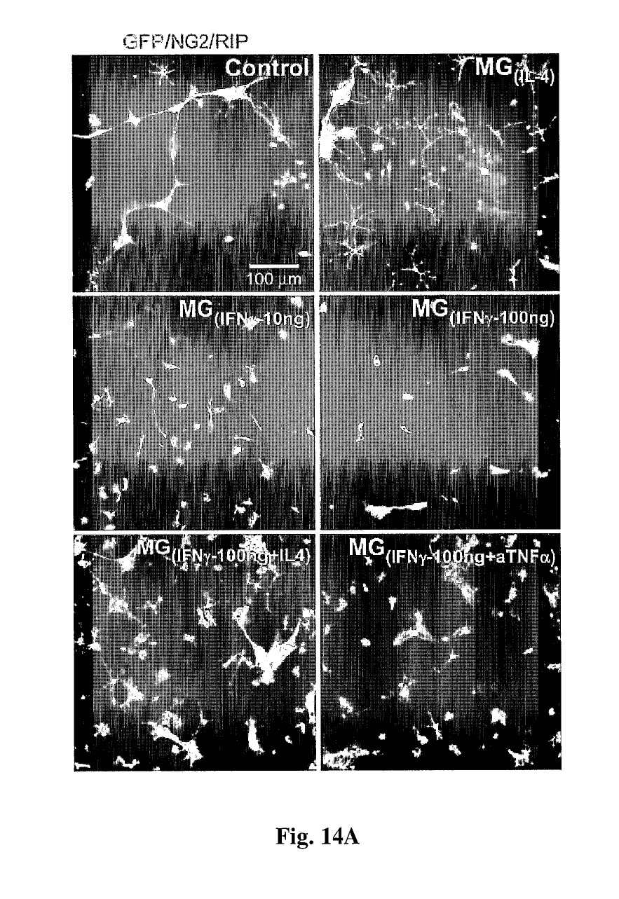

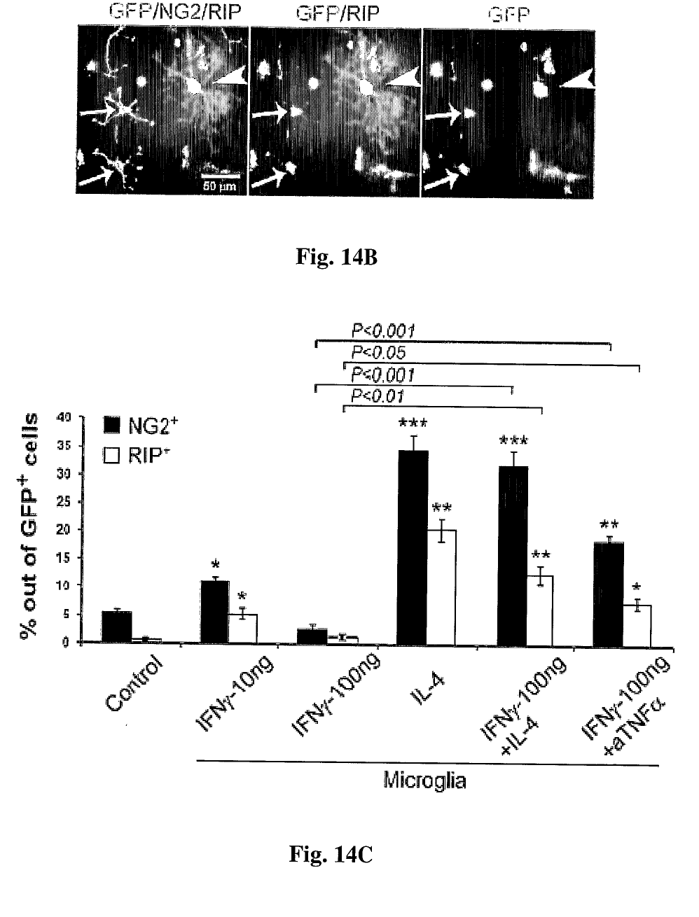

We first examined the effects on NPCs of microglia that were pre-incubated for 24 h in their optimal growth media, in the presence or absence of the cytokines IL-4 (10 ng/ml), IFN-γ (10 ng/ml or 100 ng/ml), or IL-4 (10 ng/ml) together with IFN-γ (100 ng/ml). After the growth media and cytokine residues were washed off, each of the treated microglial preparations was freshly co-cultured with dissociated NPC spheres on coverslips coated with Matrigel in the presence of differentiation medium. We used NPCs expressing GFP to verify that any differentiation of oligodendrocytes seen in the culture was derived from the NPCs rather than from contamination of the primary microglial culture. After 10 days we could discern both GFP-expressing NPCs co-labeled with the oligodendrocyte-progenitor marker NG2 and RIP-stained mature oligodendrocytes at the pre-ensheathing stage (Hsieh et al., 2004) (FIG. 14). A few GFP⁺/NG2⁺ and GFP⁺/RIP⁺ cells were seen in control NPCs cultured without microglia (Control) and in NPCs co-cultured with microglia pretreated with the low dose of IFN-γ (MG_{(IFN-γ,10 ng)}). In co-cultures of NPCs with MG_{(IL-4)}, however, the increase in numbers of GFP⁺/NG2⁺ and GFP⁺/RIP⁺ cells was dramatic. In contrast to the low dose of IFN-γ (10 ng/ml), treatment with high-dose IFN-γ (100 ng/ml) caused microglia to block oligodendrogenesis from the co-cultured NPCs. Interestingly, the addition of IL-4 (10 ng/ml) in combination with IFN-γ (100 ng/ml) overcame the adverse effect of high-dose IFN-γ, with the result that these microglia were able to induce NPCs to differentiate into oligodendrocytes. When we added neutralizing anti-TNF-(antibodies (ãTNF-α) to MG_{(IFN-γ,100 ng)}, we observed a significant increase in the numbers of NG2⁺ and (to a lesser extent) RIP⁺ cells in NPCs co-cultured with those microglia (FIG. 14A). Addition of ãTNF-α to co-cultures of NPCs with MG_{(IL-4)} had no effect, whereas the addition of anti-IGF-I antibodies (ãIGF-I) to these co-cultures blocked oligodendrogenesis (data not shown). In the presence of MG_{(IL-4)}, the newly differentiated oligodendrocytes were more branched than in the presence of MG_{(IFN-γ,10 ng)} (FIG. 14A). In each of the co-cultures, all NG2⁺ and RIP⁺ cells were also found to be co-labeled with GFP (FIG. 14B). Some GFP⁺/NG2⁺ cells co-expressed RIP⁺, whereas the well-branched mature oligodendrocytes (GFP⁺/RIP⁺) did not express NG2 at this stage (FIG. 14B). Quantitative analysis verified that IL-4 and (to a lesser extent) ãTNF-α had overcome the inhibitory effect of MG_{(IFNγ,100 ng)} on oligodendrogenesis induction (FIG. 14C). It should be noted that the effect of non-activated microglia in these experiments was neither destructive nor supportive.

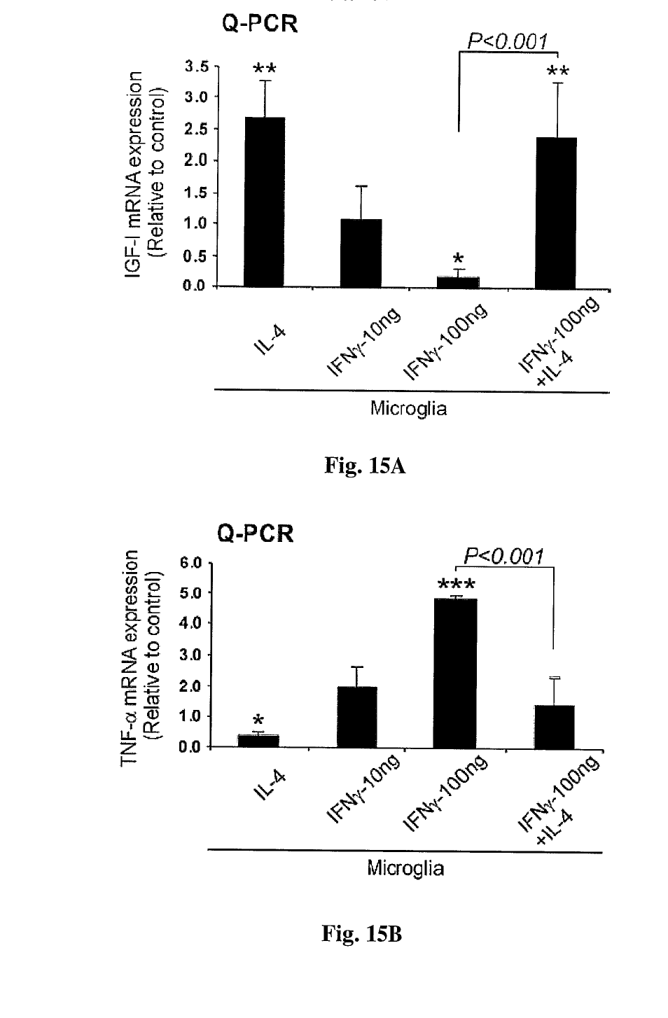

To examine the correlation between the production of IGF-I and the ability of IL-4-activated microglia to overcome the impediment to oligodendrogenesis, and to determine how this relationship is influenced by TNF-α production, we carried out quantitative real-time PCR analyses (Q-PCR) (FIG. 15). The results showed that low-dose IFN-γ (10 ng/ml) had no effect on the expression of IGF-I, whereas high-dose IFN-γ (100 ng/ml) completely arrested it. In contrast, IL-4 (10 ng/ml) had a robust effect on IGF-I expression and could overcome the negative effect of high-dose IFN-γ on IGF-I (FIG. 15A). In addition, whereas high-dose IFN-γ induced TNF-α production, IL-4 arrested it (FIG. 15B), possibly explaining the ability of IL-4 to overcome the negative effect of high-dose IFN-γ on IGF-I.

### Example 3(2)

### IL-4-Activated Microglia Induce Oligodendrogenesis from Endogenous Neural Stem/Progenitor Cells in an Acute EAE model

The finding that microglia activated by IL-4 in vitro created conditions favoring differentiation of NPCs into oligodendrocytes and overcame the negative effect of MG_{(IFN-γ,100 ng)} on oligodendrogenesis encouraged us to investigate whether MG_{(IL-4)} would promote oligodendrogenesis from endogenous adult NPCs in animals with acute or chronic EAE. To examine this possibility, we first induced acute EAE in adult Lewis rats (n=8) by immunizing them with MBP emulsified in CFA. Seven days later we introduced MG_{(IL-4)} into the CSF of these rats via stereotaxic bilateral injection into their cerebral ventricles. Control rats (n=8) were similarly injected with PBS. Seven days after injection of MG_{(IL-4)} or PBS, the rats were injected i.p. with BrdU every 12 h for 2.5 days to identify proliferating cells, and 21 days after the MBP vaccination their spinal cords were examined for the appearance of newly formed oligodendrocytes.

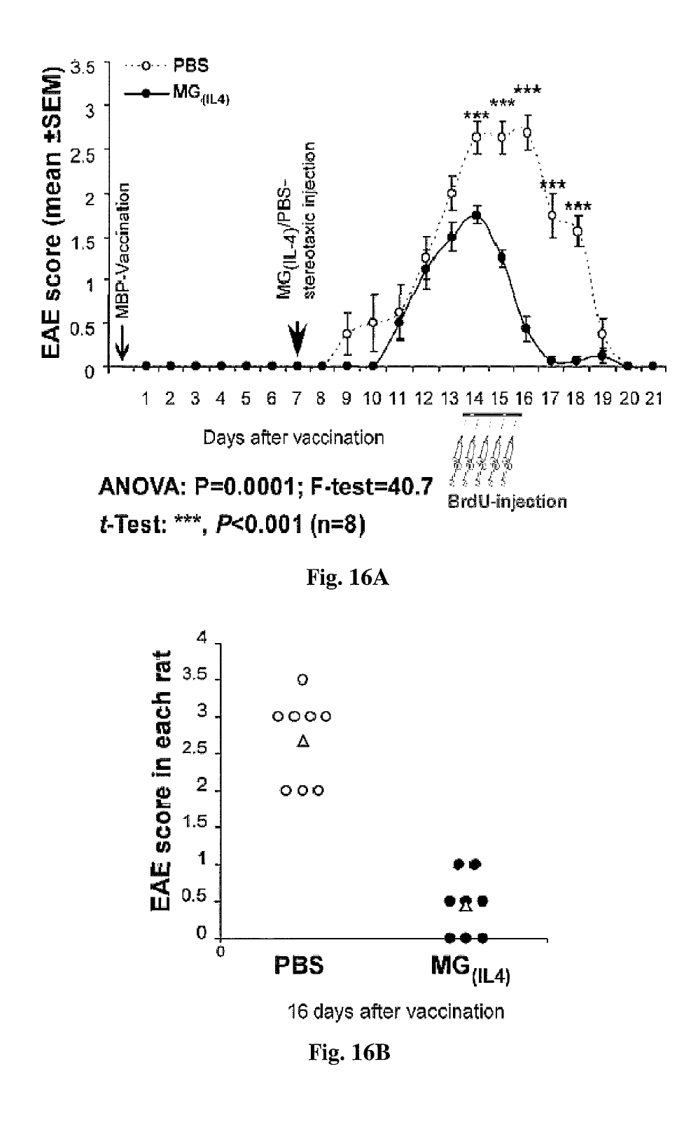

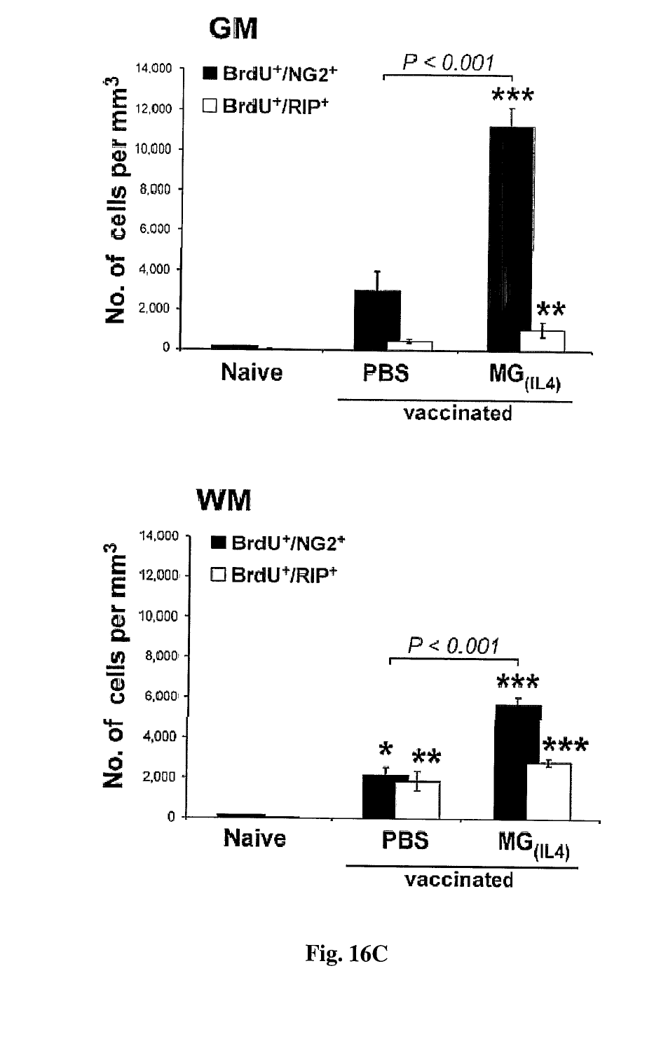

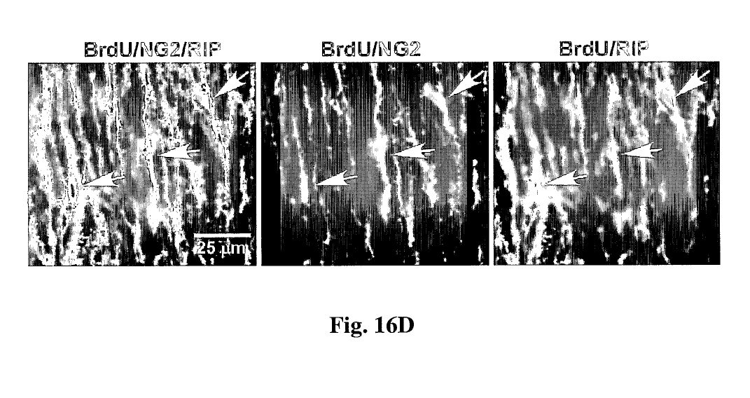

Although the symptoms of paralysis observed in this model of acute EAE do not result from demyelination, they were beneficially affected by IL-4. We therefore hypothesized that not only oligodendrogenesis but also functional integrity would benefit from IL-4-activated microglia, possibly in part through the IGF-I that these microglia are producing. Follow-up of the clinical manifestations of EAE with and without microglial treatment showed that after injection with MG_{(IL-4)}, the onset of disease was delayed and the severity and duration of clinical paralysis (FIG. 16A) and the number of rats manifesting the disease were significantly reduced (FIG. 16B). Similar results were obtained in an independent experiment carried out in the absence of BrdU, indicating that BrdU did not affect the manifestation of disease or the treatment efficacy.

Because the clinical symptoms of EAE in this model are manifested by tail and hindlimb paralysis, we assessed oligodendrogenesis in the spinal cords of these rats. Spinal cords were excised and longitudinal sections from the T8-T9 region were analyzed for newly formed oligodendrocytes and microglia. In both the untreated and the MG_{(IL-4)}-treated groups of rats with EAE we detected cells that were double-labeled for BrdU (proliferating cells) and NG2 (oligodendrocyte precursors). Some of the proliferating cells in both the gray and the white matter of MG_{(IL-4)}-treated rats were BrdU⁺/NG2⁺/RIP⁺, indicating that they had differentiated into committed oligodendrocytes. In those rats there were significantly more BrdU/NG2⁺ cells in the gray matter, and significantly more BrdU⁺/RIP⁺ cells in the white matter, than in PBS-treated rats (FIG. 16C). Some oligodendrogenesis was observed in the PBS-injected rats with EAE, leading us to consider the possibility that such oligodendrogenesis represents a self-reparative mechanism of myelin renewal that is induced by the inflammatory conditions even in the absence of any intervention (such as injection of MG_{(IL-4)}. Examination showed that the amount of oligodendrogenesis occurring in naïve rats was very small; thus, in the absence of treatment, oligodendrogenesis in PBS-injected rats with EAE was more abundant than in naïve rats, and was further augmented by MG_{(IL-4)}. (FIG. 16C). Co-expression of NG2 and RIP in the BrdU⁺ cells confirmed that the newly formed cells were oligodendrocytes (FIG. 16D).

It should be noted that BrdU in these experiments was injected at the peak of the disease, a stage at which a spontaneous feedback mechanism that limits the proinflammatory response is likely to be operating, with consequent reduction in the numbers of Th1 cells and/or the appearance of Th2 cells. It is therefore possible that the oligodendrogenesis seen in the PBS-injected rats with EAE was an outcome triggered by the Th1 cells whose numbers had declined by the time we injected the BrdU or by the spontaneous increasing numbers of Th2 (Duda et al., 2000).

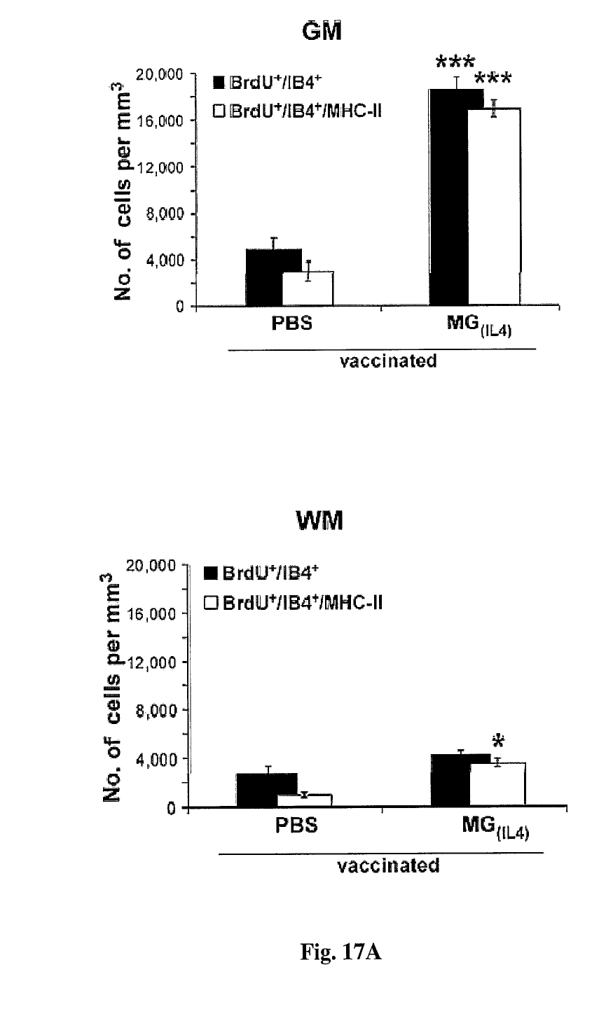

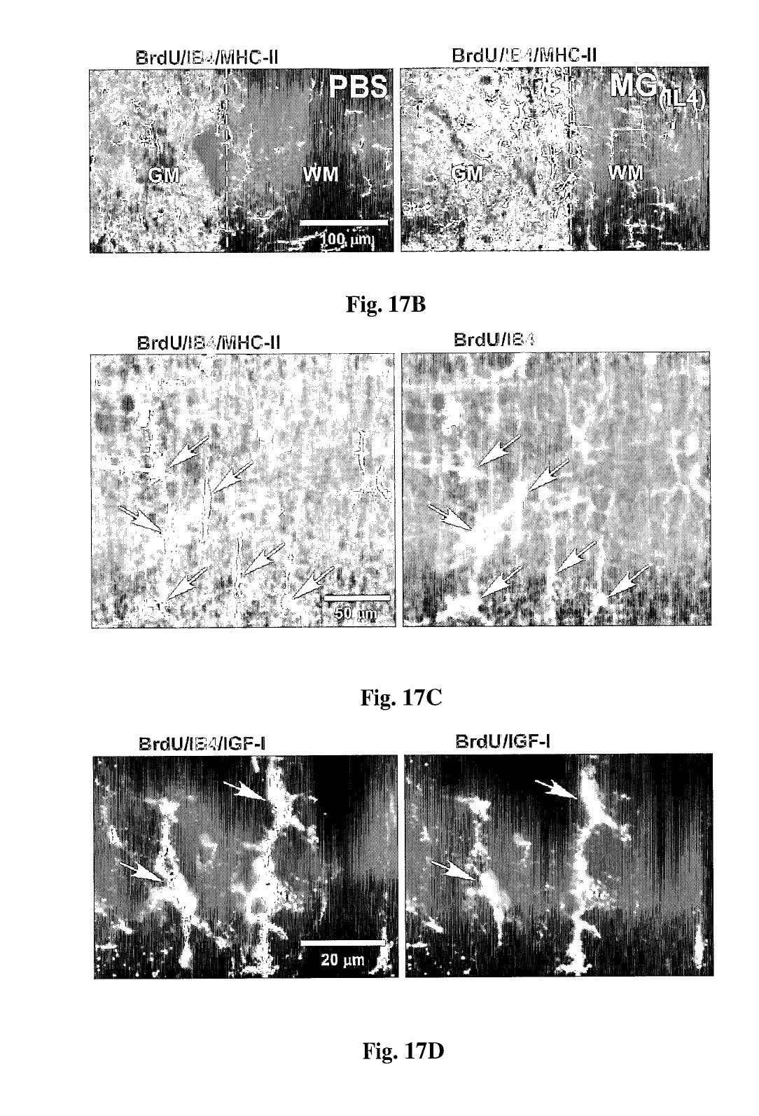

According to the traditional view, microglia that express MHC-II molecules are the activated microglia that are present in inflammation-associated diseases (Neumann et al., 1998). Recent studies by our group shown herein in the specification and by others showed, however, that not all MHC-II-expressing microglia are destructive (Li et al., 2005). For example, MHC-II-expressing microglia that are activated by low-dose IFN-γ or by IL-4 support cell survival. Analysis of consecutive sections obtained from the rats described above revealed the presence of newly formed microglia in both PBS-treated and MG_{(IL-4)}-treated rats, with the highest accumulation seen in the gray matter of the MG_{(IL-4)}-treated rats (FIG. 17A). No newly formed microglia were seen in naïve rats. Analysis of newly formed microglia (BrdU⁺/IB4⁺) in the gray matter of both PBS-treated and MG_{(IL-4)}-treated rats with EAE revealed that most of these new microglia were MHC-II⁺, and that they were significantly more numerous in the MG_{(IL-4)}-treated than in the PBS-treated rats (FIGS. 17A, 17B). Confocal scanning microscopy confirmed the expression of MHC-II by the newly formed microglia (BrdU⁺/IB4⁺) in the white matter of MG_{(IL-4)}-treated rats with EAE (FIG. 17C).

Because microglia can be induced to express IGF-I, and because the microglia in this study induced oligodendrogenesis in vitro (FIG. 14) and overcame the strongly pro-inflammatory conditions mediated by high-dose IFN-γ, we sought to identify IGF-I-expressing microglia in spinal cords of the MG_{(IL-4)}-treated rats. The newly formed BrdU⁺/IB4⁺ microglia were found to express IGF-I (FIG. 17D). Not all MHC-II⁺/IGF-I⁺ microglia were BrdU⁺, however, suggesting that those cells might be either the injected microglia or the newly formed ones.

### Example 3(3)

### IL-4-Activated Microglia Induce Oligodendrogenesis from Endogenous Neural Stem/Progenitor Cells in a Model of Chronic EAE

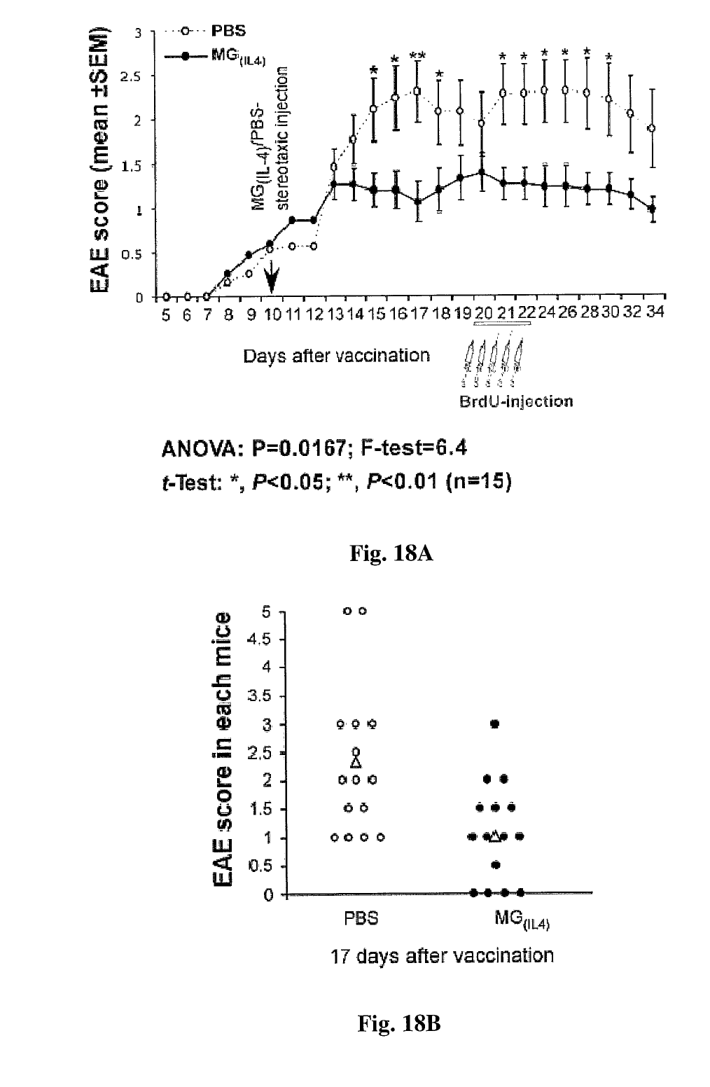

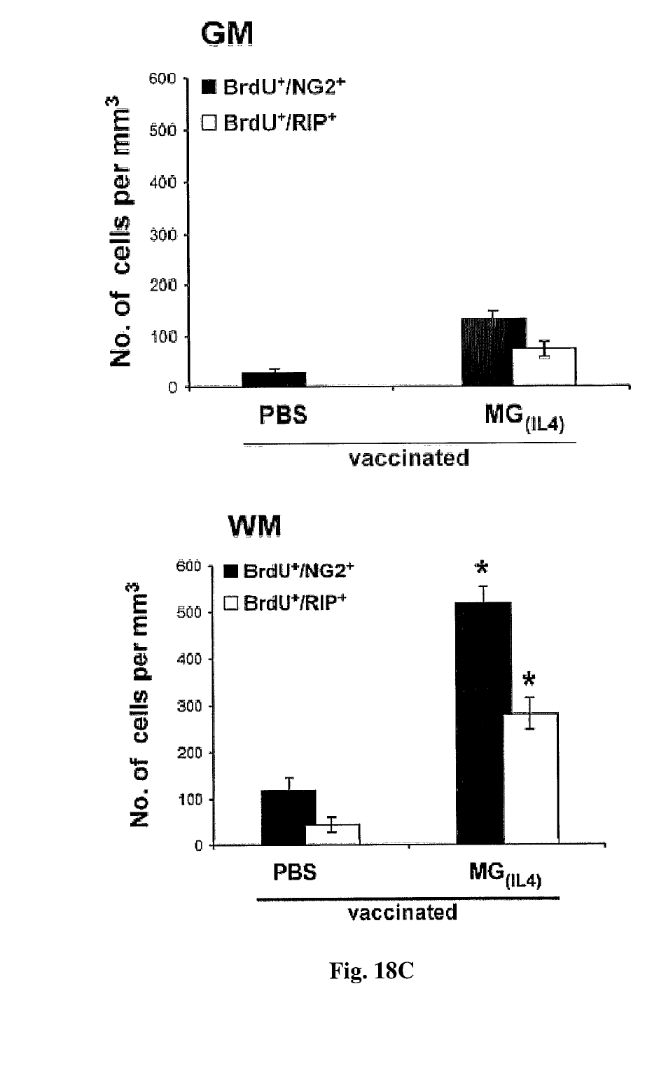

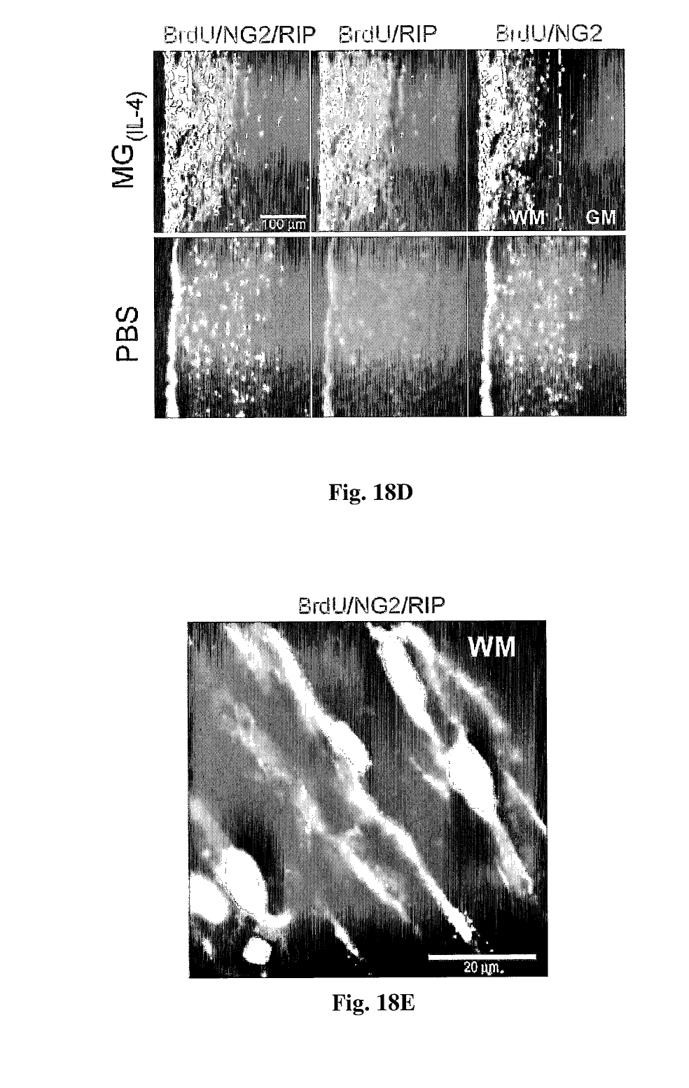

The results in vitro suggested that when Th1 cells persist, meaning that IFN-γ concentrations are high for a relatively long time, the result will be impairment of oligodendrogenesis. To verify that this is indeed the case we used the mouse model of chronic EAE to compare mice treated with MG_{(IL-4)} and with PBS. EAE was induced in C57BL/6J mice by immunization with the encephalitogenic myelin oligodendrocyte glycoprotein (MOG) peptide (35-55) emulsified in incomplete Freund's adjuvant containing Mycobacterium tuberculosis and pertussis toxin. Ten days after EAE induction we injected MG_{(IL-4)} or PBS into the CSF, via bilateral stereotaxic injection into the cerebral ventricles. The two groups of injected mice differed significantly both in severity of paralysis (FIG. 18A) and in the numbers expressing the disease (FIG. 18B). In the white matter (but not in the gray matter) of mice with EAE, significantly more newly formed oligodendrocytes (BrdU⁺/NG2⁺/RIP⁺) were observed in the group treated with MG_{(IL-4)} than in the PBS-treated group (FIGS. 18C, 18D). In contrast to PBS-treated rats with monophasic (acute) EAE, hardly any oligodendrogenesis could be seen in their counterparts in which EAE was chronic or in naïve mice (data not shown), again suggesting that when the levels of Th1 cells (IFN-γ-secreting) are large (i.e., the IFN-γ dosage is high) the conditions do not favor oligodendrogenesis.

With the aid of scanning confocal microscopy, co-expression of NG2 and RIP by the newly formed oligodendrocytes was confirmed in the white matter of the MG_{IL-4}-treated mice with EAE (FIG. 18E).

### Discussion

The results of this study lead us to attribute a novel role for microglia as both supporters and blockers of oligodendrocyte renewal from the endogenous NPC pool in the adult CNS. The in-vitro findings showed that IL-4-activated microglia, in part via production of IGF-I and down-regulation of TNF-α, were remarkably potent in counteracting the impediment to oligodendrogenesis induced by high-dose IFN-γ. In vivo, MG_{(IL-4)} supported oligodendrogenesis and clinical recovery in rats and mice in which severe inflammatory conditions are known to evoke clinical symptoms of transient or chronic EAE.

### Example 4

### Synergy Between T Cells and Adult Neural Progenitor Cells Promotes Functional Recovery from Spinal Cord Injury

Recovery from spinal cord injury evidently necessitates a local immune response that is amenable to well-controlled boosting by immunization with T lymphocytes recognizing myelin-associated antigens at the injury site. The relevant T cells can activate local microglia to express a phenotype supportive of neuronal survival and renewal. We show that recovery of mice from spinal contusion is synergistically promoted by T-cell-based vaccination with a myelin-derived peptide and injection of adult neural stem/progenitor cells (aNPCs) into the cerebrospinal fluid. Significantly more aNPCs targeted the lesion site in vaccinated than in nonvaccinated mice. Synergistic interaction between aNPCs and T cells in vitro was critically dependent on T-cell specificity and phenotype. The results suggest that controlled immune activity underlies efficient regulation of the stem-cell niche, and that stem-cell therapy necessitates autologous or histocompatibility-matched donors instead of the immunosuppressive anti-rejection drugs that would eliminate any beneficial effect of immune cells on spinal cord repair.

### Materials and Methods

(xxvii) Antigens. The following peptides were synthesized by the Synthesis Unit at the Weizmann Institute (Rehovot, Israel): MOG, residues 35-55 MEVGWYRSPFSRVVHLYRNGK (SEQ ID NO:1) and an altered MOG peptide (45D) MEVGWYRSPFDRVVHLYRNGK (SEQ ID NO:2), a peptide analog of MOG 35-55 containing a serine to aspartic acid substitution at P6. OVA was purchased from Sigma.

(xxviii) Immunization. Adult mice were immunized with MOG, 45D, or OVA (all 100 μg), each emulsified in an equal volume of CFA (Difco, Detroit, Mich.) containing Mycobacterium tuberculosis (5 mg/ml; Difco), or IFA. The emulsion (total volume 0.15 ml) was injected s.c. at one site in the flank. Control mice were injected with PBS.

(xxix) Spinal cord injury. Mice were anesthetized, their spinal cords were exposed by laminectomy at T12, and a force of 200 kdyn was placed for 1 s on the laminectomized cord using the Infinite Horizon spinal cord impactor (Precision Systems and Instrumentation, Lexington, Ky.), a device shown to inflict a well-calibrated contusive injury of the spinal cord.

(xxx) Assessment of functional recovery from spinal cord contusion. Functional recovery from spinal cord contusion in mice was determined by hindlimb locomotor performance. Recovery was scored by the Basso Mouse Scale (BMS) open-field locomotor rating scale, a scale recently developed specifically for mice, with scores ranging from 0 (complete paralysis) to 9 (normal mobility) (Engesser-Cesar et al., 2005). Blind scoring ensured that observers were not aware of the treatment received by each mouse. Twice a week locomotor activities of mice in an open field were monitored by placing the mouse for 4 min in the center of a circular enclosure (90 cm in diameter, 7 cm wall height) made of molded plastic with a smooth, non-slip floor. Before each evaluation the mice were examined carefully for perineal infection, wounds in the hindlimbs, and tail and foot autophagia.

(xxxi) Stereotaxic injection of neural progenitor cells. Mice were anesthetized and placed in a stereotactic device. The skull was exposed and kept dry and clean. The bregma was identified and marked. The designated point of injection was at a depth of 2 mm from the brain surface, 0.4 mm behind the bregma in the anteroposterior axis, and 1.0 mm lateral to the midline. Neural progenitor cells were applied with a Hamilton syringe (5×10⁵ cells in 3 μl, at a rate of 1 μl/min) and the skin over the wound was sutured.

(xxxii) Neural progenitor cell culture. Cultures of adult neural progenitor cells (aNPCs) were obtained as previously described.

(xxxiii) Co-culturing of neural progenitor cells and T cells. CD4+ T cells were purified from lymph nodes of 8-week-old C57B16/J mice as previously described (Kipnis et al., 2004). T cells were activated in RPMI medium supplemented with L-glutamine (2 mM), 2-mercaptoethanol (5×10⁻⁵ M), sodium pyruvate (1 mM), penicillin (100 IU/ml), streptomycin (100 μg/ml), nonessential amino acids (1 ml/100 ml), and autologous serum 2% (v/v) in the presence of mouse recombinant IL-2 (mrIL-2; 5 ng/ml) and soluble anti-CD28 and anti-CD3 antibodies (1 ng/ml). T cells were co-cultured (5×10⁴ cells/well) with aNPCs (5×10⁴ cells/well) for 5 d on cover slips coated with Matrigel (BD Biosciences) in 24-well plates. The cultures were then fixed with 2.5% paraformaldehyde in PBS for 30 min at room temperature and stained for neuronal markers.

(xxxiv) Immunohistochemistry. Mice subjected to SCI were re-anesthetized 14 or 60 days later and perfused with cold PBS. Their spinal cords were removed, postfixed with Bouin's fixative (75% saturated picric acid, 25% formaldehyde, 5% glacial acetic acid; Sigma-Aldrich) for 48 h, and then transferred to 70% EtOH. The tissues were hydrated through a gradient of 70%, 95%, and 100% EtOH in xylene and paraffin, and were then embedded in paraffin. For each stain, five tissue sections, each 6 μm thick, were taken from each mouse. The paraffin was removed by successive rinsing of slides for 15 min with each of the following: xylene, EtOH 100%, 95%, 70%, 50%, and PBS. Exposure of the slides to antigen was maximized by heating them to boiling point in 10 mM sodium citrate pH 6.0 in a microwave oven, then heating them at 20% microwave power for a further 10 min. The slides were blocked with 20% normal horse serum for 60 min prior to overnight incubation at room temperature (GFAP, neurofilaments, and BDNF), or for 48 h at 4° C. (CD3), with the monoclonal antibody in 2% horse serum. We used rabbit anti-mouse GFAP (1:200) (DakoCytomation, Glostrup, Denmark) for GFAP; rabbit anti-neurofilament (1:200), low and high molecular weight (Serotec, Oxford, UK) for neurofilaments; and rat anti-human CD3 (1:50) (Serotec) for CD3. For BDNF we used the monoclonal antibody chicken anti-human BDNF (1:100) (Promega, Madison, Wis.) with 0.05% saponin.

After rinsing, sections were incubated for 1 h at room temperature with the secondary antibody Cy3 donkey anti-rat (1:300) (Jackson ImmunoResearch Laboratories, West Grove, Pa.) (staining for CD3), Cy3 donkey anti-chicken (Jackson ImmunoResearch) (1:250) (staining for BDNF), or Cy3 donkey anti-rabbit (Jackson ImmunoResearch) (1:250) (staining for GFAP and neurofilaments). For IB4 staining, sections were blocked for 1 h with 20% horse serum and then incubated for 1 h at room temperature with Cy2-IB4 (1:50) (Sigma-Aldrich). All sections were stained with Hoechst (1:2000) (Molecular Probes-Invitrogen, Carlsbad, Calif.). They were then prepared for examination under a Nicon E-600 fluorescence light microscope. Results were analyzed by counting the cells (CD3-labeled) in the site of injury, or by determination of the density (IB4-labeled or BDNF-labeled), or by measurement of the unstained area (GFAP, neurofilaments). Each of the parameters was measured by an observer who was blinded to the treatment received by the mice.

### Example 4(1)

### Adult Neural Progenitor Cells Require Local Immune Activity to Promote Motor Recovery

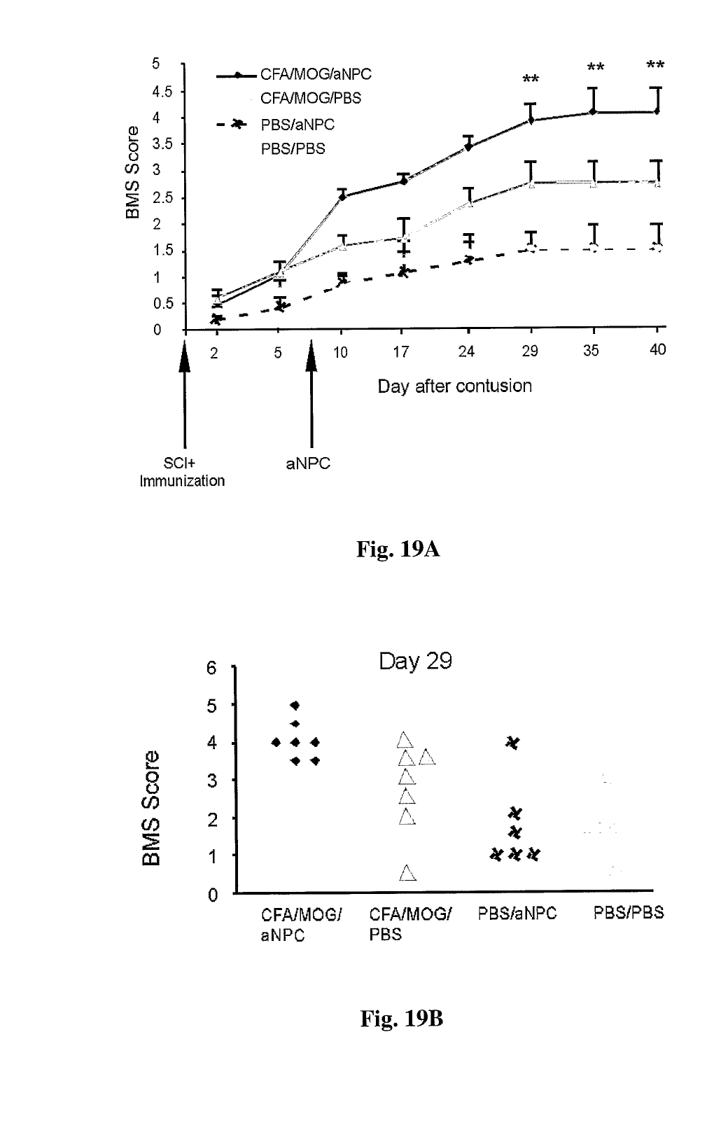

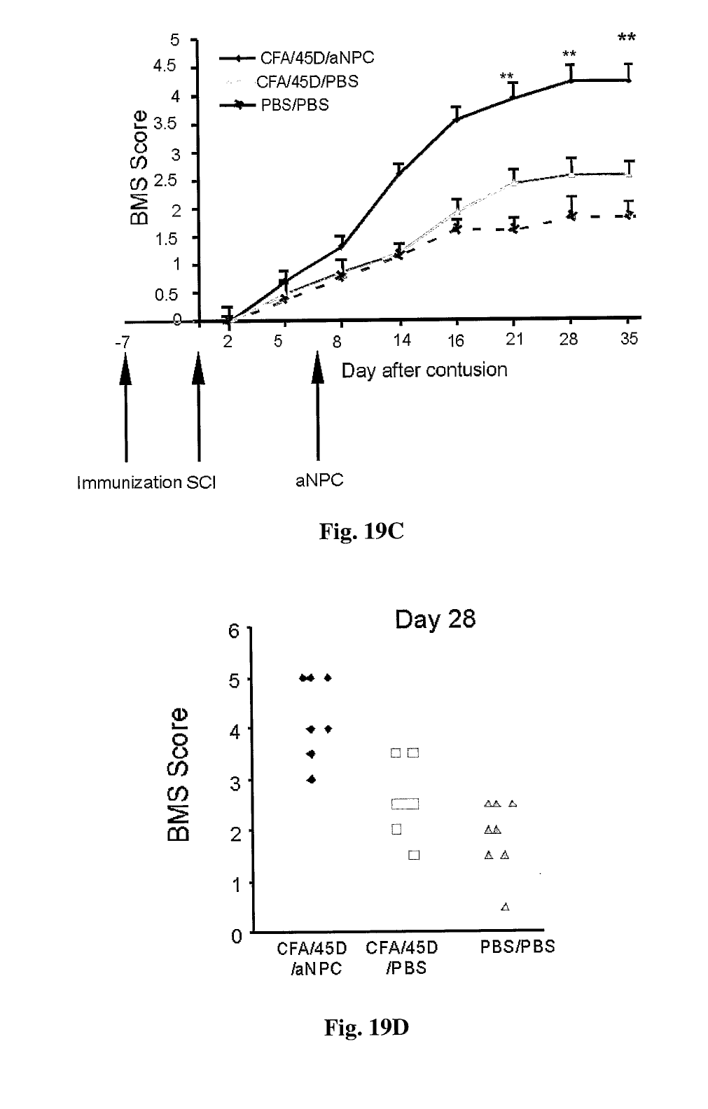

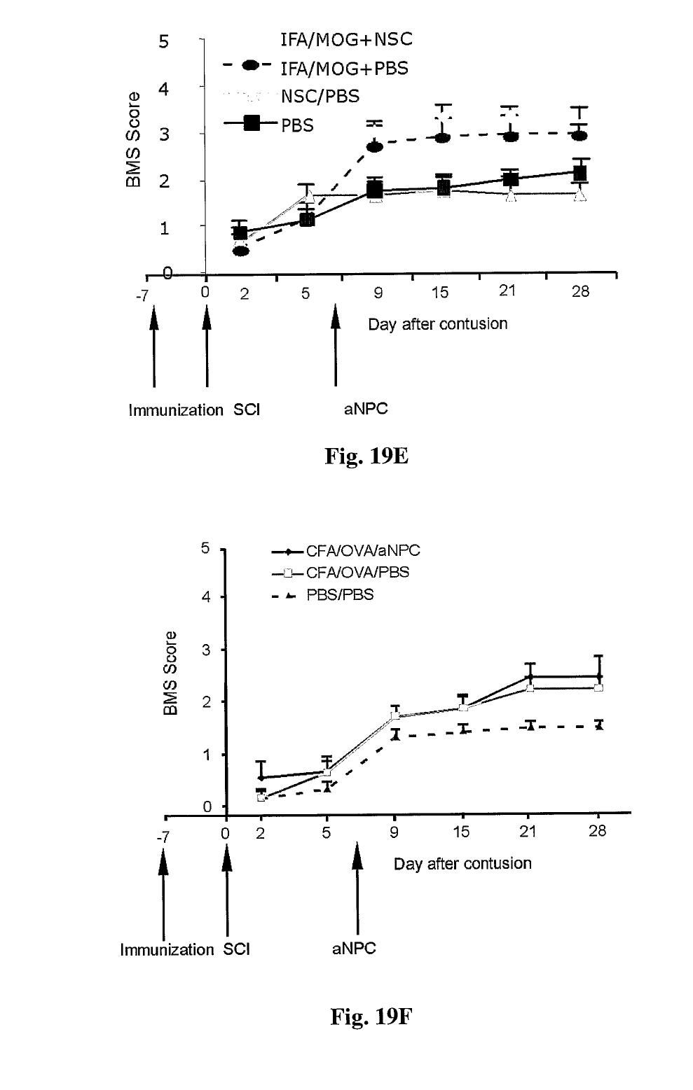

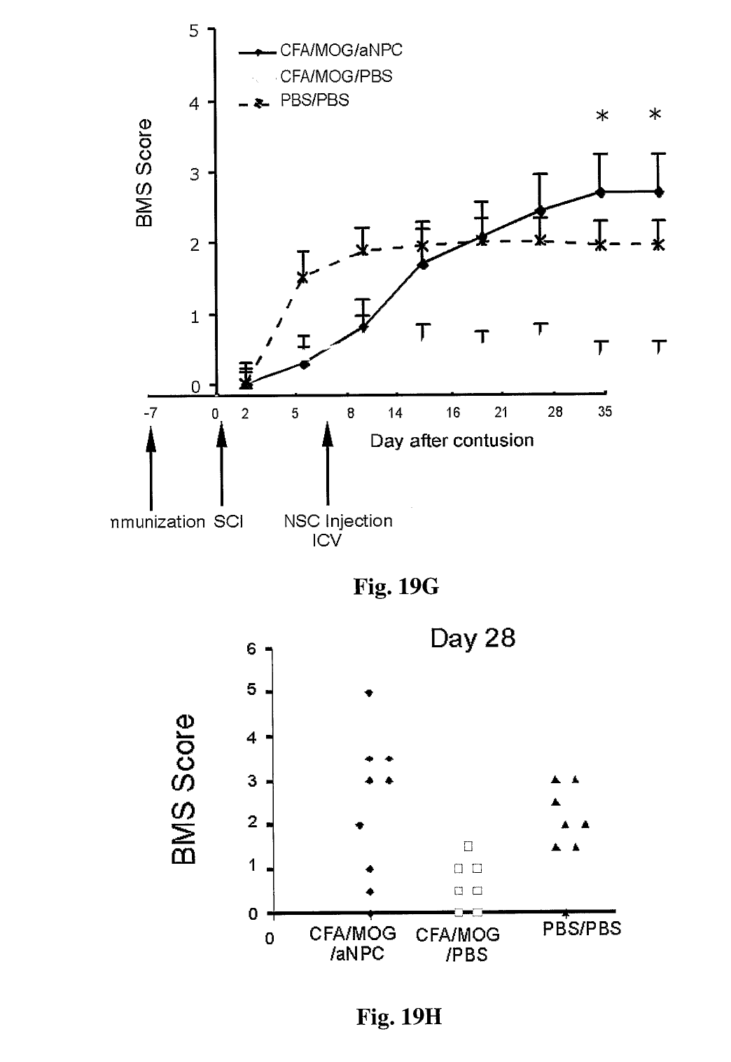

Our working hypothesis in this study was that the protective immune response evoked at a site of injury by T-cell based immunization creates a niche that supports not only cell survival but also tissue repair. We further suspected that a local T-cell mediated immune response could attract exogenously delivered aNPCs and support their contribution to recovery. To test this hypothesis we vaccinated C57B1/6J mice, immediately after SCI, with the encephalitogenic peptide pMOG 35-55 (SEQ ID NO: 1) emulsified in CFA containing 0.5% Mycobacterium tuberculosis (MOG/CFA), and 1 week later administered aNPCs via the intracerebroventricular (i.c.v.) route. Mice subjected to this dual treatment protocol (MOG/CFA/aNPC) were compared to a control group of mice that were immunized with the same MOG peptide emulsified in the same adjuvant but were not transplanted with aNPCs and instead were injected i.c.v. with PBS (MOG/CFA/PBS), or to mice that were injected with PBS and CFA (0.5%) and transplanted i.c.v. with aNPCs (PBS/CFA/aNPC) or a control group of mice that were injected with PBS/CFA and then injected i.c.v. with PBS (PBS/CFA/PBS). To assess behavioral outcome after SCI we used the Basso motor score (BMS) rating scale (Engesser-Cesar et al., 2005), in which 0 indicates complete paralysis of the hindlimbs and 9 denotes full mobility. The mean motor recovery (BMS) scores of mice receiving the MOG/CFA/aNPC (4.21±0.45; all values are mean±SEM) were higher than those of mice treated with MOG/CFA/PBS. In mice treated with PBS/CFA/aNPC, recovery was not better than in control mice treated with PBS/CFA/PBS (1.5±0.27). A BMS of 4.21 indicates extensive movement of the ankle and plantar placement of the paw (three animals showed, in addition, occasional weight support and plantar steps), whereas a score of 1.5 indicates ankle movement ranging from slight to extensive. Mice treated with MOG/CFA/PBS scored 2.71±0.5, a, significantly higher score than that of control PBS/CFA/aNPC-treated mice (1.5±0.4) or of control mice treated with PBS/CFA/PBS (FIG. 19A). These results thus demonstrated synergistic interaction between the administered aNPCs and the T cell-based immune response. Failure of the transplanted aNPCs to improve motor recovery by themselves (i.e., in the absence of MOG/CFA immunization) suggested that a site-specific immune response was necessary for aNPC activity. FIG. 19B shows the BMS of individual mice in all examined groups on day 28 postinjury. Because transplantation of aNPCs in the absence of immunization did not improve recovery from SCI, this control group (PBS/CFA/aNPC) was not included in subsequent experiments.

We have previously demonstrated that boosting of the amounts of T cells needed for promoting recovery from SCI does not necessitate the use of encephalitogenic peptides; weak agonists of encephalitogenic peptides are just as effective and do not carry the risk of inducing EAE (Hauben et al., 2000, 2001). To test whether such ‘safe’ vaccination could be utilized in combination with aNPCs transplantation we used a MOG-derived altered peptide ligand (PMOG 35-55 APL; 45D peptide, SEQ ID NO: 2), in which aspartic acid is substituted for serine. Mice were vaccinated with the 45D peptide emulsified in CFA containing 2.5% Mycobacterium tuberculosis. One week later the immunized mice were subjected to contusive SCI, and after another week were transplanted i.c.v. with aNPCs. Increased motor activity (as expressed by the BMS, mean±SEM) was seen in these mice than in control mice treated i.c.v with PBS/CFA/aNPC (4.11±0.27 compared to 1.94±0.22; FIG. 19C). Without aNPC transplantation, immunization with peptide 45D in CFA resulted in only a slight increase in motor recovery relative to the PBS-treated control (2.57±0.24). FIG. 19D shows BMS values for individual mice on day 28 postinjury. The above findings showed that the contribution of transplanted aNPCs to motor recovery after contusive SCI can also be promoted by the use of a weak agonist of the encephalitogenic peptide.

To determine the phenotype and specificity of the T cells needed for synergistic interaction with aNPCs we repeated the above experiments using different immunization protocols. Incomplete Freund's adjuvant (IFA), unlike CFA, is free of bacteria and is known to elicit a Th2-like response to encephalitogenic peptides. We found that although immunization with the MOG analog (peptide 45D) emulsified in IFA had some beneficial effect, it showed no synergy with subsequent transplantation of aNPCs (BMS of 3.2±0.76 for MOG/IFA/aNPC-treated mice compared to 2.93±1.03 for MOG/IFA/PBS-treated mice and 2.07±0.53 in the PBS/CFA/PBS-treated mice; FIG. 19E). These findings suggest that for MOG/IFA/aNPC-treated mice, the preferred T cells for synergistic interaction at the injury site between vaccinated T cells and aNPCs are Th1.

To verify that specificity to CNS-antigens is required for a synergistic effect of vaccination and stem-cell transplantation, mice were immunized with the nonself protein ovalbumin (OVA) emulsified in CFA (containing 2.5% Mycobacterium tuberculosis), and 1 week later were injected i.c.v. with aNPCs or with PBS as control. Immunization with OVA/CFA resulted in a slight, nonsignificant increase in BMS, and implantation of aNPCs did not increase the BMS any further (2.43±1.78 for the OVA/CFA/aNPC-treated mice compared to 2.2±0.68 for OVA/CFA/PBS-treated mice and 1.5±0.29 for PBS/CFA/PBS-treated control group, FIG. 19F). Taking all of the above results together, the absence of a beneficial effect after aNPC transplantation suggests that synergy between T cells and aNPCs is a function of both the antigenic specificity and the phenotype of the T cells.

Immune activation in the injured CNS, and specifically in the spinal cord, has been a major focus of research attention in recent years (Schwartz and Hauben, 2002). In some of the studies, T cell-based immune responses were shown to be protective only if their intensity and duration were well regulated. Overly strong immunization yielded excessive immune activity, which neutralized the potential benefit of the immune response for the injured spinal cord and even had a detrimental effect. We considered the possibility that if aNPCs home to the site of damage they can offset the negative effect of excessive immune activity and thus contribute to recovery. We therefore set out to determine whether administration of aNPCs can contribute to functional recovery even when the local immune activity is excessive. One week prior to SCI we immunized mice with MOG peptide 35-55 emulsified in CFA containing 2.5% Mycobacterium tuberculosis. Under conditions in which motor recovery from SCI was worse after immunization with MOG/CFA than after injection with PBS/CFA (BMS of 0.35±0.2 and 1.94±0.32, respectively), we found that recovery was improved upon administration of aNPCs (BMS of 2.68±0.51; FIGS. 19G, 19H). It thus appears that i.c.v. administration of aNPCs can contribute to functional recovery from SCI even when the local adaptive immune response is detrimental.

### Example 4(2)

### Tissue Integrity Correlates with Local Immune Activity

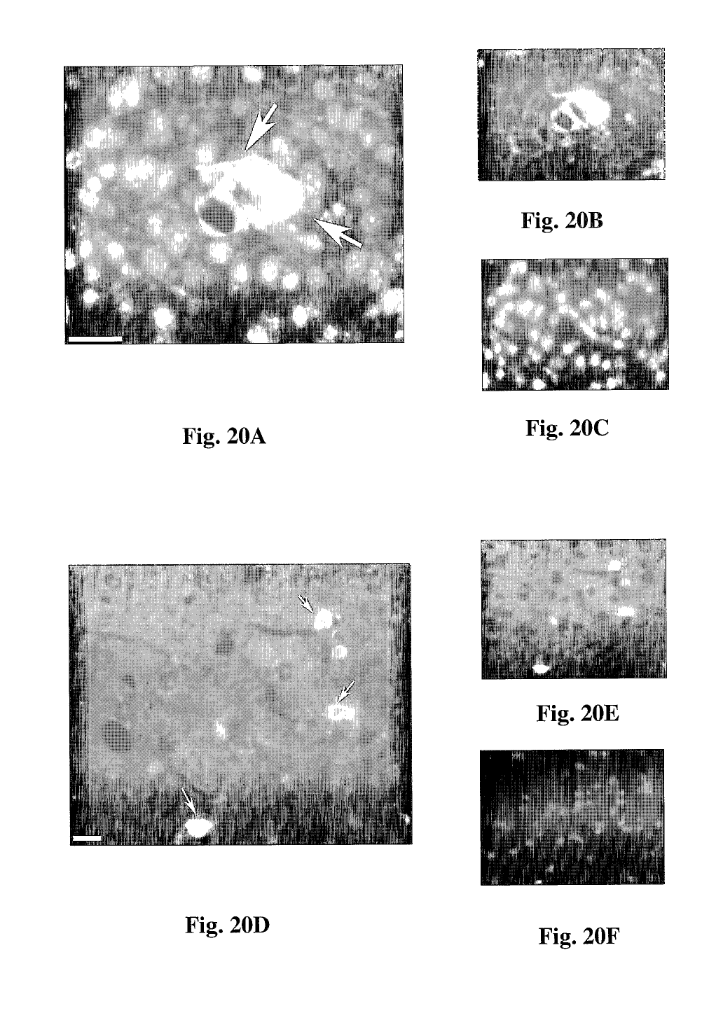

In an attempt to gain an insight into the mechanism underlying the apparent synergy between aNPCs and resident immune cells, we examined whether any of the injected aNPCs find their way to the injured spinal cord. We repeated the experiment showed on FIG. 19a using GFP-labeled aNPCs. Staining with anti-GFP antibodies revealed GFP⁺ cells in the parenchyma of the injured spinal cord only in mice that were treated with MOG-CFA/aNPC (FIG. 20). Injected GFP+ aNPCs could be seen surrounding the epicenter of the lesion and laterally in the spinal cord parenchyma adjacent to the meninges as early as 7 days after SCI (FIGS. 20A-20C), and could still be detected as late as 60 days after the injury, the last time point examined (FIGS. 20D-20F).

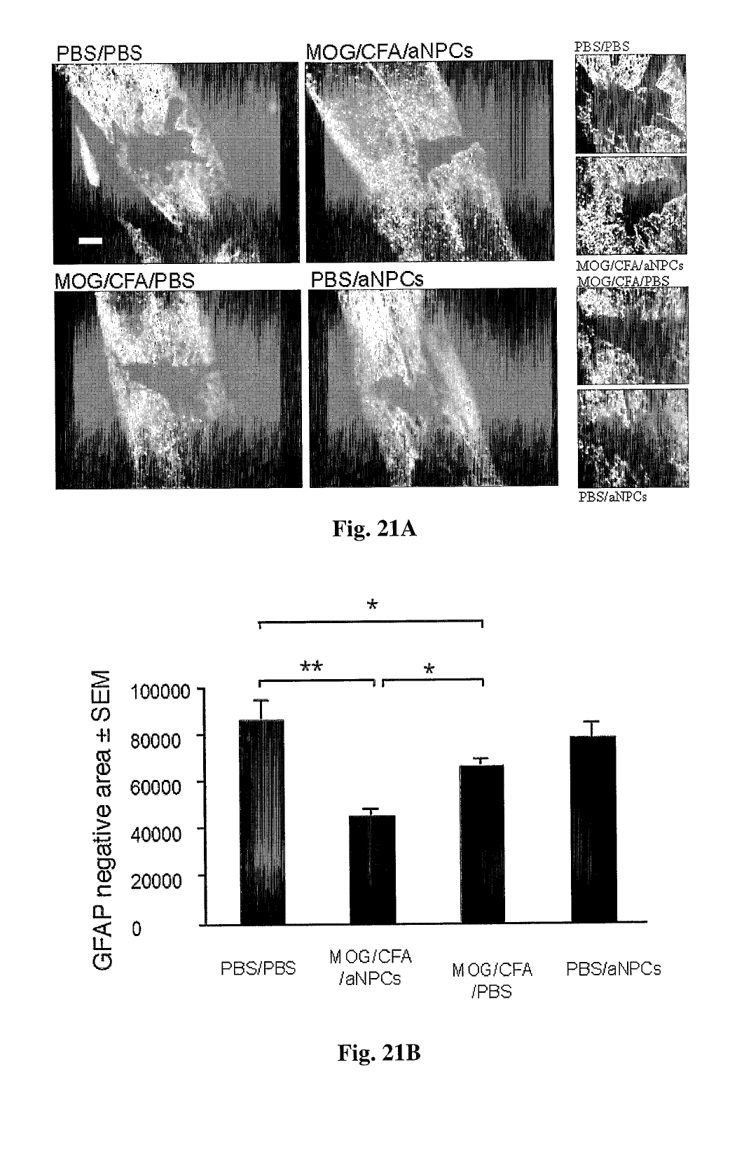

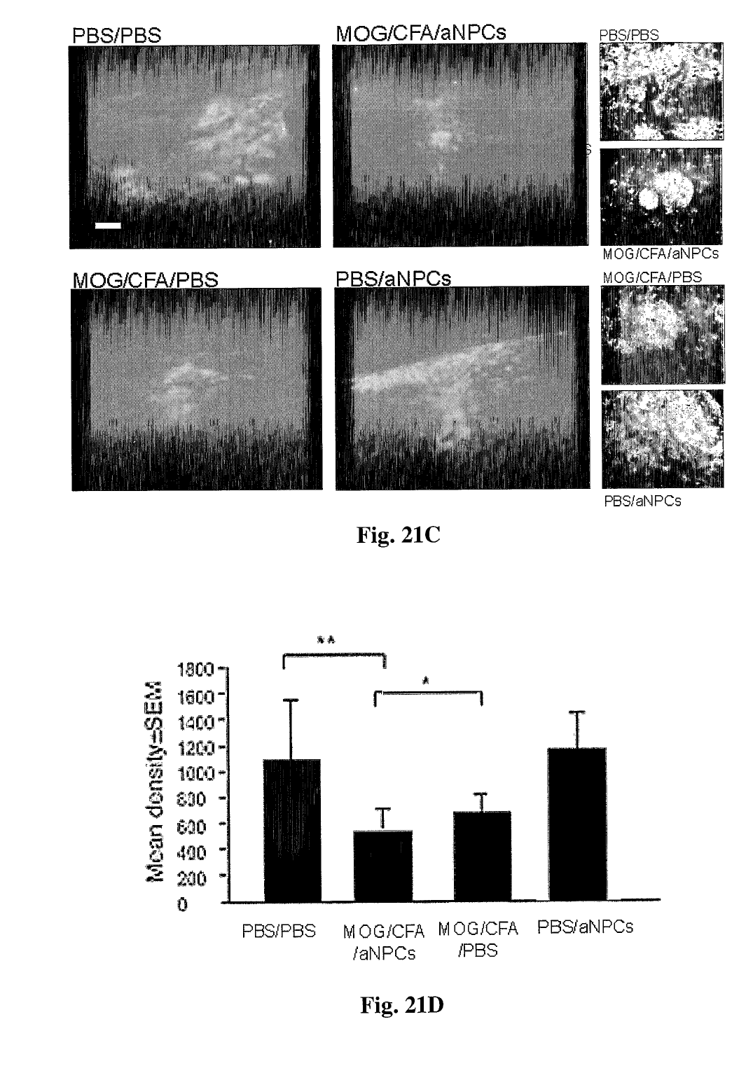

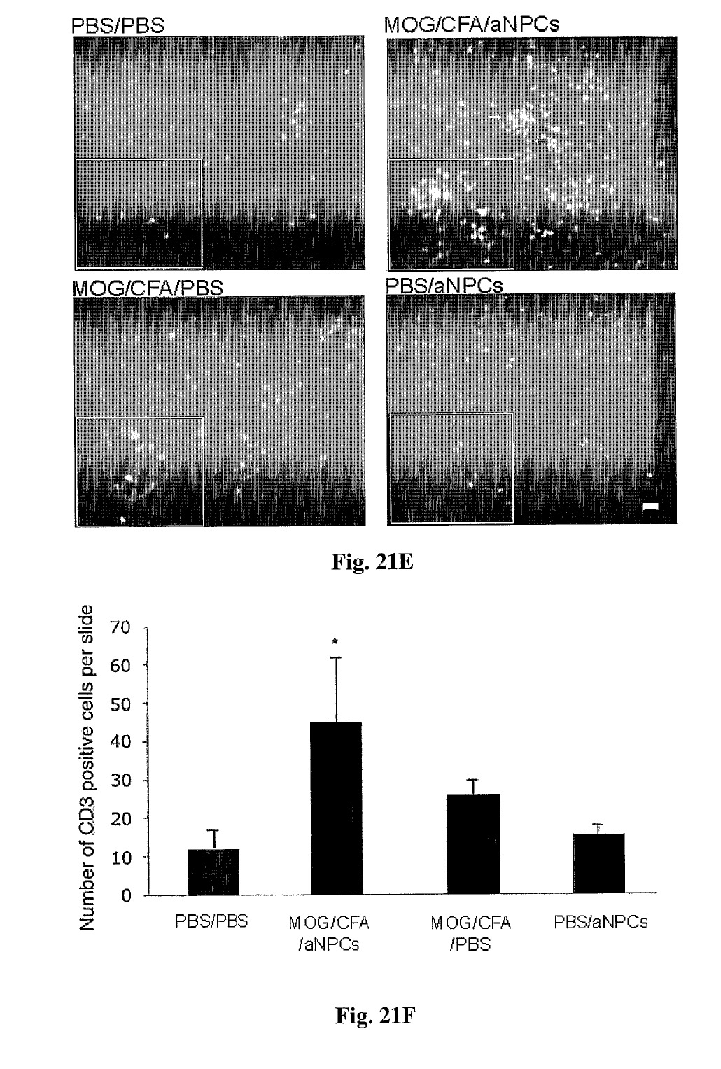

One of the morphological features that characterize recovery from SCI is the size of the lesion. To delineate the site of injury we stained longitudinal sections of the spinal cord with antibodies to glial fibrillary acidic protein (GFAP) (Blaugrund et al., 1992). We assessed the lesion size by measuring the areas that were not stained by GFAP. This analysis disclosed that as early as 7 days after aNPCs were transplanted in the MOG/CFA-vaccinated rats, the averaged size of the site of injury was significantly smaller in mice that had received both vaccination and aNPC transplantation than in mice that had only been vaccinated or had received only aNPCs (FIGS. 21A, 21B).

Next we examined whether the observed differences in the extent of recovery could be correlated with local immunological changes. Sections of spinal cord tissue were stained for markers of T cells (CD3) and accumulation of activated microglia/macrophages (IB4) (FIGS. 21C-21F). All sections were also stained with Hoechst as a nuclear marker. Tissues were excised 7 days after cell transplantation. Staining with IB4 revealed fewer microglia/macrophages in mice that had received the dual treatment protocol (pMOG 35-55 in 0.5% CFA) than in the other experimental groups (FIGS. 21C, 21D). Quantitative analysis revealed significantly more CD3+ T cells in areas surrounding the site of injury in mice that had received the dual treatment than in MOG/CFA/PBS-treated or in PBS/CFA/PBS-treated controls. Notably, immunization with the encephalitogenic pMOG 35-55 without aNPC transplantation resulted in only slightly more CD3+ cells at the injured site than in the controls (FIGS. 21E, 21F). It thus seems that transplantation of aNPCs modulated the local immune response at the injured site.

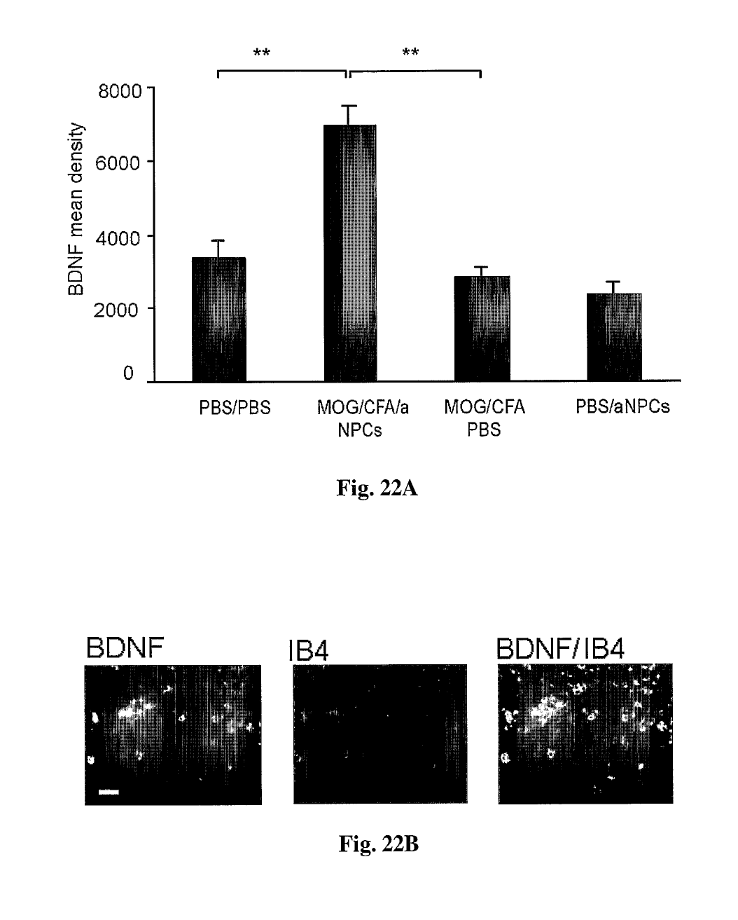

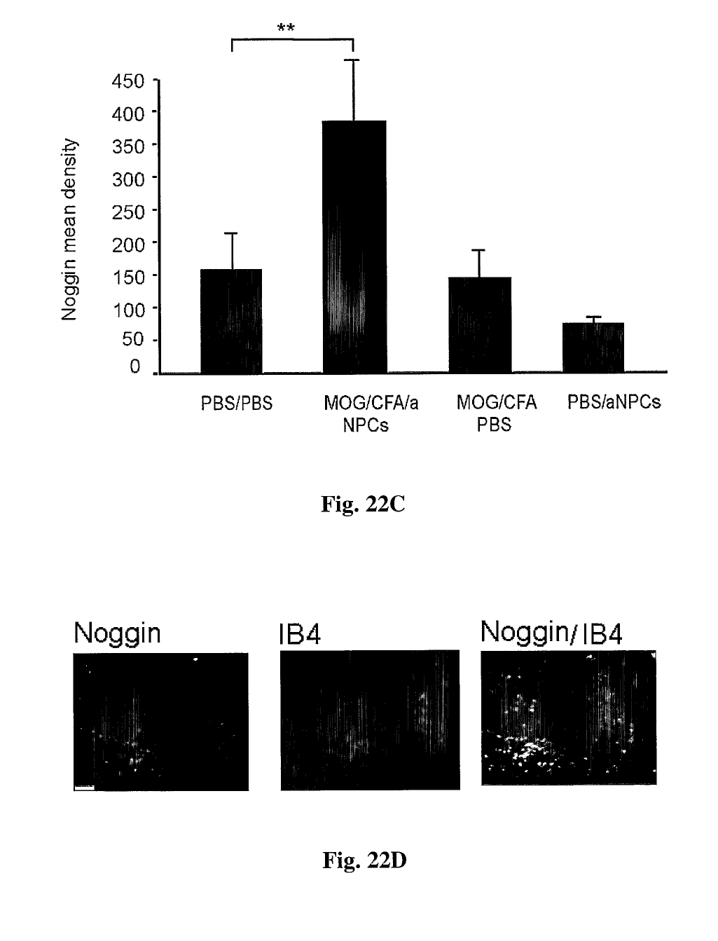

Both activated T cells and T cell-activated microglia can serve as sources of growth factors such as brain-derived neurotrophic factor (BDNF). BDNF immunoreactivity was more intense in the spinal cords of mice treated with MOG/CFA/aNPC than in the other groups (FIG. 22A). Double staining for BDNF and IB4 showed that the cellular source of BDNF in the injured site was the microglial/macrophage population (FIG. 22B).

Recent studies have shown that noggin, a bone morphogenesis protein (BMP) inhibitor, can induce neuronal differentiation from aNPCs in the injured spinal cord (Setoguchi et al., 2004). This protein was also shown to be needed to provide a neurogenic environment in the subventricular zone. We therefore assayed noggin immunoreactivity in the various experimental groups, and found that it was significantly increased in mice that received the dual treatment protocol, but was unaffected by MOG immunization alone and was slightly decreased by aNPC transplantation alone (FIG. 22C). As in the case of BDNF produced in the injured site, noggin was also localized to IB4+ cells (FIG. 22D).

### Example 4(3). Local Differentiation of Endogenous Stem Cells

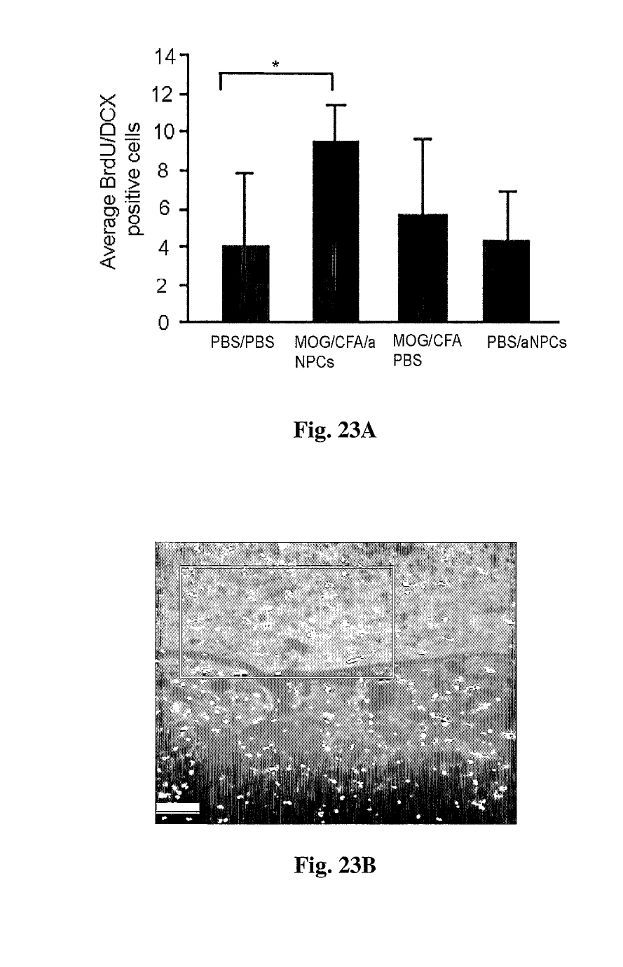

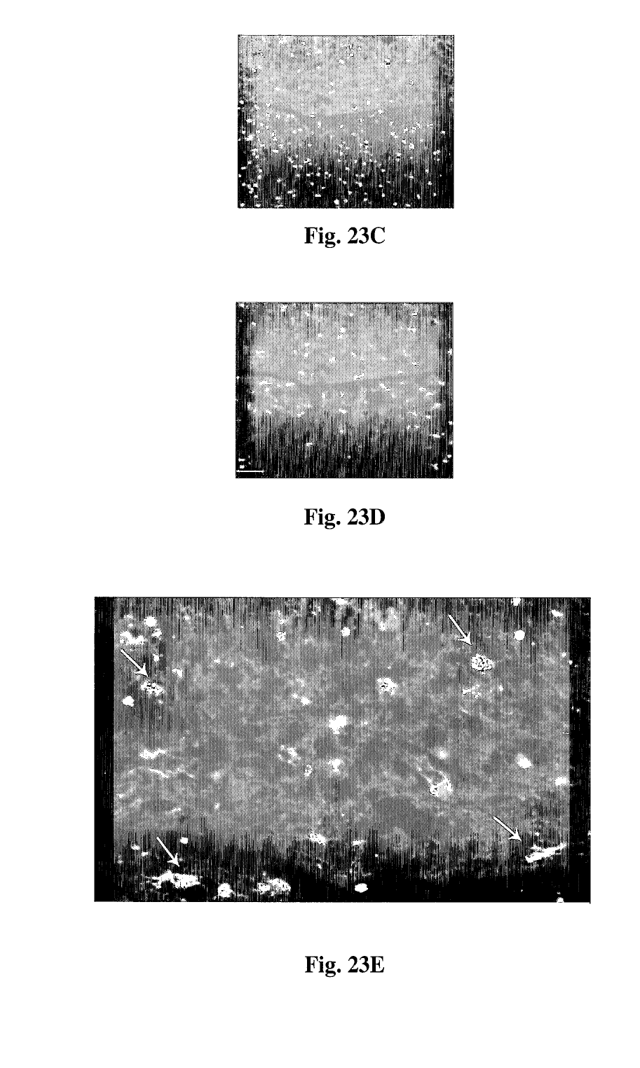

The above results raised an important question: can a T cell-based vaccination, when given in combination with aNPC transplantation, create conditions favorable for neuronal differentiation of endogenous or exogenous aNPCs?. To examine this possibility, we repeated the experiment described in FIG. 19, while also injecting the cell-proliferation marker BrdU twice daily for 3 days, starting on day 7 after aNPC transplantation (i.e., 14 days after SCl). Staining for BrdU and the early differentiation marker doublecortin (DCX) 7 days after the last BrdU injection disclosed significantly more newly formed neurons in the mice that had received the dual treatment (FIGS. 23A-23E). FIG. 23B demonstrates the distribution of DCX⁺ cells in the environment/vicinity of the injured site. Staining for the combination of BrdU and GFP and of GFP and DCX revealed virtually no double-positive cells, indicating that most of the DCX⁺ cells in the injured spinal cord had originated from endogenous aNPCs. Notably, vaccination without aNPC transplantation did not increase the formation of new neurons in the injured spinal cord.

### Example 4(4)

### Interaction of Adult Neural Progenitor Cells and T Cells In Vitro

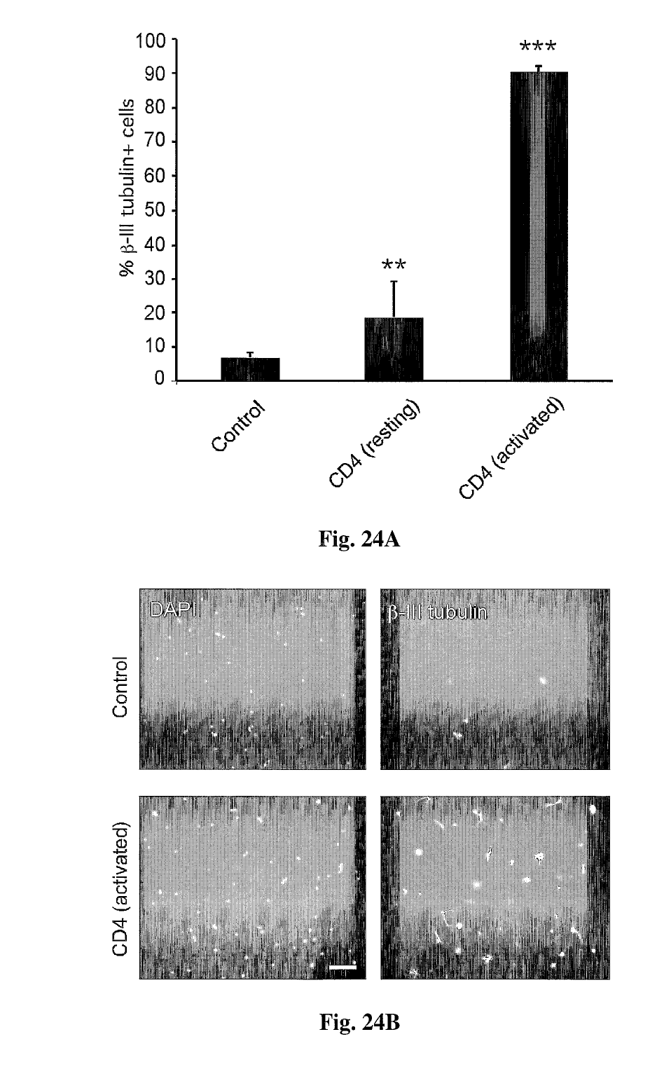

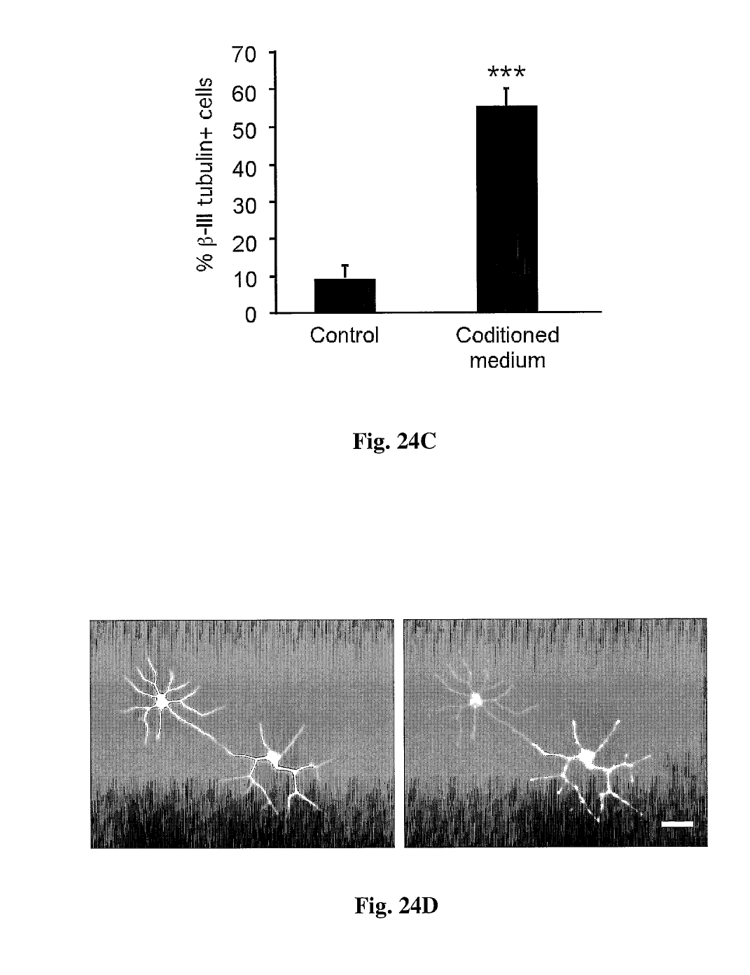

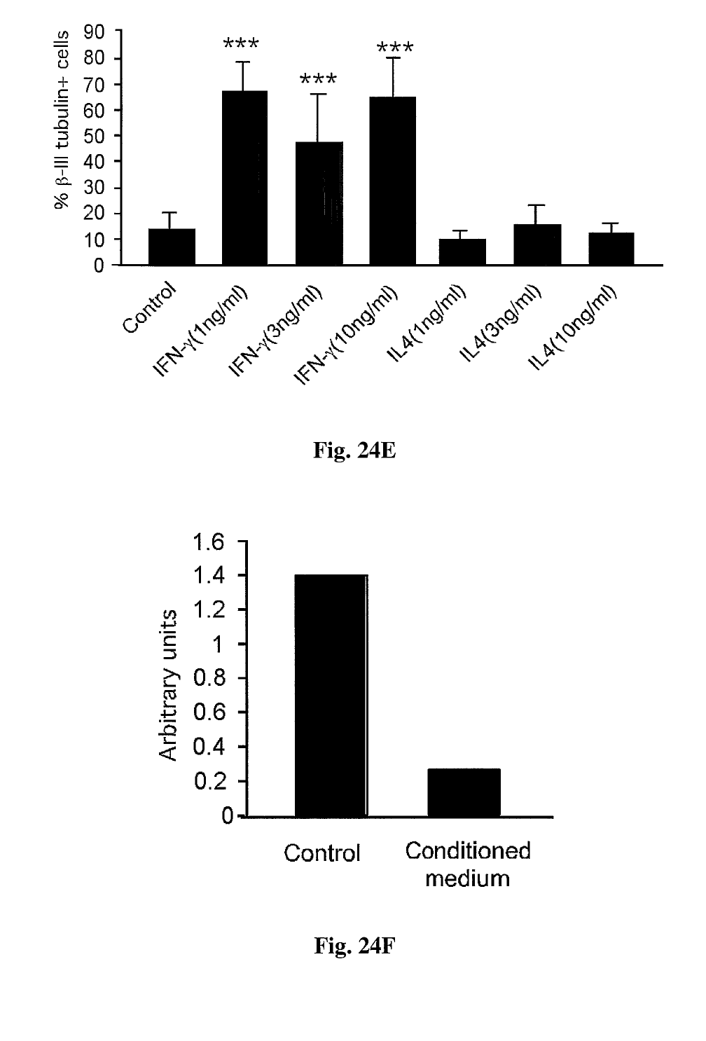

The above results indicated that cross-talk between immune cells and aNPCs was taking place at the site of injury. We showed hereinabove that microglia pre-activated with the Th1- and Th2-associated cytokines, IFN-γ and IL-4, respectively, can induce neuronal differentiation from aNPCs. We therefore sought to determine whether direct interaction between aNPCs and T cells would also result in an altered pattern of aNPC differentiation. To address this question, we activated CD4+ T cells in vitro by a cognate protocol (with anti-CD3 antibodies, anti-CD28 antibodies, and IL-2) for 24 h and then allowed their activation to continue in co-cultures with aNPCs in a transwell culture system. As controls we used cultures of aNPCs alone (in the presence of anti-CD3 antibodies, anti-CD28 antibodies and IL-2) or aNPCs cultured with CD4+ T cells in a resting state (supplemented with IL-2 only). After 5 days in culture the aNPCs in the lower chamber were fixed and analyzed for the appearance of newly formed neurons. Staining for the early neuronal marker β-III-tubulin revealed a dramatic effect of T cells on neuronal differentiation (FIG. 24A). Compared to control cultures of aNPCs alone, in which only about 7% of the cells were positive for β-III tubulin, approximately 90% of the cells expressed β-III tubulin in co-cultures of aNPCs with activated T cells (FIGS. 24A, 24B). Notably, β-III tubulin staining showed a three-fold increase (18%) relative to the control in co-cultures of aNPCs with nonactivated T cells. It thus seems that T cells induce neuronal differentiation via a soluble factor. FIG. 24B shows representative images of β-III tubulin-stained cultures of aNPCs alone (control) or of co-cultures with activated T cells. These findings imply that T cell-derived soluble factors might trigger one of the pathways of neuronal differentiation.

To exclude the possibility that the T cells had affected NPC differentiation as a consequence of encountering NPC-derived compounds, and to further substantiate our finding that the observed effect was caused by T cell-derived soluble substances, we allowed aNPCs to differentiate in the presence of medium conditioned by activated T cells. After 5 days, staining of these cultures for β-III tubulin revealed similar results to those obtained in the co-culture system (FIG. 24C), confirming that neuronal differentiation was induced by resting T cells and even more by the activated T cells. In addition to their effect on the numbers of differentiating cells, the T cell-derived soluble factors also evidently affected the cellular morphology, as manifested in the branched, elongating β-III tubulin-labeled fibers (FIG. 24D).

We next sought to determine whether the T cell-induced neurogenesis was mediated by cytokines secreted by activated T cells. aNPCs were cultured in the presence of different concentrations of the characteristic T-cell derived cytokines IFN-γ and IL-4. Analysis revealed that IFN-γ, at concentrations as low as 1 ng/ml, could induce an increase in β-III tubulin expression after 5 days in culture (FIG. 24E). In experiments described herein, we found that brief exposure to IFN-γ (24 h) was not sufficient to attain such an effect. It should be noted, however, that the morphology of the β-III-tubulin-expressing cells in IFN-γ-supplemented cultures was less developed than that seen in aNPCs cultured with activated T cells or in T cell-conditioned medium. In contrast to the effect of IFN-γ, no change in β-III tubulin expression was observed in aNPCs treated with IL-4.

These findings suggested that IFN-γ, unlike IL-4, can account in part for the T cell-induced neurogenesis. Even the effect of IFN-γ, however, was limited relative to that of the T cells or to the T cell-derived soluble factors. PCR analysis of expression of the IFN-γ receptor-1 on aNPCs disclosed that this receptor is expressed by aNPCs under all of the conditions examined here (data not shown).

Activation of the Notch pathway is essential for maintenance of aNPCs, and blockage of this pathway and its downstream transcription factors of the Hes gene family underlie the first events in neuronal differentiation. To determine whether T cell-mediated neuronal differentiation induces changes in Notch signaling, we looked for possible changes in expression of Hes genes in aNPCs following their interaction with T cell-derived substances. Real-time PCR disclosed that relative to control cultures, aNPCs cultured for 24 h in the presence of T cell-conditioned medium underwent a five-fold decrease in Hes-5 expression (FIG. 24F). Thus, differentiation induced by T cells appears to involve inhibition of the Notch pathway. Expression of Notch 1-4 by aNPCs was not altered in the presence of T cell-conditioned medium, indicating that the inhibition could not be attributed to changes in Notch expression (data not shown).

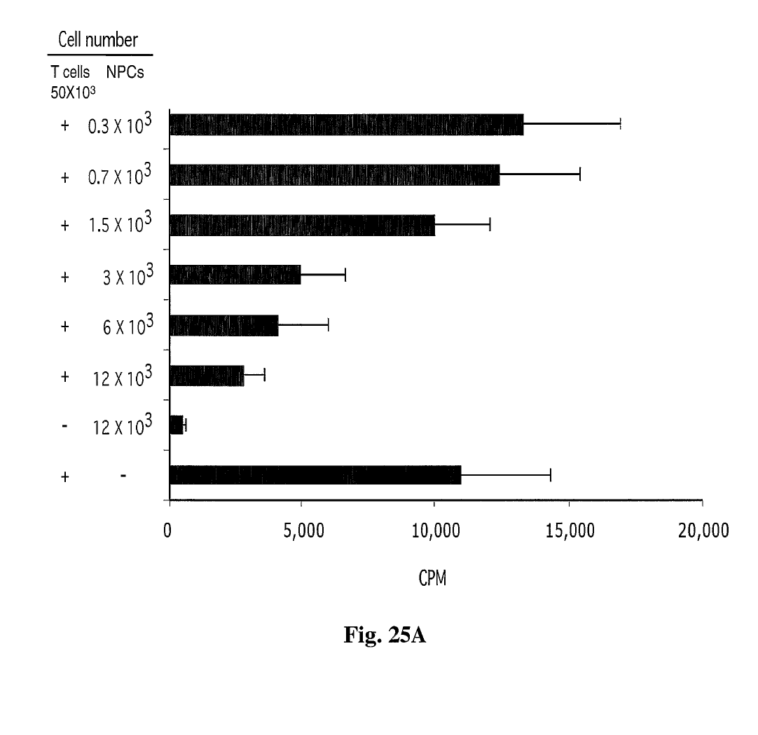

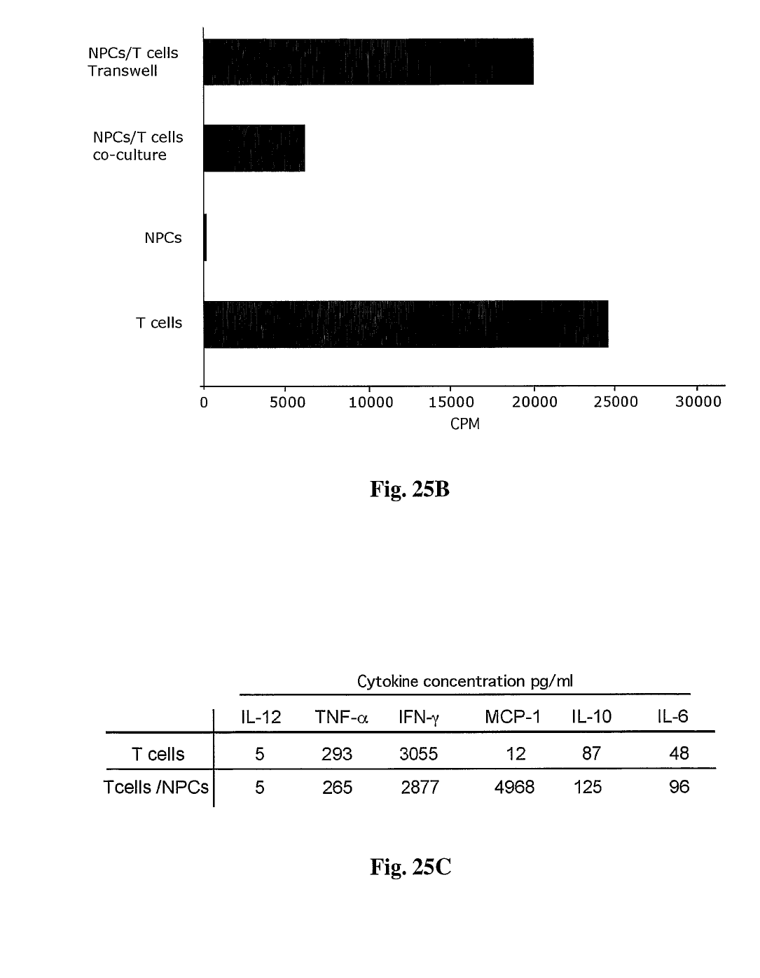

Our observation that aNPCs express an IFN-γ receptor, taken together with recent studies showing that these cells express immune-related molecules such as B-7 (Imotola et al., 2004) and CD44 (Pluchino et al., 2003), known to participate in the dialog between T cells and antigen-presenting cells (APCs), prompted us to examine whether aNPCs could affect T-cell function. First we examined the effects of aNPCs on proliferation of CD4+ T cells by assaying [³H]thymidine incorporation by the T cells. Co-culturing of T cells with aNPCs and APCs (lethally irradiated splenocytes) for 3 days resulted in a significant dose-dependent inhibition of T-cell proliferation (FIG. 25A). It is important to note that under these conditions there was only limited proliferation of aNPCs. To determine whether the inhibitory effect on T-cell proliferation is mediated by a soluble factor or requires cell-cell contact, we utilized the transwell system, plating aNPCs in the upper well. Co-culturing of T cells and aNPCs in the same well resulted in a two-fold reduction in T-cell proliferation, but this effect was diminished when the two cell populations were separated in the transwell. It thus seems that cell-cell contact is necessary for aNPCs to inhibit T-cell proliferation. To determine whether aNPCs could affect the production of cytokines by T cells, we measured the concentrations of six inflammatory cytokines in the media of co-cultured aNPCs and T cells. The concentrations of IL-12, IFN-γ, and TNF-α were similar in T-cell cultures with and without aNPCs, but the concentration of IL-10 was slightly increased (by 40%) in the co-culture. Relative to T-cell cultures alone, the concentration of IL-6 was twofold higher in the co-culture with aNPCs, and a remarkable difference was seen in MCP-1 concentration, which was higher by two orders of magnitude in the co-culture (FIG. 25C). Taken together, these results indicate that aNPCs can act directly on T cells, inhibiting their proliferative activity and changing their cytokine/chemokine production profile.

### Discussion

Local interaction between immune cells and aNPCs underlies functional recovery. In the present study we combined two different therapeutic approaches for SCI: T cell-based vaccination and transplantation of neural progenitor cells into the CSF. Each of these approaches has been shown to be potentially capable of promoting functional recovery from SCI; we show here that when combined, they operate in synergy. Our experiments, both in vivo and in vitro, demonstrated that cross-talk between immune cells and aNPCs can take place at the lesion site. The vaccination elicits a local immune response which, if well controlled, provides the cellular and molecular elements needed to attenuate degeneration and promote repair. The same response also plays a role in recruiting aNPCs to the injured site and creating niche-like compartments that support neurogenesis from endogenous aNPCs. The interaction between aNPCs and immune cells was found to be reciprocal: aNPCs could modulate the postinjury immune activity, ensuring functional recovery even under conditions of excessive immune activity (which, in the absence of aNPCs, have a detrimental effect on recovery).

### Example 5

### Immune Cells Contribute to the Maintenance of Neurogenesis and Cognition in Adulthood

Neurogenesis is known to take place in the adult brain. This work identifies T lymphocytes and microglia as pivotal players in the maintenance of hippocampal neurogenesis and spatial learning abilities in adulthood. Hippocampal neurogenesis was dramatically impaired in three different types of T cell-deficient mice, but could be restored and even boosted by T cells recognizing a specific central nervous system (CNS) antigen. Environmental enrichment did not evoke enhanced neurogenesis in immune-deficient animals, whereas in wild-type animals it led to enhanced hippocampal neurogenesis coupled with recruitment of T cells and activation of microglia. CNS-specific T cells were also found to be required for spatial learning/memory and for expression of brain-derived neurotrophic factor in the dentate gyrus, implying that a common immune-associated mechanism underlies different aspects of hippocampal plasticity and cell renewal in the adult brain.

We show here that adult mice (4-5-month-old) with severe immune deficiency experienced loss of long-term potentiation and impairment of spatial learning/memory and hippocampal neurogenesis. These functions were restored by myelin-associated autoimmune T cells. The results might shed light on age-related cognitive loss and hint at a novel approach to the maintenance of brain plasticity in adulthood.

### Materials and Methods

(xxxv) Animals. Adult male Sprague-Dawley (SPD) rats (12 weeks old) (n=6) were placed together in an enriched environment complex consisted of running wheels, tunnels, nesting material and rubber balls. Handling was limited to once a week when bedding was exchanged. Control rats were housed, three per standard cage (45×22×18 cm), under controlled conditions (23±2° C., lights on 06.00-18.00 h).

Inbred adult male C57B1/6J mice and Balb/c/OLA wildtype and SCID mice, T_{MBP}-transgenic mice, T_{OVA}-transgenic mice, RAG^{-/-} mice, and T_{MBP}-RAG^{-/-} transgenic mice, all aged 4-5 months, and B10.PL T_{MBP}-transgenic mice, and Balb/c/OLA nude and T_{OVA}-transgenic mice, all aged 8-16 weeks, were supplied by the Animal Breeding Center of the Weizmann Institute of Science (Rehovot, Israel). The B10.PL wild-type mice were purchased from The Jackson Laboratory (Bar Harbor, Me.). Mice were housed in an enriched environment complex (6 mice per cage) similar to that of the rats or in standard housing (6 mice per cage).

Both rats and mice were matched for age in each experiment and their cages were placed in a light- and temperature-controlled room.

Inbred adult male C57B1/6J mice and Balb/c/OLA wildtype and SCID mice, T_{MBP}-transgenic mice, T_{OVA}-transgenic mice, RAG^{-/-} mice, and T_{MBP}-RAG^{-/-} transgenic mice, all aged 4-5 months, and B10.PL T_{MBP}-transgenic mice, and Balb/c/OLA nude and T_{OVA}-transgenic mice, all aged 8-16 weeks, were supplied by the Animal Breeding Center of the Weizmann Institute of Science (Rehovot, Israel). The B10.PL wild-type mice were purchased from The Jackson Laboratory (Bar Harbor, Me.). Mice were housed in an enriched environment complex (6 mice per cage) similar to that of the rats or in standard housing (6 mice per cage).

(xxxvi) Administration of 5-bromo-2′-deoxyuridine and tissue preparation. Adult SPD rats were injected i.p. (50 mg/kg body weight), once daily for 5 days, with BrdU Sigma-Aldrich, St. Louis, Mo.). Rats housed under either standard or environmentally enriched conditions began receiving BrdU after being kept for 6 weeks in their respective environments, and continued to live in those environments for 1 week after the last injection. They were then deeply anesthetized and perfused transcardially, first with PBS and then with 4% paraformaldehyde. Their brains were removed, postfixed overnight, and equilibrated in phosphate-buffered 30% sucrose. Free-floating 40-m-thick coronal hippocampal sections were collected on a freezing microtome (Leica SM2000R) and stored at 4° C. prior to immunohistochemistry. Adult mice housed in an enriched environment were injected with BrdU according to the protocol used for the rats.

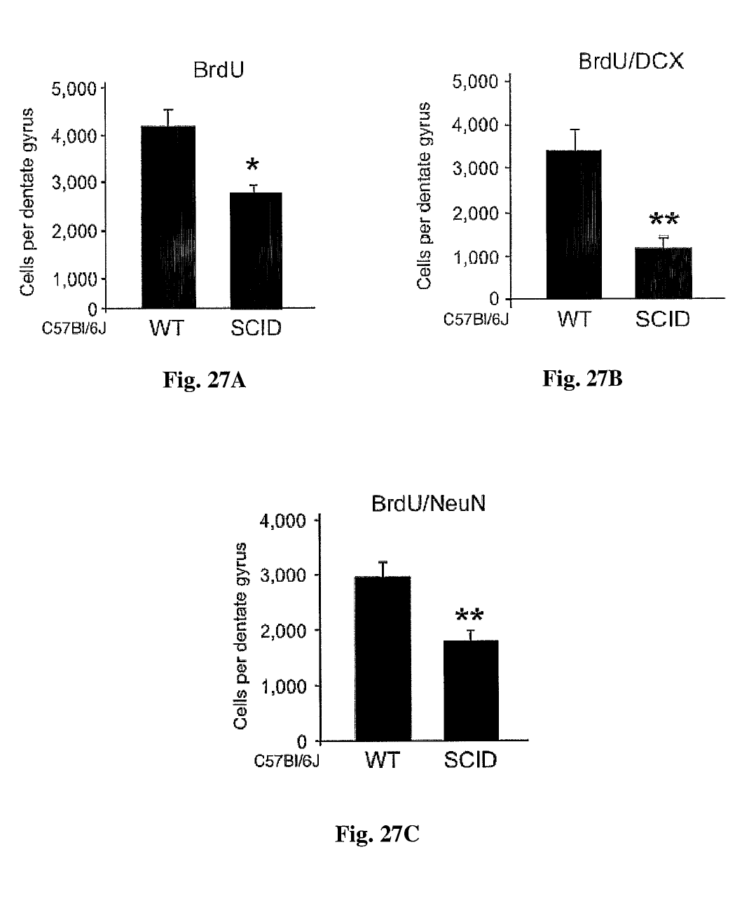

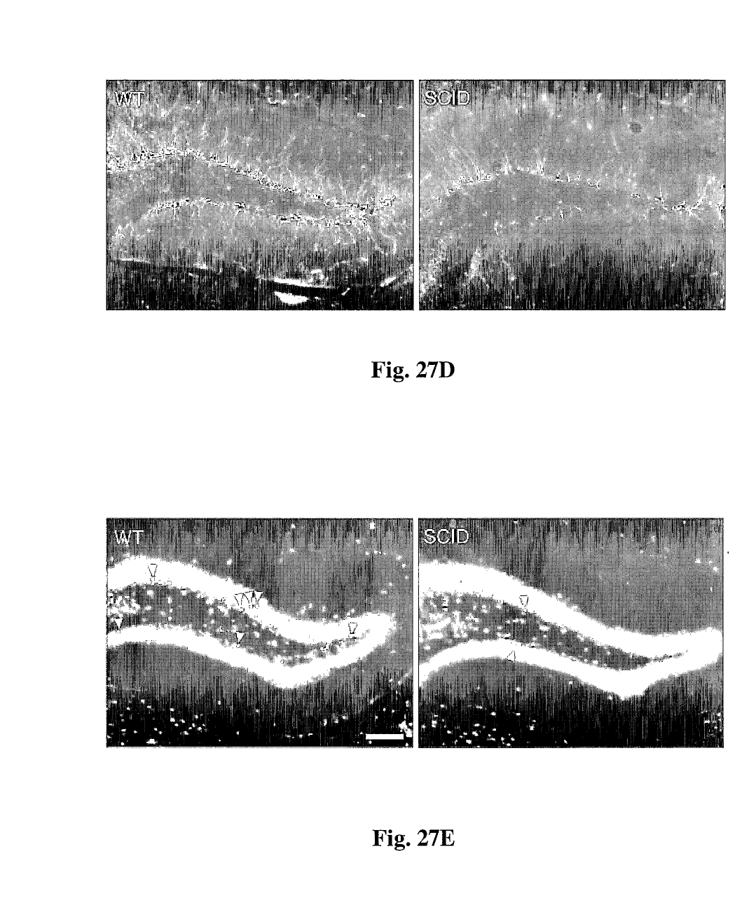

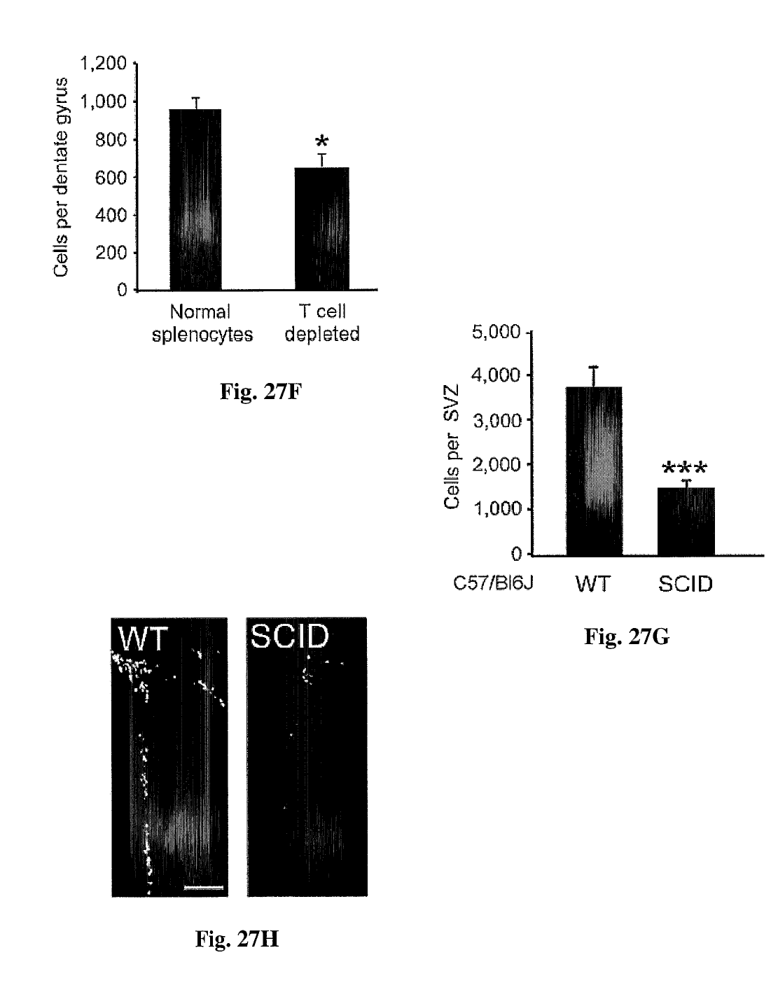

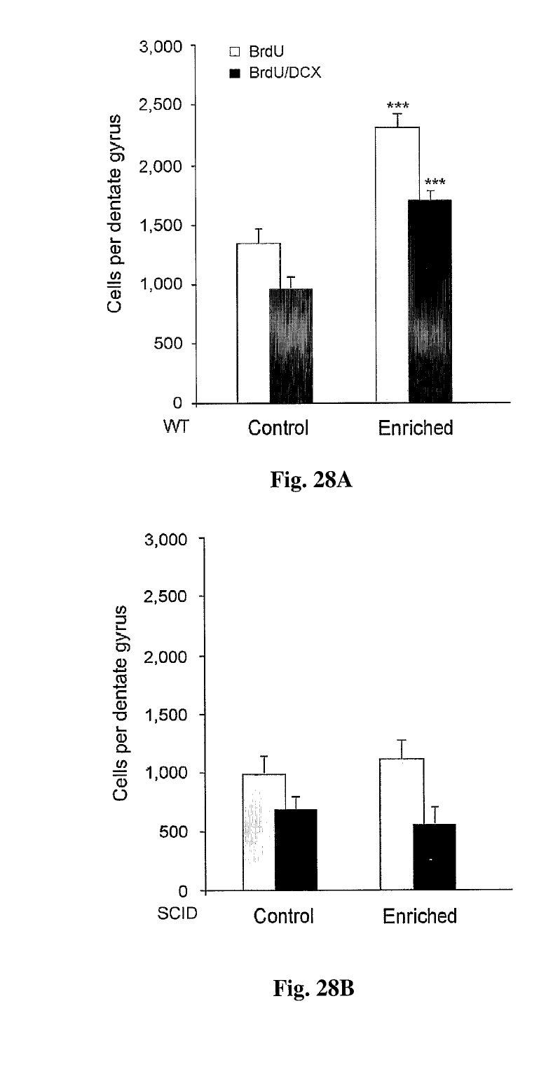

Under standard conditions, after injection of BrdU in mice (i.p., 50 mg/kg body weight) at 12-h intervals for 2 or 2.5 days (a total of 4 or 5 injections, respectively), mice were deeply anesthetized and euthanized by transcardial perfusion with PBS followed by 2.5% paraformaldehyde. Mice that received four injections were killed 48 h or 7 days after the first injection, whereas mice injected five times were killed 7 or 28 days after the first injection (for experiments presented in FIG. 27). Their brains were removed, postfixed overnight, and then equilibrated in phosphate-buffered 30% sucrose. Free-floating 16-μm or 30-μm sections were collected on a freezing microtome (Leica SM2000R) and stored at 4° C. prior to immunohistochemistry.

(xxxvii) Immunohistochemistry. Specimens obtained from rats were treated with a permeabilization/blocking solution containing 10% fetal calf serum, 2% bovine serum albumin, 1% glycine, and 0.05% Triton X-100 (Sigma-Aldrich). Primary antibodies were applied for 1 h in a humidified chamber at room temperature. For labeling of microglia we used FITC-conjugated Bandeiraea simplicifolia isolectin B4 (IB-4, 1:50; Sigma-Aldrich). To detect expression of cell-surface MHC-II proteins we used anti-MHC-II Abs (mouse, 1:50; IQ Products, Groningen, The Netherlands). T cells were detected with anti-TCR (α:/β) Abs (mouse, 1:20; Acris Antibodies, Hiddenhausen, Gmbh). Expression of IGF-I was detected by anti-IGF-I Abs (goat, 1:20; R&D Systems, Minneapolis, Minn.).

For BrdU staining, sections were washed with PBS and incubated in 2N HCl at 37° C. for 30 min. Sections were blocked for 1 h with blocking solution. The tissue was then stained with the antibodies anti-BrdU (rat, 1:200; Oxford Biotechnology, UK), anti-NeuN (mouse, 1:200; Chemicon, Temecula, Calif.), and a combination of propidium iodide (5 μg/ml) and anti-MHC-II or FITC-IB-4 diluted in PBS containing 0.05% Triton X100, 0.1% Tween 20, and 2% horse serum. Sections were incubated with the primary antibody for 24 h at 4° C., washed with PBS, and then incubated with the secondary antibodies in PBS for 1 h at room temperature while protected from light.

For BrdU staining of mouse specimens tissue sections were washed with PBS, incubated in 2N HCl at 37° C. for 30 min, and then blocked for 1 h with blocking solution (PBS containing 20% normal horse serum and 0.5% Triton X-100, or PBS containing mouse immunoglobulin blocking reagent obtained from Vector Laboratories, Burlingame, Calif.). The tissue sections were stained overnight with specified combinations of the following primary antibodies: rat anti-BrdU (1:200; Oxford Biotechnology, Kidlington, Oxfordshire, UK), goat anti-DCX (1:400; Santa Cruz Biotechnology, Santa Cruz, Calif.), mouse anti-NeuN (1:200; Chemicon, Temecula, Calif.) and rabbit anti GFAP (1:200 DAKO). Secondary antibodies used for both mouse and rat tissues were FITC-conjugated donkey anti-goat, Cy-3-conjugated donkey anti-mouse, and Cy-3- or Cy-5-conjugated donkey anti-rat (1:200; Jackson ImmunoResearch, West Grove, Pa.). For BDNF staining, tissue sections were washed with PBS and blocked for 1 h with blocking solution (PBS containing 20% normal horse serum and 0.05% saponin). They were then stained overnight at room temperature with a primary antibody solution consisting of chicken anti-BDNF (1:100; Promega, Madison, Wis.) in PBS solution containing 2% normal horse serum and 0.05% saponin. The secondary antibody was Cy-3-conjugated donkey anti-chicken (1:250; Jackson ImmunoResearch).

(xxxviii) Transfer of splenocytes to immune-deficient mice. Mice were killed and their spleens were harvested and mashed. Red blood cells were removed with a lysing buffer. The splenocyte population was depleted of T cells by the use of anti CD90 (Thy-1) microbeads (Mylteni Biotech, Bergisch Gladbach, Germany) in an autoMACS cell sorter. FACS analysis using PE-conjugated anti-CD3 antibodies (PharMingen, Becton-Dickinson, Franklin Lakes, N.J.) confirmed that fewer than 10% of the depleted splenocytes were T cells. SCID or nude mice were injected i.v. with 6×10⁷ splenocytes (nondepleted) or 2×10⁷ splenocytes that were depleted of T cells.

(xxxix) Minocycline administration. Minocycline (Sigma-Aldrich, St. Louis, Mo.) was dissolved in PBS and injected i.p into T_{MBP}-transgenic mice once a day for 14 days. The dosages injected were 50 mg/kg body weight during the first 7 days and 25 mg/kg during the last 7 days. As controls we used T_{MBP}-transgenic mice injected with corresponding volumes of PBS.

(xl) Rotarod test for basal motor ability. Mice were tested for their ability to balance on a rotating rod 7 mm in diameter (Hamilton-Kinder, Poway, Calif.). After a 1-min adaptation period the rod was accelerated by 2 rpm every 30 s, and the time that the mice remained on the rod (fall latency) was recorded. Each mouse was tested five times with a 25-min interval between tests.

(xli) Morris water maze (MWM) behavioral test. Spatial learning/memory was assessed by performance on a hippocampus-dependent visuo-spatial learning task in the MWM. Mice were given four trials per day on 4 consecutive days. In each trial they were required to find a hidden platform located 1.5 cm below the water surface in a pool 1.4 m in diameter. Within the testing room, only distal visuo-spatial cues for location of the submerged platform were available. The escape latency, i.e., the time required by the mouse to find the platform and climb onto it, was recorded for up to 60 s. Each mouse was allowed to remain on the platform for 30 s and was then moved from the maze to its home cage. If the mouse did not find the platform within 60 s, it was placed manually on the platform and returned to its home cage after 30 s. The interval between trials was 300 s. On day 5 the platform was removed from the pool and each mouse was tested by a probe trial for 60 s. On days 6-7 the platform was placed at the quadrant opposite the location on days 1-4, and the mice were then retrained in four sessions per day. Data were recorded using an EthoVision automated tracking system (Noldus).

(xlii) Quantification. For microscopic analysis we used a Zeiss LSM 510 confocal laser scanning microscope (40× magnification) or Nikon E800. Proliferation was assessed by bilateral counting of BrdU⁺ cells in the subgranular zone (SGZ) of the dentate gyrus (defined as a zone of the hilus, the width of two cell bodies, along the base of the granular layer). Neurogenesis in the dentate gyrus was evaluated by counting of cells that were double-labeled with BrdU and DCX or with BrdU and NeuN. Microglial numbers were obtained by counting of cells that were double-labeled with BrdU and markers of microglia (IB-4) or antigen-presenting cells (MHC-II). We counted the number of labeled cells in nine coronal sections (330 μm apart) per rat brain (6 rats per group) and six coronal sections (370 μm apart) per mouse brain (3-8 mouse brains per group) that were stained and mounted on coded slides. To obtain an estimate of the total number of labeled cells per dentate gyrus, the total number of cells counted in the selected coronal sections from each brain was multiplied by the volume index (the ratio between the volume of the dentate gyrus and the total combined volume of the selected sections).

For quantification of proliferation in the SVZ we used the protocol previously described (Brown et al., 2003); four coronal sections per brain (145 μm apart; 4-5 mouse brains per group), spanning from 0.38 mm anterior to bregma to 0.34 mm posterior to bregma, were stained with BrdU and mounted on coded slides. BrdU⁺ cells were counted manually in the lateral walls of both lateral ventricles Values obtained from this count were multiplied by the volume index to obtain an estimate of the total number of proliferating cells per lateral ventricle wall BDM immunoreactivity was quantified with Image-Pro Plus 4.5 software (Media Cybernetics, Silver Spring, Md.) by measuring the intensity per unit surface area at the granule cell layer of the dentate gyrus. At least three hippocampal sections per mouse were used for this analysis.

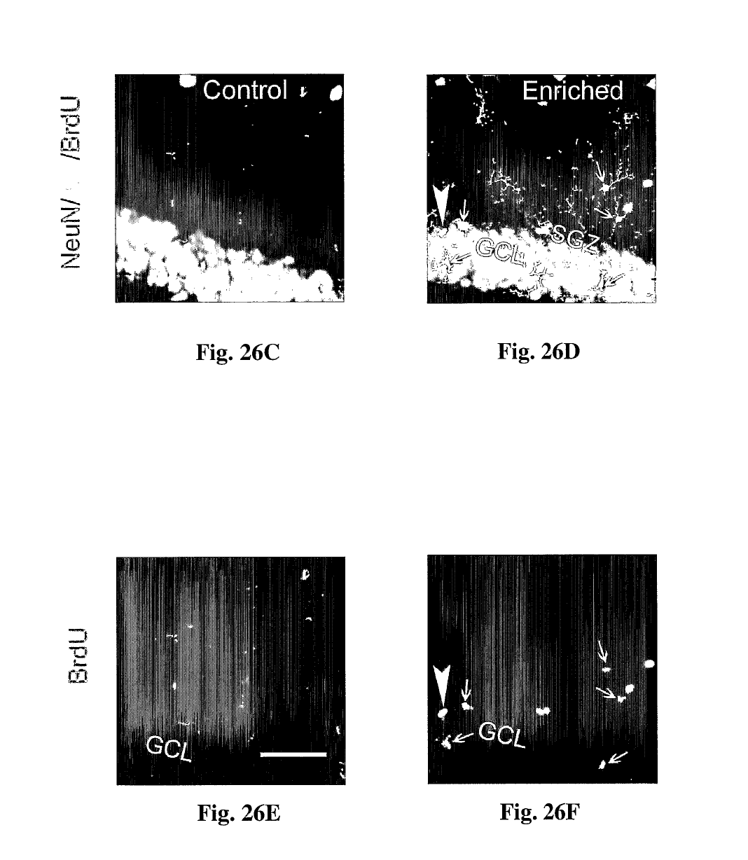

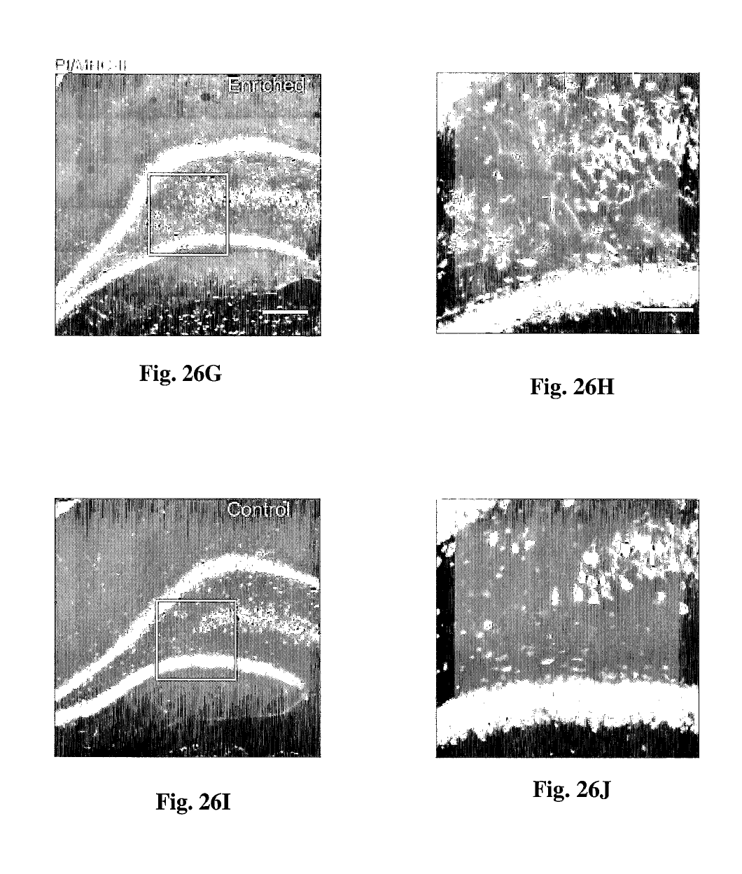

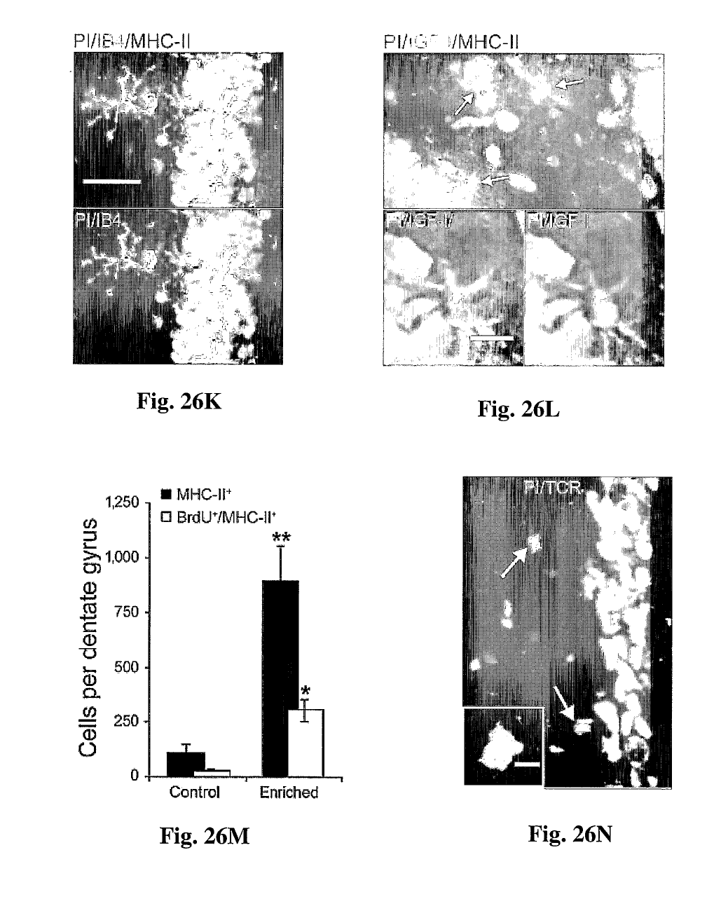

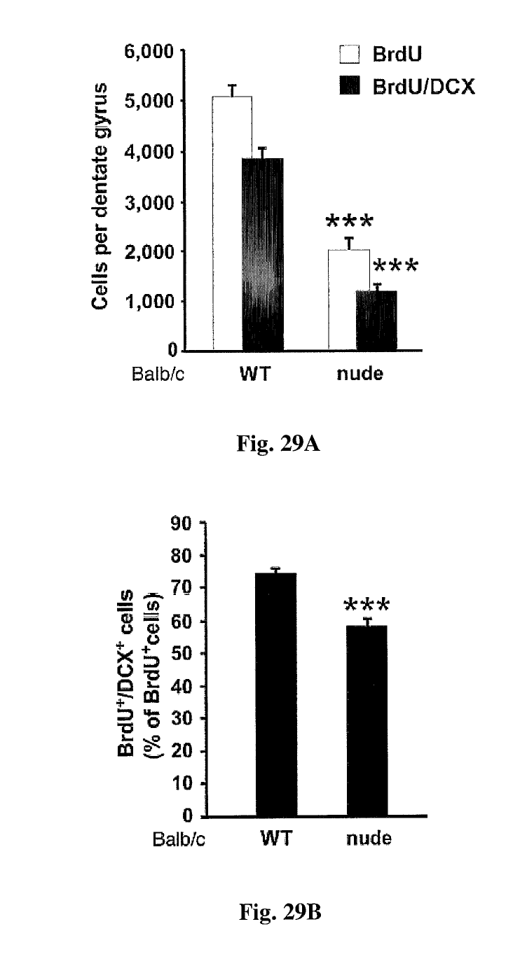

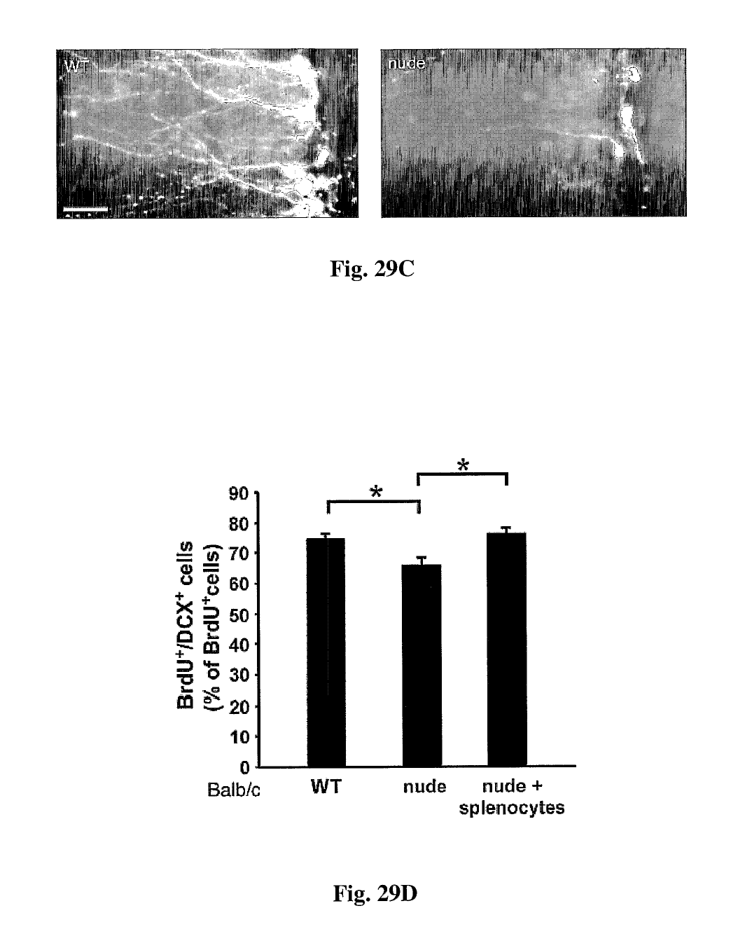

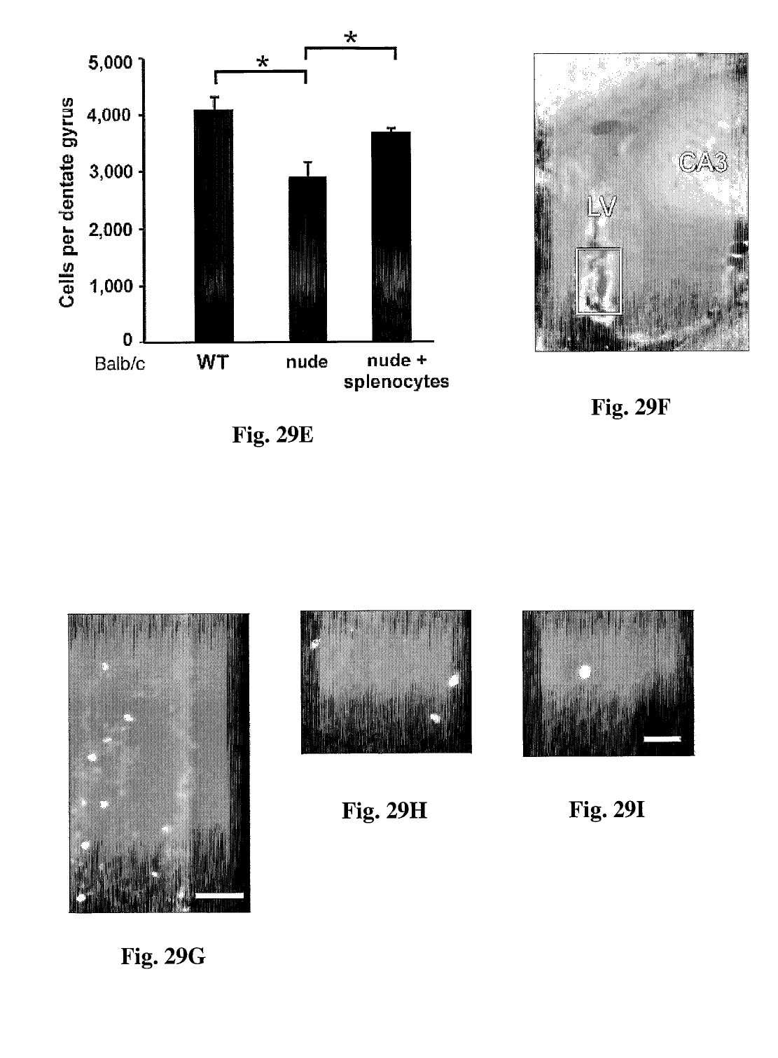

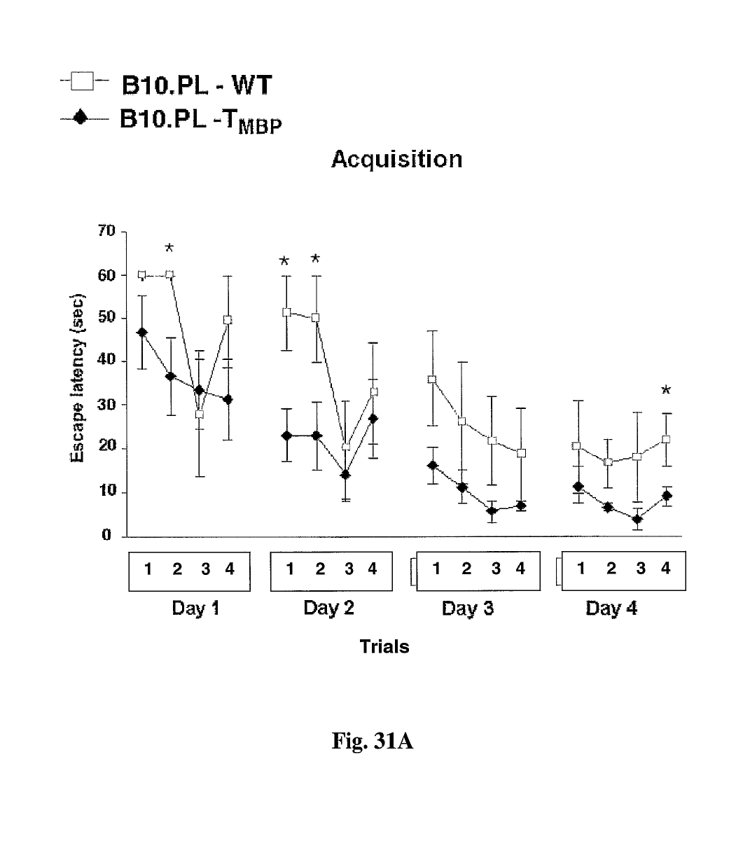

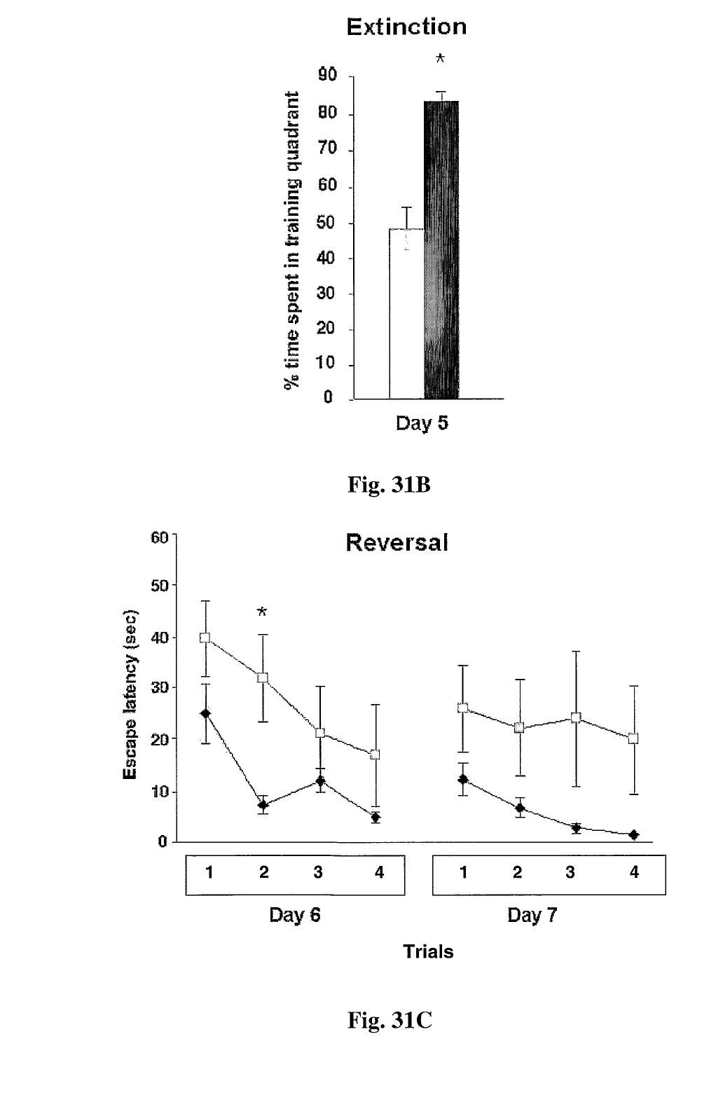

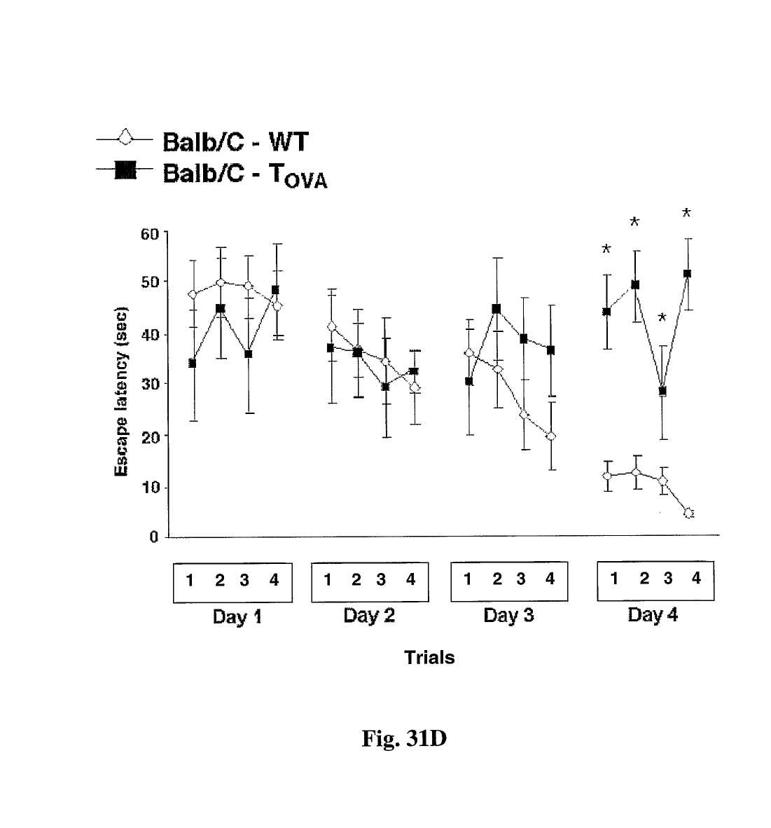

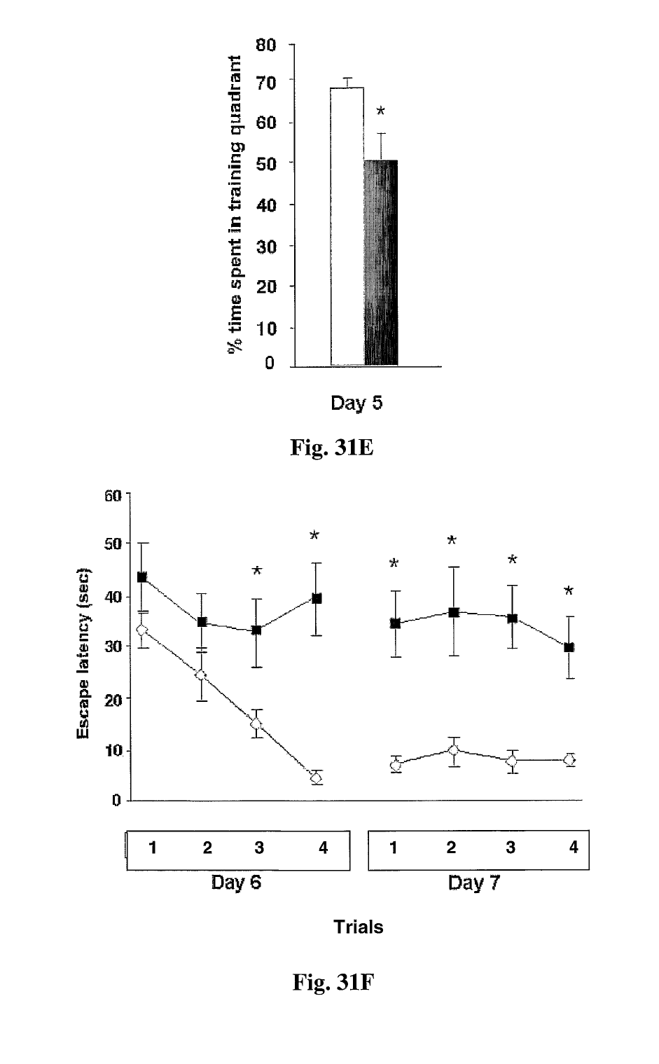

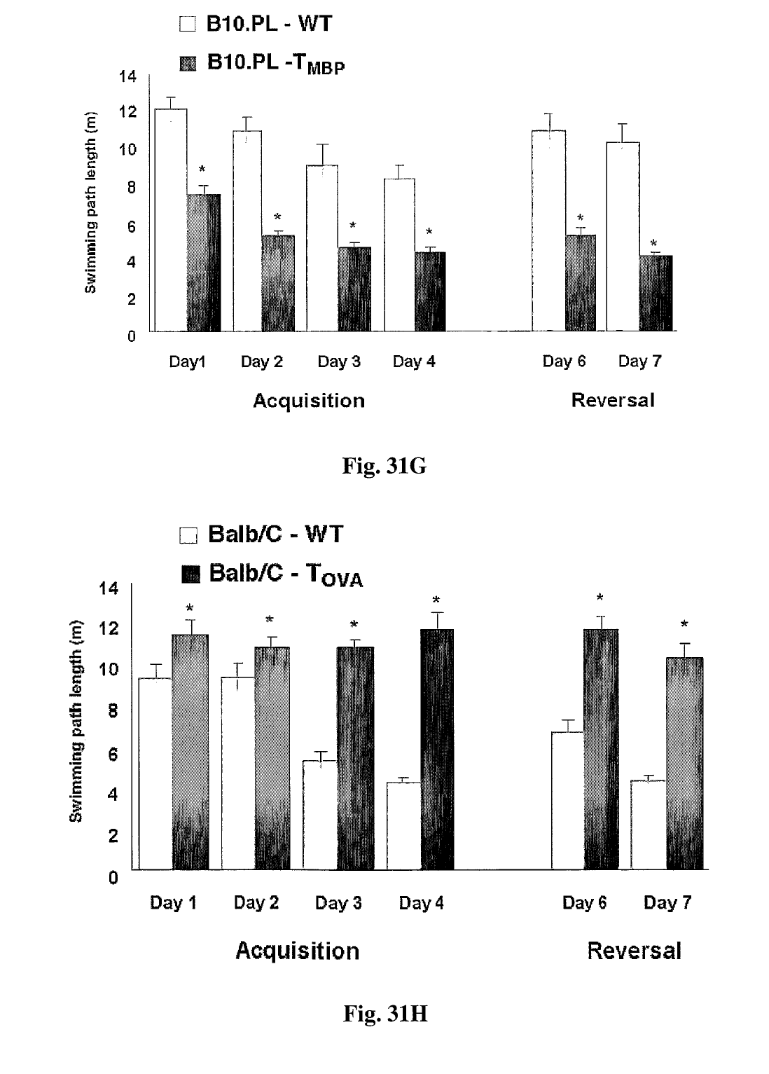

(xliii) Statistical analysis. A two-tailed unpaired Student's t-test was used for analyses of the experiments presented in FIGS. 26, 27, 30 and 32. The data from the experiments presented in FIGS. 29 and 31 were analyzed with ANOVA, as indicated in the figure legends.

(xliv) Measurements of hippocampal activity and plasticity in vitro. Mice from each group were decapitated and their brains were placed in ice-cold artificial cerebrospinal fluid (ACSF) containing 124 mM NaCl, 2 mM KCl, 1.24 mM KH₂PO₄, 2 mM MgSO₄, 2.5 mM CaCl₂, 26 mM NaHCO₃, and mM 10 glucose. pH 7.4. After removal of the hippocampi, 350-μm hippocampal slices were obtained with a McIlwain tissue slicer (Mickle Laboratory Engineering. Guildford, Surrey, UK) and incubated for 1.5 h at room temperature in ACSF saturated with 95% O₂ and 5% CO₂ gas mixture. The slices were then submerged in a perfusion chamber, heated to 30-32° C., and superfused with ACSF at a rate of 2 ml/min. A bipolar stimulating electrode (25-μm nichrome wires) was placed in the stratum radiatum of the CA1 region. Test stimuli (100-μs pulse duration) were delivered at 30-s intervals, with the intensity adjusted such that the evoked responses were approximately 50% of the maximal response. An extracellular recording electrode containing 0.75 M NaCl (2-4 MΩ) was placed in the stratum radiatum of the CA1 region for recording of excitatory postsynaptic potential (EPSP). LTP vas induced by theta-burst stimulation (TBS) of the Schaffer collaterals (10 trains of 4 pulses at 100 Hz, separated by 200-ms inter-train intervals, at the same intensity as the test stimulation). Stimulation and data acquisition were performed using the LTP software (Anderson and Collingridge, 2001).

### Example 5(1)

### Spatial Association Between Activated Microglia and T Cells in Areas of Increased Neurogenesis in Environmentally Enriched Rats

Our working hypothesis was that both CNS resident and systemic immune cells are active participants in adult hippocampal neurogenesis. With the object all examining whether this is indeed the case, we looked for a model in which neurogenesis is augmented physiologically without any intervention (Kemperman et al., 1997). Mental and physical activities have been shown to promote neurogenesis in adulthood by increasing survival and proliferation, respectively, of newly formed neurons in the dentate gyrus (van Praag et al., 1999). We anticipated that if T cells and activated resident microglia participate in neurogenesis, they would reach detectable levels under enriched conditions.

Three-month-old Sprague-Dawley rats were housed in standard capes (control; three rats per cage) or in an enriched environment (six rats per cage) that provided favorable conditions with regard to space, social interaction, sensory stimuli, and opportunities for physical activity. After 6 weeks each rat received a 5 day course of daily i.p. injections of BrdU to enable detection of newly formed cells in the dentate gyrus. One week later the rats were euthanized and the hippocampal areas of their brains were examined with antibodies against BrdU, the neuronal marker NeuN, and the microglial marker IB-4. As expected, significantly more newly formed neurons (BrdU+/NeuN+) were found in the dentate gyri of rats housed in the enriched environment than in control rats (FIG. 26A). We next examined whether among the BrdU+/NeuN− cells were cells expressing the microglial marker IB-4. Compared to control rats, in the dentate gyri of rats kept in the enriched environment there was a significant increase in the numbers of microglia (IB-4+) as well as of newly formed microglia (IB-4+/BrdU+) (FIG. 26B). FIGS. 26c-26f show representative confocal micrographs of staining for BrdU, NeuN, and IB-4 in the dentate gyri of control rats (FIGS. 26C, 26E) and of rats housed in an enriched environment (FIGS. 26D, 26F). To avoid the possibility that BrdU labeling in IB-4+ cells reflects phagocytosis of dying proliferating cell's, we took special care in our analysis to count only IB-4+ cells with the typical ramified morphology, in which the BrdU staining appeared at the center of the cell. Previous studies have distinguished between LPS-activated microglia (the classically activated microglia that apparently impair neurogenesis; Monje et al. 2003) and microglia activated by cytokines associated with adaptive immunity, which are neuroprotective. Thus, as shown in Examples hereinbefore, microglia primed with the Th2-derived cytokine IL-4 strongly express MHC-II and IGF-1. To determine whether the phenotype of microglia observed in the dentate gyri of rats housed in an enriched environment resembles that of microglia activated by T cell-derived cytokines, we stained adjacent sections from the same brains with antibodies directed against MHC-II (FIGS. 26G-26J). Analysis of the dentate gyri of the environmentally enriched rats showed that approximately 30% of the microglia (IB-4+) expressed MHC-II (FIG. 26K). These microglia, were found to be localized in the hilus and in close proximity to the subgranular zone (SGZ) of the dentate gyrus or were even incorporated within the granular cell layer itself (FIG. 26H). Many of the MHC-II⁺/IB-4+ cells were also stained for IGF-I (FIG. 26L). Quantification of MHC-II⁺ cells in the dentate gyrus revealed that their numbers were increased by approximately 5-fold under enriched conditions, demonstrating that a significant proportion of the newly formed BrdU+ cells could function as antigen-presenting cells (FIG. 26M). Expression of MHC-II suggested that local interaction of microglia with T cells was taking place. Staining with antibodies directed against a T-cell receptor revealed the presence of some T cells in the parenchyma of the hilus of the dentate gyrus of environmentally enriched rats (FIG. 26N). These results show that under conditions of environmental enrichment, increased neurogenesis in the adult hippocampus is correlated with the appearance of immune cells (T cells and MHC-II-expressing microglia) in the hilus and SGZ of the dentate gyrus, an area known to harbor the hippocampal neural stem/progenitor cells.

### Example 5(2)

### Impairment of Neurogenesis in Immune-Deficient Mice

Detection of T cells in the hippocampus of adult rats under conditions of activity-induced enhanced neurogenesis raised the question of whether T cells area needed for neurogenesis under normal conditions. To address this question, we first compared neurogenesis in the hippocampal dentate gyri of wild-type adult C57B1/6J mice and mice with the same genetic background but suffering from deficiency in adaptive immunity and thus lacking both T- and B-cell populations (severe combined immune deficiency; SCID). We examined three phases of neurogenesis: cell proliferation, early neuronal differentiation, and long-term survival of newly-formed neurons. For comparison of the numbers of proliferating cells in the SGZ of the dentate gyrus in the two groups, all mice received four BrdU injections (ever, 12 h, i.p.). The mice were euthanized 2 days after the first injection (i.e., 12 h after the last injection), their brains were excised, and sections of the hippocampus were examined immunohistochemically. Significantly fewer BrdU+ cells in the SGZ of the dentate gyrus were observed in the SCID mice than in the wild-type controls (36.4±2.2% fewer, FIG. 27A). These findings strongly imply that when adaptive immunity is deficient, proliferation in the dentate gyrus is impaired.

Next we examined whether deficiency of adaptive immunity also affects the differentiation of newly proliferating cells into neurons. SCID and wild-type mice received five BrdU injections (every 12 h, i.p.) and were euthanized 7 days or 28 days after the first injection. On day 7 their dentate gyri were analyzed immunohistochemically for the presence of proliferating cells (BrdU+) expressing the early neuronal differentiation marker doublecortin (DCX+). Significantly fewer BrdU+/DCX+ cells were found in the dentate gyri of SCID mice than in those of the wild type (65.9±8.1% fewer, FIG. 27B). The two groups did not differ significantly in the ratio of new neurons (BrdU+/DCX+ cells) to the total number of proliferating (BrdU+) cells (60.2±4.8% and 57.8±6.3%, respectively, P=0.8).

Since most of the new neurons undergo apoptosis within 3-4 weeks of their formation, we examined whether the deficiency in adaptive immunity would also be reflected in the number of surviving new neurons at that time. On day 28, significantly fewer cells double-labeled for BrdU and NeuN were seen in the SCID mice than in the wild type (39.9±7.2% fewer, FIG. 27C). Confocal micrographs illustrate differences between wild-type and SCID mice in the numbers of BrdU+/DCX+ cells on day 7 (FIG. 27D) and of BrdU⁺/NeuN⁺ cells on day 28 (FIG. 27E). Taken together, these results suggest that the effect exerted by the adaptive immune system on hippocampal neurogenesis is not transient; at all time points tested the absolute number of newly formed neurons was higher in the wild-type than in SCID mice. Double staining for BrdU and the astrocytic marker GFAP disclosed no differences in the ratio of BrdU+/GFAP+ cells to the total number of BrdU+ cells between wild-type and SCID mice (8.0±0.8% and 7.8±1.0%, respectively; P=0.9). Because the absence of adaptive immunity, as manifested in the number of newly formed neurons, was found to be most pronounced 7 days after the first BrdU injection, this was chosen as the time point of reference for hippocampal neurogenesis throughout the rest of the study.

The SCID mutation is also characterized by defective DNA-dependent protein kinase activity, with resulting impairment of DNA repair. To exclude the possibility that the impairment of neurogenesis observed here in the SCID mice was caused by this non-immunological defect, and to verify that the impaired neurogenesis in these mice could be attributed specifically to a deficiency of T cells, we compared neurogenesis in SCID mice replenished by intravenous (i.v.) injection of splenocytes from wild-type matched controls (‘normal splenocytes’) to that in SCID mice replenished with splenocytes depleted of T cells. Seventeen days after splenocyte replenishment, the mice were injected i.p. with BrdU (twice daily for 2 days) and 7 days later their hippocampi were analyzed. The T-cell content of each replenished mouse on the day of hippocampal excision was verified by FACS analysis (not shown). FIG. 27F demonstrates the presence of significantly more BrdU+/DCX+ cells in the SCID mice replenished with whole splenocytes than in the SCID mice replenished with splenocytes depleted of T cells.

Other than in the dentate gyrus, continuous neurogenesis from neural stem/progenitor cells in the adult CNS occurs in the subventricular zone (SVZ) of the lateral ventricles. We suspected that if immune deficiency causes impairment of progenitor cell proliferation in the dentate gyrus, it would have a similar effect in the SVZ. Quantification of BrdU⁺ cells in the SVZ on day 2 after BrdU injection disclosed significantly fewer in the SCID mice than in the wild type (60.6±4.8% fewer; FIGS. 27G-27H), implying that immune cells are needed for progenitor proliferation in both germinal centers of the adult brain.

The observed correlation between activated immune cells and enhanced neurogenesis in the dentate gyri of rats kept in an enriched environment (FIG. 26), coupled with the observed decrease in neurogenesis in immune-deficient mice (FIG. 27), raised a key question: Can activity-induced neurogenesis occur in immune deficient mice? To address this question, we examined whether environmental enrichment enhances hippocampal neurogenesis in SCID mice. The enrichment paradigm and BrdU injection protocol used for these series of experiments in mice were the same as those used for the rat experiments (see Methods and FIG. 26). Wild-type mice housed in the enriched environment exhibited the expected increase in neurogenesis relative to wild-type mice housed in standard conditions (77.2±7.7% increase, FIG. 27A); the SCID mice, however, did not (FIG. 27B). Inspection of the social and physical activities of the mice kept in enriched conditions disclosed no overt behavioral differences between the immune deficient and wild-type mice. These results provided evidence that enriched conditions enhance neurogenesis by a mechanism, which requires peripheral immune cells.

### Example 5(3)

### Impairment of Neurogenesis in Mice Devoid of T Cells

To further substantiate our contention that the peripheral immune cells participating in adult neurogenesis are T cells, we compared the formation of new neurons in wild-type Balb/c/OLA mice to that in strain-matched nude mice, which are deprived of their mature T-cell population but not of their B cells. As with SCID mice, 7 days after BrdU injection significantly fewer cells double-labeled with BrdU and DCX cells were seen in the dentate gyrus of the nude mice than of the wild type (68±8% fewer; FIG. 29A). Notably, unlike in the SCID mice, the ratio of new neurons (BrdU+/DCX+ cells) to the total number of proliferating (BrdU+) cells was significantly smaller in the nude mice than in the wild type (FIG. 29B), suggesting that T cells can affect not only proliferation but also neuronal differentiation. Moreover, the dendritic arborization of DCX+ cells in the two groups differed dramatically; dendrites in the nude mice were significantly less abundant and significantly shorter than those in the wild type (FIG. 29C).

To further support our contention that the impaired neurogenesis in T cell-deficient nude mice is attributable to the absence of T cells, we transferred splenocytes (containing a normal population of mature T cells) from wild-type mice to syngeneic nude mice and, as in the SCID mice, analyzed the hippocampi 17 days after splenocyte injection. However, preliminary data had suggested that the ability of transferred splenocytes to restore neurogenesis depends on the time lapse after their injection. Therefore, with the object of analyzing the results in the same splenocyte-recipient mice at an additional time point, we used two markers of cell proliferation (BrdU and PCNA—an endogenous marker of proliferating cells). We injected BrdU 10 days after splenocyte injection, and then analyzed the hippocampi (excised on day 17) for both BrdU (which detected proliferation which took place on day 10 after splenocyte injection), and for PCNA (which detected the proliferating cells at the day of excision, day 17). FIG. 29D shows that the BrdU-labeled cells in the dentate gyri of nude mice replenished with splenocytes (‘replenished nude’) contained a significantly higher percentage of cells co-labeled with DCX than in nude mice without such replenishment (‘control nude’). Whereas the total numbers of BrdU-labeled cells in the dentate gyrus did not differ significantly between the replenished nude mice and the nonreplenished nude controls (data not shown), PCNA-labeled cells (representing proliferation on the day of excision) in the replenished nude mice were significantly more numerous than in the controls, and their mean numbers indeed approached those in the wild type (P=0.3) (FIG. 29E). Thus, T cell-induced enhancement of proliferation in the nude mice was observed 17 days after replenishment (as in the case of SCID mice; FIG. 27f), but not 10 days after replenishment. These findings supported our suggestion that the impaired neurogenesis observed in nude mice was T-cell dependent and could be partially restored in the adult animal.

Using anti-CD3 antibodies, we were able to detect T cells in the brain parenchyma of both wild-type and splenocyte-replenished nude mice. The location of the T cells was not restricted, however, to areas of active adult neurogenesis, T cells could also be seen in the brain parenchyma, mainly around the walls of the ventricles adjacent to the hippocampus. FIGS. 29F-291 show the presence of T cells in the brain of a replenished nude mouse and of a wild-type mouse. These findings further substantiate the notion that the impaired neurogenesis in the immune-deficient mice is an outcome of the peripheral immune deficiency rather than of the genetic defect.

### Example 5(4)

### Adult Neurogenesis Requires T Cells that Recognize CNS-Specific Antigens, but not T Cells that Recognize Non-CNS Antigens

Previous findings from our group have shown that antigenic specificity to CNS autoantigens, such as certain peptides of MBP, is required for expression of the neuroprotective effect of T cells under traumatic or degenerative conditions (Moalem et al., 1999; Yoles et al., 2001). To determine whether the same specificity rule is also applicable to the T-cell effect on adult neurogenesis, we used transgenic mice possessing a normal B-cell population and a genetically engineered excess of monospecific T cells directed either to MBP (T_{MBP}) or to an irrelevant (nonself) antigen. Mice in the former group express a transgene encoding a T-cell receptor that recognizes an epitope (Ac1-11) of MBP, and therefore approximately 98% of the T-cell pool in these ‘T_{MBP}-transgenic’ mice consists of Ac1-11 T_{MBP} cells. The remaining 2% are endogenous T cells whose regulatory activity is apparently sufficient to prevent the T_{MBP} transgenic mice from spontaneously developing autoimmune encephalomyelitis. As a control for the antigenic specificity we used transgenic mice that possess a normal B-cell population but express a T-cell receptor that recognizes ovalbumin (‘T_{OVA}-transgenic’ mice), and thus bears mainly T_{OVA} cells. We expected that if neurogenesis requires the presence of CNS-specific T cells, such as T_{MBP} cells, the T_{OVA}-transgenic mice would behave like SCID and nude mice.

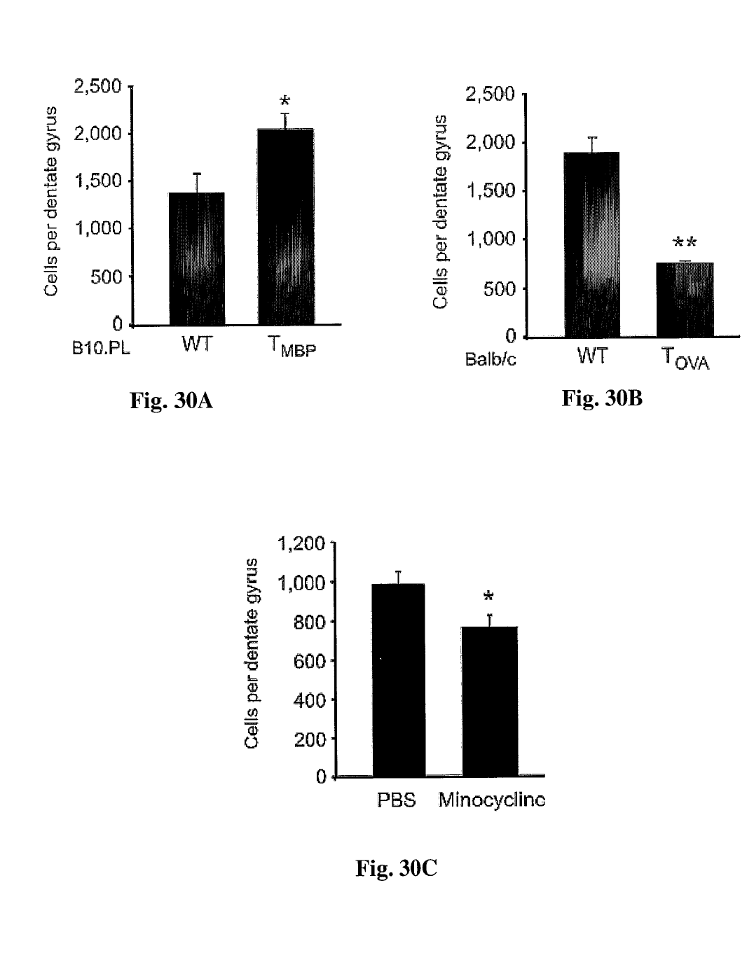

Because the two types of transgenic mice had different genetic backgrounds, we compared each of them to their matched wild-type controls. Examination of hippocampal dentate gyri from T_{MBP}-transgenic mouse brains that were excised 7 days after the first BrdU injection and then double-stained with BrdU and DCX revealed significantly more BrdU+/DCX+ cells than in their wild-type counterparts (FIG. 30A). Under the same experimental conditions, significantly fewer BrdU+/DCX+ cells were seen in the T_{OVA}-transgenic mice than in their controls (FIG. 30B). We repeated this experiment using T_{OVA}-transgenic mice that were housed in an enriched environment. Counting of BrdU+/DCX+ cells in the dentate gyri of these mice disclosed that hippocampal neurogenesis in the environmentally enriched mice and in mice housed under standard conditions did not differ (962±45 and 1054±77, respectively; P=0.5). These findings show that CNS-specific T cells are needed for activity-induced neurogenesis in adult mice. Comparison of glial differentiation in the T_{MBP} and T_{OVA} transgenic mice and their relevant wild-type controls disclosed no significant differences between them (data not shown).

To verify that the effect of CNS-specific T cells on neurogenesis is exerted via their activation of microglia, we used the antibiotic drug minocycline, which blocks microglial activity in the CNS under pathological inflammatory conditions (Ekdahl et al., 2003). The experiment was carried out with T_{MBP}-transgenic mice because of the likelihood that their higher incidence of CNS-specific T cells and their enhanced neurogenesis relative to the wild type would make it easier to detect any potential effect of the drug. Treatment of the T_{MBP}-transgenic mice with a daily dose of minocycline resulted in a significant decrease in the number of BrdU+/DCX+ cells in the dentate gyrus (FIG. 30C). This finding supported our contention that the effect of CNS-specific T cells on neurogenesis is mediated, at least in part, via their dialog with microglia

### Example 5(5)

### Spatial Learning Abilities Require T Cells that Recognize CNS-Specific Antigens, but not T Cells that Recognize Non-CNS Antigens

Recent findings point to an association between adult neurogenesis and certain hippocampal activities (Gould et al., 1999), although an association between adult neurogenesis and performance of the hippocampus-dependent task of spatial learning/memory in the MWM is still a matter of debate (Snyder et al., 2005). A study by our group showed that spatial learning/memory, as assessed by MWM performance, is impaired in immune-deficient mice (Kipnis et al., 2004). Those findings, together with the present finding that hippocampal neurogenesis is significantly impaired in mice deficient in CNS-specific T cells, prompted us to examine whether the hippocampus-dependent activity of spatial learning/memory is also dependent on the presence of autoimmune T cells. We therefore compared the MWM performance of T_{MBP} mice and of T_{OVA} mice relative to their respective matched wild-type controls. To ensure that any observed differences in performance in the MWM could be attributed to cognitive activity rather than to motor ability, we first subjected all the mice to a rotarod test. Recordings of the time that each mouse spent balancing on the rotating rod before falling disclosed no significant differences among all tested groups in fall latencies (mean±s.e.m in seconds: T_{MBP}, 39±2.3; B10.PL wild type, 37.6±1.1; T_{OVA}, 40.7±1.9; Balb/c wild type, 40.7±4.2).

When tested in the MWM, the T_{MBP} transgenic mice performed better than their controls in all three phases of the task (FIGS. 31A-31C). In the acquisition phase the T_{MBP} mice took significantly less time than the controls to find the hidden platform (FIG. 31A). In the extinction phase, in which the platform was removed from the water maze, T_{MBP} mice spent a significantly larger proportion of time than their controls in the quadrant that formerly contained the platform (FIG. 31B). In the reversal phase of the task, when the platform was replaced in a position opposite its former location, the T_{MBP} mice again took significantly less time than the controls to find it (FIG. 31C). It thus seemed that T_{MBP} transgenic mice have better spatial learning/memory abilities than the wild type. In contrast to the T_{MBP} mice, the performance of T_{OVA} transgenic mice in all phases of the MWM was significantly worse than that of their controls (FIGS. 31D-31F), apparently reflecting impairment in their spatial learning/memory ability.

Measurement of the length of the path taken by each mouse to reach the hidden platform showed that, relative to their respective wild-type controls, the path taken by the T_{MBP} mice was shorter (FIG. 31G) but the path taken by the T_{OVA} mice was longer (FIG. 31H). These results further support the contention that the poor performance of T_{OVA} mice in the MWM task resulted from a cognitive deficit rather than from a motor disability. Furthermore, the performance of T_{OVA} mice in this test was similar to that of the SCID mice in our previous study (Kipnis et al., 2004). These findings suggest that T cells, which recognize specific CNS autoantigens, unlike T cells which recognize nonself-antigens such as ovalbumin, are sufficient for maintenance of learning abilities in adulthood.

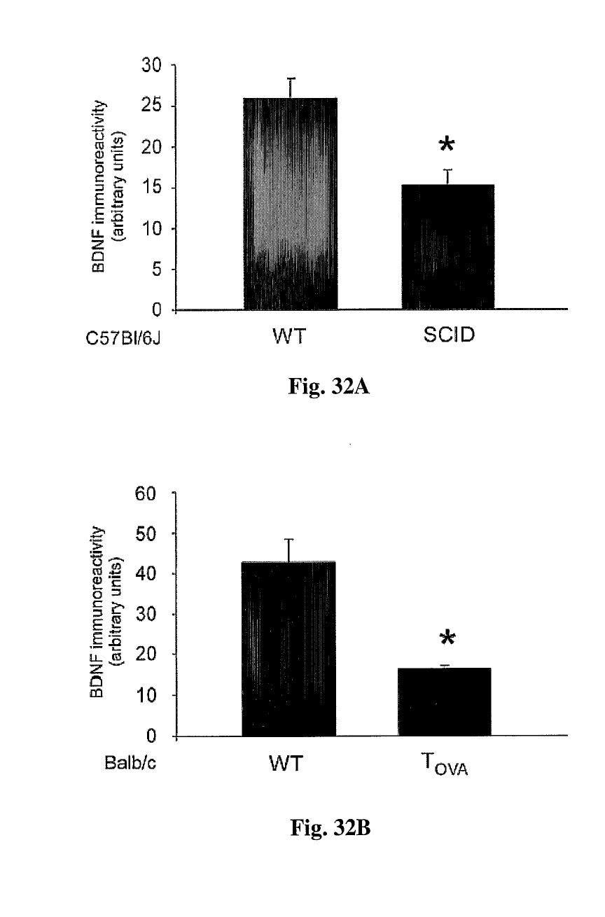

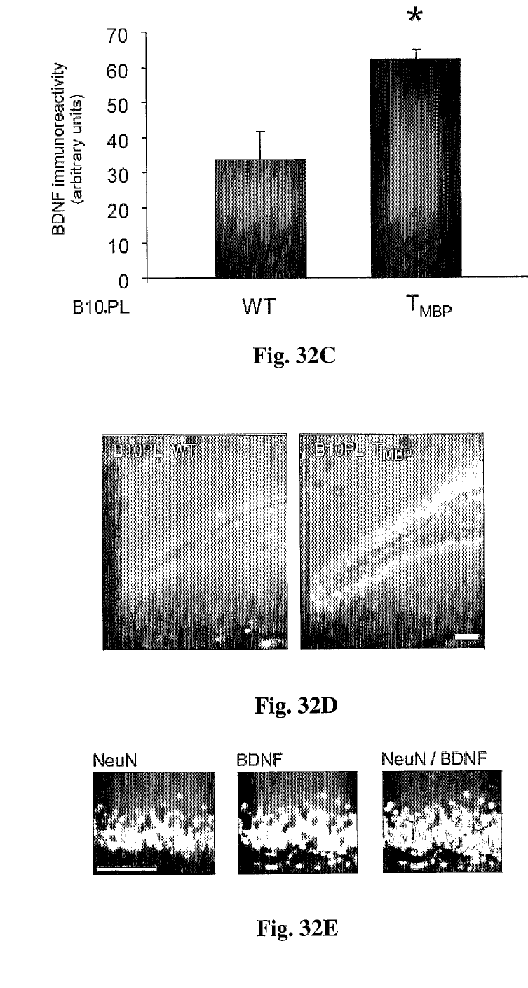

T cells can provide neurotrophic factors, such as BDNF, and can regulate, via their secreted cytokines, the production of growth factors (e.g. IGF-I) by other CNS-resident cells such as microglia. In the absence of external stimuli (for example, in an enriched environment), MHC-II- or IGF-I-expressing microglia are hardly detectable in the dentate gyrus. We therefore postulated that additional factors might play a role in the constitutive T cell-dependent neurogenesis. BDNF is an essential component of many hippocampal activities, including both spatial learning/memory and adult neurogenesis. We therefore compared the expression of BDNF in the hippocampal dentate gyri of SCID, T_{MBP} transgenic, and T_{OVA} transgenic mice to BDNF expression in their wild-type counterparts. Relative to their respective wild-type controls, BDNF immunoreactivity was significantly lower in both SCID mice and in T_{OVA} transgenic mice (FIGS. 32A, 32B), but significantly higher in T_{MBP} transgenic mice (FIG. 32C). FIG. 32D shows the pattern of BDNF immunoreactivity in the dentate gyri of T_{MBP} transgenic mice compared to that of matched wild-type mice. Staining for BDNF and NeuN disclosed that the cellular source of BDNF in the hippocampus was neurons (FIG. 32E). It should be noted, however, that BDNF expression in the nude mice did not differ significantly from that of the wild type (data not shown), suggesting the existence of a compensatory mechanism in nude mice. We found no differences between wild-type and immune-deficient mice in their hippocampal expression of additional growth factors associated with neurogenesis and hippocampal plasticity, such as vascular endothelial growth factor (VEGF) and Wnt.

### Example 5(6)

### Long-Term Potentiation (LTP) is Impaired in Immune-Deficient Mice

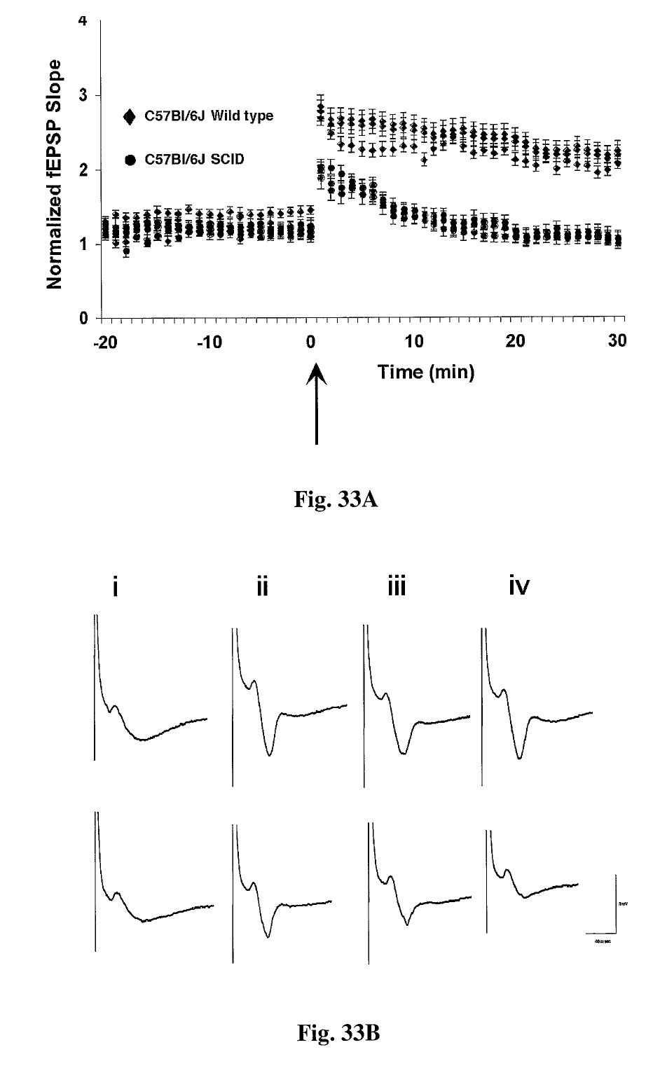

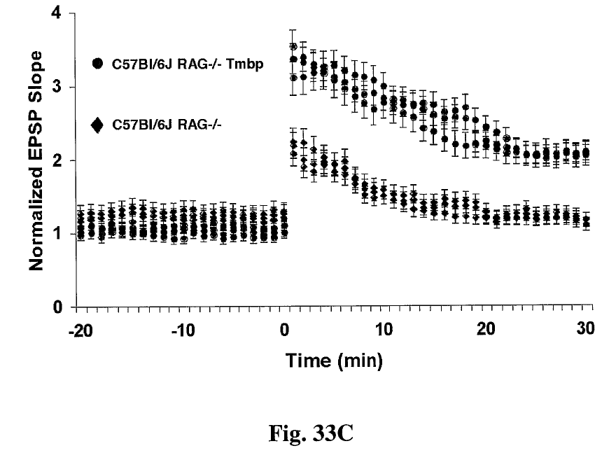

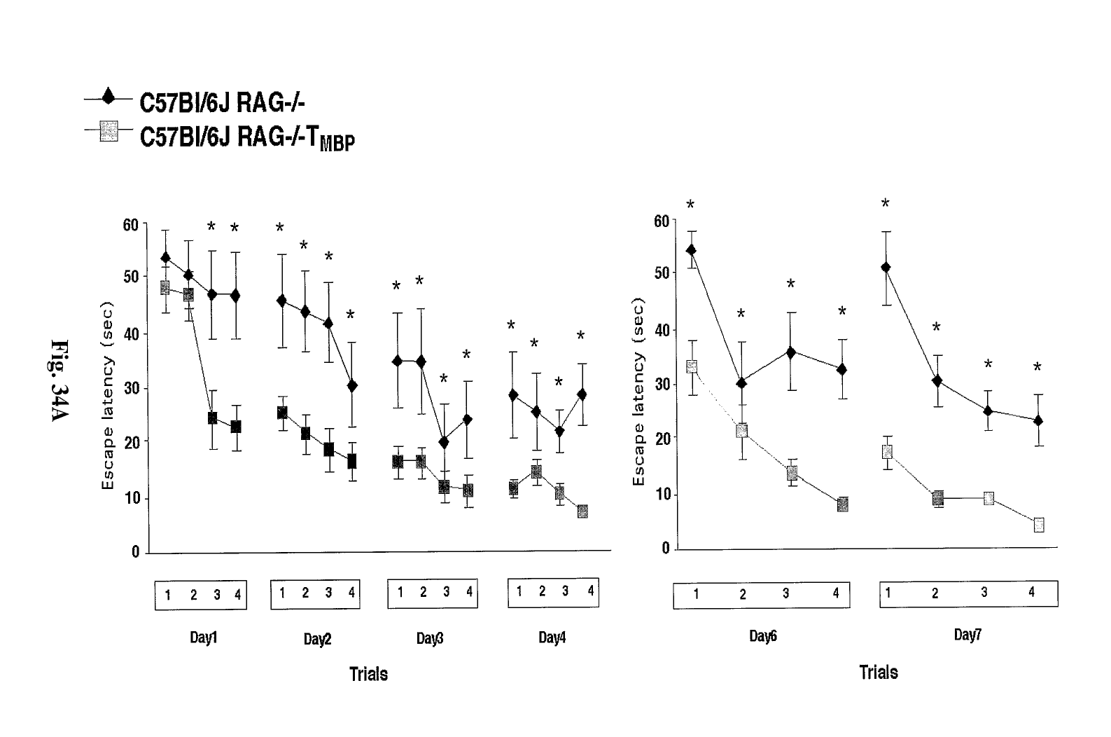

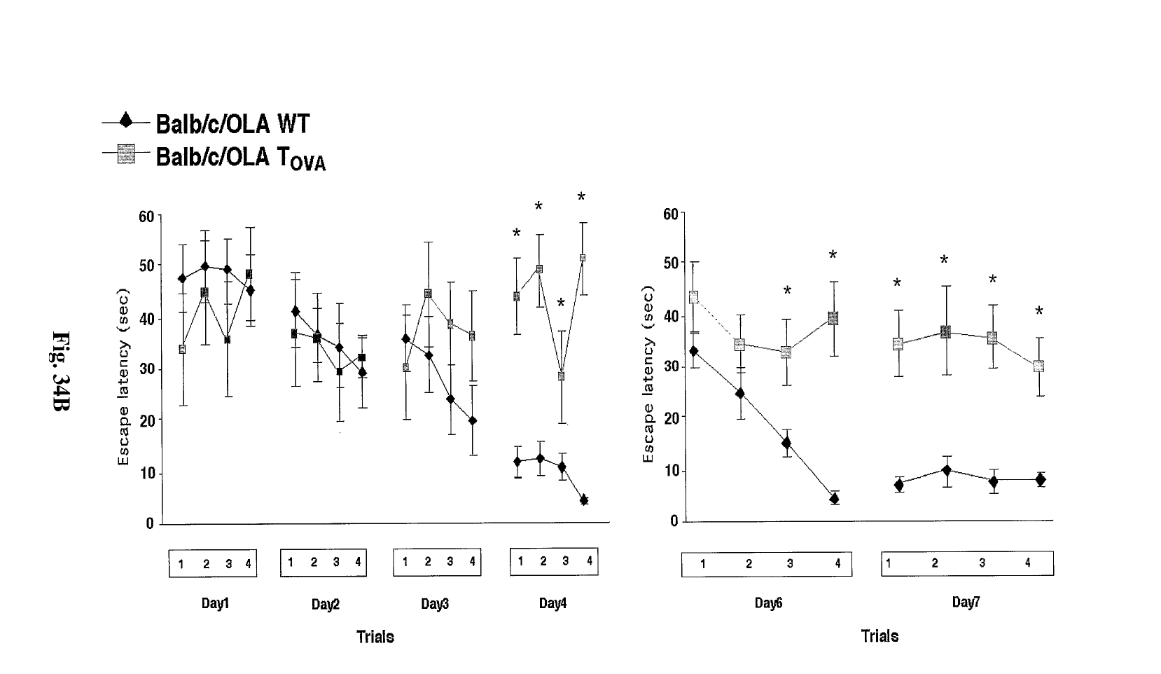

We also examined whether, in the adult animal, autoimmune T cells specific to MBP (T_{MBP} cells) affect hippocampus-dependent cognitive functions such as LTP and spatial learning/memory, the latter measured by performance of a task in the MWM. LTP provides a model of synaptic plasticity that is assumed to underlie memory formation (Bliss et al., 1993). Before examining the effect of the T_{MBP} cells on these functions, we examined whether LTP is impaired in mice with immune deficiency. This was done by comparing LTP in hippocampal slices taken from adult (12-week-old) C57B1/6J or Balb/c/OLA mice suffering from severe combined immune deficiency (SCID) to that in their respective wild-type controls. LTP was induced by theta-burst stimulation (TBS) of the Schaffer collaterals and was recorded from the CA1 region of the hippocampus. In both strains of SCID mice the post-theta potentiation declined approximately 15 min after TBS, whereas in the wild-type controls it was maintained for the duration of the experiment (approximately 30 min), indicating a capacity for LTP (FIGS. 33A, 33B). No morphological differences were apparent in brain slices from the SCID mice relative to their respective wild-type counterparts (data not shown).

To determine whether the observed impairment of LTP in SCID mice can be attributed to the lack of autoimmune T cells, we used transgenic mice expressing only T cells directed to MBP (Lafaille et al., 1994). The T_{MBP}-transgenic mice used in this experiment were on a RAG^{-/-} (immune-deficient) background, so that their entire T cell population was specific only to MBP. As controls for this group we used RAG^{-/-} mice (congenitally devoid of T cells). As with the SCID mice, LTP in the RAG1^{-/-} mice was impaired relative to that in the T_{MBP}-transgenic RAG1^{-/-} mice, in which normal LTP could be generated and sustained under the same experimental conditions (FIG. 33C).

### Example 5(7)

### Autoimmune T Cells Directed to MBP Beneficially Affect Brain Plasticity in Mice

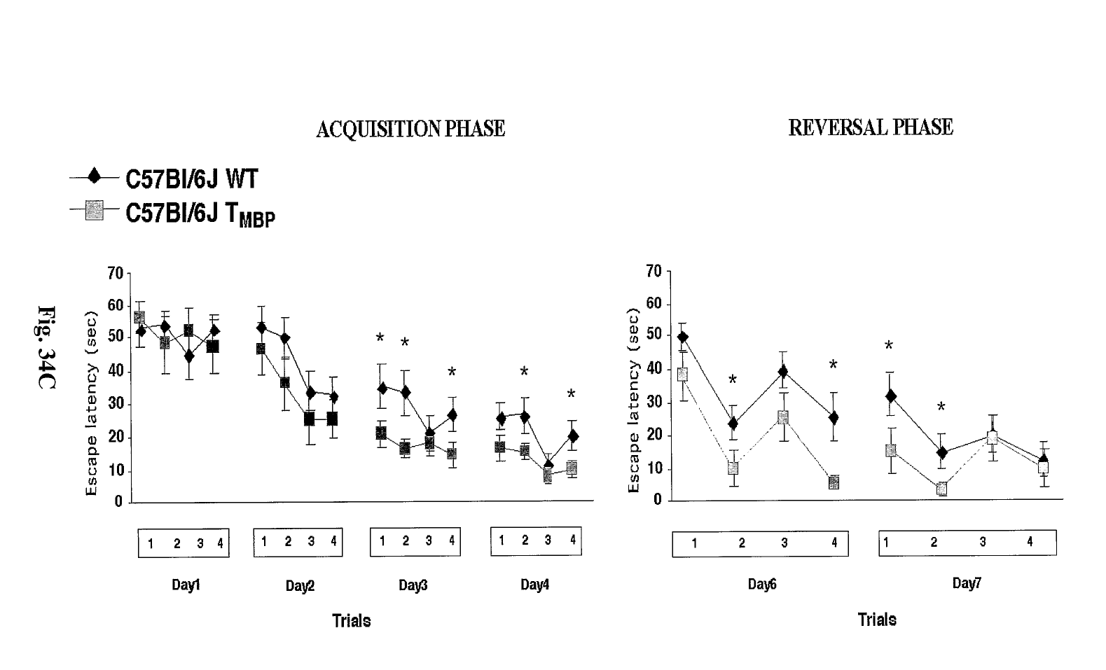

To study the behavioral manifestation(s) of the above differences, we examined the performance of the mice on a task in the MWVM. The T_{MBP}-transgenic RAG1^{-/-} mice learned to swim towards the hidden escape platform via the shortest route, whereas the ability of mice in the control group (devoid of T cells) to perform this task was significantly impaired (FIG. 34A). The superior performance of the T_{MBP}-transgenic mice was manifested in both the acquisition and the reversal phases of the task. When the escape platform was visible there were no detectable differences, in both velocity and route, between the two groups.

To exclude the possibility that any T cell population, not only autoimmune T cells, can affect hippocampal activity, we repeated the experiment with transgenic mice possessing monospecific T cells directed against the non-self antigen ovalbumin (T_{OVA}-transgenic mice). Since these transgenic mice are available only on a background of Balb/c/OLA, this was the strain used as a wild-type control for these experiments. We anticipated that if autoimmune T cells specific to myelin proteins are the cells needed for brain cognition, the T_{OVA}-transgenic mice would behave like SCID mice on the same background. Relative to the wild type, performance of the MWM task was significantly impaired in the T_{OVA} mice (FIG. 34B), whose behavior was indeed reminiscent of that of the SCID mice (Kipnis et al., 2004) Since the background of the T_{OVA} mice was wild type and not RAG^{-/-}, we also assessed the MWM performance of T_{MBP}-transgenic mice on a wild-type background. The T_{MBP} mice (FIG. 34C), like the T_{MBP} RAG^{-/-} mice (FIG. 34A), behaved similarly to the matched wild-type controls.

Taken together, these results suggested that autoimmune T cells directed to myelin-specific brain proteins such as MBP can help restore lost hippocampus-dependent LTP and spatial learning/memory in immune-defficient mice.

### Example 5(8)

### Autoimmune T Cells Specific to MBP Restores Neurogenesis in Immune-Defficient Mice

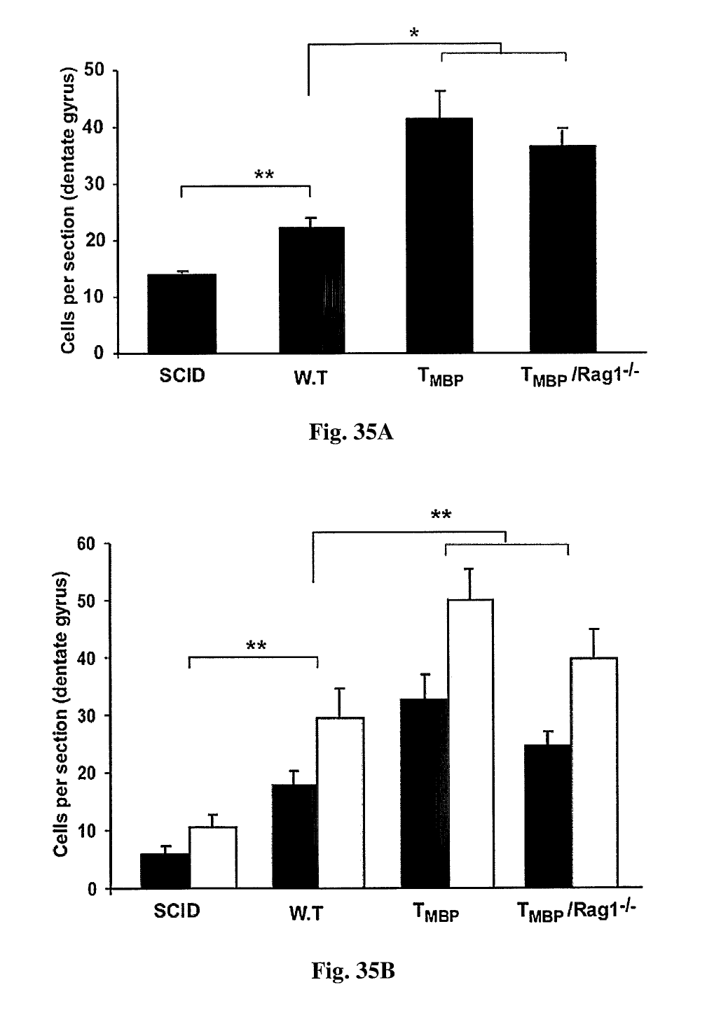

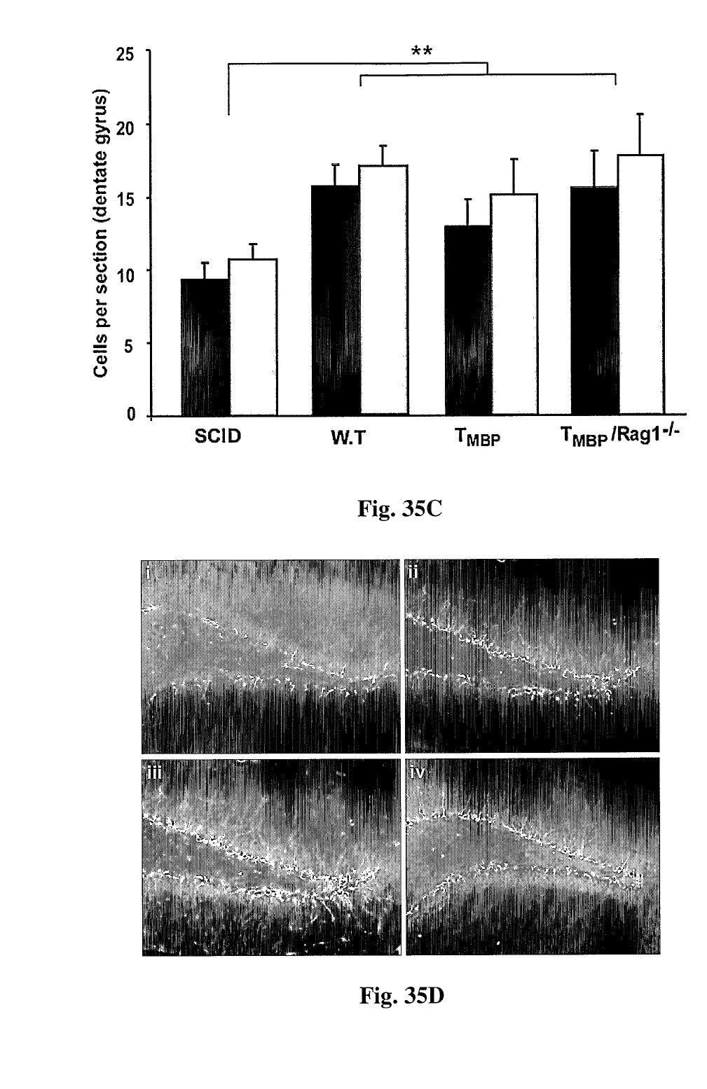

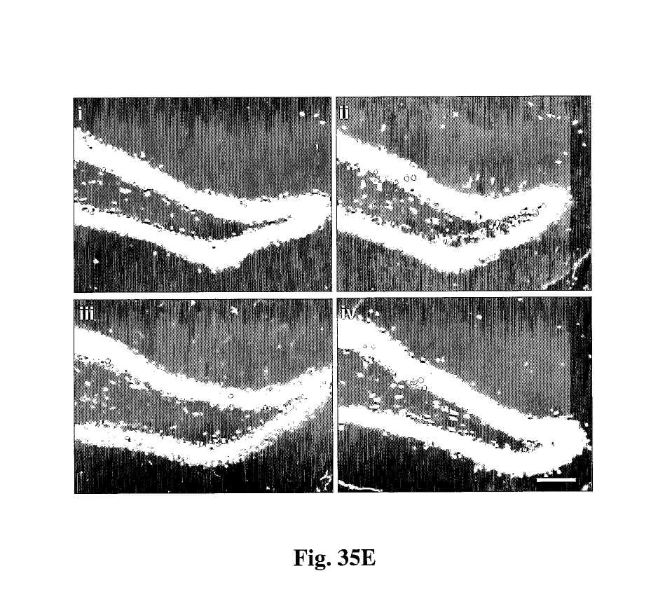

Several recent findings point to an association between adult neurogenesis and certain hippocampal activities (Shors et al., 2002). To examine the possibility that the role of T cells in adult plasticity is mediated at least in part via their effect on hippocampal neurogenesis in adulthood, we examined three pivotal phases in the process of neurogenesis: cell proliferation, early neuronal differentiation and long term survival of newly-formed neurons. We first compared the proliferation of neural precursor cells in adult (4-5-month old) wild-type mice to that in immune-deficient or in T_{MBP}-expressing mice (both on a wild-type and on a RAG^{-/-} background). After receiving four injections (once every 12-hours) of BrdU, which labels dividing cells, mice were killed (48 h after the first injection), their brains were excised, and sections of the hippocampus were examined for BrdU-positive cells. Significantly fewer labeled cells in the subgranular zone of the hippocampus were found in immune-deficient than in wild-type mice (FIG. 35A). At this time point, moreover, significantly more proliferating cells were found in the subgranular zones of T_{MBP}-transgenic mice of both backgrounds than in their wild-type counterparts (FIG. 35A).

To determine the relevance of the autoimmune T cells to neurogenesis in the hippocampus, we examined the dentate gyrus of brains that were excised 7 days after the first BrdU injection and double-stained for BrdU and the early neuronal differentiation marker doublecortin (DCX). Significantly more BrdU/DCX-positive cells were found in the wild type than in the immune-deficient mice (FIGS. 35B, 35D). Moreover, the numbers of these double-labeled cells in the T_{MBP}-transgenic mice of both backgrounds were even higher than their numbers in wild-type controls (FIGS. 35B, 35D). Interestingly, of the total numbers of BrdU-positive cells in each of the four mouse strains tested, approximately 60% were BrdU/DCX positive. This finding suggests that at least at this early postmitotic time point, and in the absence of any external stimuli that might affect brain activity (e.g. enriched environment), autoimmune T cells affect neurogenesis at the proliferative stage rather than at the stage of differentiation.

Next we examined whether the long-term survival of newly formed neurons is also affected by autoimmune T cells. Brain tissues were excised from all four groups of mice 4 weeks after the first BrdU injection, and sections were double-stained for BrdU and NeuN (a marker of mature neurons) in the dentate gyrus. Significantly fewer BrdU/NeuN-positive cells were found in the immune-deficient (SCID) mice than in their wild-type counterparts (FIGS. 35C, 35E). However, the numbers of these double-labeled cells in T_{MBP}-transgenic mice of both backgrounds were similar to the numbers in their respective wild types (FIGS. 35C, 35E). Autoimmune T cells restored the level of neurogenesis to the normal levels found in wild type controls. Since the survival of newly formed neurons is considered to be activity dependent (Gould et al., 1999), it is not surprising that in the absence of external stimuli the neurogenesis observed in mice expressing autoimmune T cells, relative to that in the wild type, was manifested only in the proliferation of the new neurons, not in their long-term survival.

### Discussion

The present work identifies CNS specific autoimmune T cells as pivotal players in adult brain plasticity and shows that their participation occurs, at least in part, via their cross-talk with resident microglia. This novel finding is in line with earlier demonstrations that CNS-specific T cells, provided that the onset, duration, and intensity of their activity are well controlled, exert a beneficial effect on neuronal survival after CNS injury (Moalem et al., 19991 Hauben et al., 2001; Schwartz and Kipnis, 2002).

The findings of this study suggest that T cells affect adult neurogenesis, both in the dentate gyrus and in the SVZ, primarily via their effect on progenitor cell proliferation. We cannot rule out the possibility that T cells play a role not only in the proliferation of progenitor cells but also in their neuronal differentiation, as indicated by the results obtained with nude mice.

Severe immune deficiency in mice was shown here to impair three aspects of hippocampal plasticity at adulthood: LTP, spatial learning/memory, and adult neurogenesis. A single population of T cells recognizing the abundant CNS self-antigen MBP was sufficient to ameliorate the impairment. The results of the present study show that T cells directed to a specific CNS self-antigen play a key role in maintaining the functional activity of the hippocampus. Accordingly, we postulated that under non-pathological conditions a primary role of such autoimmune T cells is to maintain the plasticity of the adult brain. If this is so, it would follow that the observed beneficial effect of antigen-specific anti-self T cells under degenerative conditions is an extension of their role in the healthy brain.

### Example 6

### T-Cell Based Vaccination Restores Cognition, Removes Plaques, and Induces Neurogenesis in a Mouse Model of Alzheimer's Disease

Accumulation of β-amyloid deposition (Aβ), neuronal loss, cognitive decline, and microglial activation, are characteristic features of Alzheimer's disease (AD). Using AD double-transgenic mice expressing mutant human genes encoding presenilin 1 and chimeric mouse/human amyloid precursor protein, we show that vaccination with glatiramer acetate prevented and restored cognitive decline, assessed by performance in a Morris water maze. The vaccination modulated microglial activation, eliminated plaque formation, and induced neuronal survival and neurogenesis. In vitro, Aβ-activated microglia impeded neurogenesis from adult neural stem/progenitor cells. This was counteracted by IL-4, and more so when IFN-γ was added, but not by IFN-γ alone.

### Materials and Methods

(xlv) Animals. Nineteen adult double-transgenic APP_{K670N, M671L}+PS1_{ΔE9} mice of the B6C3-Tg (APPswe, PSEN1dE9) 85 Dbo/J strain were purchased from The Jackson Laboratory (Bar Harbor, Me.) and were bred and maintained in the Animal Breeding Center of The Weizmann Institute of Science. All animals were handled according to the regulations formulated by the Weizmann Institute's Animal Care and Use Committee. Tg AD mice were produced by co-injection of chimeric mouse/human APPswe (APP695 [humanized Aβ domain] harboring the Swedish [K594M/N595L] mutation) and human PS1dE9 (deletion of exon 9) vectors controlled by independent mouse prion protein promoter (MoPrP) elements, as described (Borchelt et al., 1997).

(xlvi) Reagents. Recombinant mouse IFN-γ and IL-4 (both containing endotoxin at a concentration below 0.1 ng/μg cytokine) were obtained from R&D Systems (Minneapolis, Minn.). β-amyloid peptides [amyloid protein fragment 1-40 and 1-42 (Aβ_{1-40/1-42})] were purchased from Sigma-Aldrich, St. Louis, Mo. The Aβ peptides were dissolved in endotoxin-free water, and Aβ aggregates were formed by incubation of Aβ, as described (Ishii et al., 2000).

(xlvii) Genotyping. All mice used in this experiment were genotyped for the presence of the transgenes by PCR amplification of genomic DNA extracted from 1-cm tail clippings (Jankowsky et al., 2004). Reactions contained four primers: one anti-sense primer-matching sequence within the vector that is also present in mouse genomic PrP (5′-GTG GAT ACC CCC TCC CCC AGC CTA GAC C) (SEQ ID NO:13); a second sense primer specific for the genomic PrP coding region (which was removed from the MoPrP vector) (5′-CCT CTT TGT GAC TAT GTG GAC TGA TGT CGG) (SEQ ID NO:14); and twvo sense and anti-sense primers specific for the PS1 transgene cDNA (PS1-a: 5′-AAT AGA GAA CGG CAG GAG CA (SEQ ID NO:15), and PS1-b: 5′-GCC ATG AGG GCA CTA ATC AT) (SEQ ID NO:16). All reactions give a 750-bp product of the endogenous PrP gene as a control for DNA integrity and successful amplification; PS1 transgene-positive samples have an additional band at approximately 608 bp.

(xlviii) Glatiramer acetate vaccination. Each mouse was subcutaneously injected five times with a total of 100 μg of glatiramer acetate (GA (TV-5010), MW 13.5-18.5 kDa, average 16 kDa, Teva Pharmaceutical Industries Ltd., Petach Tikva, Israel), emulsified in 200 μl PBS×1, from experimental day 0 until day 24, twice during the first week and once a week thereafter.

(xlix) Behavioral testing. Spatial learning/memory was assessed by performance on a hippocampus-dependent visuo-spatial learning task in the Morris water maze (MWM) (Morris, 1984). Mice were given four trials per day on 4 consecutive days, during which they were required to find a hidden platform located 1.5 cm below the water surface in a pool 1.4 m in diameter. Within the testing room, only distal visuo-spatial cues for location of the submerged platform were available. The escape latency, i.e., the time required by the mouse to find the platform and climb onto it, was recorded for up to 60 s. Each mouse was allowed to remain on the platform for 30 s and was then moved from the maze to its home cage. If the mouse did not find the platform within 60 s, it was placed manually on the platform and returned to its home cage after 30 s. The interval between trials was 300 s. On day 5 the platform was removed from the pool and each mouse was tested by a probe trial for 60 s. On days 6 and 7 the platform was placed at the quadrant opposite the location chosen on days 1-4, and the mice were then retrained in four sessions per day. Data were recorded using an EthoVision automated tracking system (Noldus).

(l) Administration of BrdU and tissue preparation. BrdU was dissolved by sonication in PBS and injected i.p. into each mouse (50 mg/kg body weight; 1.25 mg BrdU in 200 μl PBS×1). Starting from experimental day 22 after the first GA vaccination, BrdU was injected i.p. twice daily, every 12 h for 2.5 days, to label proliferating cells. Three weeks after the first BrdU injection the mice were deeply anesthetized and perfused transcardially, first with PBS and then with 4% paraformaldehyde. The whole brain was removed, postfixed overnight, and then equilibrated in phosphate-buffered 30% sucrose. Free-floating 30-μm sections were collected on a freezing microtome (Leica SM2000R) and stored at 4° C. prior to immunohistochemistry.

(li) Neural progenitor cell culture. Coronal sections (2 mm thick) of tissue containing the subventricular zone of the lateral ventricle were obtained from the brains of adult C57B1/6J mice. The tissue was minced and then incubated for digestion at 37° C., 5% CO₂ for 45 min in Earle's balanced salt solution containing 0.94 mg/ml papain (Worthington, Lakewood, N.J.) and 0.18 mg/ml of L-cysteine and EDTA. After centrifugation at 110×g for 15 min at room temperature, the tissue was mechanically dissociated by pipette trituration. Cells obtained from single-cell suspensions were plated (3500 cells/cm²) in 75-cm² Falcon tissue-culture flasks (BD Biosciences, San Diego, Calif.), in NPC-culturing medium [Dulbecco's modified Eagles's medium (DMEM)/F12 medium (Gibco/Invitrogen, Carlsbad, Calif.) containing 2 mM L-glutamine, 0.6% glucose, 9.6 μg/ml putrescine, 6.3 ng/ml progesterone, 5.2 ng/ml sodium selenite, 0.02 mg/ml insulin, 0.1 mg/ml transferrin, 2 μg/ml heparin (all from Sigma-Aldrich, Rehovot, Israel), fibroblast growth factor-2 (human recombinant, 20 ng/ml), and epidermal growth factor (human recombinant, 20 ng/ml; both from Peprotech, Rocky Hill, N.J.)]. Spheres were passaged every 4-6 days and replated as single cells. Green fluorescent protein (GFP)-expressing NPCs were obtained as previously described (Pluchino et al., 2003).

(lii) Primary microglial culture. Brains from neonatal (P0-P1) C57B1/6J mice were stripped of their meninges and minced with scissors under a dissecting microscope (Zeiss, Stemi DV4, Germany) in Leibovitz-15 medium (Biological Industries, Kibbutz Beit Ha-Emek, Israel). After trypsinization (0.5% trypsin, 10 min, 37° C./5% CO₂), the tissue was triturated. The cell suspension was washed in culture medium for glial cells [DMEM supplemented with 10% fetal calf serum (FCS; Sigma-Aldrich, Rehovot), L-glutamine (1 mM), sodium pyruvate (1 mM), penicillin (100 U/ml), and streptomycin (100 mg/ml)] and cultured at 37° C./5% CO₂ in 75-cm² Falcon tissue-culture flasks (BD Biosciences) coated with poly-D-lysine (PDL) (10 mg/ml; Sigma-Aldrich, Rehovot) in borate buffer (2.37 g borax and 1.55 g boric acid dissolved in 500 ml sterile water, pH 8.4) for 1 h, then rinsed thoroughly with sterile, glass-distilled water. Half of the medium was changed after 6 h in culture and every 2^{nd} day thereafter, starting on day 2, for a total culture time of 10-14 days. Microglia were shaken off the primary mixed brain glial cell cultures (150 rpm, 37° C., 6 h) with maximum yields between days 10 and 14, seeded (10⁵ cells/ml) onto PDL-pretreated 24-well plates (1 ml/well; Corning), and grown in culture medium for microglia [RPMI-1640 medium (Sigma-Aldrich) supplemented with 10% FCS, L-glutamine (1 mM), sodium pyruvate (1 mM), P-mercaptoethanol (50 mM), penicillin (100 U/ml), and streptomycin (100 mg/ml)]. The cells were allowed to adhere to the surface of a PDL-coated culture flask (30 min, 37° C./5% CO₂), and non-adherent cells were rinsed off.

(liii) Co-culturing of mouse neural progenitor cells and mouse microglia. Cultures of treated or untreated microglia were washed twice with fresh NPC-differentiation medium (same as the culture medium for NPCs but without growth factors and with 2.5% FCS) to remove all traces of the tested reagents, then incubated on ice for 15 min, and shaken at 350 rpm for 20 min at room temperature. Microglia were removed from the flasks and immediately co-cultured (5×10⁴ cells/well) with NPCs (5×10⁴ cells/well) for 10 days on cover slips coated with Matrigel™ (BD Biosciences) in 24-well plates, in the presence of NPC differentiation medium. The cultures were then fixed with 2.5% paraformaldehyde in PBS for 30 min at room temperature and stained for neuronal and glial markers.

(liv) Immunocytochemistry and immunohistochemistry. Cover slips from co-cultures of NPCs and mouse microglia were washed with PBS, fixed as described above, treated with a permeabilization/blocking solution containing 10% FCS, 2% bovine serum albumin, 1% glycine, and 0.1% Triton X-100 (Sigma-Aldrich, Rehovot), and stained with a combination of the mouse anti-tubulin β-III-isoform C-terminus antibodies (P-II-tubulin; 1:500; Chemicon, Temecula, Calif.) and CD11b (MAC1; 1:50; BD-Pharmingen, Franklin Lakes, N.J.).

For BrdU staining, sections were washed with PBS and incubated in 2N HCl at 37° C. for 30 min. Sections were blocked for 1 h with blocking solution (PBS containing 20% normal horse serum and 0.1% Triton X-100, or PBS containing mouse immunoglobulin blocking reagent obtained from Vector Laboratories (Burlingame, Calif.)).

For immunohistochemistry, tissue sections were treated with a permeabilization/blocking solution containing 10% FCS, 2% bovine serum albumin, 1% glycine, and 0.05% Triton X-100 (Sigma-Aldrich, St. Louis). Tissue sections were stained overnight at 4° C. with specified combinations of the following primary antibodies: rat anti-BrdU (1:200; Oxford Biotechnology, Kidlington, Oxfordshire, UK), goat anti-DCX [doublecortin] (1:400; Santa Cruz Biotechnology), and mouse anti-NeuN [neuronal nuclear protein] (1:200; Chemicon, Temecula, Calif.). Secondary antibodies were FITC-conjugated donkey anti-goat, Cy-3-conjugated donkey anti-mouse, and Cy-3- or Cy-5-conjugated donkey anti-rat (1:200; Jackson ImmunoResearch, West Grove, Pa.). For labeling of microglia we used either CD11b (MAC1; 1:50; BD-Pharmingen) or FITC-conjugated Bandeiraea simplicifolia isolectin B4 (IB4, 1:50; Sigma-Aldrich, Rehovot). To detect expression of cell-surface MHC-II proteins we used anti-MHC-II Abs (rat, clone IBL-5/22; 1:50; Chemicon, Temecula, Calif.). To detect expression of human Aβ we used anti-Aβ (human amino-acid residues 1-17) (mouse, clone 6E10, Chemicon, Temecula, Calif.). Expression of IGF-I was detected by goat anti-IGF-I Abs (1:20; R&D Systems). Expression of TNF-α was detected by goat anti-TNF-α Abs (1:100; R&D Systems). T cells were detected with anti-CD3 polyclonal Abs (rabbit, 1:100; DakoCytomation, Calif.). Propidium iodide (1 μg/ml; Molecular Probes, Invitrogen, Carlsbad, Calif.) was used for nuclear staining.

Control sections (not treated with primary antibody) were used to distinguish specific staining from staining of nonspecific antibodies or autofluorescent components. Sections were then washed with PBS and coverslipped in polyvinyl alcohol with diazabicylo-octane as anti-fading agent.

(lv) Quantification and stereological counting procedure. For microscopic analysis we used a Zeiss LSM 510 confocal laser scanning microscope (40× magnification). For experiments in vitro we scanned fields of 0.053 mm² (n=8-16 from at least two different coverslips) for each experimental group. For each marker, 500-1000 cells were sampled. Cells co-expressing GFP and β-III-tubulin were counted.

For in-vivo experiments, the number of Aβ⁺ plaques and CD11b⁺/IB-4⁺ microglia in the hippocampus were counted at 300-μm intervals from 6-8 coronal sections (30 μm) from each mouse. Neurogenesis in the DG was evaluated by counting of premature neurons (DCX⁺), proliferating cells (BrdU⁺), and newly formed mature neurons (BrdU⁺/NeuN⁺) in six coronal sections (30 μm) from each mouse. Specificity of BrdU⁺/NeuN⁺ co-expression was assayed using the confocal microscope (LSM 510) in optical sections at 1-μm intervals/. Cell counts, numbers of Aβ⁺ plaques, plaque areas, and intensity of NeuN staining per unit area in the DG were evaluated automatically using Image-Pro Plus 4.5 software (Media Cybernetics).

(lvi) Statistical analysis. MWM behavior scores were analyzed using 3-way ANOVA, with treatment group and trial block as sources of variation, was used to evaluate the significance of differences between mean scores during acquisition trial blocksin the MWM. When the P-value obtained was significant, a pairwise Fisher's least-significant-difference multiple comparison test was run to determine which groups were significantly different.

The in-vitro results were analyzed by the Tukey-Kramer multiple comparisons test (ANOVA) and are expressed as means±SEM. In-vivo results were analyzed by Student's t-test or 1-way ANOVA and are expressed as means±SEM.

### Example 6(1)

### T Cell-Based Vaccination Counteracts Cognitive Decline in AD

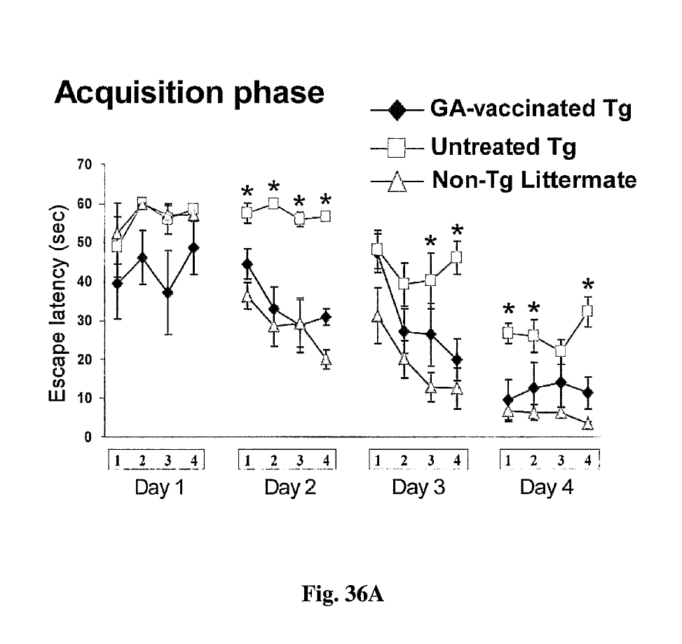

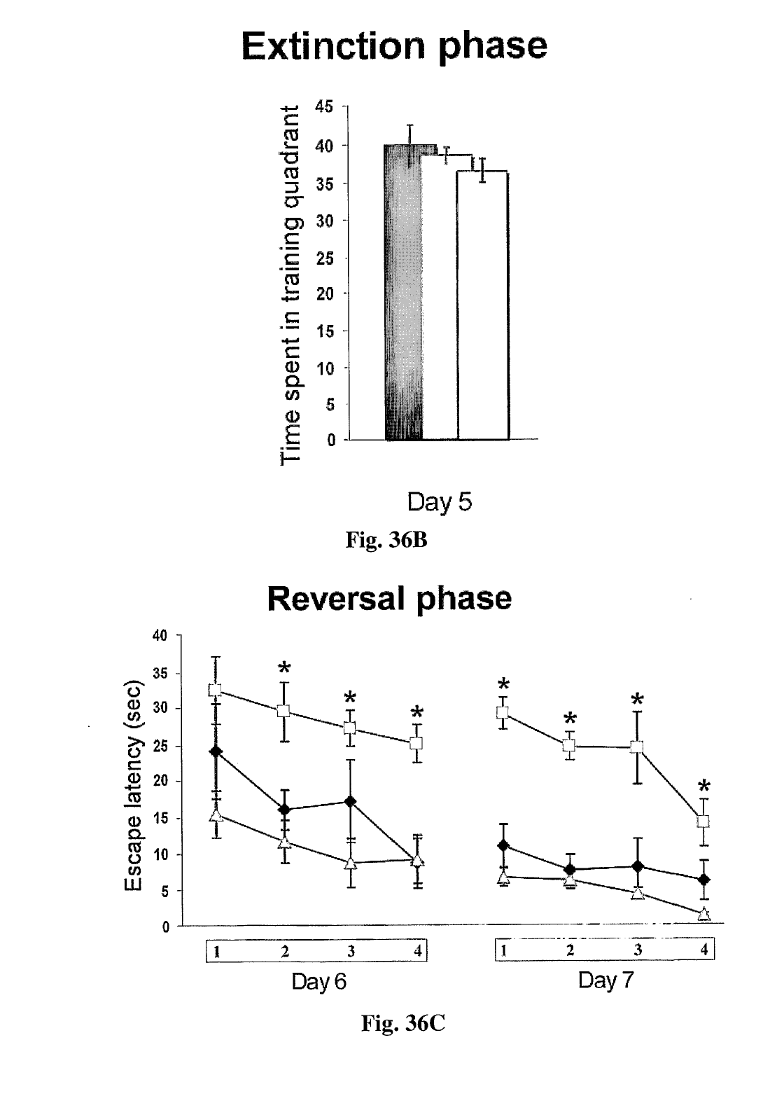

We examined the effect of GA in AD double-transgenic mice (Tg mice) expressing a mutant human presenilin 1 gene (PS1dE9) and a chimeric mouse/human amyloid precursor protein (APPswe), leading to learning/memory impairment and accumulation of Aβ plaques mainly in the cortex and the hippocampus, both characteristic features of early-onset familial AD (Borchelt et al., 1997). Expression of both transgenes in each mouse was verified by PCR amplification of genomic DNA. APP/PS1 Tg mice aged approximately 8 months were then vaccinated subcutaneously with GA (n=6) twice during the first week and once a week thereafter. Age-matched Tg mice (n=7) and non-Tg littermates that did not carry the transgenes (n=6), were not treated and served as untreated Tg and wild-type non-Tg controls, respectively. Five weeks after the first GA injection all mice were assessed in a Morris water maze (MWM) for cognitive activity, as reflected by performance of a hippocampus-dependent spatial learning/memory task. The MWM performance of the untreated Tg mice was significantly worse, on average, than that of the age-matched non-Tg littermates (FIG. 36). However, the performance of Tg mice that were vaccinated with GA was superior to that of the untreated Tg mice and did not differ significantly from that of their non-Tg littermates (FIG. 36), suggesting that the GA vaccination had prevented further cognitive loss and even reversed part of the earlier functional deficit. Cognitive losses or improvements were manifested in both the acquisition and the reversal tasks (FIGS. 36A-36C).

### Example 6(2)

### T Cell-Based Vaccination Modulates the Immune Activity of Microglia, Eliminates β-Amyloid Plaque Formation, Supports Neuronal Survival and Induces Neurogenesis

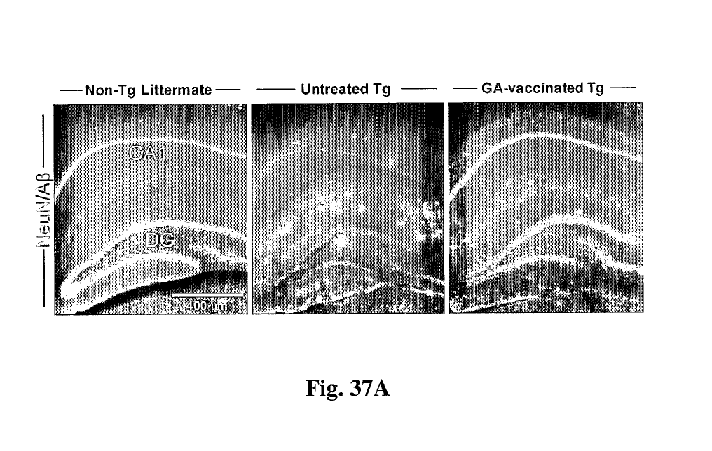

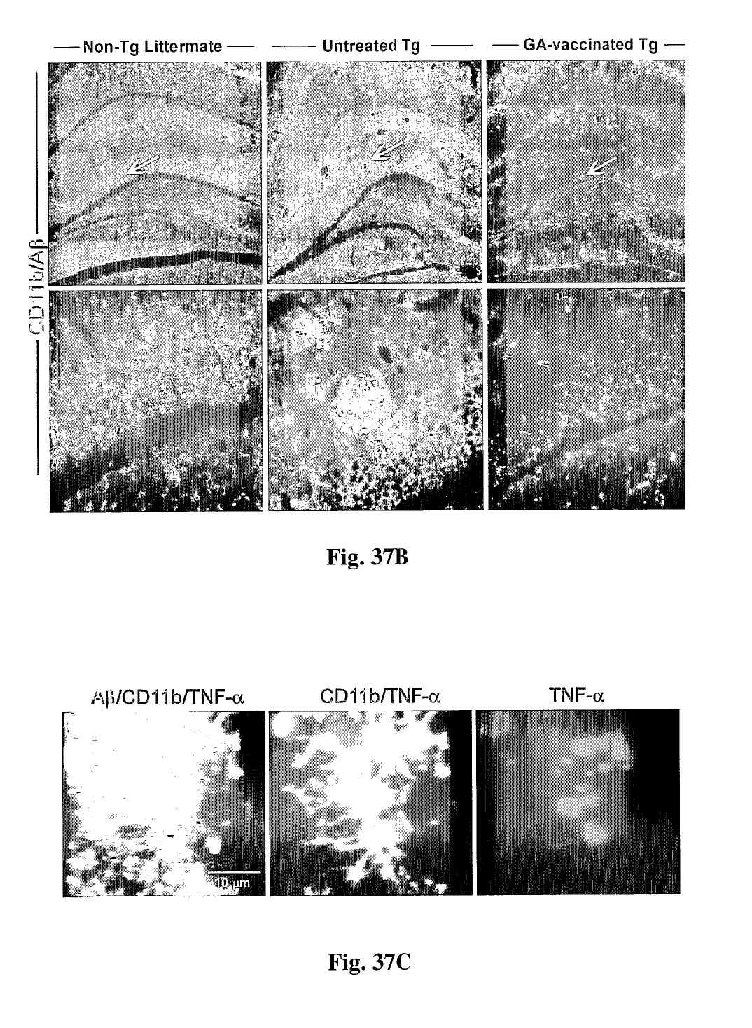

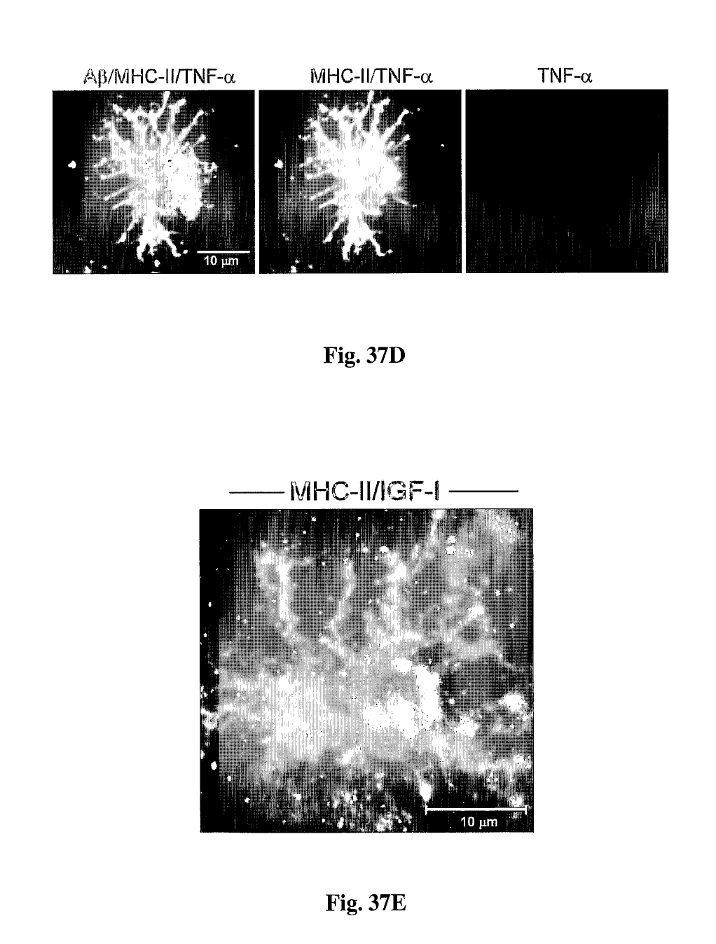

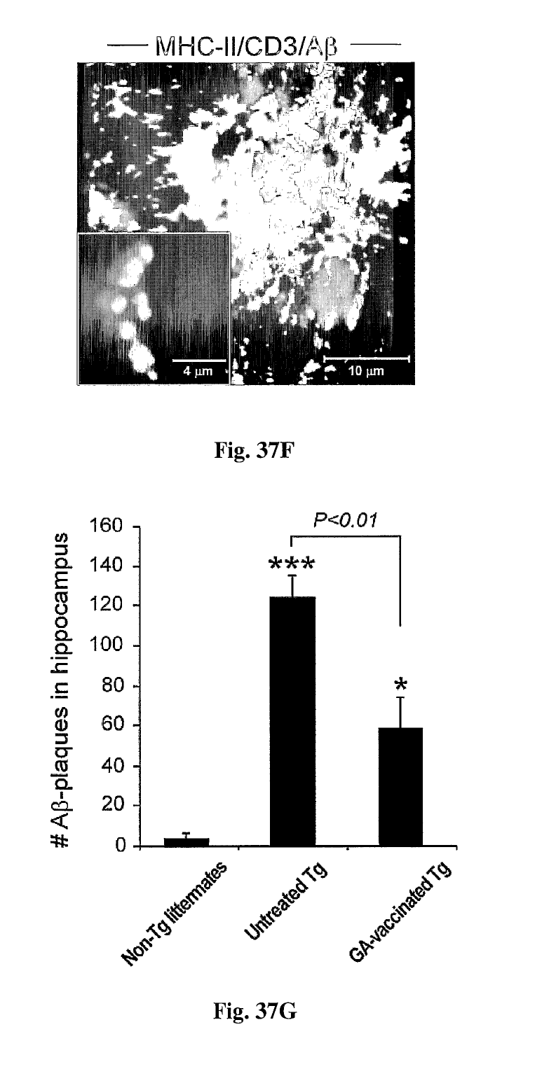

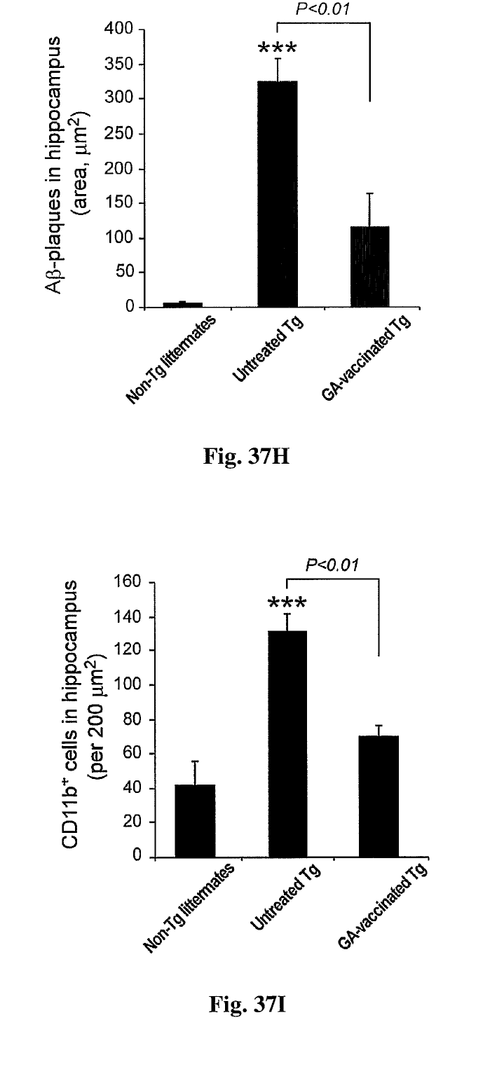

The above results prompted us to examine the possibility that the observed arrest and reversal of cognitive loss was related to reduction of Aβ plaques and survival of neurons in the hippocampus. Staining of brain cryosections from Tg mice with antibodies specific to human Aβ disclosed numerous plaques in the untreated Tg mice but very few in those vaccinated with GA (FIG. 37A). No plaques were seen in their respective non-Tg littermates (FIG. 37A). Examination of NeuN immunoreactivity disclosed loss of neurons in the untreated Tg mice but preservation of neurons in the GA-vaccinated Tg mice (FIG. 37A).

Activated microglia are known to play a role in the pathogenesis of AD. We have shown in the Examples hereinbefore that, unlike microglia seen in association with inflammatory and neurodegenerative diseases, the microglia associated with neural tissue survival express MHC-II, produce IGF-I, and express little or no TNF-α. We therefore examined brain sections from GA-vaccinated and untreated Tg mice for the presence of microglia that stain positively for CD 11b or TNF-α (markers of activation associated with a cytotoxic inflammatory phenotype). The presence of plaques was found to be correlated with the appearance of CD 11b+microglia (FIG. 37B) expressing TNF-α (FIG. 37C and Movie S1 (prepared by the inventors but not shown here) that depicts a 3-D reconstruction of an Aβ plaque and CD11b+microglia expressing TNF-α. This movie presents a representative 3-D confocal image of a microglial cell embedded within an Aβ plaque in the hippocampus of an untreated Tg mouse shown in FIG. 37C, in which the high TNF-α-immunoreactivity and engulfed Aβ in the cytoplasm can be noted), and was abundant in the untreated Tg mice. Significantly fewer CD11b+microglia were detectable in the GA-vaccinated Tg mice (FIG. 37B). Staining with anti-MHC-II antibodies disclosed that in the untreated Tg mice almost no microglia expressed MHC-II (indicating their inability to act as APCs; data not shown), whereas in the GA-vaccinated Tg mice most of the microglia adjacent to residual Aβ⁺ plaques expressed MHC-II, and hardly any of them expressed TNF-α (FIG. 37D and Movie S2 (prepared by the inventors but not shown here) that depicts 3-D reconstruction of an Aβ-immunoreactivity associated with MHC-II⁺ microglia in a GA-vaccinated Tg mouse. This movie also shows a representative 3-D confocal image of microglia shown in FIG. 37D, expressing marginal levels of TNF-α and high levels of MHC-II). These latter microglia also expressed IGF-I (FIG. 37E and Movie S3 (prepared by the inventors but not shown here) that depicts a 3-D reconstruction of a microglial cell co-expressing IGF-1 and MHC-II⁺. This movie shows a representative 3-D confocal image of MHC-II⁺ microglia from the IGF-I-expressing GA-vaccinated Tg mouse shown in FIG. 37E.), indicating their potential for promoting neuroprotection and neurogenesis and for beneficially affecting learning and memory. All of the MUC-II⁺ cells were co-labeled with IB4, identifying them as microglia (data not shown). It is important to note that the CD11b⁺ microglia (seen mainly in the untreated Tg mice) showed relatively few ramified processes, whereas such processes were abundant in the MHC-II⁺ microglia in the GA-vaccinated Tg mice, giving them a bushy appearance (depicted in Movies S1 and S2, not shown).

In addition, unlike in the untreated Tg mice, in the GA-vaccinated Tg mice numerous T cells (identified by anti-CD3 antibodies) were seen in close proximity to MHC-II⁺ microglia. Any Aβ-immunoreactivity seen in these mice appeared to be in association with MHC-II⁺ microglia, creating an immune synapse with CD3⁺ T cells (FIG. 37F and Movie S4 (prepared by the inventors but not shown here) that depicts a 3-D reconstruction of an Aβ plaque associated with CD3⁺ cells (T cells) in close proximity to MHC-II⁺ microglia. This movie depicts a representative 3-D confocal image of MHC-II⁺ microglia from the GA-vaccinated Tg mouse shown in FIG. 37F, with an immunological synapse between a CD3⁺ cell and a complex of MHC-II and Aβ.).

Quantitative analysis confirmed that mice vaccinated with GA showed significantly fewer plaques than untreated Tg mice when examined 6 weeks later (FIG. 37G), and that the area occupied by the plaques was significantly smaller than in their age-matched untreated counterparts (FIG. 37H). In addition, GA-vaccinated Tg mice showed significantly fewer CD11b⁺ microglia and significantly more intense staining for NeuN than their corresponding groups of untreated Tg mice (FIGS. 37I, 37J).

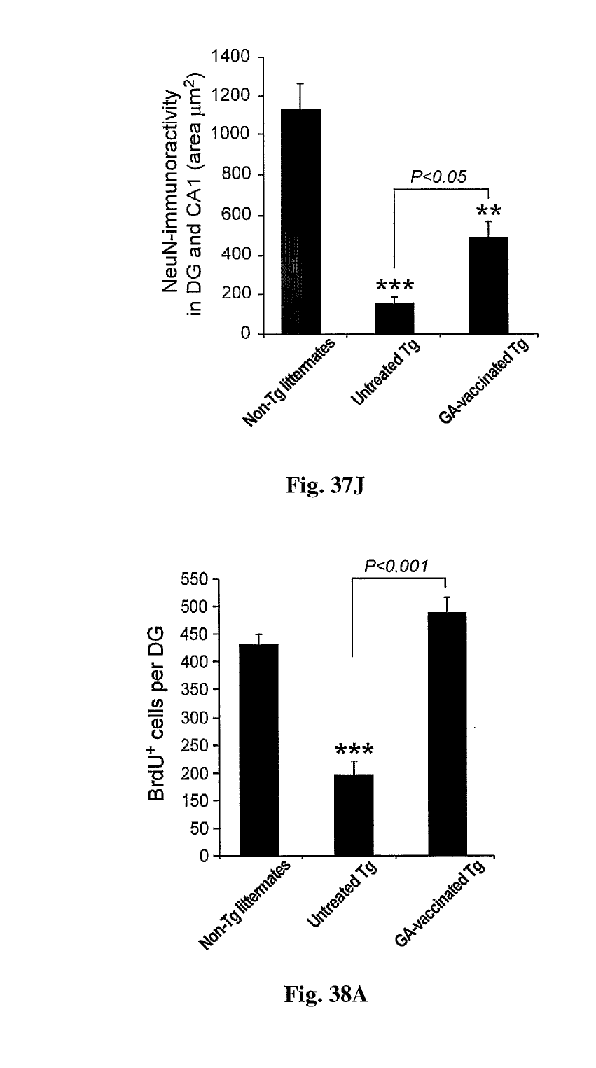

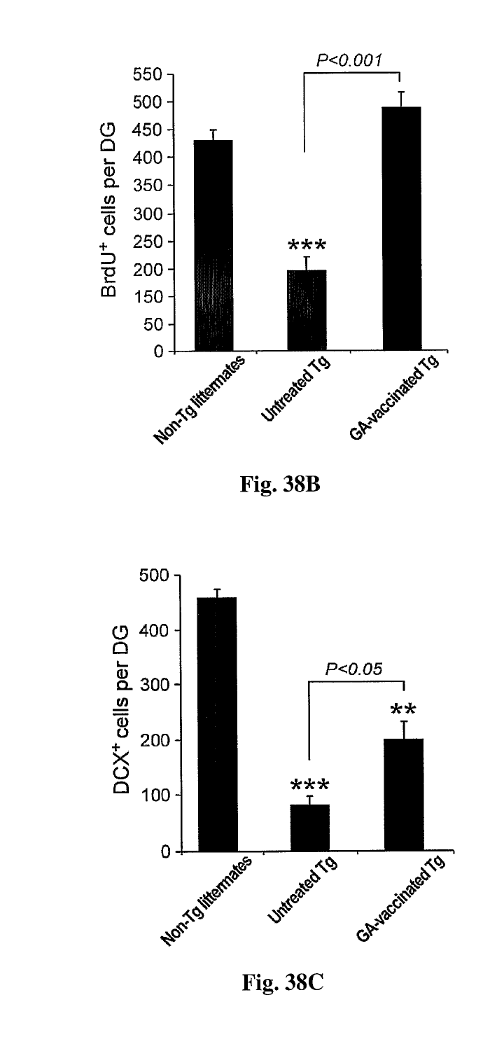

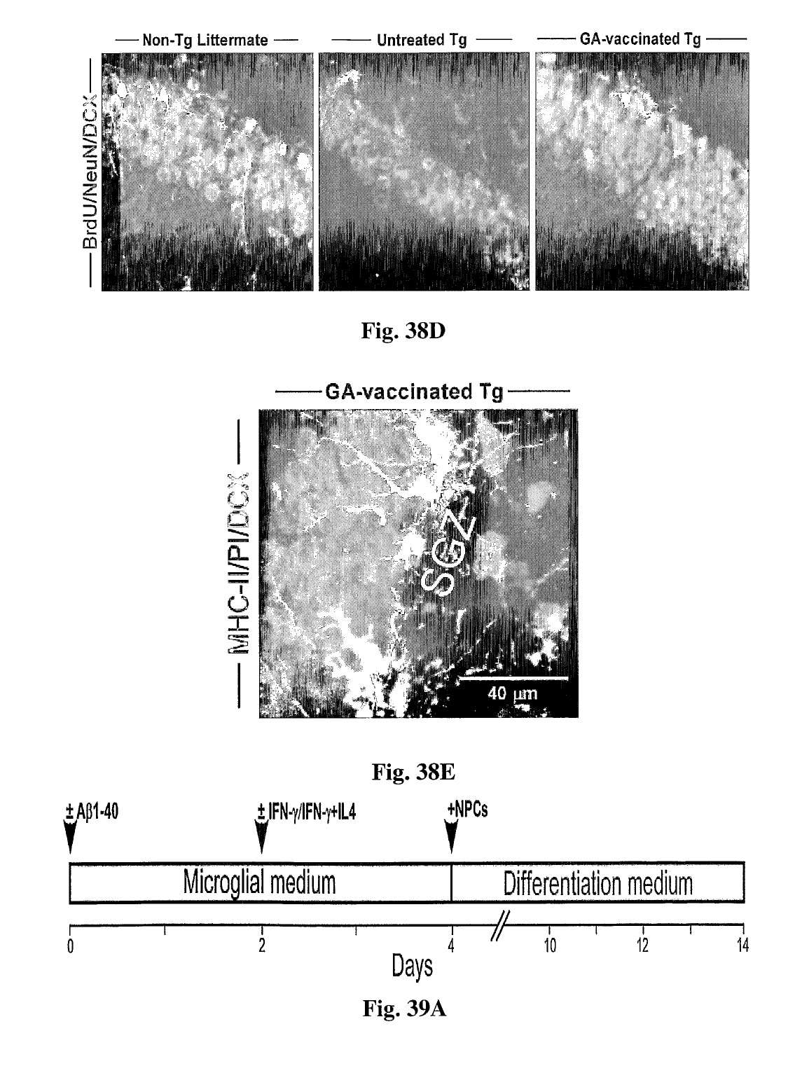

Because MHC-II-expressing microglia are also associated with neurogenesis in vitro, we examined the same sections for the formation of new neurons in the dentate gyrus (DG) of the hippocampus. This was possible because all mice had been injected with BrdU, a marker of proliferating cells, 3 weeks before tissue excision. Quantitative analysis disclosed significantly more BrdU⁺ cells in GA-vaccinated Tg mice (FIG. 38A) than in their untreated counterparts. In addition, compared to the numbers of newly formed mature neurons (BrdU⁺/NeuN⁺) in their respective non-Tg littermates the numbers were significantly lower in the untreated Tg group, but were similar in the vaccinated group, indicating that the neurons had been at least partially restored by the GA vaccination (FIG. 38B). Analysis of corresponding sections for doublecortin (DCX), a useful marker for analyzing the absolute number of newly generated pre-mature neurons in the adult DG, disclosed that relative to the non-Tg littermates there were significantly fewer DCX⁺ cells in the DGs of untreated Tg mice, and slightly but significantly more in the DGs of Tg mice vaccinated with GA (FIG. 38C). Confocal micrographs illustrate the differences in the numbers of BrdU⁺/NeuN⁺ cells or of DCX⁺ cells and their dendritic processes between non-Tg littermates, untreated Tg mice, and GA-vaccinated Tg mice (FIG. 38D). The results showed that neurogenesis was indeed more abundant in the GA-vaccinated mice than in untreated Tg mice. Interestingly, however, in both untreated and GA-vaccinated Tg mice the processes of the DCX⁺-stained neurons in the subgranular zone of the DG were short, except in those GA-vaccinated mice in which the DCX⁺ cells were located adjacent to MHC-II⁺ microglia (FIG. 38E).

### Example 6(3)

### Aggregated β-Amyloid Induces Microglia to Express a Phenotype that Blocks Neurogenesis, and the Blocking is Counteracted by IL-4

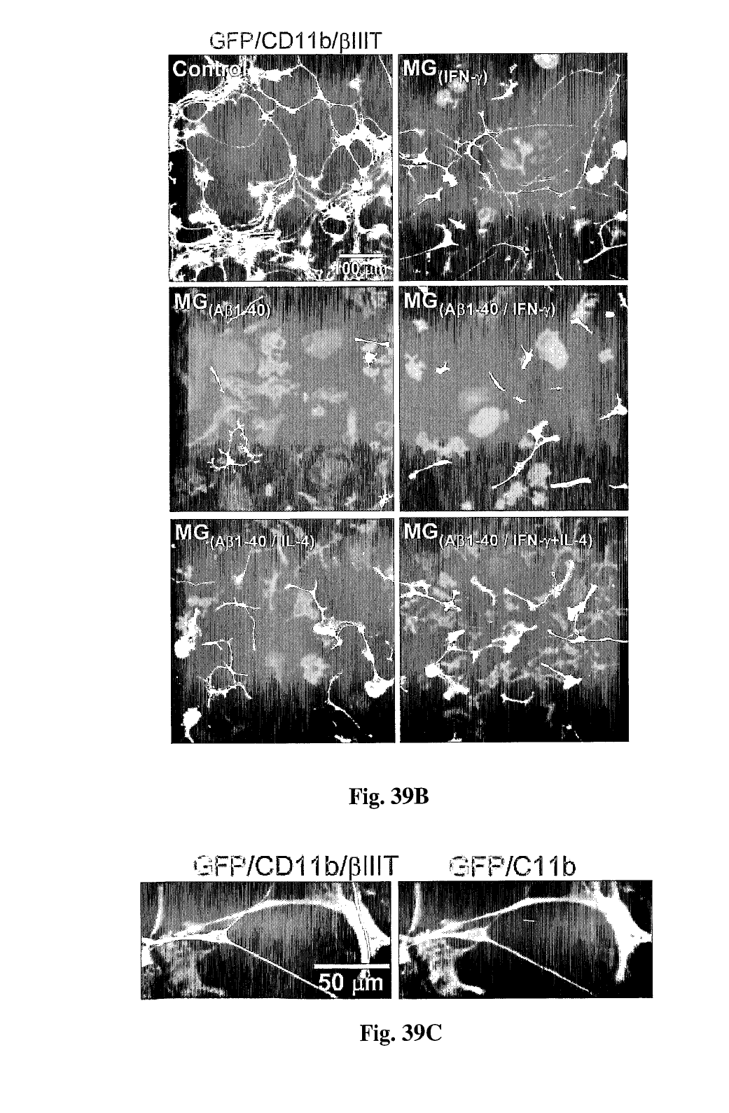

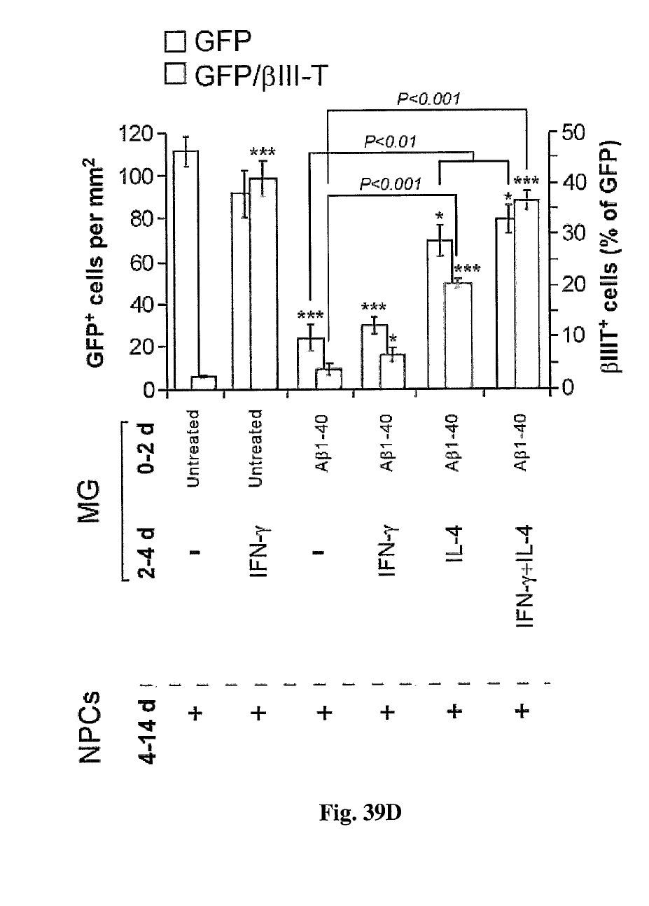

The in-vivo results presented above point to a relationship between arrested neurogenesis, aggregated AD, and the phenotype of the activated microglia. To determine whether aggregated Aβ-activated microglia block neurogenesis, and whether T cell-derived cytokines can counteract the inhibitory effect, we co-cultured GFP-expressing NPCs with microglia that had been pre-incubated for 48 h in their optimal growth medium in the presence or absence of aggregated Aβ peptide 1-40/1-42 (Aβ_{(1-40/1-42)}; 5 μM) and subsequently treated with IFN-γ (10 ng/ml), or with IL-4 (10 ng/ml) together with IFN-γ (10 ng/ml), for an additional 48 h. Growth media and cytokine residues were then washed off, and each of the treated microglial preparations was freshly co-cultured with dissociated NPC spheres on coverslips coated with Matrigel™ in the presence of differentiation medium (FIG. 39A). Expression of GFP by NPCs confirmed that any differentiating neurons seen in the cultures were derived from the NPCs rather than from contamination of the primary microglial culture. After 10 days we could discern GFP-positive NPCs expressing the neuronal marker β-III-tubulin (βIIIT) (FIGS. 39B, 39C). No βIIIT⁺ cells were seen in microglia cultured without NPCs. Significantly fewer GFP⁺/βIIIT⁺ cells were seen in control NPCs cultured without microglia (control). In co-cultures of NPCs with microglia previously activated by incubation with IFN-γ (10 ng/ml), however, the increase in numbers of GFP⁺/βIIIT⁺ cells was dramatic. In contrast, microglia activated by aggregated Aβ₍₁₋₄₀₎ (5 μM) blocked neurogenesis and decreased the number of NPCs. This negative effect was not exhibited by microglia activated by 5 μM Aβ₍₁₋₄₂₎ (data not shown). Interestingly, the addition of IL-4 (10 ng/ml) to microglia pre-treated with aggregated Aβ₍₁₋₄₀₎ partially counteracted the adverse effect of the aggregated Aβ on NPC survival and differentiation, with the result that these microglia were able to induce NPCs to differentiate into neurons (FIG. 39D). It thus appears that aggregated Aβ₍₁₋₄₀₎ impaired the ability to support neurogenesis, and that its effect could be counteracted to some extent by IL-4 and more strongly by the combination of IL-4 and IFN-γ.

### Discussion

In this study of APP/PS1 double-transgenic AD mice suffering from decline in cognition and accumulation of Aβ plaques, a T cell-based vaccination, by altering the microglial phenotype, ameliorated cognitive performance, reduced plaque formation, rescued cortical and hippocampal neurons, and induced hippocampal neurogenesis.

### Example 6

### Immune-Modulation Enhances Hippocampal and Cortical Neurogenesis Following Stroke: Effect of Poly YE on Neurogenesis

Using a rat model of permanent middle cerebral artery occlusion, we show herein that systemic immune-modulation with pYE enhances hippocampal and cortical neurogenesis from aNPCs following stroke. We show that the sate of neurogenic unpermissiveness in brain regions such as the cortex can be changed by the local immune response. These findings offer a new immune-based therapeutic approach for augmenting cell renewal from endogenous NPCs.

### Materials and Methods:

(lvii) Animals. Sprague-Dawley (SPD) male rats, aged 8-12 weeks, were supplied by Harlan Biotech (Jerusalem, Israel) and were handled according to the ARVO resolution on the use of animals in research.

(lviii) Permanent middle cerebral artery occlusion in rats. Rats were anesthetized by i.p. injection of ketamine HCl 100 mg/kg (Fort Dodge Laboratories, Fort Dodge, Iowa) and xylazine 2%, 10 mg/kg (Vitamed, Benyamina, Israel). A temperature control unit was used to maintain the body temperature of the rats at approximately 37° C. during surgery. Following a mid-line longitudinal incision in the cervical area, the right common carotid artery (CCA) was dissected from the surrounding tissue and the right internal carotid artery (ICA) and the right external carotid artery (ECA) were exposed. Branches of the right ECA were occluded by electrocoagulation and the distal portion was ligated. The right CCA and ICA were temporarily occluded by clamping. The tip of a 4-0 nylon filament was blunted by heating near a flame, to form a bead. The filament was then coated with poly-L-lysine solution (0.1% in water, Sigma, St. Louis, Mo.) and dried at 60° C. for 1 h prior to use. The beaded filament was inserted into the right ICA through a puncture in the right ECA and advanced over a distance of 2.0 cm to the right anterior cerebral artery, bypassing and occluding the origin of the right middle cerebral artery (MCA). The protruding tip of the filament was cut, the blood vessels and tissues were returned to their usual positions ands the incision was sutured. After recovering from the anesthesia the rats were returned to their home cages and allowed free access to food and water.

(lix) Treatment with Poly YE. Upon completion of surgery the rats were randomly assigned to different treatment groups. All rats were injected once subcutaneously (s.c.) at the indicated times either with the designated dosages of PN277 (Poly YE, Sigma, St. Louis, Mo.) in a final volume of 1 ml phosphate-buffered saline (PBS) or with PBS only (control group). Rats were weighed before MCAO and twice weekly thereafter over the indicated time period.

(lx) Administration of 5-bromo-2′-deoxyuridine. MCAO was performed as described above, and PN277 (1 mg/rat) was subcutaneously injected 24 h later. The cell proliferation marker BrdU was dissolved by sonication in PBS (6.25 mg/ml), and injected i.p. into rats (50 mg/kg body weight), twice daily from day 7 to day 9 after MCAO. On day 28 the rats were deeply anesthetized and killed by transcardial perfusion with PBS followed by 4% paraformaldehyde. Brains were removed, fixed overnight, and then equilibrated in phosphate-buffered sucrose (30% sucrose). Free-floating 25-μm sections were collected on a freezing microtome (Leica SM2000R) and stored at 4° C. prior to immunohistochemistry. Since BrdU is known to exert cytotoxic effects on proliferating lymphocytes, we chose in our experiments to inject BrdU with a short pulse of 3 days, starting on day 7 after MCAO to coincide with the peak of spontaneous proliferation. Thus the results of our analysis might even underestimate the absolute level of proliferation because of a lack of constantly available BrdU along the experimental period.

(lxi) Immunohistochemistry. For BrdU staining, tissue sections were washed with PBS and incubated for 30 min in 2N HCl at 37° C. Sections were blocked for 1 h with blocking solution (PBS containing 20% normal horse serum and 0.5% Triton X-100). Tissue sections were then stained overnight with the primary antibodies rat anti-BrdU (1:100; Oxford Biotechnology, Kidlington, Oxfordshire, UK), mouse anti nestin (1:1000; Chemicon, Temecula, Calif.), rabit anti MAP-2 (1:200; Chemicon, Temecula, Calif.) and mouse anti-NeuN (1:150; Chemicon, Temecula, Calif.). Secondary antibodies were Cy-2-conjugated donkey anti-mouse, Cy-3-conjugated donkey anti-mouse, and Cy-3- or Cy-5-conjugated donkey anti-rat (1:200; Jackson ImmunoResearch, West Grove, Pa.).

(lxii) Quantification. For analysis of cell proliferation and neurogenesis, six coronal hippocampal sections per rat brain (five rats per group), spanning the entire dentate gyrus, were taken for immunohistochemistry. Proliferation was assessed in a Nikon E800 fluorescent microscope by counting BrdU⁺ cells in the dentate gyrus. The numbers obtained were multiplied by the distance between each section to obtain an estimate of the total number of proliferating cells per dentate gyrus. Neuronal differentiation in the dentate gyrus was evaluated using confocal microscopy by counting cells that were double-labeled with BrdU and NeuN. Cortical neurogenesis was assessed by staining with BrdU together with MAP-2 or nestin. At least 2 coronal sections from 4 animals per group were included in this analysis. Measurements of intensity per surface area were obtained with ImageProPlus software.

### Example 7(1)

### Effect of PN277 on Hippocampal Neurogenesis after Stroke

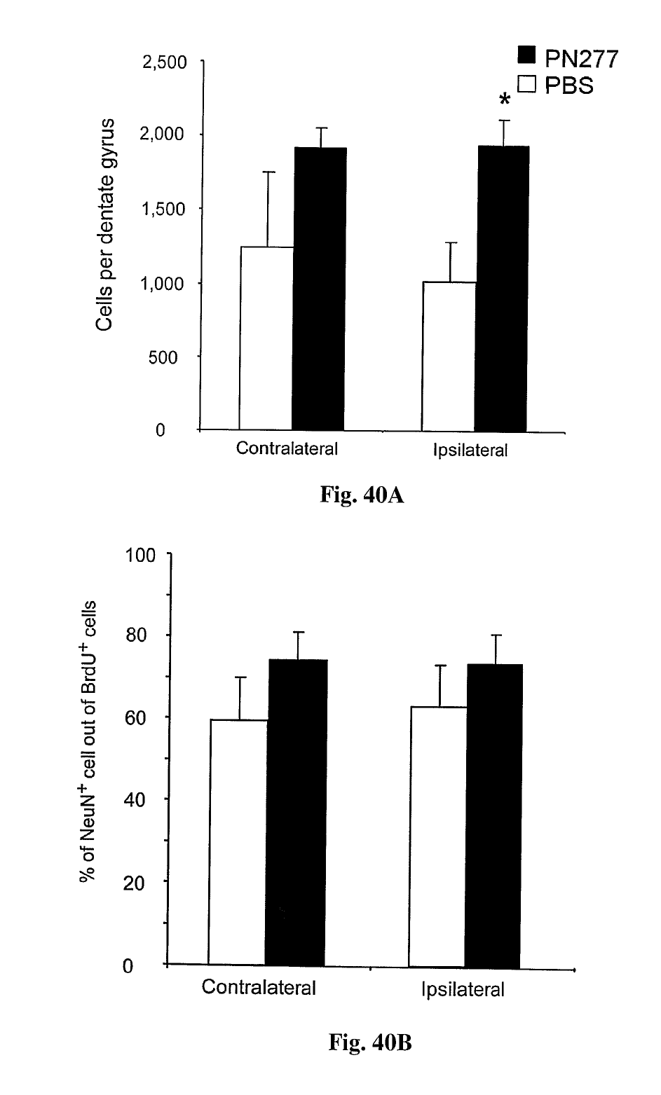

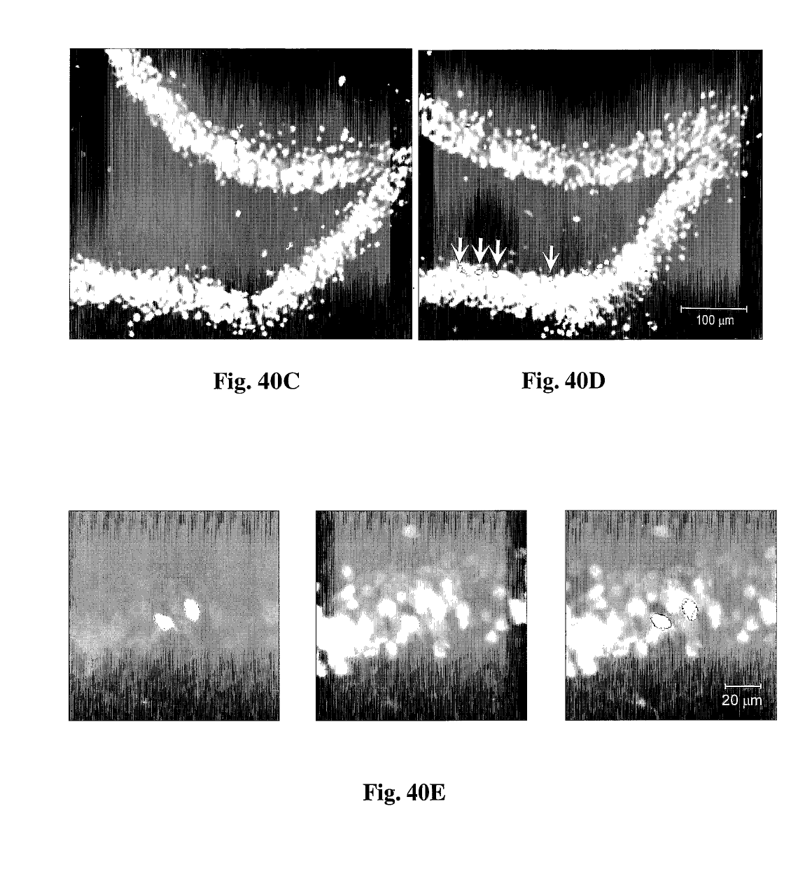

We first examined the effect of PN277 on hippocampal neurogenesis after stroke. Stem-cell proliferation and survival in the hippocampus were assessed by injecting control and PN277-treated rats with the cell proliferation marker BrdU for 3 days starting at day 7 after MCAO. The rats were killed 28 d after MCAO, and neurogenesis was evaluated by staining of coronal hippocampal sections with antibodies against BrdU and NeuN (a marker of post-mitotic neurons). Treatment with PN277 (1 mg/rat) immediately after stroke resulted in a two-fold increase in the number of newly formed neurons in the ipsilateral dentate gyrus relative to PBS treated controls (FIG. 40A, P<0.05, t-test). An increase in neurogenesis observed after PN277 treatment could be a result of increased proliferation of progenitor cells, enhanced neuronal differentiation, or increased survival of newly formed neurons. There was only a slight, statistically insignificant, increase in the ratio between the number of new neurons (BrdUW/NeuN cells) and the total number of proliferating cells (BrdU⁺ only) in the PN277-treated group compared to the PBS-treated controls (FIG. 40B), which cannot account for the significant increase in neurogenesis after treatment with PN277. It therefore seems reasonable to assume that the increased numbers of new neurons in the dentate gyrus of the PN277-treated group on day 28 after MCAO was a result of increased proliferation of progenitor cells, increased survival of newly formed neurons, or both.

Newly formed BrdU⁺/NeuN⁺ cells were observed in the granular cell layer of the dentate gyrus in PN277-treated rats (FIGS. 40B, 40D, 40E). Interestingly, some increase in hippocampal neurogenesis was observed in the contralateral hemisphere as well, suggesting that a CNS-specific T-cell response, facilitated by PN277 treatment after stroke, supports neurogenesis in a site not directly affected by MCAO.

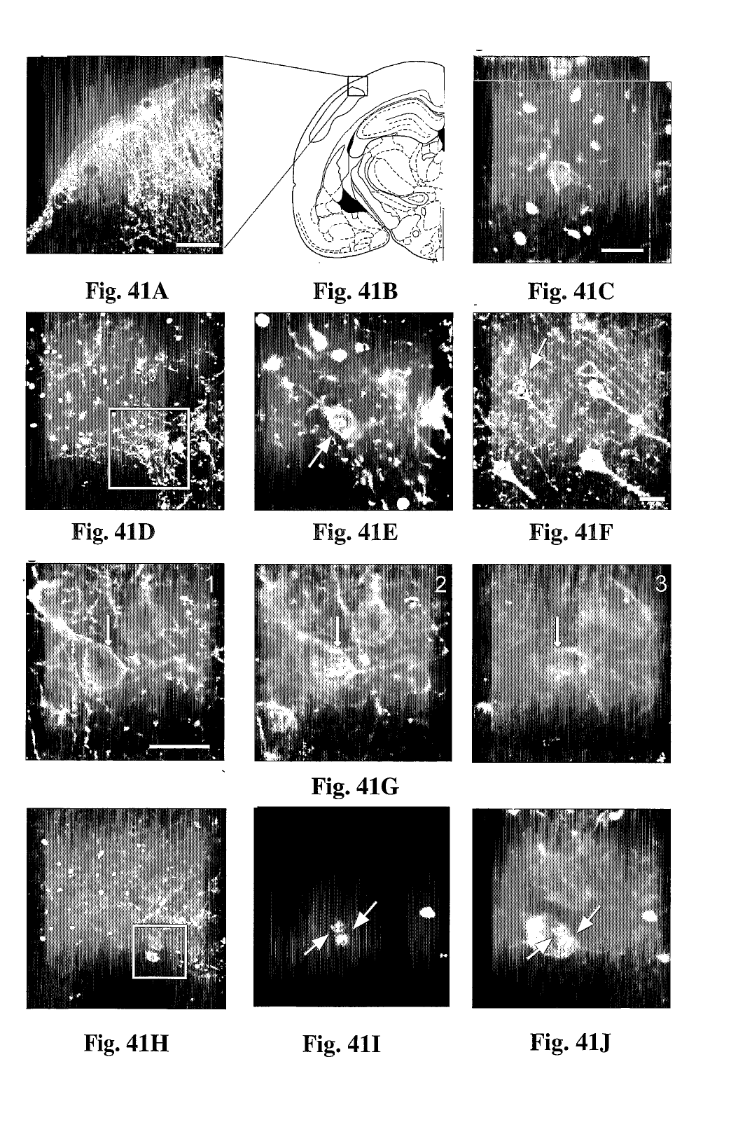

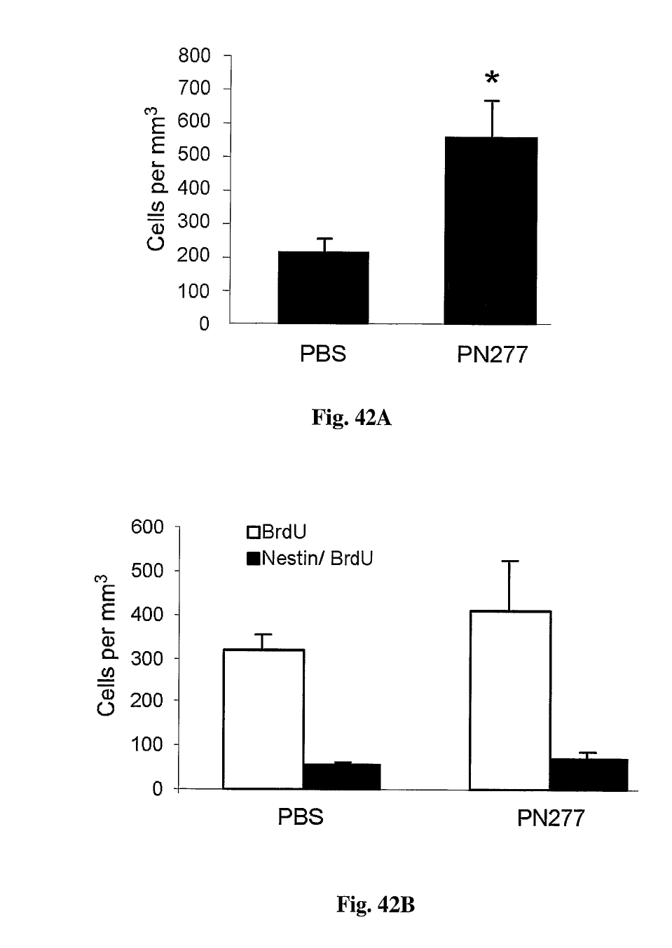

The ischemic injury imposed by MCAO causes severe damage to striatal and cortical structures. Recent studies demonstrated that such ischemic or traumatic injury can induce neurogenesis in regions of the brain that are normally non-neurogenic (Arvidsson et al., 2002; Nakatomi et al., 2002; Emsley et al., 2005). We next asked to determine whether in our model of stroke neurogenesis was induced in cortical areas that were affected by the stroke. We thus stained coronal sections containing the cortical lesion site for BrdU, the progenitor cell marker Nestin and neuronal markers such as β-III tubulin and MAP-2. A large number of BrdU⁺ cells and Nestin+ cells could be seen surrounding the lesion site (FIGS. 41A, 41B). No viable neurons (expressing MAP-2) could be detected within the lesion site (FIG. 41A). Since the feasibility of cortical neurogenesis has been a matter of debate, we used in our analysis several neuronal markers and examined co-localization with BrdU using 3-D confocal scanning. We could find newly-formed neurons co-labeled with BrdU and the early neuronal marker β-III tubulin (FIG. 41C). These cells expressed low levels of the mature neuronal marker MAP-2. We could also detect substantial amounts of newly formed neurons with mature phenotype (BrdU⁺/MAP-2⁺). Some of these newly formed mature neurons appeared within the area adjacent to the lesion (FIG. 41D), and had only short processes (FIG. 41E), implying that conditions in that area were not ideal for cell growth and connectivity. On the contrary, newly formed mature neurons with highly developed morphology were found in cortical areas that were more remote from the lesion (150-400 μm away) (FIGS. 41F-41G). A minority (less than 1%) of these BrdU⁺/MAP-2+ cells appeared to contain two BrdU labeled nuclei.

We next asked to determine whether treatment with PN277 could enhance cortical neurogenesis. To avoid staining artifacts caused by non-specific staining of myelin debris we choose MAP2 instead of NeuN. We took special care to count only MAP-2+ cells that exhibit the typical morphology of pyramidal neurons in the cortex and in which BrdU appeared in the center of the cell. Brains in which the cortex was not appear to be damaged were excluded from the analysis. Quantitative analysis revealed a 2-fold increase in the number newly formed cortical neurons in the PN277 treated rats relative to PBS-treated rats (FIG. 42A). To determine whether PN277 treatment also affected the number of proliferating/progenitor cells we also counted the numbers of BrdU⁺ and BrdU/Nestin+ cells found in area delineating the lesion (FIGS. 42A, 42B). No significant differences were found in the numbers of BrdU⁺ and BrdU/Nestin+ cells between the two experimental groups (FIG. 42B).

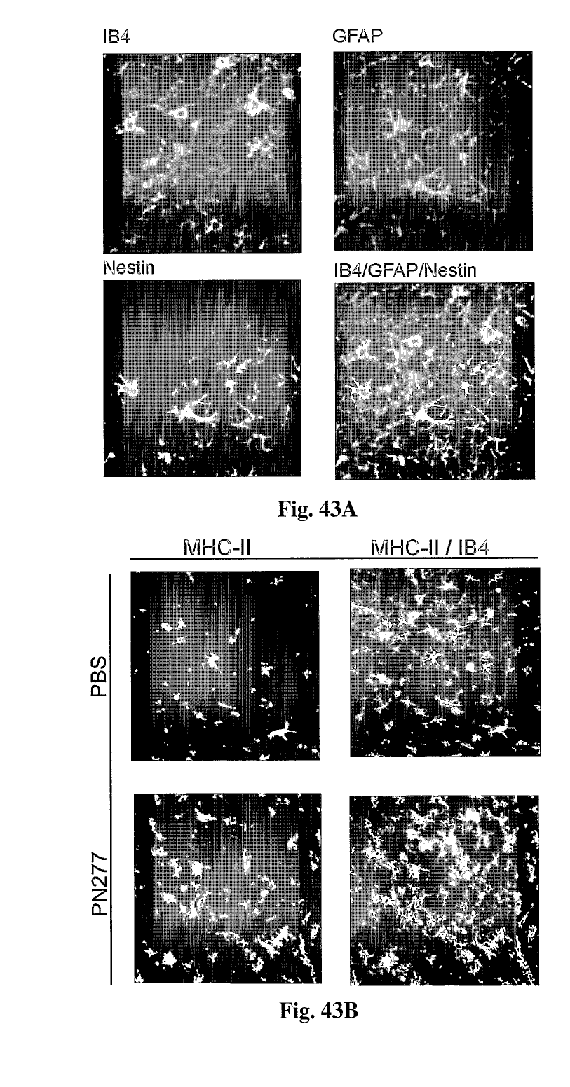

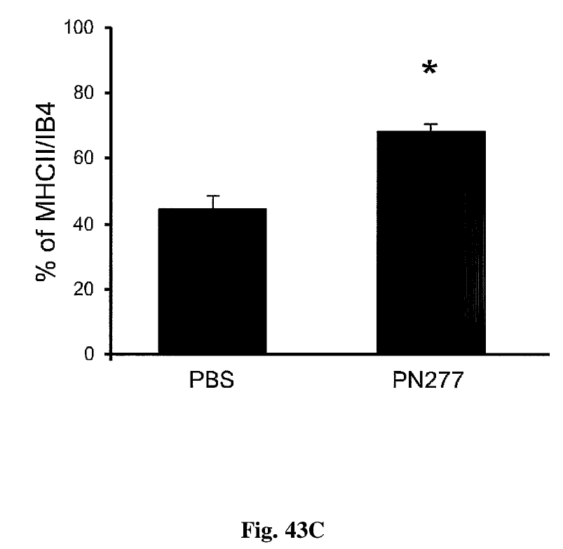

Nestin expressing cells with similar morphology to those detected in the surrounding of the cortical lesion were also detected in the striatum adjacent to the lateral ventricles. We found that the great majority of these Nestin+ cells were also expressing the astrocytic marker GFAP. Interestingly, we found IB-4+microglia/macrophages within the striatum area occupied by the GFAP+/Nestin+ cells (FIG. 43A). Previous studies by our group have characterized the phenotype of microglia that are supportive of cell renewal (ref). These microglia (IB-4+) are of ramified morphology and express high levels of MHC-II. Whereas the numbers IB-4+ cells were similar in both experimental groups, higher proportion of these cells were stained for MLC-II in the PN277 treated group (FIG. 43B). Quantitative analysis confirmed that indeed a significant higher proportion of the IB-4+ cells were also MLC-II+ in the PN277 treated rats (FIG. 43C). This result suggests that neuroprotective and neurogenesis-supporting effects of treatment with PN277 could be mediated, as our previous studies have showed, via MHC-II+ microglia/macrophages.

### Discussion

Boosting T-cell mediated immune response following insult to the CNS can be done by either vaccinating with a CNS-specific antigen or by abolishing regultory T cells (Treg) activity (Moalem et al., 1999; Hauben et al., 2000; Schwartz et al., 2003). We chose to use PN277, an immuno-modulator that was recently shown to be capable of reducing Treg suppressive activity and confer neuroprotection. While vaccination induce T-cell response primarily to one antigen, reducing Treg suppressive activity facilitate a broader T-cell response against various antigens residing in the injured site. Thus, following stroke, mostly T cells specific for various CNS— derived antigens would be propagated in response to PN277 treatment.

The finding of enhanced hippocampal neurogenesis in the PN277 treated rats is consistent with our finding herein that neurogenesis was enhanced in transgenic mice over-expressing T-cell receptor for MBP. The fact that this effect was seen in both ipsilateral and contralateral sides further imply that elevation in neurogenesis is caused by a systemic event (T-cell response). Such autoimmune T cells can locally interact with microglia, which in turn can recruit NPCs and direct their differentiation. This hypothetical scenario is supported by our findings herein showing that neurogenesis from aNPCs could be induced by microglia activated by cytokines (IL-4 or IFN-γ) associated with T-helper cells.

The effect of PN277 treatment on neurogenesis was even more robust with regard to cortical neurogenesis. In conclusion, the results of this study suggest that proper immune modulation can increase neurogenesis, and that lack of neurogenic permissiveness in brain regions such as the cortex can be changed by the local immune response. These findings introduce a new therapeutic approach for augmenting spontaneously occurring neurogenesis following an ischemic insult.

### Example 8

### Combination of Glatiramer Acetate (Cop-1) Vaccination and Stem Cells in an Animal Model of Amyotrophic Lateral Sclerosis (ALS)

### Materials and Methods

Animals. Transgenic mice overexpressing the defective human mutant SOD1 allele containing the Gly93→Ala (G93A) gene (B6SJL-TgN (SOD1-G93A)1Gur (herein “ALS mice”) were purchased from The Jackson Laboratory (Bar Harbor, Me., USA).

Immunization. Adult mice were immunized with Cop-1, 100 μg in 200 μl PBS s.c.

Neural progenitor cell culture. Cultures of adult neural progenitor cells (aNPCs) were obtained as described in orevious examples

Stereotaxic injection of neural progenitor cells. Mice were anesthetized a week after the first immunization and placed in a stereotactic device. The skull was exposed and kept dry and clean. The bregma was identified and marked. The designated point of injection was at a depth of 2 mm from the brain surface, 0.4 mm behind the bregma in the anteroposterior axis, and 1.0 mm lateral to the midline. Neural progenitor cells were applied with a Hamilton syringe (5×10⁵ cells in 3 μl, at a rate of 1 μl/min) and the skin over the wound was sutured.

Motor dysfunction. Motor dysfunction of the mice was evaluated using the rotarod task twice a week from 60 d of age onward. Animals were placed on a horizontal accelerating rod [accelerating rotarod (Jones and Roberts) for mice 7650] and time it took for each mouse to fall from the rod was recorded. We performed three trials at each time point for each animal and recorded the longest time taken. A cut-off time point was set to 180 sec and mice remaining on the rod for at least 180 sec were deemed asymptomatic. Onset of disease symptoms was determined as a reduction in rotarod performance between weekly time points. Animals were killed by euthanization when no longer able to right themselves within 30 seconds of being placed on their sides.

### Example 4

The animals were treated with Cop-i starting from day 59: in the first two weeks twice a week Cop-1, thereafter they received a weekly injection of Cop-1. The stem cells were given into the CSF: 500,000 cells (single injection of adult neural stem cells).

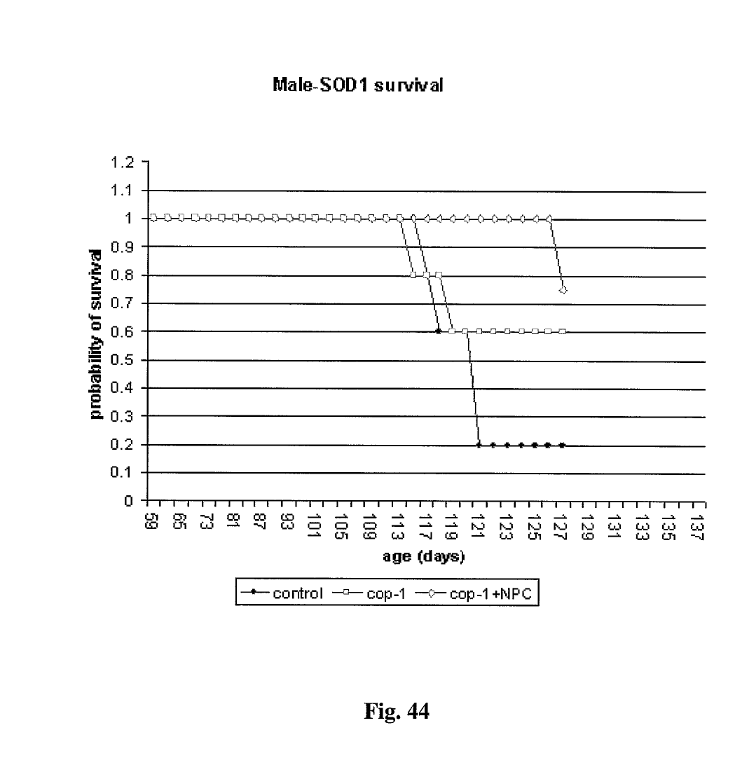

The experiment was carried out in order to explore whether administration of a combination of Cop-i vaccination and stem cells has beneficial effect in a mice model of ALS (herein “ALS mice”). For this purpose, 59 days old ALS mice were treated as follows: group 1 (FIG. 44, Cop-1+NPC) 4 males were immunized s.c. with 100 μg/200 μl Cop-1/PBS twice a week for 2 weeks (the first immunization was at age 59 days) and received thereafter one immunization per week until euthanization. With the third immunization, the mice received 100,000 NPC^{GFP} i.c.v (CSF) into the right cerebral ventricle; group 2 (FIG. 44, Cop-1) 5 males were immunized with 100 μg/200 μl Cop-1/PBS twice a week for 2 weeks and received thereafter one immunization per week until euthanization; group 3 (FIG. 44, control) 5 males were immunized with 200 μl PBS twice a week for 2 weeks and received thereafter one immunization per week until euthanization.

Mice from each of the groups were weighted (twice a week) and examined routinely for vital signs, and for signs of motor dysfunction. The results obtained in FIG. 44 show the probability of survival of each group of ALS mice. The results show that the combined treatment of Cop-I vaccination and NPC results in increased survival of ALS mice.

### REFERENCES

Aberg, M. A., Aberg, N. D., Hedbacker, H., Oscarsson, J. & Eriksson, P. S. Peripheral infusion of IGF-I selectively induces neurogenesis in the adult rat hippocampus. J Neurosci 20, 2896-903 (2000).
Anderson W W, Collingridge G L. The LTP Program: a data acquisition program for on-line analysis of long-term potentiation and other synaptic events. J Neurosci Methods. July 15; 108(1):71-83 (2001).
Arvidsson A, Collin T, Kirik D, Kokaia Z, Lindvall O. Neuronal replacement from endogenous precursors in the adult brain after stroke. Nat. Med. September;8(9):963-70 (2002).
Avidan H, Kipnis J, Butovsky O, Caspi R R, Schwartz M. Vaccination with autoantigen protects against aggregated beta-amyloid and glutamate toxicity by controlling microglia: effect of CD4+ CD25+ T cells. Eur J. Immunol. December;34(12):3434-45 (2004).
Benner E J, Mosley R L, Destache C J, Lewis T B, Jackson-Lewis V, Gorantla S, Nemachek C, Green S R, Przedborski S, Gendelman H E. Therapeutic immunization protects dopaminergic neurons in a mouse model of Parkinson's disease. Proc Natl Acad Sci USA. June 22; 101(25):9435-40 (2004).
Blaugrund E, Duvdevani R, Lavie V, Solomon A, Schwartz M. Disappearance of astrocytes and invasion of macrophages following crush injury of adult rodent optic nerves: implications for regeneration. Exp Neurol. October; 118(1):105-15 (1992).
Bliss, T. V. & Collingridge, G. L. A synaptic model of memory: long-term potentiation in the hippocampus. Nature 361, 31-9 (1993).
Boje, K. M. & Arora, P. K. Microglial-produced nitric oxide and reactive nitrogen oxides mediate neuronal cell death. Brain Res. 587, 250-6 (1992).
Borchelt D R, Ratovitski T, van Lare J, Lee M K, Gonzales V, Jenkins N A, Copeland N G, Price D L, Sisodia S S. Related Accelerated amyloid deposition in the brains of transgenic mice coexpressing mutant presenilin 1 and amyloid precursor proteins. Neuron. October; 19(4):939-45 (1997).
Brown, J. et al. Enriched environment and physical activity stimulate hippocampal but not olfactory bulb neurogenesis. Eur J Neurosci 17, 2042-6 (2003).
Butovsky, O., Hauben, E. & Schwartz, M. Morphological aspects of spinal cord autoimmune neuroprotection: colocalization of T cells with B7-2 (CD86) and prevention of cyst formation. Faseb J. 15, 1065-7 (2001).
Butovsky O, Talpalar A E, Ben-Yaakov K, Schwartz M. Activation of microglia by aggregated beta-amyloid or lipopolysaccharide impairs MHC-II expression and renders them cytotoxic whereas IFN-gamma and IL-4 render them protective. Mol Cell Neurosci. July; 29(3):381-93 (2005).
Cameron, H. A. & McKay, R. D. Restoring production of hippocampal neurons in old age. Nat Neurosci 2, 894-7 (1999).
Cameron, H. A. & McKay, R. D. Adult neurogenesis produces a large pool of new granule cells in the dentate gyrus. J Comp Neurol 435, 406-17 (2001).
Cammer, W. & Zhang, H. Maturation of oligodendrocytes is more sensitive to TNF alpha than is survival of precursors and immature oligodendrocytes. J Neuroimmunol 97, 37-42 (1999).
Cummings, B. J., Uchida, N., Tamaki, S. J., Salazar, D. L., Hooshmand, M., Summers, R., Gage, F. H. & Anderson, A. J. Proc Natl Acad Sci USA 102, 14069-74 (2005).
Dawson, M. R., Polito, A., Levine, J. M. & Reynolds, R. NG2-expressing glial progenitor cells: an abundant and widespread population of cycling cells in the adult rat CNS. Mol Cell Neurosci 24, 476-88 (2003).
Dijkstra, C. D., De Groot, C. J. & Huitinga, I. The role of macrophages in demyelination. J Neuroimmunol 40, 183-8 (1992).
Duda, P. W., Schmied, M. C., Cook, S. L., Krieger, J. I., and Hafler, D. A. Glatiramer acetate (Copaxone) induces degenerate, Th2-polarized immune responses in patients with multiple sclerosis. J Clin Invest 105:967-976 (2000).
Ekdahl, C. T., Claasen, J. H., Bonde, S., Kokaia, Z. & Lindvall, O. Inflammation is detrimental for neurogenesis in adult brain. Proc Natl Acad Sci USA 100, 13632-7 (2003).
Emsley J G, Mitchell B D, Kempermann G, Macklis J D. Adult neurogenesis and repair of the adult CNS with neural progenitors, precursors, and stem cells. Prog Neurobiol. April; 75(5):321-41 (2005).
Engesser-Cesar, C., Anderson, A. J., Basso, D. M., Edgerton, V. R. & Cotman, C. W. Voluntary wheel running improves recovery from a moderate spinal cord injury. J Neurotrauma 22, 157-71 (2005).
Enzmann, G. U., Benton, R. L., Woock, J. P., Howard, R. M., Tsoulfas, P. & Whittemore, S. R. Exp Neurol 195, 293-304 (2005).
Eriksson P S, Perfilieva E, Bjork-Eriksson T, Alborn A M, Nordborg C, Peterson D A, Gage F H. Neurogenesis in the adult human hippocampus. Nat. Med. November; 4(11):1313-7 (1998).
Fricker J. Human neural stem cells on trial for Parkinson's disease. Mol Med Today. April; 5(4):144 (1999).
Gage F. H., Ray J, Fisher L J. Isolation, characterization, and use of stem cells from the CNS. Annu Rev Neurosci.; 18:159-92 (1995).
Gould, E., Beylin, A., Tanapat, P., Reeves, A. & Shors, T. J. Learning enhances adult neurogenesis in the hippocampal formation. Nat Neurosci 2, 260-5 (1999).
Hanisch, U. K. Microglia as a source and target of cytokines. Glia 40, 140-55 (2002).
Hauben E, Schwartz M. Therapeutic vaccination for spinal cord injury: helping the body to cure itself. Trends Pharmacol Sci 24:7-12 (2003).
Hauben, E., Gothilf, A., Cohen, A., Butovsky, O., Nevo, U., Smirnov, I., Yoles, E., Akselrod, S. & Schwartz, M. J Neurosci 23, 8808-19 (2003).
Hauben, E., Agranov, E., Gothilf, A., Nevo, U., Cohen, A., Smirnov, I., Steinman, L. & Schwartz, M. Posttraumatic therapeutic vaccination with modified myelin self-antigen prevents complete paralysis while avoiding autoimmune disease. J. Clin. Invest. 108, 591-9 (2001).
Hauben, E., Butovsky, O., Nevo, U., Yoles, E., Moalem, G., Agranov, G., Mor, F., Leibowitz-Amit, R., Pevsner, E., Akselrod, S., Neeman, M., Cohen, I. R. & Schwartz, M. Passive or active immunization with myelin basic protein promotes recovery from spinal cord contusion. J. Neurosci. 20, 6421-6430 (2000).
Hsieh, J., Aimone, J. B., Kaspar, B. K., Kuwabara, T., Nakashima, K., and Gage, F. H. 2004. IGF-I instructs multipotent adult neural progenitor cells to become oligodendrocytes. J Cell Biol 164, 111-22 (2004).
Imitola, J., Raddassi, K., Park, K. I., Mueller, F. J., Nieto, M., Teng, Y. D., Frenkel, D., Li, J., Sidman, R. L., Walsh, C. A., Snyder, E. Y. & Khoury, S. J. Proc Natl Acad Sci USA 101, 18117-22 (2004a).
Imitola, J., Comabella, M., Chandraker, A. K., Dangond, F., Sayegh, M. H., Snyder, E. Y. & Khoury, S. J. Neural stem/progenitor cells express costimulatory molecules that are differentially regulated by inflammatory and apoptotic stimuli. Am J Pathol 164, 1615-25 (2004b).
Ishii K, Muelhauser F, Liebl U, Picard M, Kuhl S, Penke B, Bayer T, Wiessler M, Hennerici M, Beyreuther K, Hartmann T, Fassbender K. Subacute NO generation induced by Alzheimer's beta-amyloid in the living brain: reversal by inhibition of the inducible NO synthase. FASEB J. August; 14(11):1485-9, (2000).

Jankowsky J L, Fadale D J, Anderson J, Xu G M, Gonzales V, Jenkins N A, Copeland N G, Lee M K, Younkin L H, Wagner S L, Younkin S G, Borchelt D R. Mutant presenilins specifically elevate the levels of the 42 residue beta-amyloid peptide in vivo: evidence for augmentation of a 42-specific gamma secretase. Hum Mol. Genet. January 15; 13(2):159-70 (2004).
Jones, L. L., Yamaguchi, Y., Stallcup, W. B. & Tuszynski, M. H. NG2 is a major chondroitin sulfate proteoglycan produced after spinal cord injury and is expressed by macrophages and oligodendrocyte progenitors. J Neurosci 22, 2792-803 (2002).
Kempermann, G., Jessberger, S., Steiner, B. & Kronenberg, G. Milestones of neuronal development in the adult hippocampus. Trends Neurosci 27, 447-52 (2004).
Kempermann, G., Kuhn, H. G. & Gage, F. H. More hippocampal neurons in adult mice living in an enriched environment. Nature 386, 493-5 (1997).
Kipnis J, Yoles E, Porat Z, Cohen A, Mor F, Sela M, Cohen I R, Schwartz M. T cell immunity to copolymer 1 confers neuroprotection on the damaged optic nerve: possible therapy for optic neuropathies. Proc Natl Acad Sci USA. June 20; 97(13):7446-51 (2000).
Kipnis, J., Mizrahi, T., Yoles, E., Ben-Nun, A., Schwartz, M. & Ben-Nur, A. J Neuroimmunol 130, 78-85 (2002a).
Kipnis, J., Mizrahi, T., Hauben, E., Shaked, I., Shevach, E. & Schwartz, M. Neuroprotective autoimmunity: naturally occurring CD4+ CD25+ regulatory T cells suppress the ability to withstand injury to the central nervous system. Proc. Natl. Acad. Sci. USA 99, 15620-5 (2002b).
Kipnis, J., Cohen, H., Cardon, M., Ziv, Y. & Schwartz, M. T cell deficiency leads to cognitive dysfunction: Implications for therapeutic vaccination for schizophrenia and other psychiatric conditions. Proc. Natl. Acad. Sci. USA 101, 8180-8185 (2004).
Kuhn H G, Winkler J, Kempermann G, Thal L J, Gage F H. Epidermal growth factor and fibroblast growth factor-2 have different effects on neural progenitors in the adult rat brain. J Neurosci. August 1; 17(15):5820-9 (1997).
Kukekov V G, Laywell E D, Suslov O, Davies K, Scheffler B, Thomas L B, O'Brien T F, Kusakabe M, Steindler D A. Multipotent stem/progenitor cells with similar properties arise from two neurogenic regions of adult human brain. Exp Neurol. April; 156(2):333-44 (1999).
Lafaille, J. J., Nagashima, K., Katsuki, M. & Tonegawa, S. High incidence of spontaneous autoimmune encephalomyelitis in immunodeficient anti-myelin basic protein T cell receptor transgenic mice. Cell 78, 399-408 (1994).
Lepore, A. C. & Fischer, I. Exp Neurol 194, 230-42 (2005).
Li, W. W., Setzu, A., Zhao, C., and Franklin, R. J. Minocycline-mediated inhibition of microglia activation impairs oligodendrocyte progenitor cell responses and remyelination in a non-immune model of demyelination. J Neuroimmunol 158:58-66 (2005).
Lu, P., Jones, L. L. & Tuszynski, M. H. Exp Neurol 191, 344-60 (2005).
Mason, J. L., Ye, P., Suzuki, K., D'Ercole, A. J. & Matsushima, G. K. Insulin-like growth factor-i inhibits mature oligodendrocyte apoptosis during primary demyelination. J Neurosci 20, 5703-8 (2000).
McDonald, J. W., Becker, D., Holekamp, T. F., Howard, M., Liu, S., Lu, A., Lu, J., Platik, M. M., Qu, Y., Stewart, T. & Vadivelu, S. J Neurotrauma 21, 383-93 (2004).
Merrill, J. E., Ignarro, L. J., Sherman, M. P., Melinek, J. & Lane, T. E. Microglial cell cytotoxicity of oligodendrocytes is mediated through nitric oxide. J Immunol 151, 2132-41 (1993).
Moalem, G., Leibowitz-Amit, R., Yoles, E., Mor, F., Cohen, I R. & Schwartz M Autoimmune T cells protect neurons from secondary degeneration after central nervous system axotomy. Nat. Med. 5, 49-55 (1999).
Moalem, G., Gdalyahu, A., Shani, Y., Otten, U., Lazarovici, P., Cohen, I R. & Schwartz, M. Production of neurotrophins by activated T cells: implications for neuroprotective autoimmunity. J Autoimmun 15, 331-45 (2000).
Monje, M. L., Mizumatsu, S., Fike, J. R. & Palmer, T. D. Irradiation induces neural precursor-cell dysfunction. Nat Med 8, 955-62 (2002).
Monje, M. L., Toda, H. & Palmer, T. D. Inflammatory blockade restores adult hippocampal neurogenesis. Science 302, 1760-5 (2003).
Morshead, C. M. et al. Neural stem cells in the adult mammalian forebrain: a relatively quiescent subpopulation of subependymal cells. Neuron 13, 1071-82 (1994).
Nakatomi, H., Kuriu, T., Okabe, S., Yamamoto, S., Hatano, O., Kawahara, N., Tamura, A., Kirino, T. & Nakafuku, M. Cell 110, 429-41 (2002).
Ness, J. K. & Wood, T. L. Insulin-like growth factor I, but not neurotrophin-3, sustains Akt activation and provides long-term protection of immature oligodendrocytes from glutamate-mediated apoptosis. Mol Cell Neurosci 20, 476-88 (2002).
Neumann, H., Misgeld, T., Matsumuro, K., and Wekerle, H. Neurotrophins inhibit major histocompatibility class II inducibility of microglia: involvement of the p75 neurotrophin receptor. Proc. Natl. Acad. Sci. USA 95:5779-5784 (1998).
O'Kusky, J. R., Ye, P. & D'Ercole, A. J. Insulin-like growth factor-I promotes neurogenesis and synaptogenesis in the hippocampal dentate gyrus during postnatal development. J Neurosci 20, 8435-42 (2000).
Packer, M. A. et al. Nitric oxide negatively regulates mammalian adult neurogenesis. Proc Natl Acad Sci USA 100, 9566-71 (2003).
Park K I, Liu S, Flax J D, Nissim S, Stieg P E, Snyder E Y. Transplantation of neural progenitor and stem cells: developmental insights may suggest new therapies for spinal cord and other CNS dysfunction. J. Neurotrauma. August; 16(8):675-87. (1999).
Pluchino, S., Quattrini, A., Brambilla, E., Gritti, A., Salani, G., Dina, G., Galli, R., Del Carro, U., Amadio, S., Bergami, A., Furlan, R., Comi, G., Vescovi, A. L. & Martino, G. Injection of adult neurospheres induces recovery in a chronic model of multiple sclerosis. Nature 422, 688-94 (2003).
Popovich, P. G., Wei, P. & Stokes, B. T. Cellular inflammatory response after spinal cord injury in Sprague-Dawley and Lewis rats. J Comp Neurol 377, 443-64 (1997).
Popovich, P. G. et al. The neuropathological and behavioral consequences of intraspinal microglial/macrophage activation. J. Neuropathol. Exp. Neurol. 61, 623-33 (2002).
Rapalino, O., Lazarov-Spiegler, O., Agranov, E., Velan, G. J., Yoles, E., Fraidakis, M., Solomon, A., Gepstein, R., Katz, A., Belkin, M., Hadani, M. & Schwartz, M. Nat Med 4, 814-21 (1998).
Schori, H., Yoles, E. & Schwartz, M. T-cell-based immunity counteracts the potential toxicity of glutamate in the central nervous system. J. Neuroimmunol. 119, 199-204 (2001).
Schwartz, M. & Hauben, E. T cell-based therapeutic vaccination for spinal cord injury. Prog Brain Res 137, 401-6 (2002).
Schwartz, M. & Kipnis, J. Harm or heal—divergent effects of autoimmune neuroinflammation?. Response from Schwartz and Kipnis. Trends Immunol 24, 7-8 (2003).
Schwartz M, and Kipnis J. Protective autoimmunity: regulation and prospects for vaccination after brain and spinal cord injuries. Trends Mol. Med. June; 7(6):252-8.(2001)
Schwartz, M. & Kipnis, J. Autoimmunity on alert: naturally occurring regulatory CD4(+)CD25(+) T cells as part of the evolutionary compromise between a ‘need’ and a ‘risk’. Trends Immunol. 23, 530-4 (2002).
Schwartz, M., Shaked, I., Fisher, J., Mizrahi, T. & Schori, H. Protective autoimmunity against the enemy within: fighting glutamate toxicity. Trends Neurosci. 26, 297-302 (2003).
Setoguchi, T., Nakashima, K., Takizawa, T., Yanagisawa, M., Ochiai, W., Okabe, M., Yone, K., Komiya, S. & Taga, T. Treatment of spinal cord injury by transplantation of fetal neural precursor cells engineered to express BMP inhibitor. Exp Neurol 189, 33-44.(2004)
Shaked, I., Porat, Z., Gersner, R., Kipnis, J. & Schwartz, M. Early activation of microglia as antigen-presenting cells correlates with T cell-mediated protection and repair of the injured central nervous system. J Neuroimmunol 146, 84-93 (2004).
Shaked, I., Tchoresh, D., Gersner, R., Meiri, G., Mordechai, S., Xiao, X., Hart, R. P. & Schwartz, M. J Neurochem 92, 997-1009 (2005).
Shors, T. J. et al. Neurogenesis in the adult is involved in the formation of trace memories. Nature 410, 372-6 (2001).
Shors, T. J., Townsend, D. A., Zhao, M., Kozorovitskiy, Y. & Gould, E. Neurogenesis may relate to some but not all types of hippocampal-dependent learning. Hippocampus 12, 578-84 (2000).
Snyder, J. S., Hong, N. S., McDonald, R. J. & Wojtowicz, J. M. A role for adult neurogenesis in spatial long-term memory. Neuroscience 130, 843-52 (2005).
van Praag, H., Christie, B. R., Sejnowski, T. J. & Gage, F. H. Running enhances neurogenesis, learning, and long-term potentiation in mice. Proc Natl Acad Sci USA 96, 13427-31 (1999).
Yoles, E., Hauben, E., Palgi, O., Agranov, E., Gothilf, A., Cohen, A., Kuchroo, V., Cohen, I. R., Weiner, H. & Schwartz, M. Protective autoimmunity is a physiological response to CNS trauma. J Neurosci 21, 3740-8 (2001a).
Yoles, E., Friedmann, I., Barouch, R., Shani, Y. & Schwartz, M. I Neurotrauma 18, 339-49 (2001b).
Zhao, X., Ueba, T., Christie, B R., Barkho, B., McConnell, M J., Nakashima, K., Lein, E S., Eadie, B D., Willhoite, A R., Muotri, A R., Summers, R G., Chun, J., Lee, K F. & Gage, F H. Mice lacking methyl-CpG binding protein 1 have deficits in adult neurogenesis and hippocampal function. Proc Natl Acad Sci USA 100, 6777-82 (2003).
Zielasek J, Tausch M, Toyka K V, Hartung H P. Production of nitrite by neonatal rat microglial cells/brain macrophages. Cell Immunol. 15; 141(1):111-20. (1992).

1. A method for inducing and enhancing neurogenesis and/or oligodendrogenesis from endogenous as well as from exogenously administered stem cells, which comprises administering to an individual in need a neuroprotective agent selected from the group consisting of:
(i) a nervous system (NS)-specific antigen or an analog thereof;
(ii) a peptide derived from an NS-specific antigen or from an analog thereof, or an analog or derivative of said peptide;
(iii) poly-YE and poly-YE related peptides;
(iv) T cells activated with an agent (i) to (iii);
(v) antigen-presenting cells that have been pulsed with an agent (i) to (iii);
(vi) activated mononuclear phagocytic leukocytes;
(vii) dopamine, a dopamine precursor, an agonist of the dopamine receptor type I family (D1-R agonist), a combination of dopamine and a dopamine precursor, or a combination of dopamine, a dopamine precursor, or a D1-R agonist with an antagonist of the dopamine receptor type 2 family (D2-R antagonist);
(viii) microglia activated by IFN-γ or IL-4, or both;
(ix) TFN-γ or IL-4, or both; and
(x) any combination of (i) to (ix).

2. The method according to claim 1 for inducing and enhancing neurogenesis from endogenous or exogenously applied stem cells, by immune modulation, which comprises administering to an individual in need a neuroprotective agent selected from the group consisting of said agents (i) to (x).

3. The method according to claim 1 for inducing and enhancing oligodendrogenesis from endogenous or exogenously applied stem cells, by immune modulation, which comprises administering to an individual in need a neuroprotective agent selected from the group consisting of said agents (i) to (x).

4. The method according to claim 1, wherein the exogenous stem cells are adult stem cells, embryonic stem cells, umbilical cord blood stem cells, hematopoietic stem cells, peripheral blood stem cells, mesenchimal stem cells, multipotent stem cells, neural stem cells, stromal stem cells, progenitor cells, or precursors thereof.

5. The method according to claim 1 for inducing and enhancing endogenous neurogenesis and/or oligodendrogenesis, said method further including proliferation, differentiation and survival of newly formed neurons or oligodendrocytes.

6. The method according to claim 1 wherein said individual in need suffers from an injury, disease, disorder or condition of the central nervous system (CNS) or peripheral nervous system (PNS).

7. The method according to claim 6 wherein said CNS injury is selected from the group consisting of spinal cord injury, closed head injury, blunt trauma, penetrating trauma, hemorrhagic stroke, ischemic stroke, cerebral isehemia, optic nerve injury, myocardial infarction and injury caused by tumor excision.

8. The method according to claim 6 wherein said disease, disorder or condition is Parkinson's disease, Huntington's disease, Alzheimer's disease, multiple sclerosis, amyotrophic lateral sclerosis (ALS), facial nerve (Bell's) palsy, glaucoma, Alper's disease, Batten disease, Cockayne syndrome, Guillain-Barré syndrome, Lewy body disease, Creutzfedt-Jakob disease, or a peripheral neuropathy such as a mononeuropathy or polyneuropathy selected from the group consisting of adrenomyeloneuropathy, alcoholic neuropathy, amyloid neuropathy or polyneuropathy axonal neuropathy, chronic sensory ataxic neuropathy associated with Sjogren's syndrome, diabetic neuropathy, an entrapment neuropathy nerve compression syndrome, carpal tunnel syndrome, a nerve root compression that may follow cervical or lumbar intervertebral disc herniation, giant axonal neuropathy, hepatic neuropathy, ischemic neuropathy, nutritional polyneuropathy due to vitamin deficiency, malabsomtion syndromes or alcoholism porphyric polyneuropathy, a toxic neuropathy caused by organophosphates, uremic polyneuropathy, a neuropathy associated with a disease or disorder selected from the group consisting of acromealy, ataxia telangiectasia, Charcot-Mar-Tooth disease, chronic obstructive pulmonary diseases, Fabry's disease, Friedreich ataxia, Guillain-Barré syndrome, hypoglycemia, IgG or IgA monoclonal gammopathy (non-malignant or associated with multiple myeloma or with osteoscierotic myeloma), lipoproteinemia, polycythemia vera Refsum's syndrome, Reye's syndrome, and Sjogren-Larsson syndrome, a polyneurotpathy associated with various drugs, with hypoglycemia, with infections such as HIV infection, or with cancer, epilepsy, amnesia, anxiety, hyperalgesia, psychosis, seizures, oxidative stress opiate tolerance and dependence, a psychosis or psychiatric disorder selected from the group consisting of an anxiety disorder, a mood disorder, schizophrenia or a schizophrenia-related disorder, drug use and dependence and withdrawal, or a memory loss or cognitive disorder.

9-10. (canceled)

11. A method of stem cell therapy comprising transplantation of stern cells in combination with a neuroprotective agent to an individual that suffers from an injury, disease, disorder or condition of the central nervous system (CNS) or peripheral nervous system (PNS).

12. The method according to claim 11 wherein said neuroprotective agent is selected from the group consisting of:
(i) a nervous system (NS)-specific antigen or an analog thereof,
(ii) a peptide derived from an NS-specific antigen or from an analog thereof, or an analog or derivative of said peptide;
(iii) poly-YE and poly-YE related peptides;
(iv) T cells activated with an agent (i) to (iii);
(v) antigen-presenting cells that have been pulsed with an agent (i) to (iii);
(vi) activated mononuclear phagocytic leukocytes;
(vii) dopamine, a dopamine precursor, an agonist of the dopamine receptor type 1 family (D1-R agonist), a combination of dopamine and a dopamine precursor, or a combination of dopamine, a dopamine precursor, or a D1-R agonist with an antagonist of the dopamine receptor type 2 family (D2-R antagonist);
(viii) microglia activated by IFN-γ or IL-4, or both;
(ix) IFN-γ or IL-4, or both; and
(x) any combination of (i) to (ix).

13. The method according to claim 11 wherein said individual suffers from an injury selected from spinal cord injury, closed head injury, blunt trauma, penetrating trauma, hemorrhagic stroke, ischemic stroke, cerebral ischemia, optic nerve injury, myocardial infarction and injury caused by tumor excision.

14. The method according to claim 11 wherein said individual suffers from a disease, disorder or condition selected from Parkinson's disease and Parkinsonian disorders, Huntington's disease, Alzheimer's disease, multiple sclerosis, amyotrophic lateral sclerosis (ALS), facial nerve (Bell's) palsy, glaucoma, Alper's disease, Batten disease, Cockayne syndrome, Guillain-Barré syndrome, Lewy body disease, Creutzfeldt-Jakob disease, or a peripheral neuropathy such as a mononeuropathy or polyneuropathy selected from the group) consisting of adrenomyeloneuropathy, alcoholic neuropathy, amyloid neuropathy or polyneuropathy, axonal neuropathy, chronic sensory ataxic neuropathy associated with Sjogren's syndrome, diabetic neuropathy, an entrapment neuropathy nerve compression syndrome, carnal tunnel syndrome, a nerve root compression that may follow cervical or lumbar intervertebral disc herniation, giant axonal neuropathy hepatic neuropathy, ischemic neuropathy, nutritional polyneuropathy due to vitamin deficiency, malabsorption syndromes or alcoholism, porphyric polyneuropathy, a toxic neuropathy caused by organophosphates, uremic polyneuropathy, a neuropathy, associated with a disease or disorder selected from the group consisting of acromegaly, ataxia telangiectasia, Charcot-Marie-Tooth disease, chronic obstructive pulmonary diseases, Fabry's disease, Friedreich ataxia, Guillain-Barré syndrome, hypoglycemia, IgG or IgA monoclonal gammopathy (non-malignant or associated with multiple myeloma or with osteosclerotic myeloma), lipoproteinemia, polycythemia vera, Refsum's syndrome, Reye's syndrome, and Sjogren-Larsson syndrome, a polyneuropathy associated with various drugs, with hypoglycemia, with infections such as HIV infection, or with cancer, epilepsy, amnesia, anxiety, hyperalgesia, psychosis, seizures, oxidative stress, opiate tolerance and dependence, and for the treatment of a psychosis or psychiatric, disorder selected from the group consisting of an anxiety disorder, a mood disorder, schizophrenia or a schizophrenia-related disorder, drug use and dependence and withdrawal, and a memory loss or cognitive disorder.

15-16. (canceled)

17. The method according to claim 11 wherein said neuroprotective agent is administered to the individual before, concomitantly or after the transplantation of the stem cells to said individual.

18. The method according to claim 11 wherein the individual is transplanted with the stem cells combined with the neuroprotective agent.

19. The method according to claim 11 wherein the stem cells are adult stem cells, embryonic stem cells, umbilical cord blood stem cells, hematopoietic stem cells, peripheral blood stem cells, mesenchimal stem cells, multipotent stem cells, neural stem cells, neural progenitor cells, stromal stem cells, progenitor cells, or precursors thereof.

20. The method according to claim 12 wherein said agent is a NS-specific antigen selected from the group consisting of myelin basic protein (MBP), myelin oligodendrocyte glycoprotein (MOG), proteolipid protein (PLP), myelin-associated glycoprotein (MAG), S-100, β-amyloid, Thy-1, a peripheral myelin protein including P0, P2 and PMP22, neurotransmitter receptors, the protein Nogo including Nogo-A, Nogo-B and Nogo-C and the Nogo receptor, or a NS-specific antigen analog.

21. The method according to claim 12 wherein said agent is a peptide derived from a NS-specific antigen, or an analog thereof.

22. The method according to claim 21 wherein said peptide is an immunogenic epitope or a cryptic epitope derived from said NS-specific antigen.

23. The method according to claim 21 wherein said peptide analog is obtained by modification of a self-peptide derived from a CNS-specific antigen, which modification consists in the replacement of one or more amino acid residues of the self-peptide by different amino acid residues or by deletion or addition of one or more amino acid residues, said modified peptide still being capable of recognizing the T-cell receptor recognized by the self-peptide but with less affinity.

24. The method according to claim 12 wherein said agent are T cells activated with an agent selected from a NS-specific antigen, a peptide derived from a NS-specific antigen and an analog of said peptide.

25. The method according to claim 12 wherein said agent is activated mononuclear phagocytic leukocytes, obtained by culturing the cells together with at least one stimulatory tissue, stimulatory cells, a medium conditioned by at least one stimulatory tissue or cells, or with medium to which at least one stimulatory biologically active agent has been added.

26. The method according to claim 25 wherein said stimulatory tissue is skin, dermis or a nerve segment, and said at least one stimulatory biologically active agent is neurotrophic factor 3 (NT-3), nerve growth factor (NGF), brain-derived neurotrophic factor (BDNF) and transforming growth factors (TGF-β).

27. The method according to claim 12 wherein said agent is microglia activated by IL-4 or IFN-γ, or both.

28-44. (canceled)


SEQUENCE LISTING (16 sequences)

SEQ ID NO: 1 - protein, 21 aa, Artificial Sequence
  Synthetic
MEVGWYRSPFSRVVHLYRNGK

SEQ ID NO: 2 - protein, 21 aa, Artificial Sequence
  Synthetic
MEVGWYRSPFDRVVHLYRNGK

SEQ ID NO: 3 - DNA, 25 bp, Artificial Sequence
  Synthetic
aggaggcgctccccaaaaagatggg

SEQ ID NO: 4 - DNA, 25 bp, Artificial Sequence
  Synthetic
gtacatgggctcataccagggcttg

SEQ ID NO: 5 - DNA, 20 bp, Artificial Sequence
  Synthetic
caggctcctagcatacctgc

SEQ ID NO: 6 - DNA, 20 bp, Artificial Sequence
  Synthetic
gctggtaaaggtgagcaagc

SEQ ID NO: 7 - DNA, 24 bp, Artificial Sequence
  Synthetic
ttgtaaccaactgggacgatatgg

SEQ ID NO: 8 - DNA, 24 bp, Artificial Sequence
  Synthetic
gatcttgatcttcatggtgctagg

SEQ ID NO: 9 - DNA, 19 bp, Artificial Sequence
  Synthetic
ccggaccagagaccctttg

SEQ ID NO: 10 - DNA, 23 bp, Artificial Sequence
  Synthetic
cctgtgggcttgttgaagtaaaa

SEQ ID NO: 11 - DNA, 19 bp, Artificial Sequence
  Synthetic
acaaggctgccccgactat

SEQ ID NO: 12 - DNA, 23 bp, Artificial Sequence
  Synthetic
ctcctggtatgaagtggcaaatc

SEQ ID NO: 13 - DNA, 28 bp, Artificial Sequence
  Synthetic
gtggataccccctcccccagcctagacc

SEQ ID NO: 14 - DNA, 30 bp, Artificial Sequence
  Synthetic
cctctttgtgactatgtggactgatgtcgg

SEQ ID NO: 15 - DNA, 20 bp, Artificial Sequence
  Synthetic
aatagagaacggcaggagca

SEQ ID NO: 16 - DNA, 20 bp, Artificial Sequence
  Synthetic
gccatgagggcactaatcat